(19) United States
(12) Patent Application Publication
(10) Pub. No.: US 2026/0365603 A1
(43) Pub. Date: Nov. 19, 2026

(54) METHOD FOR PRODUCING 1,3-BUTYLENE GLYCOL, AND 1,3-BUTYLENE GLYCOL PRODUCT

(71) Applicant: Daicel Corporation, Osaka (JP)

(72) Inventors: Masahiko SHIMIZU, Tokyo (JP); Yasuteru KAJIKAWA, Tokyo (JP); Yuuichirou HIRAI, Tokyo (JP); Keisuke ONO, Tokyo (JP); Midori UMEHARA, Tokyo (JP); Yuki TESHIMA, Tokyo (JP); Tetsuro NAKANISHI, Tokyo (JP)

(73) Assignee: Daicel Corporation, Osaka (JP)

(21) Appl. No.: 19/388,900

(22) Filed: Nov. 13, 2025

Related U.S. Application Data
(63) Continuation-in-part of application No. 17/788,968, filed on Dec. 23, 2020, now Pat. No. 12,472,133.
(63) Continuation-in-part of application No. 17/788,991, filed on Dec. 23, 2020, now Pat. No. 12,472,134.
(63) Continuation-in-part of application No. 17/788,803, filed on Dec. 23, 2020, now Pat. No. 12,478,568.
(63) Continuation-in-part of application No. 17/789,067, filed on Dec. 23, 2020.
(63) Continuation-in-part of application No. 17/789,087, filed on Dec. 23, 2020.
(63) Continuation-in-part of application No. 17/789,113, filed on Dec. 23, 2020.
(63) Continuation-in-part of application No. 17/789,093, filed on Dec. 23, 2020.
(63) Continuation-in-part of application No. 17/788,823, filed on Dec. 23, 2020.
(63) Continuation-in-part of application No. 17/788,809, filed on Dec. 23, 2020.

(30) Foreign Application Priority Data
Dec. 28, 2019 (JP) 2019-239974
Dec. 28, 2019 (JP) 2019-239975
Dec. 28, 2019 (JP) 2019-239976
Dec. 28, 2019 (JP) 2019-239977
Dec. 28, 2019 (JP) 2019-239978
Dec. 28, 2019 (JP) 2019-239979
Jan. 20, 2020 (JP) 2020-006660
Feb. 6, 2020 (JP) 2020-018910

(51) Int. Cl. C07C 31/20; A61K 8/34; C07C 29/82

(52) U.S. Cl. CPC C07C 31/207; C07C 29/82; A61K 8/345

(57) ABSTRACT

The present disclosure provides a method for producing 1,3-butylene glycol to yield purified 1,3-butylene glycol from a reaction crude liquid containing 1,3-butylene glycol.

The present disclosure relates to a method for producing 1,3-butylene glycol, and a 1,3-butylene glycol product.

U.S. Patent Application Publication Nos. US2023/0033469, US2023/0035233, US2023/0055591, US2022/0354760, US2023/0048638, US2023/0046811, US2023/0098112, US2023/0087989, US2023/0338248 are hereby incorporated by reference in their entireties.

### BACKGROUND ART

1,3-Butylene glycol is a colorless, transparent, and odorless liquid and has properties, such as low volatility, low toxicity, and high hygroscopicity, and has excellent chemical stability. 1,3-butylene glycol has a wide range of applications, including raw materials for various synthetic resins and surfactants, as well as materials for cosmetics, hygroscopic agents, high boiling point solvents, and antifreezes, etc. Particularly in recent years, 1,3-butylene glycol has been attracting attention for having excellent properties as a moisturizer, and demand is growing in the cosmetic industry.

A reaction crude liquid during production of 1,3-butylene glycol contains a large number of low boiling point impurities such as ethanol, butanol, acetaldehyde, crotonaldehyde, and esters. Dimerization or polymerization to a higher degree of acetaldehyde, crotonaldehyde, and the like, among them, form high boiling point impurities. In addition, even in purification of 1,3-butylene glycol, low boiling point impurities and high boiling point impurities are produced by heat or the like. In products of 1,3-butylene glycol, less impurities are desirable. JP 06-329664 A discloses a method of controlling an impurity derived from crotonaldehyde, which is a low boiling point impurity. JP 2001-213828 A discloses a high-yield, economically advantageous production of a high-purity 1,3-butylene glycol product in which a reaction crude liquid of 1,3-butylene glycol synthesized by hydrogenation of acetaldols is made basic, followed by distillation of an alcohol, and then this liquid is distilled. Furthermore, this document describes distilling off ethanol, isopropyl alcohol, and butanol as low boiling point impurities by distillation.

One of the product standards for 1,3-butylene glycol includes a potassium permanganate test value (abbreviation: PMT). Patent Document 1 discloses 1,3-butylene glycol having a potassium permanganate fading time of 5 minutes or longer after 3 months of manufacture.

1,3-Butylene glycol produced by a known production method has had a problem of acid concentration (acidity) increase when the 1,3-butylene glycol is left for a long period of time in a state containing water. The cause of the acid concentration increase has been uncertain but thought to be related to by-products contained in crude 1,3-butylene glycol. Cosmetics typically contain water and require a long period of time from production to actual use by general consumers. In addition, from the viewpoint, such as storage stability of cosmetics, liquidity is strictly controlled. When 1,3-butylene glycol produced by a known method is used in cosmetics, an acid concentration increase can disrupt the liquidity balance of the cosmetics, and this can lead to a loss of the intended effect. In addition, the acid concentration increase of cosmetics can cause rough skin or the like of the users. Furthermore, there was a case where cosmetics containing no water also have absorbed water during use or storage, and this has also increased the acid concentration accordingly. Thus, removing by-products from crude 1,3-butylene glycol to yield high-purity 1,3-butylene glycol has been required.

Also, 1,3-butylene glycol produced by a known production method have sometimes had an odor due to effects from by-products. In addition, 1,3-butylene glycol transparent immediately after production may be colored over time, which is a problem in storing for a long period of time. For example, during use and/or storage after use of a cosmetic, the cosmetic is exposed to air. In addition, in production of cosmetics, the production is usually performed in air atmosphere, and the product may be heated for the purposes of, for example, sterilization. When 1,3-butylene glycol produced by a known method is used in cosmetics, sometimes coloration occurs due to the presence of air or effects of heating. To solve such problems, removing by-products from crude 1,3-butylene glycol to yield high-purity 1,3-butylene glycol has been required.

For a method to produce high-purity 1,3-butylene glycol, a method including adding sodium hydroxide to crude 1,3-butylene glycol produced by hydrogen reduction of acetaldols and performing distillation has been proposed. In addition, other methods have been proposed, including a method in which an alkali metal base is added to crude 1,3-butylene glycol from which a high boiling point substance has been removed, the mixture is heat-treated, then 1,3-butylene glycol is distilled, and the alkali metal compound and a high boiling point substance are separated as residues, and subsequently a low boiling point substance is distilled off from the 1,3-butylene glycol distillate (Patent Documents 1 to 6). Various methods for purifying 1,3-butylene glycol have been thus proposed to produce high-purity 1,3-butylene glycol.

### SUMMARY OF INVENTION

### Technical Problem

However, with the known methods, a low boiling point component and a high boiling point component are not sufficiently removed from 1,3 butylene glycol. Cosmetics, which are an important application of 1,3 butylene glycol, typically contain water and require a long period of time from production to actual use by general consumers. In addition, from the viewpoint, such as storage stability of cosmetics, liquidity is strictly controlled.

When 1,3-butylene glycol containing a low boiling point component and a high boiling point component is used in cosmetics, an acid concentration increase can disrupt the liquidity balance of the cosmetics, and this can lead to a loss of the intended effect. In addition, the acid concentration increase of cosmetics can cause rough skin or the like of the users.

Further, during use and/or storage after use of a cosmetic, the cosmetic is exposed to air. In addition, in manufacturing cosmetics, the manufacturing work is usually performed in air atmosphere, and the product may be heated for the purposes of, for example, sterilization. When 1,3-butylene glycol containing a low boiling point component and a high boiling point component is used in cosmetics, sometimes coloration occurs in the presence of air or by effects of heating.

To solve such problems, removing by-products from crude 1,3-butylene glycol to obtain high-purity 1,3-butylene glycol has been required.

On the other hand, one of quality standards for 1,3-butylene glycol is dry point. The lower the dry point, the better the quality. When the high boiling point impurities produced in the reaction and the purification processes are mixed into products, not only their purity is reduced, but also the dry point increases, resulting in a problem that the products do not meet the product quality standards. However, a method of separation and removal of high boiling point impurities has hardly been studied so far.

Therefore, the first aspect of the present disclosure is to provide a method capable of producing high-purity 1,3-butylene glycol having a very low content of a high boiling point component and a low dry point, with a high recovery ratio.

The first aspect of the present disclosure is to provide a high-purity 1,3-butylene glycol product having a very low content of a high boiling point component and a low dry point.

Still the first aspect of the present disclosure is to provide a moisturizer and a cosmetic product that has an excellent moisturizing performance and can retain high quality for a long period of time.

Moreover, on the other hand, one of quality standards for 1,3-butylene glycol is initial boiling point. The higher the initial boiling point, the better the quality. However, technical studies aimed at improving the initial boiling point have been hardly conducted so far.

Therefore, the second aspect of the present disclosure is to provide a method capable of producing high-purity 1,3-butylene glycol having a very low content of a low boiling point component and a high initial boiling point, with a high recovery ratio.

Another second aspect of the present disclosure is to provide a high-purity 1,3-butylene glycol product having a very low content of a low boiling point component and a high initial boiling point.

Still the second aspect of the present disclosure is to provide a moisturizer and a cosmetic product that have an excellent moisturizing performance and can retain high quality for a long period of time.

Moreover, however, in the known method described above, the potassium permanganate test value of the obtained 1,3-butylene glycol product is not always fully satisfactory. Also, the substance that caused to reduce the potassium permanganate test value of 1,3-butylene glycol products has not been identified.

Therefore, the third aspect of the present disclosure thus is to provide a method capable of manufacturing 1,3-butylene glycol having a high potassium permanganate test value.

The third aspect of the present disclosure is to provide a 1,3-butylene glycol product having a high potassium permanganate test value.

Still the third aspect of the present disclosure is to provide a moisturizer and a cosmetic product that have excellent moisturizing performance and can maintain high quality for a long period of time.

Moreover, however, almost no technical studies for the purpose of improving the initial boiling point have been conducted so far. Furthermore, however, the potassium permanganate test value of the resulting 1,3-butylene glycol product obtained through known methods is not always fully satisfactory. Also, the substance that caused to reduce the potassium permanganate test value of 1,3-butylene glycol products has not been identified.

Thus, the fourth aspect of the present disclosure is to provide a method capable of manufacturing high-purity 1,3-butylene glycol having a high potassium permanganate test value, a very low content of low boiling point components, and a high initial boiling point with a high recovery rate.

The fourth aspect of the present disclosure is to provide a high-purity 1,3-butylene glycol product having a high potassium permanganate test value, a very low content of low boiling point components and a high initial boiling point.

Still the fourth aspect of the present disclosure is to provide a moisturizer and a cosmetic product that have excellent moisturizing performance and can maintain high quality for a long period of time.

Moreover, however, almost no technical studies for the purpose of improving the initial boiling point have been conducted so far. Furthermore, however, not many studies on methods for separating and removing high boiling point impurities have been done. Also, however, the potassium permanganate test value of the resulting 1,3-butylene glycol product obtained through known methods is not always fully satisfactory. Also, the substance that caused to reduce the potassium permanganate test value of 1,3-butylene glycol products has not been identified. That is, there has been no industrially efficient method for manufacturing 1,3-butylene glycol products that are excellent in not only the first and second characteristics, which are initial boiling point and dry point, but excellent in also all three characteristics: the initial boiling point, dry point and potassium permanganate test value.

Therefore, the fifth aspect of the present disclosure is to provide a method capable of industrially efficiently manufacturing high-purity 1,3-butylene glycol having a very low content of low boiling point components and high boiling point components, a high initial boiling point and a low dry point.

The fifth aspect of the present disclosure is to provide a method capable of industrially efficiently manufacturing high-purity 1,3-butylene glycol having a high potassium permanganate test value, and a very low content of low boiling point components and high boiling point components, a high initial boiling point, and a low dry point.

Still the fifth aspect of the present disclosure is to provide a high-purity 1,3-butylene glycol product having a very low content of low boiling point components and high boiling point components, a high initial boiling point and a low dry point.

The fifth aspect of the present disclosure is to provide a high-purity 1,3-butylene glycol product having a high potassium permanganate test value, and a very low content of low boiling point components and high boiling point components, a high initial boiling point, and a low dry point.

Still the fifth aspect of the present disclosure is to provide a moisturizer and a cosmetic product that have excellent moisturizing performance and can maintain high quality for a long period of time.

Moreover, however, almost no technical studies for the purpose of improving the initial boiling point have been conducted so far. Furthermore, when the high boiling point impurities produced in the reaction and purification processes are mixed into products, not only their purity is reduced, but also the dry point is raised, resulting in issues that the products will not meet the product quality standards. However, not many studies on methods for separating and removing high boiling point impurities have been done. Also, however, the potassium permanganate test value of the resulting 1,3-butylene glycol product obtained through known methods is not always fully satisfactory. Also, the substance that caused to reduce the potassium permanganate test value of 1,3-butylene glycol products has not been identified. That is, there has been no industrially efficient method for manufacturing a 1,3-butylene glycol product excellent in two characteristics of initial boiling point and dry point, two characteristics of initial boiling point and potassium permanganate test value, and further three characteristics of initial boiling point, dry point, and potassium permanganate test value.

Therefore, the sixth aspect of the present disclosure is to provide a method capable of industrially efficiently manufacturing high-purity 1,3-butylene glycol having a very low content of low boiling point components and high boiling point components, a high initial boiling point and a low dry point.

The sixth aspect of the present disclosure is to provide a method capable of industrially efficiently manufacturing high-purity 1,3-butylene glycol having a very low content of low boiling point components, a high initial boiling point, and, besides, a high potassium permanganate test value.

The sixth aspect of the present disclosure is to provide a method capable of industrially efficiently manufacturing high-purity 1,3-butylene glycol having a high potassium permanganate test value, and a very low content of low boiling point components and high boiling point components, a high initial boiling point, and a low dry point.

However, 1,3-butylene glycol products produced from these purification methods still contain by-products and have a problem of having an odor, a problem of increasing acid concentration over time when containing water, and a problem that coloration occurs over time. Also, however, 1,3-butylene glycol products produced from these purification methods still contain by-products and have a problem of having an odor, a problem of causing odor over time even when having no odor immediately after manufacture, a problem of an increase in acid concentration over time when the 1,3-butylene glycol product contains water, a problem of coloration, and a problem of an increase in coloration over time. 1,3-Butylene glycol is produced, for example, by a method of (1) reduction (hydrogenation) of acetaldols, (2) hydrolysis of 1,3-butylene oxide, (3) selective hydrocracking of erythritol, (4) selective hydration of butadiene, (5) hydrogen addition to n-butanol-3-one, (6) hydrogen addition to 1-butanol-3-one, (7) hydrogen addition to 3-hydroxy-1-butanoic acid, (8) hydrogen addition to β-butyrolactone, or (9) hydrogen addition to diketene.

Of the production methods, the method of (1) reduction (hydrogenation) of acetaldols to yield 1,3 butylene glycol is preferable. Among them, the method of reduction of acetaldols in a liquid phase is preferable in terms of yield. The reasons for this are that acetaldols have a high boiling point, that acetaldols are unstable to heat and readily undergo a dehydration reaction at high temperatures to form crotonaldehyde or the like, and furthermore, that a reaction rate of the dehydration reaction is greater than that of the reduction reaction (hydrogenation reaction) at high temperatures. That is, when acetaldols are subjected to vapor-phase reduction, the temperature inside the reaction system needs to be increased to high temperature, but subjecting the acetaldols to high temperature causes a dehydration reaction to produce crotonaldehyde or the like, and the subsequent reduction reaction produces by-products, such as butanol. Thus, this results in relatively reduced yield of the target 1,3-butylene glycol. Thus, to produce a high-purity 1,3-butylene glycol product, a liquid phase reduction rather than a gas phase reduction is preferably performed.

In production of 1,3-butylene glycol, by-products are generally produced in the production process. For example, in production of 1,3-butylene glycol by hydrogen reduction of an acetaldol, a low boiling point substance (low boiling point compound) having an unsaturated bond, such as acetaldehyde, butylaldehyde, crotonaldehyde, acetone, and methyl vinyl ketone; a condensate of these (e.g., a trimer of acetaldehyde); a hydride of the above condensate; a condensate of 1,3-butylene glycol and the above low boiling point substance (e.g., an acetal of 1,3-butylene glycol and acetaldol); or the like is produced as a by-product.

In addition, an acetal of crotonaldehyde and 1,3-butylene glycol; an acetal of acetaldehyde and 1,3-butylene glycol; an acetal of a hydride of a trimer of acetaldol, acetaldehyde, and acetaldehyde; or the like is produced as a by-product. Furthermore, in addition to the above, acetic acid may be present as an impurity in an acetaldol used as a raw material or is used to neutralize sodium hydroxide used in producing an acetaldol, and a condensate of the acetic acid and 1,3-butylene glycol (ester of acetic acid and 1,3-butylene glycol) is formed as a by-product. Moreover, these by-products may have properties as a substance that causes coloration, a substance that causes odor or a substance that causes acidity.

Whether the acetal is a substance that causes coloration, a substance that causes odor or a substance that causes acidity is uncertain, and the acetal is also considered to have all the properties but is considered to have strong properties as a substance that causes odor. Specifically, the acetal itself is unlikely to be a substance that causes odor but can form a substance that causes odor by a change over time or heating. Furthermore, the acetal may be hydrolyzed to form acetaldol, which is a substance that causes odor and has an oxidation (coloration) promoting effect. Thus, the acetal can also be said to be a substance that causes coloration. Here, the substance that causes coloration is defined as including not only a substance actually having a hue itself but also a substance changing over time into a substance having a hue. The substance that causes odor is defined as including not only a substance actually emitting an odor itself but also a substance changing over time into a substance emitting an odor. The substance that causes acidity is defined as a substance increasing acid concentration over time when containing water.

Whether the ester is a substance that causes coloration, a substance that causes odor or a substance that causes acidity is uncertain, and the ester is also considered to have all the properties but is considered to have strong properties as both a substance that causes odor and a substance that causes acidity. This is because when the ester is hydrolyzed by water, acetic acid is produced.

In production of 1,3-butylene glycol, it is considered that by-products in the production process may include, in addition to the acetal and ester, a wide variety of by-products corresponding to a substance that causes coloration, a substance that causes odor, or a substance that causes acidity. For example, the hydride of a trimer of acetaldehyde described above is considered to possibly correspond to any of a substance that causes coloration, a substance that causes odor or a substance that causes acidity.

The by-products described above are difficult to completely remove even using a purification means, such as distillation known in the art. This is considered to be because crude 1,3-butylene glycol is subjected to high temperature conditions and alkali treatment in the purification stage of the crude 1,3-butylene glycol, and new by-products are produced accordingly. For these reasons, as described above, the 1,3-butylene glycol products of Patent Documents 1 to 6 contain a wide variety of by-products, thus have an odor and are colored over time, and furthermore, increase acid concentration over time in a state containing water.

Thus, the seventh aspect of the present disclosure is to provide a high-purity 1,3-butylene glycol product that is colorless and odorless (or almost colorless and odorless), unlikely to cause coloration and odor over time, and, besides, unlikely to cause an acid concentration increase over time also in a state containing water.

Furthermore, the seventh aspect of the present disclosure is to provide a moisturizer and a cosmetic product that have excellent moisturizing performance and can retain high quality for a long period of time.

Further, the seventh aspect of the present disclosure is to provide a method for industrially efficiently producing high-purity 1,3-butylene glycol having properties of being colorless and odorless (or almost colorless and odorless), unlikely to cause coloration over time, and, besides, unlikely to cause an acid concentration increase over time also in a state containing water.

Thus, the eighth aspect of the present disclosure is to provide a high-purity 1,3-butylene glycol product that is colorless and odorless (or almost colorless and odorless), unlikely to cause or increase coloration and odor over time, and/or unlikely to cause an acid concentration increase over time even in a state containing water.

Furthermore, the eighth aspect of the present disclosure is to provide a moisturizer and a cosmetic product that have excellent moisturizing performance and can retain high quality for a long period of time.

Thus, the ninth aspect of the present disclosure is to provide a high-purity 1,3-butylene glycol product that is colorless and odorless (or almost colorless and odorless), and hardly causes or increases odor over time.

The ninth aspect of the present disclosure is to provide a high-purity 1,3-butylene glycol product that is colorless and odorless (or almost colorless and odorless), hardly causes or increases coloration and odor over time, and/or is unlikely to cause an acid concentration increase over time even in a state containing water.

Still the ninth aspect of the present disclosure is to provide a moisturizer and a cosmetic product that have excellent moisturizing performance and can retain high quality for a long period of time.

### Solution to Problem

In the first aspect of the present disclosure, as a result of diligent research to achieve the above-described purpose, the inventors of the present disclosure have found that, when a reflux ratio and a bottom ratio of a distillation column for removal of a high boiling point component are controlled, a high boiling component is concentrated and removed from below a charging plate of the high boiling point component removal column, and 1,3-butylene glycol which has a reduced high boiling component concentration is extracted from above the charging plate of the high boiling point component removal column. Thus, three effects of: reducing a high boiling point impurity concentration of a product to improve the purity; controlling a dry point within a standard value; and maintaining or improving the recovery ratio of 1,3-butylene glycol, can be attained at the same time. The first aspect of the present disclosure was completed based on these findings.

In the second aspect of the present disclosure, as a result of diligent research to achieve the purpose described above, the inventors of the present disclosure have found that, when a content of water in a charged liquid into a product column is specified, a reflux ratio in the product column is controlled, and a product column distillate is recycled to a dehydration column and a low boiling point component removal column, it is possible to efficiently remove the low boiling component mixed in the charged liquid into the product column, and maintain and improve the recovery ratio of 1,3-butylene glycol while maintaining the initial boiling point of 1,3-butylene glycol within a defined value range. The second aspect of the present disclosure was completed based on these findings.

In the third aspect of the present disclosure, as a result of diligent research to achieve the above-described purpose, the inventors according to the present disclosure have found that, when a liquid feed having a concentration of 1,3-butylene glycol and an acetaldehyde content within predetermined ranges is fed to a product column and distilled at a predetermined reflux ratio, a potassium permanganate test value of a 1,3-butylene glycol product can be greatly improved; and also that, when a distillate from the product column is recycled to a step prior to a product distillation step, a potassium permanganate test value of the 1,3-butylene glycol product can be further improved; and a recovery rate of 1,3-butylene glycol can be maintained and improved even when such a method is performed. The third aspect of the present disclosure has been completed based on these findings.

In the fourth aspect of the present disclosure, as a result of diligent research to achieve the above-described purpose, the inventors of the present disclosure have found that, by specifying a content of acetaldehyde and crotonaldehyde in a liquid feed into a dehydration column and controlling a reflux ratio in the dehydration column, specifying a content of acetaldehyde and crotonaldehyde in a 1,3-butylene glycol liquid feed into a product column and controlling a reflux ratio in the product column, and preferably, also by recycling a product column distillate to a step prior to the product distillation step, it is possible to efficiently remove low boiling components mixed in the liquid feed into the product column and to maintain and improve a recovery rate of 1,3-butylene glycol with an initial boiling point of 1,3-butylene glycol and a potassium permanganate test value being maintained within standard values. The fourth aspect of the present disclosure has been completed by conducting further studies based on these findings.

In the fifth aspect of the present disclosure, as a result of diligent research to achieve the above purposes, the inventors of the present disclosure have found that, by performing distillation under a predetermined reflux ratio in a high boiling point substance removal column with the content of acetaldehyde and crotonaldehyde in the liquid feed into a dehydration column being specified and a reflux ratio in the dehydration column, high-purity 1,3-butylene glycol having a very low content of low boiling point components being controlled, it is possible to obtain high boiling point components from above the feed plate; and also that, by further feeding a liquid taken out from above the feed plate of the high boiling point substance removal column into a product column and distilling at a predetermined reflux ratio, higher-quality 1,3-butylene glycol is obtained from below the feed plate of the product column. The fifth aspect of the present disclosure has been completed by conducting further studies based on these findings.

In the sixth aspect of the present disclosure, as a result of diligent research to achieve the purposes, the inventors of the present disclosure have found that, by specifying an acetaldehyde content and a crotonaldehyde content in a liquid feed into a dehydration column, and distilling a liquid feed having not higher than predetermined content of acetaldehyde and crotonaldehyde in a predetermined reflux ratio in a product column, high-purity 1,3-butylene glycol having a very low content of low boiling point components and high boiling point components is obtained from below a feed plate. Also, the inventors have found that, by specifying a content acetaldehyde and crotonaldehyde in a liquid feed into a dehydration column, and distilling a liquid feed containing 1,3-butylene glycol in a predetermined reflux ratio in a high boiling substance removal column, high-purity 1,3-butylene glycol having a very low content of low boiling point components and high boiling point components is obtained from above a feed plate. The sixth aspect of the present disclosure has been completed by conducting further studies based on these findings.

In the seventh aspect of the present disclosure, as a result of diligent research to achieve the above purposes, the inventors of the present disclosure have found that specific nine compounds and, further, acetaldehyde and crotonaldehyde are substances that cause coloration, odor, an increase in coloration over time, an increase in odor over time, and an acid concentration increase. Then, the inventors have found a method for suppressing mixing of these compounds into a 1,3-butylene glycol product. More specifically, the inventors have found that, by adjusting contents of specific impurities and a concentration of 1,3-butylene glycol in charged liquids into a product column and a high boiling point component removal column and, further, by adjusting reaction conditions (particularly, increasing a partial pressure of hydrogen in a reactor) in a reaction step (e.g., hydrogenation of an acetaldol), it is possible to produce 1,3-butylene glycol having the properties of being colorless and odorless (or almost colorless and odorless), unlikely to cause coloration and odor over time, and, besides, unlikely to cause an acid concentration increase over time also in a state containing water. The seventh aspect of the present disclosure has been completed by further studying based on these findings.

In the eighth aspect of the present disclosure, as a result of diligent research to achieve the purpose described above, the present inventors have found that storage of the 1,3-butylene glycol product for a long period of time causes deteriorations in qualities such as increase in coloration, increase in odor, decrease in initial boiling point over time, rise in dry point, and decrease in potassium permanganate test value over time, and that a content of a specific chemical substance increases over time in the 1,3-butylene glycol product correspondingly. Therefore, as a result of subjecting the 1,3-butylene glycol product to a heating test (accelerated test) under specific conditions, and reviewing correlation between the content of the specific chemical substance in a sample after the test and the deteriorations in qualities (coloration, odor, initial boiling point, dry point, potassium permanganate test value, acid concentration, etc.) of the product over time, the present inventors have found a clear correlation therebetween. Having studied a pathway of the specific chemical substance formation at the same time, the present inventors have found a method of suppressing a specific chemical substance and its precursor from being mixed into the 1,3-butylene glycol product. More specifically, the present inventors have found that, by adjusting distillation conditions (content of specific impurities in a charged liquid, reflux ratio, etc.) of a product column, a high boiling point component removal column, a dehydration column, etc., and further, by adjusting reaction conditions (particularly, increasing a partial pressure of hydrogen in a reactor) in a reaction step (e.g., hydrogenation of an acetaldol), it is possible to suppress the specific chemical substance and its precursor from being mixed into the 1,3-butylene glycol product, and to produce 1,3-butylene glycol that is characteristically unlikely to cause or increase coloration and odor over time, and unlikely to cause an acid concentration increase over time even in a state containing water. The invention according to the eighth aspect of the present disclosure has been completed by further studying based on these findings.

In the ninth aspect of the present disclosure, as a result of diligent research to achieve the purpose, the inventors of the present disclosure have found that: storage of a 1,3-butylene glycol product for a long period of time causes deteriorations in qualities such as increase in coloration, increase in odor over time, decrease in initial boiling point, rise in dry point, and decrease in potassium permanganate test value over time; and that increase in contents of specific impurities in the 1,3-butylene glycol product to certain values or greater not only deteriorates qualities (coloration, odor, initial boiling point, dry point, potassium permanganate test value, acid concentration, etc.) of the 1,3-butylene glycol product, but also further deteriorates the qualities during storage of the product for a long period of time. Also, the present inventors have found a method for producing 1,3-butylene glycol in which the contents of the specific impurities can be reduced as much as possible. The ninth aspect of the present disclosure has been completed by further studying based on these findings.

Specifically, the first aspect of the present disclosure provides a method for producing 1,3-butylene glycol to yield purified 1,3-butylene glycol from a reaction crude liquid containing 1,3-butylene glycol,
the method including: performing dehydration for removing water by distillation, removing a high boiling point component including removing a high boiling point component by distillation, and performing product distillation to yield purified 1,3-butylene glycol,
wherein, in a high boiling point component removal column for use in the removing the high boiling point component, a charged liquid including 1,3-butylene glycol is distilled under a condition that a reflux ratio is greater than 0.02 in a case where a concentration of 1,3-butylene glycol in the charged liquid is 95% or less, or a reflux ratio is greater than 0.01 in a case where the concentration of 1,3-butylene glycol in the charged liquid is greater than 95%, and high-purity 1,3-butylene glycol is distilled off from above a charging plate, and a liquid in which a high boiling point component is concentrated is extracted from below the charging plate.

The reaction crude liquid containing 1,3-butylene glycol may be a reaction crude liquid formed by hydrogen reduction of acetaldols.

The producing method further includes alkaline treatment including treating a process stream containing 1,3-butylene glycol with a base.

The producing method may further include desalting including removing a salt in a process stream containing 1,3-butylene glycol.

The producing method may further include performing dealcoholization (low boiling point component removal) including removing a low boiling point component including an alcohol, the low boiling point component being in the process stream containing 1,3-butylene glycol.

The reflux ratio of the high boiling point component removal column may be greater than 0.02.

The concentration of 1,3-butylene glycol in the charged liquid into the high boiling point component removal column may be 95% or greater.

A content of the high boiling point component in the charged liquid into the high boiling point component removal column may be 4% or less.

The content of the high boiling point component in the charged liquid into the high boiling point component removal column may be 0.1% or less.

A bottom ratio in the high boiling point component removal column may be less than 30 wt. %.

The bottom ratio in the high boiling point component removal column may be 0.01 wt. % or greater.

A theoretical number of plates in the high boiling point component removal columns is, for example, from 1 to 100 plates.

At least a portion of a bottom from the high boiling point component removal column may be recycled to the step prior to the removing a high boiling point component.

A recycled amount of the bottom from the high boiling point component removal column recycled to the step prior to the removing a high boiling point component may be less than 30 wt. % with respect to a charged amount into the high boiling point component removal column, in a range not greater than an amount of the bottom from the high boiling point component removal column.

A recycled amount of the bottom from the high boiling point component removal column recycled to the step prior to the removing a high boiling point component may be 0.01 wt. % or greater with respect to the charged amount into the high boiling point component removal column, in a range of not greater than an amount of the bottom from the high boiling point component removal column.

The present disclosure also provides a 1,3-butylene glycol product, having a dry point of 209 °C or lower and, according to a gas chromatographic analysis performed under conditions set forth below:
an area ratio of a 1,3-butylene glycol peak of greater than 98.5% and a total area ratio of peaks longer in retention time than the 1,3-butylene glycol peak of less than 1.2%,
   in which the conditions for the gas chromatographic analysis are as follows:
Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

The area ratio of the 1,3-butylene glycol peak may be 99.0% or greater.

The total area ratio of the peaks longer in retention time than the 1,3-butylene glycol peak may be 0.6% or less.

The present disclosure further provides a moisturizer containing the 1,3-butylene glycol product described above.

The present disclosure further provides a cosmetic product containing the moisturizer described above.

Moreover, specifically, the second aspect of the present disclosure provides a method for producing 1,3-butylene glycol to obtain purified 1,3-butylene glycol from a reaction crude liquid comprising 1,3-butylene glycol,
the method including: dehydration for removing water by distillation, removing a high boiling point component including removing a high boiling point component by distillation, and performing product distillation to yield purified 1,3-butylene glycol,
wherein, in a product column for use in the performing product distillation, a charged liquid containing 1,3-butylene glycol is distilled off under a condition of a reflux ratio of greater than 0.1, a liquid in which a low boiling point component is concentrated is distilled off from above a charging plate, and 1,3-butylene glycol is extracted from below the charging plate.

The reaction crude liquid containing 1,3-butylene glycol may be a reaction crude liquid formed by hydrogen reduction of acetaldols.

The producing method further includes alkaline treatment including treating a process stream containing 1,3-butylene glycol with a base.

The producing method may further include desalting including removing a salt in a process stream containing 1,3-butylene glycol.

The production method may further include performing dealcoholization including removing a low boiling point substance containing an alcohol in a process stream containing 1,3-butylene glycol.

The reflux ratio of the product column may be 0.2 or greater.

A concentration of 1,3-butylene glycol in the charged liquid into the product column may be 90% or greater.

A content of water in the charged liquid into the product column may be 3 wt. % or less.

A content of the low boiling point component other than water in the charged liquid into the product column may be 1.8% or less.

A distillation ratio in the product column may be less than 30 wt. %.

A theoretical number of plates in the product column may be from 1 to 100 plates.

At least a portion of a distillate from the product column may be recycled to the dehydration, dealcoholization, low boiling point component removal prior to the performing product distillation, or a step prior to the steps.

An recycled amount of the distillate from the product column recycled to the step prior to the performing product distillation may be less than 30 wt. % with respect to a charged amount into the product column, in a range of not greater than a distilled amount in the product column.

The recycled amount of the distillate from the product column recycled to the step prior to the performing product distillation may be 0.01 wt. % or greater with respect to the charged amount into the product column, in the range of not greater than the distilled amount in the product column.

The present disclosure also provides a 1,3-butylene glycol product having an initial boiling point of higher than 203 °C and a water content of less than 0.4 wt. %.

The 1,3-butylene glycol product according to the present disclosure may have, in a gas chromatographic analysis performed under conditions,
an area ratio of a 1,3-butylene glycol peak of greater than 98.5% and a total area ratio of peaks shorter in retention time than the 1,3-butylene glycol peak of less than 0.3%,
in which the conditions for the gas chromatographic analysis are as follows:
Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

The present disclosure further provides a moisturizer containing the 1,3-butylene glycol product described above.

The present disclosure further provides a cosmetic product containing the moisturizer described above.

Moreover, specifically, the third aspect of the present disclosure provides a method for manufacturing 1,3-butylene glycol, which is a method for obtaining purified 1,3-butylene glycol from a crude reaction liquid containing 1,3-butylene glycol, the method including: a dehydration step of removing water by distillation; a high boiling substance removal step of removing a high boiling point component by distillation; and a product distillation step of obtaining purified 1,3-butylene glycol,
wherein in the product distillation step, a product column is used in which a liquid feed having a 1,3-butylene glycol concentration of 97% or higher, an acetaldehyde content of 500 ppm or lower, and a crotonaldehyde content of 200 ppm or lower is distilled under a condition of a reflux ratio of higher than 0.1, and a liquid concentrated with acetaldehyde and crotonaldehyde is distilled off from above a feed plate, and 1,3-butylene glycol is extracted from below the feed plate.

The crude reaction liquid containing 1,3-butylene glycol may be a crude reaction liquid obtained by hydrogen reduction of an acetaldol.

The manufacturing method may further include an alkaline treatment step of treating a process stream containing 1,3-butylene glycol with a base.

Furthermore, the manufacturing method may further include a desalting step of removing a salt in a process stream containing 1,3-butylene glycol.

Furthermore, the manufacturing method may further include a dealcoholization step of removing a low boiling substance containing alcohols in a process stream containing 1,3-butylene glycol.

A reflux ratio of the product column may be 0.2 or higher.

The content of acetaldehyde in the liquid feed into the product column may be 205 ppm or lower.

The content of crotonaldehyde in the liquid feed into the product column may be 110 ppm or lower.

A distillation rate in the product column may be lower than 20 wt. %.

The number of theoretical plates of the product columns is, for example, from 1 to 100 plates.

At least a portion of a distillate from the product column may be recycled to a step prior to the product distillation step, namely the dehydration step, a dealcoholization step, a low boiling substance removal step, or another step prior to these steps.

The crude reaction liquid containing 1,3-butylene glycol may be a crude reaction liquid obtained by hydrogen reduction of acetaldols, and at least a portion of the distillate from the product column may be recycled to the hydrogen reduction of acetaldols or a step upstream of the hydrogen reduction.

An amount of the distillate from the product column being recycled to a step prior to the product distillation step may be lower than 30 wt. % with respect to a charged amount into the product column within a range not higher than a distilled amount in the product column.

The amount of the distillate from the product column being recycled to a step prior to the product distillation step may be 0.01 wt. % or higher with respect to a charged amount into the product column within a range not higher than the distilled amount in the product column.

The present disclosure also provides a 1,3-butylene glycol product having a potassium permanganate test value of longer than 10 minutes.

In the 1,3-butylene glycol product, a peak area ratio of 1,3-butylene glycol is preferably higher than 98.5%, according to a gas chromatographic analysis performed under conditions set forth below.

### Conditions for the Gas Chromatographic Analysis are as Follows:

Analytical Column: a column with dimethylpolysiloxane as a stationary phase, having a length of 30 m, an inner diameter of 0.25 mm, and a film thickness of 1.0 μm
Heating Conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

The 1,3-butylene glycol product preferably has an acetaldehyde content of 2 ppm or lower.

The 1,3-butylene glycol product preferably has a crotonaldehyde content of 1.2 ppm or lower.

The present disclosure further relates to a moisturizer containing the 1,3-butylene glycol product.

Furthermore, the present disclosure further provides a cosmetic product containing the moisturizer.

Moreover, specifically, the fourth aspect of the present disclosure provides a method for manufacturing 1,3-butylene glycol, which is a method for obtaining purified 1,3-butylene glycol from a crude reaction liquid containing 1,3-butylene glycol,
the method including: a dehydration step of removing water by distillation; a high boiling substance removal step of removing a high boiling point component by distillation; and a product distillation step of obtaining purified 1,3-butylene glycol,
wherein in the dehydration step, a dehydration column is used in which a liquid feed containing 1,3-butylene glycol and water with an acetaldehyde content of 1000 ppm or lower and a crotonaldehyde content of 400 ppm or lower is distilled under a condition of a reflux ratio of higher than 0.3, and a liquid concentrated with a low boiling point component containing water is distilled off from above a feed plate, and
in the product distillation step, a product column is used in which a 1,3-butylene glycol liquid feed with an acetaldehyde content of 500 ppm or lower and a crotonaldehyde content of 200 ppm or lower is distilled under a condition of a reflux ratio of higher than 0.1, a liquid concentrated with a low boiling point component is distilled off from above a feed plate, and 1,3-butylene glycol is extracted from below the feed plate.

The crude reaction liquid containing 1,3-butylene glycol may be a crude reaction liquid obtained by hydrogen reduction of an acetaldol.

The manufacturing method may further include an alkaline treatment step of treating a process stream containing 1,3-butylene glycol with a base.

Furthermore, the manufacturing method may further include a desalting step of removing a salt in a process stream containing 1,3-butylene glycol.

Furthermore, the manufacturing method may further include a dealcoholization step of removing a low boiling substance containing alcohols in a process stream containing 1,3-butylene glycol.

The content of acetaldehyde in the liquid feed into the dehydration column may be 155 ppm or lower, and the content of crotonaldehyde in the liquid feed may be 117 ppm or lower.

A content of water in the liquid feed into the dehydration column may be 90 wt. % or lower.

The dehydration column has the number of theoretical plates of, for example, from 1 to 100.

A concentration of 1,3-butylene glycol in the liquid feed into the product column may be 90 GC area % or higher, and a content of water in the liquid feed may be 3 wt. % or lower.

A content of the low boiling point component other than water in the liquid feed into the product column may be 1.8 GC area % or lower.

The content of acetaldehyde in the liquid feed into the product column may be 205 ppm or lower, and the content of crotonaldehyde in the liquid feed may be 110 ppm or lower.

A distillation rate in the product column may be less than 30 wt. %.

The number of theoretical plates of the product columns is, for example, from 1 to 100 plates

At least a portion of a distillate from the product column may be recycled to a step prior to the product distillation step, namely the dehydration step, a dealcoholization step, a low boiling substance removal step, or another step prior to these steps.

The crude reaction liquid containing 1,3-butylene glycol may be a crude reaction liquid obtained by hydrogen reduction of acetaldols, and at least a portion of the distillate from the product column may be recycled to the hydrogen reduction of acetaldols or a step upstream of the hydrogen reduction.

An amount of the distillate from the product column being recycled to the step prior to the product distillation step may be lower than 30 wt. % with respect to a charged amount into the product column within a range not higher than a distilled amount in the product column.

The present disclosure also provides a 1,3-butylene glycol product having an initial boiling point of higher than 203 °C and a potassium permanganate test value of 30 minutes or longer.

The 1,3-butylene glycol product may have, according to a gas chromatographic analysis performed under conditions set forth below, an area ratio of a 1,3-butylene glycol peak of higher than 98.7%, a total area ratio of peaks having shorter retention times than the 1,3-butylene glycol peak of lower than 0.3%, and a water content of lower than 0.4 wt. %, and have, according to GC-MS analysis performed under conditions set forth below, an acetaldehyde content of lower than 2 ppm and a crotonaldehyde content of lower than 1.2 ppm,
in which the conditions for the gas chromatographic analysis are as follows:
Analytical Column: a column with dimethylpolysiloxane as a stationary phase, having a length of 30 m, an inner diameter of 0.25 mm, and a film thickness of 1.0 μm
Heating Conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

### Conditions for GC-MS Analysis

Analytical Column: HP-1MS [18]30 m—0.25 mm i.d.—1.0 μm Oven Temperature: 80 °C (0 min)—5 °C/min—120 °C (0 min)—2 °C/min—160 °C (2 min)—10 °C/min—230 °C (18 min)
Carrier Gas: He 0.61 mL/min, const. flow (line speed: 29 cm/sec)
Split: 20:1
Inj: 250 °C
AUX: 280 °C
Injection Volume: 1 μL (ALS)
Ion Source Temperature: EI 230 °C, CI 250 °C
Q Pole temperature: 150 °C
Sample: subjected to analysis as it was

The present disclosure further relates to a moisturizer containing the 1,3-butylene glycol product.

Furthermore, the present disclosure further provides a cosmetic product containing the moisturizer.

Moreover, specifically, the fifth aspect of the present disclosure provides a method for manufacturing 1,3-butylene glycol, which is a method for obtaining purified 1,3-butylene glycol from a crude reaction liquid containing 1,3-butylene glycol,
the method including: a dehydration step of removing water by distillation, a high boiling point substance removal step of removing a high boiling point component by distillation, and a product distillation step of obtaining purified 1,3-butylene glycol,
wherein in the dehydration step, a dehydration column is used in which a liquid feed containing 1,3 butylene glycol and water with an acetaldehyde content of 1000 ppm or lower and a crotonaldehyde content of 400 ppm or lower is distilled under a condition of a reflux ratio of higher than 0.3, and a liquid concentrated with a low boiling point component containing water is distilled off from above a feed plate,
in the high boiling point substance removal step, a high boiling point substance removal column is used in which a liquid feed containing 1,3-butylene glycol is distilled under a condition of a reflux ratio of higher than 0.02, higher-purity 1,3-butylene glycol is distilled off from above a feed plate, and a liquid concentrated with a high boiling point component is extracted from below the feed plate, and
in the product distillation step, a product column is used in which a 1,3-butylene glycol liquid feed with an acetaldehyde content of 500 ppm or lower and a crotonaldehyde content of 200 ppm or lower is distilled under a condition of a reflux ratio of higher than 0.1, a liquid concentrated with a low boiling point component is distilled off from above a feed plate, and 1,3-butylene glycol is extracted from below the feed plate (hereinafter sometimes referred to as “manufacturing method 1 of the present disclosure”).

In the manufacturing method, a concentration of 1,3-butylene glycol in the liquid feed into the product column may be 90 GC area % or higher, and a content of water in the liquid feed may be 3 wt. % or lower.

A content of the low boiling point component other than water in the liquid feed into the product column may be 1.8 GC area % or lower.

The content of acetaldehyde in the liquid feed into the product column may be 205 ppm or lower, and wherein the content of crotonaldehyde in the liquid feed may be 110 ppm or lower.

The number of theoretical plates of the product columns is, for example, from 1 to 100 plates

At least a portion of a distillate from the product column may be recycled to a step prior to the product distillation step, namely the dehydration step, a dealcoholization step, a low boiling substance removal step, or another step prior to these steps.

The present disclosure also provides a method for manufacturing 1,3-butylene glycol, which is a method for obtaining purified 1,3-butylene glycol from a crude reaction liquid containing 1,3-butylene glycol,
the method including: a dehydration step of removing water by distillation; and a high boiling substance removal step of removing a high boiling point component by distillation,
wherein in the dehydration step, a dehydration column is used in which a liquid feed containing 1,3 butylene glycol and water with an acetaldehyde content of 1000 ppm or lower and a crotonaldehyde content of 400 ppm or lower is distilled under a condition of a reflux ratio of higher than 0.3, and a liquid concentrated with a low boiling point component containing water is distilled off from above a feed plate, and
in the high boiling substance removal step, a high boiling substance removal column is used in which a liquid feed containing 1,3-butylene glycol is distilled under a condition of a reflux ratio of higher than 0.02, higher-purity 1,3-butylene glycol is distilled off from above a feed plate, and a liquid concentrated with a high boiling point component is extracted from below the feed plate (hereinafter sometimes referred to as “manufacturing method 2 of the present disclosure”).

In each of these manufacturing methods according to the present disclosure, the crude reaction liquid containing 1,3-butylene glycol may be a crude reaction liquid obtained by hydrogen reduction of an acetaldol.

Furthermore, these manufacturing methods may further include an alkaline treatment step of treating a process stream containing 1,3-butylene glycol with a base.

Furthermore, these manufacturing methods may further include a desalting step of removing a salt in a process stream containing 1,3-butylene glycol.

Furthermore, the manufacturing method may further include a dealcoholization step of removing a low boiling substance containing an alcohol in a process stream containing 1,3-butylene glycol.

A content of water in the liquid feed into the dehydration column may be 90 wt. % or lower.

In the dehydration column and/or the high boiling substance removal column, the number of theoretical plates in at least one of these distillation columns may be from 1 to 100 plates.

A concentration of 1,3-butylene glycol in the liquid feed into the high boiling substance removal column may be 95% or higher.

A content of the high boiling point component in the liquid feed into the high boiling substance removal column may be 4% or lower.

The present disclosure also provides a 1,3-butylene glycol product, having an initial boiling point of higher than 203 °C and a dry point of 209 °C or lower.

The 1,3-butylene glycol product may have a potassium permanganate test value of 30 minutes or longer.

The 1,3-butylene glycol product may have, according to a gas chromatographic analysis performed under conditions set forth below:
an area ratio of a 1,3-butylene glycol peak being higher than 98.7%;
a total area ratio of peaks having shorter retention times than the 1,3-butylene glycol peak being lower than 0.3%; and
a total area ratio of peaks having longer retention times than the 1,3-butylene glycol peak being lower than 1.2%, and
also may have:
a water content of lower than 0.4 wt. %;
an acetaldehyde content of lower than 2 ppm; and
a crotonaldehyde content of lower than 1.2 ppm,
in which the conditions for the gas chromatographic analysis are as follows:
Analytical Column: a column with dimethylpolysiloxane as a stationary phase, having a length of 30 m, an inner diameter of 0.25 mm, and a film thickness of 1.0 μm
Heating Conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

The present disclosure further relates to a moisturizer containing the 1,3-butylene glycol product.

Furthermore, the present disclosure provides a cosmetic product containing the moisturizer.

Moreover, specifically, the sixth aspect of the present disclosure provides a method for manufacturing 1,3-butylene glycol, which is a method for obtaining purified 1,3-butylene glycol from a crude reaction liquid containing 1,3-butylene glycol, the method including:
a dehydration step of removing water by distillation; a high boiling substance removal step of removing a high boiling point component by distillation; and a product distillation step of obtaining purified 1,3-butylene glycol,
wherein in the dehydration step, a dehydration column is used in which a liquid feed containing 1,3-butylene glycol and water with an acetaldehyde content of 1000 ppm or lower and a crotonaldehyde content of 400 ppm or lower is distilled, and a liquid concentrated with a low boiling point component containing water is distilled off from above a feed plate, and
in the product distillation step, a product column is used in which a 1,3-butylene glycol liquid feed having an acetaldehyde content of 500 ppm or lower and a crotonaldehyde content of 200 ppm or lower is distilled under a condition of a reflux ratio of higher than 0.1, a liquid concentrated with a low boiling point component is distilled off from above a feed plate, and 1,3-butylene glycol is extracted from below the feed plate (hereinafter sometimes referred to as “manufacturing method 1 of the present disclosure”).

It is preferable that, in the manufacturing method, in the high boiling substance removal step, a high boiling substance removal column is used in which a liquid feed containing 1,3-butylene glycol is distilled under a condition of a reflux ratio of 0.02 or higher, 1,3-butylene glycol having further improved purity is distilled off from above a feed plate, and a liquid concentrated with a high boiling point component is extracted from below the feed plate.

It is preferable that a concentration of 1,3-butylene glycol in the liquid feed into the product column is 90 GC area % or higher, and that a content of water in the liquid feed is 3 wt. % or lower.

The present disclosure also provides a method for manufacturing 1,3-butylene glycol, which is a method for obtaining purified 1,3-butylene glycol from a crude reaction liquid containing 1,3-butylene glycol, the method including:
a dehydration step of removing water by distillation; and a high boiling substance removal step of removing a high boiling point component by distillation,
wherein in the dehydration step, a dehydration column is used in which a liquid feed containing 1,3-butylene glycol and water with an acetaldehyde content of 1000 ppm or lower and a crotonaldehyde content of 400 ppm or lower is distilled, and a liquid concentrated with a low boiling point component containing water is distilled off from above a feed plate, and
in the high boiling substance removal step, a high boiling substance removal column is used in which a liquid feed containing 1,3-butylene glycol is distilled under a condition of a reflux ratio of higher than 0.02, higher-purity 1,3-butylene glycol is distilled off from above a feed plate, and a liquid concentrated with a high boiling point component is extracted from below the feed plate (hereinafter sometimes referred to as “manufacturing method 2 of the present disclosure”).

In each of the manufacturing methods, a reflux ratio in the dehydration column is preferably 0.03 or higher.

Also, the crude reaction liquid containing 1,3-butylene glycol may be a crude reaction liquid obtained by hydrogen reduction of an acetaldol.

Each of the manufacturing methods may further include at least one step of: an alkaline treatment step of treating a process stream containing 1,3-butylene glycol with a base, a desalting step of removing a salt in the process stream containing 1,3-butylene glycol, or a dealcoholization step of removing a low boiling substance containing alcohols in the process stream containing 1,3-butylene glycol.

Moreover, that is, the seventh aspect of the present disclosure provides a 1,3-butylene glycol product in which at least one of eight contents: a content of methyl vinyl ketone, a content of acetone, a content of butylaldehyde, a content of acetaldol, a content of a compound represented by Formula (1) below, a content of a compound represented by Formula (2) below, a content of a compound represented by Formula (3) below, and a total content of a compound represented by Formula (4) below and a compound represented by Formula (5) below, is less than 8 ppm.

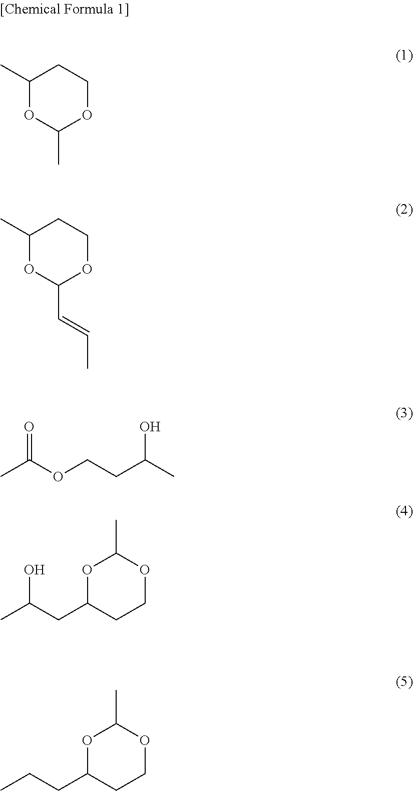

In the 1,3-butylene glycol product, a sum of the content of methyl vinyl ketone, the content of acetone, the content of butylaldehyde, the content of acetaldol, the content of the compound represented by Formula (1), the content of the compound represented by Formula (2), the content of the compound represented by Formula (3), the content of the compound represented by Formula (4), and the compound represented by Formula (5), may be less than 71 ppm.

In the 1,3-butylene glycol product, at least the content of acetaldol is preferably less than 8 ppm.

In the 1,3-butylene glycol product, at least the content of the compound represented by Formula (3) is preferably less than 8 ppm.

In the 1,3-butylene glycol product, a total content of methyl vinyl ketone, acetone, and butylaldehyde is preferably 24 ppm or less.

In the 1,3-butylene glycol product, a total content of the compound represented by Formula (1), the compound represented by Formula (2), the compound represented by Formula (4), and the compound represented by Formula (5) is preferably 24 ppm or less.

In the 1,3-butylene glycol product, a content of acetaldehyde is preferably less than 4 ppm and a content of crotonaldehyde is preferably less than 2 ppm.

In the 1,3-butylene glycol product, an acid concentration is preferably less than 11 ppm in terms of acetic acid, and, after a 90 wt. % aqueous solution has been kept at 100 °C for 1 week, an acid concentration is preferably less than 23 ppm in terms of acetic acid.

In the 1,3-butylene glycol product, an APHA is preferably 6 or less, and, after the 1,3-butylene glycol product has been kept at 180 °C for 3 hours in air atmosphere, an APHA is 78 or less.

Further, in the 1,3-butylene glycol product, an initial boiling point is preferably higher than 203 °C and/or a dry point is preferably 209 °C or lower.

In the 1,3-butylene glycol product, a potassium permanganate test value is preferably 30 minutes or longer.

In addition, the present disclosure provides a moisturizer containing the 1,3-butylene glycol product.

Furthermore, the present disclosure provides a cosmetic product containing the moisturizer.

The present disclosure further provides a method for producing 1,3-butylene glycol to yield purified 1,3-butylene glycol from a reaction crude liquid containing 1,3-butylene glycol,
the method (hereinafter sometimes referred to as “producing method 1 of the present disclosure”) including: performing dehydration including removing water by distillation, removing a high boiling point component including removing a high boiling point component by distillation, and performing product distillation to yield purified 1,3-butylene glycol,
wherein, in a product column for use in the product distillation, a 1,3-butylene glycol charged liquid is subjected to distillation under a condition that a reflux ratio is 0.3 or greater, the 1,3-butylene glycol charged liquid having a content of acetaldehyde of 500 ppm or less, a content of crotonaldehyde of 200 ppm or less, a content of water of 0.7 wt. % or less, and a concentration of 1,3-butylene glycol of 97.6 area %, according to a gas chromatographic analysis performed under conditions set forth below.

The conditions for the gas chromatographic analysis are as follows:
Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

In the method for producing 1,3-butylene glycol, at least a portion of a distillate of the product column may be recycled to a step before the performing product distillation, the step including dehydration, dealcoholization, low boiling point component removal, or a step before these steps.

The present disclosure also provides a method for producing 1,3-butylene glycol to yield purified 1,3-butylene glycol from a reaction crude liquid containing 1,3-butylene glycol,
the method (hereinafter sometimes referred to as “producing method 2 of the present disclosure”) including: performing dehydration including removing water by distillation and removing a high boiling point component including removing a high boiling point component by distillation,
wherein, in a high boiling point component removal column for use in the removing a high boiling point component, a charged liquid containing 1,3-butylene glycol is subjected to distillation under a condition that a reflux ratio is 0.03 or greater, the charged liquid having a content of acetaldehyde of 500 ppm or less, a content of crotonaldehyde of 200 ppm or less, a content of water of 3 wt. % or less, and a concentration of 1,3-butylene glycol of 96.7 area % or greater, according to a gas chromatographic analysis performed under conditions set forth below.

The conditions for the gas chromatographic analysis are as follows:
Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

In each of the producing methods, the reaction crude liquid containing 1,3-butylene glycol may be a reaction crude liquid produced by hydrogen reduction of an acetaldol.

Each of the producing methods may further include at least one step selected from alkali treatment including treating a process stream containing 1,3-butylene glycol with a base, desalting including removing a salt in a process stream containing 1,3-butylene glycol, and dealcoholization including removing a low boiling point substance containing an alcohol in a process stream containing 1,3-butylene glycol.

Moreover, that is, the eighth aspect of the present disclosure provides a 1,3-butylene glycol product containing 1,3-butylene glycol, in which, after the 1,3-butylene glycol product has been kept at 180 °C for 3 hours in air atmosphere, at least one of contents of compounds represented by the following Formula (A) or (B) is less than 8 ppm:

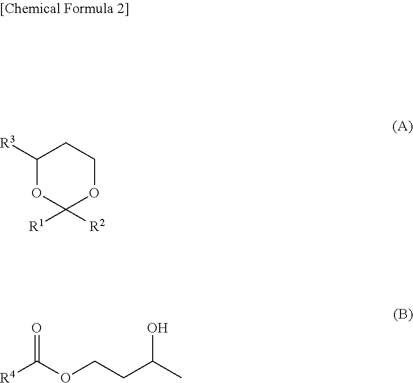

where R¹ to R⁴ are the same as or different from each other, and each of R¹ to R⁴ is a hydrogen atom, an alkyl group with from 1 to 4 carbon atoms which may be substituted with a hydroxy group, or an alkenyl group with 2 to 4 carbon atoms which may be substituted with a hydroxy group.

In the 1,3-butylene glycol product, after the 1,3-butylene glycol product has been kept at 180 °C for 3 hours in air atmosphere, a sum of the contents of the compounds represented by Formula (A) is preferably less than 66 ppm.

Also, after the 1,3-butylene glycol product has been kept at 180 °C for 3 hours in air atmosphere, a sum of the contents of the compounds represented by Formula (B) is preferably less than 31 ppm.

The compound represented by Formula (A) is, for example, any of compounds represented by the following Formulas (1) to (7), and the compound represented by Formula B is any of compounds represented by the following Formulas (8) to (10).

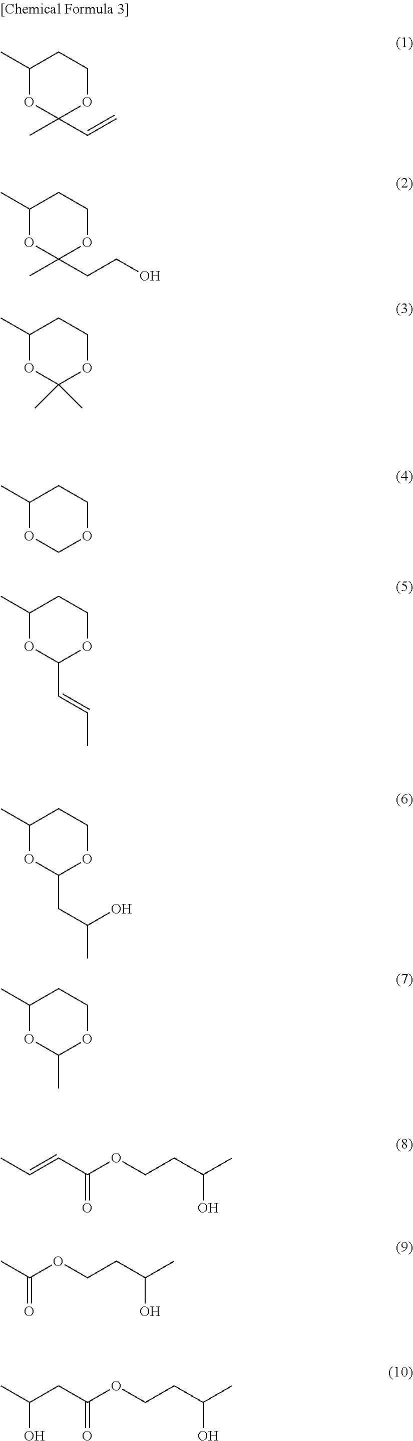

Also, in this case, after the 1,3-butylene glycol product has been kept at 180 °C for 3 hours in air atmosphere, a total content of the compounds represented by Formulas (1) to (4) is preferably less than 34 ppm.

Also, after the 1,3-butylene glycol product has been kept at 180 °C for 3 hours in air atmosphere, a total content of the compounds represented by Formulas (5) to (10) is preferably less than 63 ppm.

Further, after the 1,3-butylene glycol product has been kept at 180 °C for 3 hours in air atmosphere, a total content of the compounds represented by Formulas (1) to (10) is preferably less than 97 ppm.

After the 1,3-butylene glycol product has been kept at 180 °C for 3 hours in air atmosphere, in the 1,3-butylene glycol product, a content of methyl vinyl ketone is preferably less than 8 ppm, and/or a content of 1-hydroxy-3-butanone is preferably less than 1 ppm, and/or a content of acetone is preferably less than 6 ppm, and/or a content of formaldehyde is preferably less than 2 ppm, and/or a content of crotonaldehyde is preferably less than 1 ppm, and/or a content of acetaldol is preferably less than 1 ppm, and/or a content of acetaldehyde is preferably less than 5 ppm, and/or a content of butylaldehyde is preferably less than 4 ppm.

After the 1,3-butylene glycol product has been kept at 180 °C for 3 hours in air atmosphere, a total content of methyl vinyl ketone, 1-hydroxy-3-butanone, acetone, formaldehyde, crotonaldehyde, acetaldol and acetaldehyde is preferably less than 24 ppm.

Furthermore, after the 1,3-butylene glycol product has been kept at 180 °C for 3 hours in air atmosphere, a total content of methyl vinyl ketone, acetone, and butylaldehyde is preferably less than 18 ppm.

Furthermore, after the 1,3-butylene glycol product has been kept at 180 °C for 3 hours in air atmosphere, an APHA is preferably less than 79.

In addition, after the 1,3-butylene glycol product has been kept at 180 °C for 3 hours in air atmosphere, an initial boiling point is higher than 203 °C and/or a dry point is 209 °C or lower.

Furthermore, after the 1,3-butylene glycol product has been kept at 180 °C for 3 hours in air atmosphere, a potassium permanganate test value is 30 minutes or longer.

Further, after the 1,3-butylene glycol product has been kept at 180 °C for 3 hours in air atmosphere, an acid concentration (in terms of acetic acid) is less than 16 ppm.

In a state where the 1,3-butylene glycol product has not undergone a test in which 1,3-butylene glycol product is kept at 180 °C for 3 hours in air atmosphere, a total content of 1-hydroxy-3-butanone and acetaldol is less than 19 ppm.

In a state where the 1,3-butylene glycol product has not undergone a test in which 1,3-butylene glycol product is kept at 180 °C for 3 hours in air atmosphere, a content of methyl vinyl ketone is preferably less than 10 ppm, and/or a content of 1-hydroxy-3-butanone is preferably less than 9 ppm, and/or a content of acetone is preferably less than 9 ppm, and/or a content of formaldehyde is preferably less than 3 ppm, and/or a content of crotonaldehyde is preferably less than 2 ppm, and/or a content of acetaldol is preferably less than 10 ppm, and/or a content of acetaldehyde is preferably less than 4 ppm, and/or a content of butylaldehyde is preferably less than 8 ppm.

Further, in a state where the 1,3-butylene glycol product has not undergone a test in which 1,3-butylene glycol product is kept at 180 °C for 3 hours in air atmosphere, a total content of methyl vinyl ketone, 1-hydroxy-3-butanone, acetone, formaldehyde, crotonaldehyde, acetaldol and acetaldehyde is preferably less than 47 ppm.

In addition, the present disclosure also provides a moisturizer containing the 1,3-butylene glycol product.

Furthermore, the present disclosure also provides a cosmetic product containing the moisturizer.

Moreover, specifically, the ninth aspect of the present disclosure provides a 1,3-butylene glycol product containing 1,3-butylene glycol, wherein a sum of contents of acetaldehyde, crotonaldehyde, methyl vinyl ketone, acetone, formaldehyde, butylaldehyde, acetaldol, 1-hydroxy-3-butanone, 2-butanol, a compound represented by Formula (1) below, a compound represented by Formula (2) below, a compound represented by Formula (3) below, a compound represented by Formula (4) below, a compound represented by Formula (5) below, a compound represented by Formula (6) below, a compound represented by Formula (7) below, a compound represented by Formula (8) below, a compound represented by Formula (9) below, and a compound represented by Formula (10) below is less than 65 ppm.

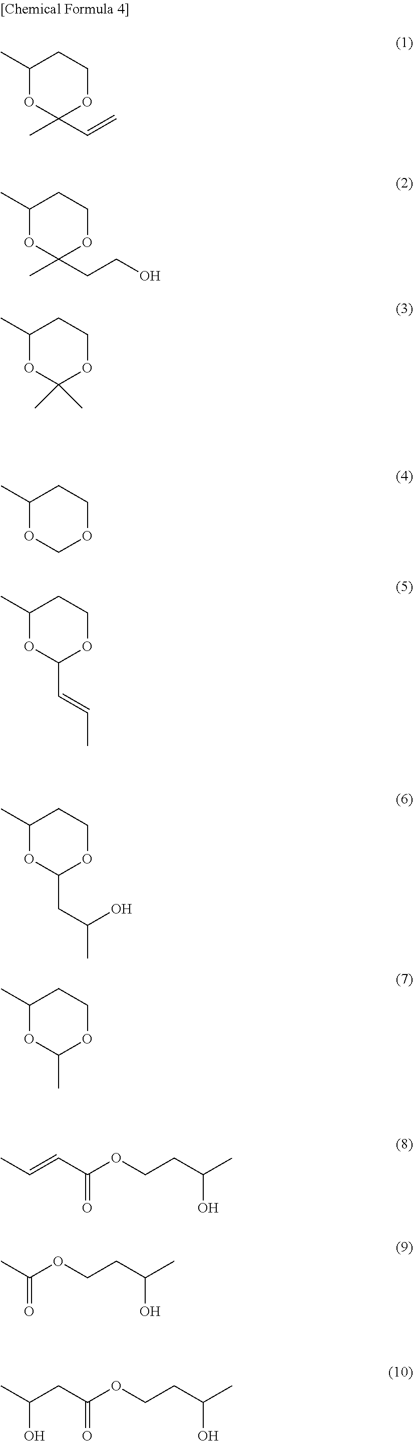

In the 1,3-butylene glycol product, the sum of the contents of acetaldehyde, crotonaldehyde, methyl vinyl ketone, acetone, formaldehyde, butylaldehyde, acetaldol, 1-hydroxy-3-butanone, 2-butanol, the compound represented by Formula (1) above, the compound represented by Formula (2) above, the compound represented by Formula (3) above, the compound represented by Formula (4) above, the compound represented by Formula (5) above, the compound represented by Formula (6) above, the compound represented by Formula (7) above, the compound represented by Formula (8) above, the compound represented by Formula (9) above, and the compound represented by Formula (10) above is preferably less than 70 ppm, after the 1,3-butylene glycol product has been kept at 180 °C for 3 hours in air atmosphere.

In the 1,3-butylene glycol product, a sum of the contents of the compound represented by Formula (1) above, the compound represented by Formula (2) above, the compound represented by Formula (3) above, the compound represented by Formula (4) above, the compound represented by Formula (5) above, the compound represented by Formula (6) above, and the compound represented by Formula (7) above is preferably less than 28 ppm.

Further, in the 1,3-butylene glycol product, the sum of the contents of the compound represented by Formula (1) above, the compound represented by Formula (2) above, the compound represented by Formula (3) above, the compound represented by Formula (4) above, the compound represented by Formula (5) above, the compound represented by Formula (6) above, and the compound represented by Formula (7) above is preferably less than 40 ppm, after the 1,3-butylene glycol product has been kept at 180 °C for 3 hours in air atmosphere.

In addition, the present disclosure also provides a moisturizer containing the 1,3-butylene glycol product.

Furthermore, the present disclosure also provides a cosmetic product containing the moisturizer.

In the present disclosure, “1,3-butylene glycol product” means a composition in which 1,3-butylene glycol accounts for a majority among the components (e.g., a 1,3-butylene glycol content is 95 wt. % or greater, preferably 98 wt. % or greater).

### Advantageous Effects of Invention

According to the producing method of the first aspect of the present disclosure, it is possible to produce high-purity 1,3-butylene glycol having a very low content of a high boiling point component and a low dry point, with a high recovery ratio.

In addition, the 1,3-butylene glycol product of the first aspect of the present disclosure has a very low content of a high boiling point component, a low dry point, and high purity. Therefore, it is suitably used as a moisturizer and in an application as a raw material for cosmetics.

Further, the moisturizer and cosmetic product of the first aspect of the present disclosure are excellent in moisturizing performance and have an extremely low content of a high boiling point component, and thus can maintain high quality for a long period of time.

According to the production method of the second aspect of the present disclosure, it is possible to produce high-purity 1,3-butylene glycol having a very low content of a low boiling point component and a high initial boiling point, with a high recovery ratio.

In addition, the 1,3-butylene glycol product of the second aspect of the present disclosure has a very low content of the low boiling point component a high initial boiling point, and high purity. Therefore, it is suitably used as a moisturizer and in an application as a raw material for cosmetics.

Further, the moisturizer and cosmetic product of the second aspect of the present disclosure are excellent in moisturizing performance and have an extremely low content of the low boiling point component, and thus can maintain high quality for a long period of time.

The manufacturing method of the third aspect of the present disclosure is capable of manufacturing 1,3-butylene glycol having a high potassium permanganate test value.

The 1,3-butylene glycol product of the third aspect of the present disclosure has a high potassium permanganate test value. Therefore, it is suitably used as a moisturizer, and in an application as a raw material for cosmetics.

Further, the moisturizers and cosmetic product of the third aspect of the present disclosure are excellent in moisturizing performance and have an extremely low content of reducing substances, and thus can maintain high quality for a long period of time.

According to the manufacturing method of the fourth aspect of the present disclosure, it is possible to industrially efficiently manufacture high-purity 1,3-butylene glycol having a high potassium permanganate test value, a very low content of low boiling point components, and a high initial boiling point.

Further, the 1,3-butylene glycol product of the fourth aspect of the present disclosure has a high potassium permanganate test value, a very low content of low boiling point components, a high initial boiling point, and high purity. Therefore, it is suitably used as a moisturizer, and in an application as a raw material for cosmetics.

Further, the moisturizer and cosmetic product of the fourth aspect of the present disclosure are excellent in moisturizing performance and have an extremely low content of reducing materials and low boiling point components, and thus can maintain high quality for a long period of time.

The manufacturing method of the fifth aspect of the present disclosure can be used to industrially efficiently manufacture a high-purity 1,3-butylene glycol having a very low content of low boiling point components and high boiling point components, a high initial boiling point, and a low dry point, and further high-purity 1,3-butylene glycol having a high potassium permanganate test value, a very low content of low boiling point components and high boiling point components, a high initial boiling point, and a low dry point.

Also, the 1,3-butylene glycol product of the fifth aspect of the present disclosure has a very low content of low boiling point components and high boiling point components, a high initial boiling point, a low dry point, and high purity. Further, the 1,3-butylene glycol product of the present disclosure has a high potassium permanganate test value. Therefore, it is suitably used as a moisturizer, and in an application as a raw material for cosmetics.

Further, the moisturizers and cosmetic product of the fifth aspect of the present disclosure are excellent in moisturizing performance, have an extremely low content of low boiling point components and high boiling point components, and preferably have an extremely low content of reducing substances, and thus can maintain high quality for a long period of time.

The manufacturing method of the sixth aspect of the present disclosure can be used to industrially efficiently manufacture high-purity 1,3-butylene glycol having a very low content of low boiling point components and high boiling point components, a high initial boiling point, and a low dry point, high-purity 1,3-butylene glycol having a very low content of low boiling point components, a high initial boiling point, a high potassium permanganate test value, and further high-purity 1,3-butylene glycol having a high potassium permanganate test value, a very low content of low boiling point components and high boiling point components, a high initial boiling point, and a low dry point.

According to the seventh aspect of the present disclosure, a high-purity 1,3-butylene glycol product that is colorless and odorless (or almost colorless and odorless), unlikely to cause coloration and odor over time, and, besides, unlikely to cause an acid concentration increase over time also in a state containing water is provided.

Furthermore, the present disclosure provides a moisturizer and a cosmetic product that have excellent moisturizing performance and can retain high quality for a long period of time.

Further, the method for producing 1,3-butylene glycol of the present disclosure can be used to industrially efficiently produce high-purity 1,3-butylene glycol having properties of being colorless and odorless (or almost colorless and odorless), unlikely to cause coloration over time, and, besides, unlikely to cause an acid concentration increase over time also in a state containing water.

The eighth aspect of the present disclosure provides a high-purity 1,3-butylene glycol product that is colorless and odorless (or almost colorless and odorless), unlikely to cause or increase coloration and odor over time, and/or unlikely to cause acid concentration increase over time even in a state containing water.

Furthermore, the present disclosure provides a moisturizer and a cosmetic product that have excellent moisturizing performance and can retain high quality for a long period of time.

The ninth aspect of the present disclosure provides a high-purity 1,3-butylene glycol product that is colorless and odorless (or almost colorless and odorless), and hardly causes or increases odor over time.

The present disclosure also provides a high-purity 1,3-butylene glycol product that is colorless and odorless (or almost colorless and odorless), hardly causes or increases coloration and odor over time, and/or is unlikely to cause an acid concentration increase over time also in a state containing water.

Furthermore, the present disclosure provides a moisturizer and a cosmetic product that have excellent moisturizing performance and can retain high quality for a long period of time.

### BRIEF DESCRIPTION OF DRAWINGS

FIG. 1 is a flowchart illustrating a producing method (purification method) for a 1,3-butylene glycol product of the first aspect of the present disclosure.

FIG. 2 is a chromatogram showing a chart for gas chromatographic analysis of a 1,3-butylene glycol product in Example 10 of the first aspect of the present disclosure.

FIG. 3 is a chromatogram showing a chart for gas chromatographic analysis of a 1,3-butylene glycol product in Comparative Example 2 of the first aspect of the present disclosure.

FIG. 4 is a flowchart illustrating a producing method (purification method) for a 1,3-butylene glycol product of the seventh aspect of the present disclosure.

FIG. 5 is a chromatogram showing a gas chromatographic analysis for a 1,3-butylene glycol product in Example 1 of the seventh aspect of the present disclosure.

FIG. 6 is a chromatogram showing a gas chromatographic analysis for a 1,3-butylene glycol product in Example 12 of the seventh aspect of the present disclosure.

FIG. 7 is a chromatogram showing a gas chromatographic analysis for a 1,3-butylene glycol product in Comparative Example 2 of the seventh aspect of the present disclosure.

### DESCRIPTION OF EMBODIMENTS

### [Method for Producing 1,3-Butylene Glycol]

The method for producing 1,3-butylene glycol of the first aspect of the present disclosure is a method for producing 1,3-butylene glycol to yield purified 1,3-butylene glycol from a reaction crude liquid containing 1,3-butylene glycol (1,3 BG) (hereinafter sometimes referred to as “crude 1,3-butylene glycol”), the method including: performing dehydration including removing water by distillation, removing high boiling point component including removing a high boiling point component by distillation, and performing product distillation to yield purified 1,3-butylene glycol. In a high boiling point component removal column for use in the removing a high boiling point component, a charged liquid containing 1,3-butylene glycol is distilled under a condition that a reflux ratio is greater than 0.02 in a case where a concentration of 1,3-butylene glycol in the charged liquid is 95% or less, or a reflux ratio is greater than 0.01 in a case where the concentration of 1,3-butylene glycol in the charged liquid is greater than 95%, high-purity 1,3-butylene glycol is distilled off from above a charging plate, and a liquid in which a high boiling point component is concentrated is extracted from below the charging plate.

The method for producing 1,3-butylene glycol of the second aspect of the present disclosure is a method for producing 1,3-butylene glycol to yield purified 1,3-butylene glycol from a reaction crude liquid containing 1,3-butylene glycol (1,3 BG) (hereinafter sometimes referred to as “crude 1,3-butylene glycol”), the method including: performing dehydration including removing water by distillation, removing high boiling point component including removing a high boiling point component by distillation, and performing product distillation to yield purified 1,3-butylene glycol. In the product column for use in the product distillation, a charged liquid containing 1,3-butylene glycol is distilled under a condition of a reflux ratio of greater than 0.1, a liquid in which the low boiling point component is concentrated is distilled off from above a charging plate, and 1,3-butylene glycol is extracted from below the charging plate. The 1,3-butylene glycol has a very low content of the low boiling point component and a high initial boiling point, and thus can be made into a 1,3-butylene glycol product.

The method for manufacturing 1,3-butylene glycol of the third aspect of the present disclosure is a method for manufacturing 1,3-butylene glycol, in which purified 1,3-butylene glycol is obtained from a crude reaction liquid containing 1,3-butylene glycol (1,3 BG) (hereinafter sometimes referred to as “crude 1,3-butylene glycol”), the method including: a dehydration step of removing water by distillation; a high boiling substance removal step of removing a high boiling point component by distillation; and a product distillation step of obtaining purified 1,3-butylene glycol. In the product distillation step, a product column is used in which a liquid feed having a 1,3-butylene glycol concentration of 97% or higher, an acetaldehyde content of 500 ppm or lower, and a crotonaldehyde content of 200 ppm or lower is distilled under a condition of a reflux ratio of higher than 0.1, and a liquid concentrated with acetaldehyde and crotonaldehyde is distilled off from above a feed plate, and 1,3-butylene glycol is extracted from below the feed plate. The 1,3-butylene glycol has a high potassium permanganate test value, and thus can be made into a 1,3-butylene glycol product.

The manufacturing method of the fourth aspect of the present disclosure is a method for manufacturing 1,3-butylene glycol, in which purified 1,3-butylene glycol is obtained from a crude reaction liquid containing 1,3-butylene glycol (1,3 BG) (hereinafter sometimes referred to as “crude 1,3-butylene glycol”), the method including: a dehydration step of removing water by distillation; a high boiling substance removal step of removing a high boiling point component by distillation; and a product distillation step of obtaining purified 1,3-butylene glycol. In a dehydration column for use in the dehydration step, a liquid feed containing 1,3-butylene glycol and water with an acetaldehyde content of 1000 ppm or lower and a crotonaldehyde content of 400 ppm or lower is distilled under a condition of a reflux ratio of higher than 0.3, and a liquid concentrated with a low boiling point component containing water is distilled off from above a feed plate. In a product column for use in the product distillation step, a 1,3-butylene glycol liquid feed with an acetaldehyde content of 500 ppm or lower and a crotonaldehyde content of 200 ppm or lower is distilled under a condition of a reflux ratio of higher than 0.1, a liquid concentrated with a low boiling point component is distilled off from above a feed plate, and 1,3-butylene glycol is extracted from below the feed plate. The thus obtained 1,3-butylene glycol can be made into a 1,3-butylene glycol product since it has a high potassium permanganate test value, a very low content of low boiling point components, and a high initial boiling point.

A manufacturing method 1 according to the fifth aspect of the present disclosure is a method for manufacturing 1,3-butylene glycol, in which purified 1,3-butylene glycol is obtained from a crude reaction liquid containing 1,3-butylene glycol (1,3 BG) (hereinafter sometimes referred to as “crude 1,3-butylene glycol”), the method including: a dehydration step of removing water by distillation, a high boiling substance removal step of removing a high boiling point component by distillation, and a product distillation step of obtaining purified 1,3-butylene glycol. In the dehydration step, a dehydration column is used in which a liquid feed containing 1,3 butylene glycol and water with an acetaldehyde (AD) content of 1000 ppm or lower and a crotonaldehyde (CR) content of 400 ppm or lower is distilled under a condition of a reflux ratio of higher than 0.3, and a liquid concentrated with a low boiling point component containing water is distilled off from above a feed plate. In the high boiling substance removal step, a high boiling substance removal column is used in which a liquid feed containing 1,3-butylene glycol is distilled under a condition of a reflux ratio of higher than 0.02, and higher-purity 1,3-butylene glycol is distilled off from above a feed plate, and a liquid concentrated with a high boiling point component is extracted from below the feed plate. In the product distillation step, a product column is used in which a 1,3-butylene glycol liquid feed with an acetaldehyde content of 500 ppm or lower and a crotonaldehyde content of 200 ppm or lower is distilled under a condition of a reflux ratio of higher than 0.1, a liquid concentrated with a low boiling point component is distilled off from above a feed plate, and 1,3-butylene glycol is extracted from below the feed plate. The thus obtained 1,3-butylene glycol can be made into a 1,3-butylene glycol product since it has a high potassium permanganate test value, a very low content of low boiling point components and high boiling point components, a high initial boiling point, and a low dry point.

The manufacturing method 2 of the fifth aspect of the present disclosure is a method for manufacturing 1,3-butylene glycol, in which purified 1,3-butylene glycol is obtained from a crude reaction liquid containing 1,3-butylene glycol (crude 1,3-butylene glycol), the method including: a dehydration step of removing water by distillation and a high boiling substance removal step of removing a high boiling point component by distillation. In the dehydration step, a dehydration column is used in which a liquid feed containing 1,3 butylene glycol and water with an acetaldehyde content of 1000 ppm or lower and a crotonaldehyde content of 400 ppm or lower is distilled under a condition of a reflux ratio of higher than 0.3, and a liquid concentrated with a low boiling point component containing water is distilled off from above a feed plate. In the high boiling substance removal step, a high boiling substance removal column is used in which a liquid feed containing 1,3-butylene glycol is distilled under a condition of a reflux ratio of higher than 0.02, higher-purity 1,3-butylene glycol is distilled off from above a feed plate, and a liquid concentrated with the high boiling point component is extracted from below the feed plate. Since the thus obtained 1,3-butylene glycol has a very low content of low boiling point components and high boiling point components, it can be subjected to alkaline treatment as necessary, and thus made into a 1,3-butylene glycol product.

The “manufacturing method 1 of the fifth aspect of the present disclosure” and “manufacturing method 2 of the present disclosure” are sometimes collectively referred to as the “manufacturing method of the present disclosure”.

A manufacturing method 1 according to the sixth aspect of the present disclosure is a method for manufacturing 1,3-butylene glycol, in which purified 1,3-butylene glycol is obtained from a crude reaction liquid containing 1,3-butylene glycol (1,3 BG) (hereinafter sometimes referred to as “crude 1,3-butylene glycol”), the method including: a dehydration step of removing water by distillation, a high boiling substance removal step of removing a high boiling point component by distillation, and a product distillation step of obtaining purified 1,3-butylene glycol. In a dehydration column for use in the dehydration step, a liquid feed containing 1,3-butylene glycol and water with a content of acetaldehyde (AD) of 1000 ppm or lower and a content of crotonaldehyde (CR) of 400 ppm or lower is distilled, and a liquid concentrated with a low boiling point component containing water is distilled off from above a feed plate. In the product distillation step, a product column is used in which a 1,3-butylene glycol liquid feed having an acetaldehyde content of 500 ppm or lower and a crotonaldehyde content of 200 ppm or lower is distilled under a condition of a reflux ratio of higher than 0.1, a liquid concentrated with a low boiling point component is distilled off from above a feed plate, and 1,3-butylene glycol is extracted from below the feed plate. The thus obtained 1,3-butylene glycol can be made into a 1,3-butylene glycol product since it has a high potassium permanganate test value, a very low content of low boiling point components and high boiling point components, a high initial boiling point, and a low dry point.

The manufacturing method 2 of the sixth aspect of the present disclosure is a method for manufacturing 1,3-butylene glycol, in which purified 1,3-butylene glycol is obtained from a crude reaction liquid containing 1,3-butylene glycol (crude 1,3-butylene glycol), the method including: a dehydration step of removing water by distillation and a high boiling substance removal step of removing a high boiling point component by distillation. In a dehydration column for use in the dehydration step, a liquid feed containing 1,3-butylene glycol and water with an acetaldehyde content of 1000 ppm or lower and a crotonaldehyde content of 400 ppm or lower is distilled, and a liquid concentrated with a low boiling point component containing water is distilled off from above a feed plate. In a high boiling substance removal column for use in the high boiling substance removal step, a liquid feed containing 1,3-butylene glycol is distilled under a condition of a reflux ratio of higher than 0.02, higher-purity 1,3-butylene glycol is distilled off from above a feed plate, and a liquid concentrated with the high boiling point component is extracted from below the feed plate. Since the thus obtained 1,3-butylene glycol has a very low content of low boiling point components and high boiling point components, it can be subjected to alkaline treatment as necessary, and thus made into a 1,3-butylene glycol product.

The “manufacturing method 1 of the sixth aspect of the present disclosure” and “manufacturing method 2 of the present disclosure” are sometimes collectively referred to as the “manufacturing method of the present disclosure”.

### Method for Producing 1,3-Butylene Glycol

The 1,3-butylene glycol product according to the seventh aspect of the present disclosure can be produced by the producing method of the present disclosure.

The 1,3-butylene glycol product of the eighth and ninth aspects of the present disclosure can be produced, for example, by a method 1 of producing 1,3-butylene glycol which will be described below, or a method 2 of producing 1,3-butylene glycol which will be described below.

### Method 1 of Producing 1,3-Butylene Glycol:

A method for producing 1,3-butylene glycol to yield purified 1,3-butylene glycol from a reaction crude liquid containing 1,3-butylene glycol,
the method including: performing dehydration including removing water by distillation, removing a high boiling point component including removing a high boiling point component by distillation, and performing product distillation to yield purified 1,3-butylene glycol,
wherein, in a product column for use in the performing product distillation, a 1,3-butylene glycol charged liquid having a content of acetaldehyde of 500 ppm or less, a content of crotonaldehyde of 200 ppm or less, a content of water of 0.7 wt. % or less, and a concentration of 1,3-butylene glycol of 97.6 area % by gas chromatographic analysis under the following conditions is subjected to distillation under a condition that a reflux ratio is 0.3 or greater.

The conditions for the gas chromatographic analysis are as follows:
Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

### Method 2 of Producing 1,3-Butylene Glycol:

A method for producing 1,3-butylene glycol to yield purified 1,3-butylene glycol from a reaction crude liquid containing 1,3-butylene glycol,
the method including: performing dehydration including removing water by distillation and removing a high boiling point component including removing a high boiling point component by distillation,
wherein, in a high boiling point component removal column for use in the removing a high boiling point component, a charged liquid containing 1,3-butylene glycol, which has a content of acetaldehyde of 500 ppm or less, a content of crotonaldehyde of 200 ppm or less, a content of water of 3 wt. % or less, and a concentration of 1,3-butylene glycol of 96.7 area % or greater by gas chromatographic analysis under the following conditions is subjected to distillation under a condition that a reflux ratio is 0.03 or greater.

The conditions for the gas chromatographic analysis are as follows:
same as in the method 1 of producing 1,3-Butylene glycol.

The producing method 1 of the seventh, eighth and ninth aspects of the present disclosure is a method for producing 1,3-butylene glycol to yield purified 1,3-butylene glycol from a reaction crude liquid containing 1,3-butylene glycol (1,3 BG) (hereinafter, sometimes referred to as “crude 1,3-butylene glycol”), the method including: performing dehydration including removing water by distillation, removing a high boiling point component including removing a high boiling point component by distillation, and performing product distillation to yield purified 1,3-butylene glycol. Then, in the product column for use in the performing product distillation, a 1,3-butylene glycol charged liquid is subjected to distillation under a condition that a reflux ratio is 0.3 or greater, the 1,3-butylene glycol charged liquid having a content of acetaldehyde of 500 ppm or less, a content of crotonaldehyde of 200 ppm or less, a content of water of 0.7 wt. % or less, and a concentration of 1,3-butylene glycol of 97.6 area % according to the gas chromatographic analysis performed under conditions set forth below. A liquid with a concentrated low boiling point component is distilled from above a charging plate, and 1,3-butylene glycol is extracted from below the charging plate. The thus-produced 1,3-butylene glycol is colorless and odorless (or almost colorless and odorless), unlikely to cause or increase coloration and odor over time, and, besides, unlikely to cause an acid concentration increase over time even in a state that the 1,3-butylene glycol contains water. Thus, such 1,3-butylene glycol can constitute a 1,3-butylene glycol product. Also, the thus-obtained 1,3-butylene glycol is colorless and odorless (or almost colorless and odorless), unlikely to cause or increase coloration and odor over time, and, in addition, unlikely to cause an acid concentration increase over time also in a state containing water, and thus can constitute a 1,3-butylene glycol product.

The conditions for the gas chromatographic analysis are as follows:
Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

The producing method 2 of the seventh, eighth and ninth aspects of the present disclosure is a method for producing 1,3-butylene glycol to yield purified 1,3-butylene glycol from a reaction crude liquid containing 1,3-butylene glycol, the method including: performing dehydration including removing water by distillation and removing a high boiling point component including removing a high boiling point component by distillation. In a high boiling point component removal column for use in the removing a high boiling point component, a charged liquid containing 1,3-butylene glycol is subjected to distillation under a condition that a reflux ratio is 0.03 or greater, the charged liquid having a content of acetaldehyde of 500 ppm or less, a content of crotonaldehyde of 200 ppm or less, a content of water of 3 wt. % or less, and a concentration of 1,3-butylene glycol of 96.7 area % or greater according to the gas chromatographic analysis performed under conditions set forth below. The higher-purity 1,3-butylene glycol is distilled from above the charging plate, and a liquid in which a high boiling point component is concentrated is extracted from below the charging plate. The thus-produced 1,3-butylene glycol is colorless and odorless (or almost colorless and odorless), unlikely to cause or increase coloration and odor over time, and, besides, unlikely to cause an acid concentration increase over time even in a state that the 1,3-butylene glycol contains water. Thus, such 1,3-butylene glycol can constitute a 1,3-butylene glycol product. Also, the thus-produced 1,3-butylene glycol is colorless and odorless (or almost colorless and odorless), hardly causes or increases coloration over time, and, besides, is unlikely to cause an acid concentration increase over time even in a state that the 1,3-butylene glycol contains water. Thus, such 1,3-butylene glycol can constitute a 1,3-butylene glycol product. Also, the thus-obtained 1,3-butylene glycol is colorless and odorless (or almost colorless and odorless), unlikely to cause or increase coloration and odor over time, and, in addition, unlikely to cause an acid concentration increase over time also in a state containing water, and thus can constitute a 1,3-butylene glycol product.

The “producing method 1 of the present disclosure” and the “producing method 2 of the present disclosure” are collectively referred to as “producing method of the present disclosure” in some cases.

[Crude 1,3-butylene glycol]

Examples of the crude 1,3-butylene glycol include (1) a reaction crude liquid produced by reduction (hydrogenation) of acetaldols, (2) a reaction crude liquid produced by hydrolysis of 1,3-butylene oxide, (3) a reaction crude liquid produced by selective hydrocracking of erythritol, (4) a reaction crude liquid produced by selective hydration to butadiene, (5) a reaction crude liquid produced by hydrogen addition to n-butanal-3-one, (6) a reaction crude liquid produced by hydrogen addition to 1-butanol-3-one, (7) a reaction crude liquid produced by hydrogen addition to 3-hydroxy-1-butanoic acid, (8) a reaction crude liquid produced by hydrogen addition to β-butyrolactone, and (9) a reaction crude liquid produced by hydrogen addition to diketene. In the present disclosure, the crude 1,3-butylene glycol may be one type or a mixture of two or more types of the above (1) to (9). The crude 1,3-butylene glycol is preferably (1) the reaction crude liquid produced by reduction (in particular, liquid phase reduction) of acetaldols.

Hereinafter, a case where the reaction crude liquid produced by reduction (hydrogenation) of acetaldols is used as the crude 1,3-butylene glycol will be mainly described. Note that the reduction (hydrogenation) of acetaldols is sometimes referred to as “hydrogenation”.

The acetaldols used as a raw material in the hydrogenation is not particularly limited as long as it is a compound that is converted to 1,3-butylene glycol by hydrogen reduction. Examples of the raw material acetaldols include acetaldol; its cyclic dimer paraldol; aldoxane as a cyclic trimer of acetaldehyde; and mixtures of these.

The method of manufacturing the acetaldols (e.g., acetaldol or paraldol) is not particularly limited, but the acetaldols may be, for example, those produced by an aldol condensation reaction of acetaldehyde in the presence of a basic catalyst or those produced by pyrolysis or the like of aldoxane. Note that the production of the acetaldols is sometimes referred to as “acetaldol production” or “acetaldehyde polymerization”.

A reaction crude liquid produced by the reaction described above and containing acetaldols may be neutralized with an acid and used in the manufacturing of 1,3-butylene glycol. Such a reaction crude liquid may contain, in addition to acetaldols, acetaldehyde (AD), crotonaldehyde (CR), another aldehyde component; a low boiling point substance; a high boiling point substance, such as an aldehyde dimer or trimer; water; a salt; and the like. In the present specification, a compound with a lower boiling point than that of 1,3-butylene glycol may be referred to as a “low boiling point substance” or “low boiling substance”, and a compound with a higher boiling point than that of 1,3-butylene glycol may be referred to as a “high boiling point substance” or “high boiling substance”.

The reaction crude liquid containing acetaldols may be subjected to a pretreatment, such as dealcoholization distillation, dehydration distillation, desalting, alkaline treatment and dealkalization treatment, or impurity removal, as necessary, and a liquid produced by removing by-products, such as unreacted acetaldehyde and crotonaldehyde, may be used. Examples of the pretreatment method include distillation, adsorption, ion exchange, conversion to a high boiling point substance by heating, and decomposition. For the distillation, a distillation method of various types, such as reduced pressure, normal pressure, increased pressure, azeotropic, extraction, or reaction, can be used. In particular, it is preferred that the reaction crude liquid containing acetaldols is subjected to simple evaporation, distillation, or hydrogen addition to remove aldehydes such as acetaldehyde and crotonaldehyde, followed by the hydrogenation.

The content of the acetaldols in the raw material for hydrogenation is not particularly limited but is, for example, preferably 30 wt. % or greater (e.g., from 30 to 99 wt. %), more preferably 40 wt. % or greater (for example, from 40 to 98 wt. %), 50 wt. % or greater (for example, from 50 to 97 wt. %) or 60 wt. % or greater (for example, from 60 to 95 wt. %), and even more preferably from 65 to 90 wt. %, particularly preferably from 70 to 90 wt. %, and most preferably from 75 to 90 wt. %. With the content of the acetaldols within the above ranges, impurities contained in the reaction crude liquid containing 1,3-butylene glycol (crude 1,3-butylene glycol) tend to be reduced.

The raw material for hydrogenation may or may not contain water but preferably contains water from the viewpoint of the purity of 1,3-butylene glycol product. The water content in the raw material for hydrogenation is not particularly limited but is, for example, preferably 2 wt. % or greater, more preferably 5 wt. % or greater, even more preferably 10 wt. % or greater, and particularly preferably 15 wt. % or greater. The upper limit may be, for example, 90 wt. %, 80 wt. %, 70 wt. %, 60 wt. %, 50 wt. %, 40 wt. %, 30 wt. % or 20 wt. %. With the water content within the above ranges, the acetal of 1,3-butylene glycol and acetaldol contained in the resulting crude 1,3-butylene glycol is reduced, and thus this tends to increase the purity of the 1,3-butylene glycol product finally produced. This is because the raw material for hydrogenation contains water to a certain extent, and the acetal is hydrolyzed into 1,3-butylene glycol accordingly as well as coexisting acetaldol is reduced into 1,3-butylene glycol.

Examples of the hydrogenation catalyst include Raney nickel. The hydrogenation catalyst can also be used in a suspended state, and can also be filled in a reaction vessel and used. The amount of the hydrogenation catalyst to be used is not particularly limited but is, for example, preferably from 1 to 30 parts by weight, more preferably from 4 to 25 parts by weight, even more preferably from 8 to 20 parts by weight, and particularly preferably from 12 to 18 parts by weight relative to 100 parts by weight of the raw material for hydrogenation. The amount of hydrogen to be used in the reduction reaction is not particularly limited but is, for example, preferably from 0.5 to 40 parts by weight, more preferably from 1 to 30 parts by weight, even more preferably from 4 to 20 parts by weight, and particularly preferably from 8 to 12 parts by weight relative to 100 parts by weight of the raw material for hydrogenation. A pressure (total pressure; gauge pressure) in a reaction system in the reduction reaction is not particularly limited but is, for example, preferably from 9 to 70 MPa and more preferably from 10 to 40 MPa. A hydrogen pressure (partial pressure of hydrogen) in the reaction system is not particularly limited, but is, for example, from 7 to 60 MPa, and preferably from 10 to 30 MPa. Note that, from the perspective of reducing the reducing materials such as acetaldehyde and crotonaldehyde, it is better to increase the hydrogen pressure in the reaction system, and the hydrogen pressure is preferably 10 MPa or higher, and may be 100 MPa. The higher hydrogen pressure in the reaction system makes acetaldehyde, crotonaldehyde, and other reducing substances essentially harmless, and the concentrations of acetaldehyde, crotonaldehyde, and other reducing substances, as well as other impurities such as trace acetals in the liquid feed into the dehydration column, which will be described later, can also be reduced. Thus, the product quality can be advantageously maintained even when the reflux ratios and distilled amounts of the dehydration column, high boiling substance removal column, and product column in the 1,3-butylene glycol purification are relatively small. A reaction temperature in the reduction reaction is not particularly limited but is, for example, from 40 to 150 °C, preferably from 50 to 140 °C, and more preferably from 60 to 130 °C. A reaction time (residence time) in the reduction reaction is not particularly limited but is, for example, from 10 to 500 minutes, preferably from 20 to 400 minutes, more preferably from 30 to 300 minutes, even more preferably from 50 to 280 minutes, and particularly preferably from 80 to 250 minutes. The present reaction can be carried out in any of a batch, semi-batch, or continuous manner.

For example, the thus-produced crude 1,3-butylene glycol contains a low boiling point substance (low boiling point compound) having an unsaturated bond, such as acetaldehyde (AD), butylaldehyde, crotonaldehyde (CR), acetone, and methyl vinyl ketone; a condensate of these; a condensate of 1,3-butylene glycol and the low boiling point substance above (e.g., an acetal of 1,3-butylene glycol and acetaldol); an alcohol such as ethanol, isopropyl alcohol, or butanol; water (solvent and the like), a salt produced by neutralization or the like, a catalyst (when used in suspension) or the like. Removal of these impurities in the purification can yield a 1,3-butylene glycol product (purified 1,3-butylene glycol).

### [Purification of Crude 1,3-Butylene Glycol]

The producing method according to the present disclosure includes, at least, performing dehydration including removing water by distillation, removing high boiling point component (high boiling point substance removal distillation) including removing a high boiling point component by distillation, and performing product distillation to yield purified 1,3-butylene glycol. The production method 1 according to the present disclosure includes, at least, performing dehydration including removing water by distillation, removing a high boiling point component including removing a high boiling point component by distillation (high boiling point component removal distillation), and performing product distillation to yield purified 1,3-butylene glycol. In the production method of the present disclosure, the order of the performing dehydration and the removing a high boiling point component does not matter. The dehydration and the high boiling point component removal are both provided before the product distillation, but the order of the dehydration and the high boiling point component removal is not particularly limited. The producing method according to the present disclosure may include, in addition to these steps, desalting, alkaline reaction (alkaline treatment), and dealkalization. Additionally, prior to the dehydration, catalyst separation, neutralization by alkali, dealcoholization (low boiling point component removal), and the like can be provided. These steps may be performed in the order described above, but the order of the steps may be changed as appropriate except that the dealkalization is provided after the alkaline reaction. For example, the dealcoholization (low boiling point component removal), the desalting, the alkaline reaction, and the dealkalization can be provided at appropriate points, but are usually provided after the hydrogenation. Note that, among the steps described above, the catalyst separation, the neutralization by alkali, the dealcoholization (low boiling point component removal), the desalting, the alkaline reaction, and the dealkalization may be provided as necessary, and do not necessarily have to be provided.

The manufacturing method 1 according to the fifth and sixth aspects of the present disclosure includes, at least, a dehydration step of removing water by distillation, a high boiling substance removal step of removing a high boiling point component by distillation (high boiling substance removal distillation), and a product distillation step of obtaining purified 1,3-butylene glycol. In the manufacturing method 2 of the fifth and sixth aspects of the present disclosure includes, at least a dehydration step of removing water by distillation and a high boiling substance removal step of removing a high boiling point component by distillation (high boiling point substance removal distillation). In the manufacturing method of the fifth and sixth aspects of the present disclosure, the order of the dehydration step and the high boiling substance removal step does not matter. In the manufacturing method 1 of the fifth and sixth aspects of the present disclosure, both the dehydration step and the high boiling substance removal step are provided prior to the product distillation step.

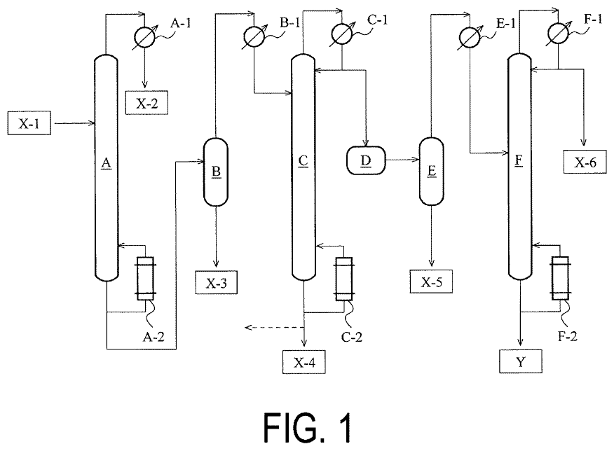

FIG. 1 is a flow sheet of an apparatus illustrating an example of an embodiment of a method for producing 1,3-butylene glycol according to the present disclosure. A is a dehydration column and is related to the dehydration. B is a desalting column and is related to the desalting. C is a distillation column for removing a high boiling point substance (high boiling point component removal column) and is related to the distillation for removal of a high boiling point substance (high boiling point component removal). D is an alkaline reactor and is related to the alkaline reaction. E is a dealkalization column and is related to the dealkalization. F is a product distillation column (product column) and is related to the product distillation. A-1, B-1, C-1, E-1, and F-1 are condensers. A-2, C-2, and F-2 are reboilers. Hereinafter, an example of an embodiment of a method for producing 1,3-butylene glycol according to the present disclosure will be described using the present flow sheet.

Crude 1,3-butylene glycol (corresponding to “X-1”) produced by hydrogen reduction of a raw material for hydrogenation is fed to the dehydration column A. In the dehydration column A, water is distilled off from the top of the column by distillation, and from the bottom of the column, a crude 1,3-butylene glycol stream containing 1,3-butylene glycol is produced. Note that the crude 1,3-butylene glycol (corresponding to “X-1”) may undergo the dealcoholization (distillation by a dealcoholization column) for removal of an alcohol such as ethanol and a low boiling point substance, and then may be supplied to the dehydration column A.

The crude 1,3-butylene glycol stream is fed to the desalting column B. In the desalting column B, the crude 1,3-butylene glycol stream after the desalting is produced from the top of the column, and a salt, a high boiling point substance, or the like is discharged from the bottom of the column. The bottom ratio (%) of the desalting column B [(desalting column bottom amount (part)/desalting column charge amount (part)×100] is, for example, from 0.1 to 40 wt. %, preferably from 1 to 35 wt. %, more preferably from 2 to 30 wt. %, even more preferably from 3 to 25 wt. %, and particularly preferably from 5 to 20 wt. %, and may be from 7 to 15 wt. %. At least a portion of the bottom in the desalting column may be recycled to the step prior to the desalting.

The crude 1,3-butylene glycol stream after the desalting described above or below is fed to the high boiling point component removal column C. In the high boiling point component removal column C, a high boiling point component (also referred to as high boiling point substance) is discharged from below the charging plate (preferably from the bottom of the column). On the other hand, the crude 1,3-butylene glycol stream after high boiling point substance removal (high-purity 1,3-butylene glycol) is produced from above the charging plate.

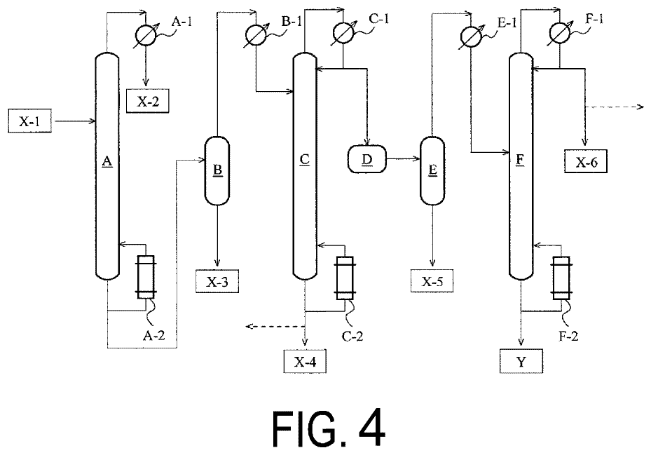

In the manufacturing method of the fourth and fifth aspects of the present disclosure, in the dehydration column A for use in the dehydration step, a liquid feed containing 1,3-butylene glycol and water with an acetaldehyde content of 1000 ppm or lower and a crotonaldehyde content of 400 ppm or lower is distilled under a condition of a reflux ratio of higher than 0.3, and a liquid concentrated with a low boiling point component containing water is distilled off from above a feed plate (preferably, top of the column) (corresponding to “X-2” in FIG. 1 of U.S. Patent Application Publication No. US2022/0354760 and US2023/0048638). Further, a crude 1,3-butylene glycol stream containing 1,3-butylene glycol can be obtained from below the feed plate (preferably, the bottom of the column). In the manufacturing method according to the sixth aspect of the present disclosure, similarly, in the dehydration column A for use in the dehydration step, a liquid feed containing 1,3-butylene glycol and water with an acetaldehyde content of 1000 ppm or lower and a crotonaldehyde content of 400 ppm or lower is distilled, and a liquid concentrated with a low boiling point component containing water is distilled off from above a feed plate (preferably, top of the column) (corresponding to “X-2” in FIG. 1 of U.S. Patent Application Publication No. US2023/0046811). Further, a crude 1,3-butylene glycol stream containing 1,3-butylene glycol can be obtained from below the feed plate (preferably, the bottom of the column). In the production method of the seventh aspect of the present disclosure, in the dehydration column A used in the dehydration, for example, a charged liquid containing 1,3-butylene glycol and water is subjected to distillation, and a liquid having a concentrated low boiling point component containing water is distilled from above the charging plate (preferably, the top of the column) (corresponding to “X-2” in FIG. 4). Further, a crude 1,3-butylene glycol stream containing 1,3-butylene glycol can be produced from below the charging plate (preferably, the bottom of the column).

The high boiling point component removal column C, the dehydration column A and any other distillation column for separating 1,3-butylene glycol can be, for example, a perforated-plate column, a bubble column, or the like, but is more preferably a packed column with a low pressure loss, filled with Sulzer Packing, Melapack (both are trade names of Sumitomo Heavy Industries, Ltd.). This is because 1,3-butylene glycol and trace impurities would be thermally decomposed at a high temperature (e.g., 150 °C or higher) and produce a low boiling point substance, which is a coloring component, and thus the distillation temperature is to be lowered. In addition, this is also because a long thermal history (residence time) for 1,3-butylene glycol would also have a similar effect. Thus, the reboiler employed is preferably one with a short residence time of the process side fluid, for example, a thin-film evaporator, such as a natural downward flow thin-film evaporator or a forced-stirring thin-film evaporator.

The number of theoretical plates of the dehydration column A is, for example, from 1 to 100 plates, preferably from 2 to 80 plates, from 3 to 80 plates, from 4 to 60 plates, from 5 to 40 plates, from 6 to 30 plates or from 7 to 20 plates, and more preferably from 8 to 15 plates. A feed position for the liquid feed is, for example, from 10 to 90%, preferably from 20 to 80%, and more preferably from 30 to 70%, and even more preferably from 40 to 60% of a height of the column facing downward from the top of the column. During the distillation in the dehydration column A, the pressure (absolute pressure) of the top of the column is, for example, 101 kPa or lower, preferably from 0.1 to 90 kPa, more preferably from 0.5 to 70 kPa, and even more preferably from 1 to 50 kPa, from 2 to 30 kPa or from 3 to 20 kPa, and particularly preferably from 4 to 10 kPa. Note that the distillation in the dehydration column A may be performed under pressure, in which case the pressure (gauge pressure) at the top of the column may be, for example, 0.2 MPaG or lower, or 0.1 MPaG or lower.

A concentration of 1,3-butylene glycol in the liquid feed into the dehydration column A is, for example, 9 wt. % or higher, preferably 10 wt. % or higher, more preferably 15 wt. % or higher, even more preferably 20 wt. % or higher, 25 wt. % or higher, 30 wt. % or higher, 35 wt. % or higher, 40 wt. % or higher, 45 wt. % or higher, 50 wt. % or higher, 55 wt. % or higher, or 60 wt. % or higher, and particularly preferably 70% or higher. An upper limit of the concentration of 1,3-butylene glycol in the liquid feed into the dehydration column A is, for example, 90 wt. %, 85 wt. %, or 80 wt. %. However, in consideration of the hydrogen addition reaction and the like in the step prior to the dehydration step, the concentration of water in the liquid feed into the dehydration column A is preferably higher in some cases. With these things taken into consideration, the concentration of 1,3-butylene glycol in the liquid feed into the dehydration column A may be, for example, 1 wt. % or higher, 5 wt. % or higher, 10 wt. % or higher, 15 wt. % or higher, 20 wt. % or higher, 25 wt. % or higher, 30 wt. % or higher, 35 wt. % or higher, 40 wt. % or higher, 50 wt. % or higher, 60 wt. % or higher, 70 wt. % or higher, 80 wt. % or higher, or 90 wt. % or higher. Furthermore, the concentration of 1,3-butylene glycol in liquid feed into the dehydration column A may be, for example, 99 wt. % or lower, 95 wt. % or higher, 90 wt. % or lower, 85 wt. % or lower, 80 wt. % or lower, 75 wt. % or lower, 70 wt. % or lower, 65 wt. % or lower, 60 wt. % or lower, 55 wt. % or lower, 50 wt. % or lower, or 45 wt. % or lower. The concentration of 1,3-butylene glycol in the liquid feed into the dehydration column A can be, for example, in the range described above by adjusting the reaction conditions in the hydrogenation (for example, the concentration of acetaldols used as a raw material) and the distillation conditions of the dealcoholization column (low boiling substance removal column) provided before the dehydration column as needed.

The concentration (wt. %) of 1,3-butylene glycol is a value determined according to the following formula by determining an area proportion (GC area %) of the peak of 1,3-butylene glycol relative to a total peak area in the gas chromatographic analysis under the following conditions. Note that the concentration (wt. %) of water in the liquid feed into the dehydration column A is a value measured by the method which will be described below (Karl Fischer method).

Concentration (wt. %) of 1,3-butylene glycol in liquid feed into dehydration column A=(1−concentration (wt. %) of water in liquid feed into dehydration column A/100)×GC area % of 1,3-butylene glycol described above,

### Conditions for the Gas Chromatographic Analysis are as Follows:

Analytical Column: a column with dimethylpolysiloxane as a stationary phase, having a length of 30 m, an inner diameter of 0.25 mm, and a film thickness of 1.0 μm
Heating Conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

In the manufacturing method of the present disclosure, the content of acetaldehyde in the liquid feed into the dehydration column A is 1000 ppm or lower, and the content of crotonaldehyde therein is 400 ppm or lower. The content of acetaldehyde in the liquid feed into the dehydration column A is preferably 900 ppm or lower, more preferably 800 ppm or lower, 700 ppm or lower, 600 ppm or lower, or 500 ppm or lower, and even more preferably 400 ppm or lower, 300 ppm or lower, 200 ppm or lower, 155 ppm or lower, or 140 ppm or lower, and may be 100 ppm or lower, 90 ppm or lower, 80 ppm or lower, 70 ppm or lower, 60 ppm or lower, 50 ppm or lower, 40 ppm or lower, or 30 ppm or lower, 20 ppm or lower, 10 ppm or lower, 5 ppm or lower, 3 ppm or lower, 2 ppm or lower, or 1 ppm or lower. In the production method of the other present disclosure, a content of acetaldehyde in the charged liquid into the dehydration column A is, for example, 1000 ppm or less.

The content of crotonaldehyde in the liquid feed into the dehydration column A is preferably 300 ppm or lower, more preferably 200 ppm or lower, and even more preferably 150 ppm or lower, 130 ppm or lower, 117 ppm or lower, or 100 ppm or lower, and may be 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, 3 ppm or lower, 2 ppm or lower, or 1 ppm or lower. The content of crotonaldehyde in charged liquid into the dehydration column A may be, for example, 400 ppm or less.

The acetaldehyde content and the crotonaldehyde content in the liquid feed into the dehydration column A can be reduced, for example, by providing a dealcoholization column (low boiling substance removal column) upstream of the dehydration column A, and adjusting the distillation conditions of the dealcoholization column (low boiling substance removal column). For example, increasing the reflux ratio and the number of plates, and the distillation rate in the dealcoholization column (low boiling substance removal column) can reduce the acetaldehyde content and the crotonaldehyde content of the liquid feed into the dehydration column A. Furthermore, the content can be adjusted according to the conditions for the hydrogen addition reaction in the hydrogenation step, and when hydrogen addition is fully performed, the concentrations of acetaldehyde and crotonaldehyde can be lowered to below the detection limit, but there are disadvantages such as a high reaction pressure, an increase in size of the reaction tank and the like.

Note that the acetaldehyde content and the crotonaldehyde content of the liquid feed into the dehydration column A can be quantified by GC-MS analysis (gas mass spectrometry).

In the manufacturing method of the present disclosure, the content of water in the liquid feed into the dehydration column A is, for example, 90 wt. % or lower, 85 wt. % or lower, 80 wt. % or lower, 70 wt. % or lower, 60 wt. % or lower, 50 wt. % or lower, or 40 wt. % or lower, and preferably 35 wt. % or lower, more preferably 30 wt. % or lower, and even more preferably 25 wt. % or lower. A lower limit of the water content in the liquid feed into the dehydration column A is, for example, 1 wt. %, 5 wt. %, 10 wt. % or 15 wt. %. Note that, when the hydrogen addition reaction in the hydrogenation step is taken into consideration, higher water concentration and lower the viscosity are advantageous for the hydrogen addition reaction because they increase the solubility and degree of dispersion of hydrogen in the liquid. The water content of the liquid feed into the dehydration column A can be reduced, for example, by providing a dealcoholization column (low boiling substance removal column) upstream of the dehydration column A, and adjusting the distillation conditions of the dealcoholization column (low boiling substance removal column). For example, increasing the reflux ratio and the number of plates, and the distillation rate in the dealcoholization column (low boiling substance removal column) can reduce the water content of the liquid feed into the dehydration column A. Note that the water content of the liquid feed into the dehydration column A can be quantified by the Karl Fischer moisture measurement instrument.

In the manufacturing method of the present disclosure, the content of the low boiling point component (excluding water) in the liquid feed into the dehydration column A is, for example, 20% or lower, preferably 10% or lower, more preferably 8% or lower, even more preferably 5% or lower, and particularly preferably 3% or lower or 2% or lower, and may be 1% or lower, 0.5% or lower, or 0.10% or lower. The content of the low boiling point component (also referred to as “low boiling point substance” or “low boiling substance”) excluding water in the liquid feed into the dehydration column A is a total area proportion (area %) of peaks having shorter retention times than the peak of 1,3-butylene glycol relative to the total peak area in the gas chromatographic analysis under the above conditions. The content of the low boiling point component (excluding water) in the liquid feed into the dehydration column A can be reduced, for example, by providing a dealcoholization column (low boiling substance removal column) upstream of the dehydration column A, and adjusting the distillation conditions of the dealcoholization column (low boiling substance removal column). For example, increasing the reflux ratio and the number of plates, and the distillation rate in the dealcoholization column (low boiling substance removal column) can reduce the content of the low boiling point component (excluding water) in the liquid feed into the dehydration column A. Furthermore, the concentration of the low boiling point component (excluding water) in the liquid feed into the dehydration column A can be reduced, for example, by reaction conditions (e.g., reaction temperature) in the hydrogenation step.

The content of the high boiling point component in the liquid feed into the dehydration column A is, for example, 20% or lower, preferably 10% or lower, more preferably 7% or lower, 4% or lower, 3% or lower, or 2% or lower, even more preferably 1% or lower, 0.5% or lower, 0.4% or lower, 0.3% or lower, 0.2% or lower, 0.1% or lower, or 0.05% or lower, and particularly preferably 0.01% or lower. The content of the high boiling point component in the liquid feed into the dehydration column A can be adjusted, for example, by reaction conditions (e.g., reaction temperature) in the hydrogenation step. The content of the high boiling point component in the liquid feed into the dehydration column A is a total area proportion (area %) of peaks having longer retention times than the peak of 1,3 BG to the total peak area in the gas chromatographic analysis under the above conditions.

The content of the components other than 1,3-butylene glycol, acetaldehyde, crotonaldehyde and water in the liquid feed into the dehydration column A is, for example, 10 wt. % or lower, preferably 8 wt. % or lower, and more preferably 6 wt. % or lower.

In the manufacturing method of the other present disclosure, the reflux ratio in the dehydration column A [dehydration column reflux amount/dehydration column distilled amount (discharge amount to outside of distillation column) is a value higher than 0.3. The reflux ratio is preferably 0.04 or higher, 0.05 or higher, 0.06 or higher, 0.07 or higher, 0.08 or higher, 0.09 or higher, 0.1 or higher, 0.12 or higher, 0.14 or higher, 0.16 or higher, 0.18 or higher, 0.2 or higher, 0.22 or higher, 0.24 or higher, 0.26 or higher, 0.28 or higher, 0.3 or higher, 0.33 or higher, 0.36 or higher, 0.4 or higher, 0.45 or higher, 0.5 or higher, 0.6 or higher, or 0.7 or higher, 0.8 or higher, 0.9 or higher, 1 or higher, 1.1 or higher, 1.2 or higher, 1.3 or higher, 1.4 or higher, 1.5 or higher, 1.6 or higher, 1.7 or higher, 1.8 or higher, 1.9 or higher, 2 or higher, 3 or higher, 4 or higher, 5 or higher, 6 or higher, 7 or higher, 8 or higher, 9 or higher, 10 or higher, 15 or higher, 20 or higher, or 25 or higher, and more preferably 30 or higher (for example, 40 or higher), from the perspective of reducing the content of the low boiling point substance (containing water) in the crude 1,3-butylene glycol stream containing 1,3-butylene glycol taken out from below the feed plate of the dehydration column A (preferably, the bottom of the column). In particular, in the manufacturing method 2 of the fifth and seventh aspects of the present disclosure, the reflux ratio in the dehydration column A is preferably 10 or higher, more preferably 20 or higher, even more preferably 30 or higher, and particularly preferably 50 or higher. An upper limit of the reflux ratio is, for example, 100, preferably 50 from the point of energy cost. In a case where the number of theoretical plates of the dehydration column A is large, sufficient separation can be performed, if the reflux ratio is 10 or 20, or lower.

In the manufacturing method of the sixth, eighth and ninth aspects of the present disclosure, the reflux ratio in the dehydration column A [dehydration column reflux amount/dehydration column distilled amount (discharge amount to outside of distillation column) is, for example, 0.03 or higher. Note that, during the reflux to the dehydration column A, a condensate of dehydration column top vapor is ordinarily refluxed to the dehydration column, and a part or all of the reflux may be replaced by a water-containing liquid (e.g., pure water) charged into the dehydration column. In that case, the “dehydration column reflux amount” refers to a sum of an amount of the condensate of dehydration column top vapor refluxed to the dehydration column and an amount of the water-containing liquid (e.g., pure water) charged into the dehydration column.

In the manufacturing method 2 of the sixth, eighth and ninth aspects of the present disclosure, the reflux ratio in the dehydration column A is preferably 0.04 or higher, 0.05 or higher, 0.06 or higher, 0.07 or higher, 0.08 or higher, 0.09 or higher, 0.1 or higher, 0.12 or higher, 0.14 or higher, 0.16 or higher, 0.18 or higher, 0.2 or higher, 0.22 or higher, 0.24 or higher, 0.26 or higher, 0.28 or higher, 0.3 or higher, 0.33 or higher, 0.36 or higher, 0.4 or higher, 0.45 or higher, 0.5 or higher, 0.6 or higher, 0.7 or higher, 0.8 or higher, 0.9 or higher, 1 or higher, 1.1 or higher, 1.2 or higher, 1.3 or higher, 1.4 or higher, 1.5 or higher, 1.6 or higher, 1.7 or higher, 1.8 or higher, 1.9 or higher, 2 or higher, 3 or higher, 4 or higher, 5 or higher, 6 or higher, 7 or higher, 8 or higher, or 9 or higher, and more preferably 10 or higher (for example, 15 or higher, 20 or higher, 25 or higher, 30 or higher, or 40 or higher), from the perspective of reducing the content of the low boiling point product (containing water) in the crude 1,3-butylene glycol stream containing 1,3-butylene glycol taken out from below the feed plate of the dehydration column A (preferably, the bottom of the column). In particular, in the manufacturing method 2 of the present disclosure, the reflux ratio in the dehydration column A is preferably 0.2 or higher, more preferably 0.3 or higher, 0.4 or higher, 0.5 or higher, 0.6 or higher, 0.7 or higher, 0.8 or higher, 0.9 or higher, 1 or higher, 1.5 or higher, 2 or higher, 3 or higher, or 4 or higher, even more preferably 5 or higher, and particularly preferably 10 or higher. An upper limit of the reflux ratio is, for example, 50 (lower than 50), preferably 20 (lower than 20), and more preferably 10 (lower than 10), from the point of view of energy cost. In a case where the number of theoretical plates of the dehydration column A is large, sufficient separation can be performed, even when the reflux ratio is approximately 0.03.

In the manufacturing method of the present disclosure, the distillation rate in the dehydration column A can be appropriately set in accordance with the concentration of water in the liquid feed into the dehydration column A. Desirably, the distillation rate is a sufficient distillation rate for the total amount of water in the liquid feed to be distilled. For example, in a case where the concentration of water in the liquid feed into the dehydration column A is X wt. %, the distillation rate in the dehydration column A is preferably X wt. % or higher. Therefore, the distillation rate in the dehydration column A is, for example, 95 wt. % or lower, 90 wt. % or lower, 85 wt. % or lower, 80 wt. % or lower, 75 wt. % or lower, 70 wt. % or lower, 65 wt. % or lower, 60 wt. % or lower, 55 wt. % or lower, 50 wt. % or lower, 45 wt. % or lower, 40 wt. % or lower, 35 wt. % or lower, 30 wt. % or lower, 25 wt. % or lower, 20 wt. % or lower, 15 wt. % or lower, 10 wt. % or lower, or 5 wt. % or lower. The distillation rate refers to a proportion (wt. %) of an amount of liquid extracted from above the feed plate of the dehydration column A (e.g., the top of the column) to the outside of the distillation column with respect to a charged amount into the dehydration column A.

In the manufacturing method of the present disclosure, the 1,3 BG recovery rate in the dehydration column A is, for example, 99.3% or higher. Note that, in the present specification, the 1,3 BG recovery rate in the dehydration column A is a value (%) determined by the following formula.

{1−[concentration (wt. %) of 1,3 BG in distillate×(distilled amount (part)−recycled amount (part))]/(concentration (wt. %) of 1,3 BG in liquid feed×charged amount (part))}×100

Note that the low boiling point substance and the high boiling point substance may be hydrolyzed by water to produce 1,3 BG, while the high boiling point substance may be produced by polymerization of 1,3 BG. Further, trace impurities may be produced or disappear. Thus, the mass balance in the dehydration column may not always be made. This applies to the dealcoholization column (low boiling substance removal column), the high boiling substance removal column, the product column, and other distillation columns.

Next, the crude 1,3-butylene glycol stream containing 1,3-butylene glycol taken out from below the feed plate of the dehydration column A (preferably, the bottom of the column) is fed to the desalting column B. In the desalting column B, the crude 1,3-butylene glycol stream after the desalting is obtained from the top of the column, and a salt, a high boiling point substance, or the like is discharged from the bottom of the column. The bottom rate (%) of the desalting column B [(desalting column bottom amount (part)/desalting column charged amount (part)×100] is, for example, from 0.1 to 40 wt. %, preferably from 1 to 35 wt. %, more preferably from 2 to 30 wt. %, even more preferably from 3 to 25 wt. %, and particularly preferably from 5 to 20 wt. %, and may be from 7 to 15 wt. %. At least a portion of the bottom in the desalting column may be recycled to the step prior to the desalting.

A theoretical number of plates of the high boiling point component removal column C is, for example, from 1 to 100 plates, preferably from 2 to 90 plates, more preferably from 3 to 80 plates, more preferably from 4 to 70 plates, from 5 to 60 plates, from 8 to 50 plates, or from 10 to 40 plates, and particularly preferably from to 30 plates. A feed position for the charged liquid is, for example, from 10 to 90%, preferably from 20 to 80%, and more preferably from 30 to 70 plates, and even more preferably from 40 to 60% of a height of the column downward from the top of the high boiling point component removal column. In the distillation in the high boiling point component removal column C, a pressure (absolute pressure) at the top of the column is, for example, from 0.01 to 50 kPa, preferably from 0.1 to 30 kPa, more preferably from 0.3 to 20 kPa, and even more preferably from 0.5 to 10 kPa.

A concentration of 1,3 BG in the charged liquid into the high boiling point component removal column C is, for example, 95% or greater, preferably 96% or greater, 96.7% or greater, more preferably 97% or greater, even more preferably 98% or greater, and particularly preferably 99% or greater. In particular, in the manufacturing method 2 of the fifth, sixth and seventh aspects of the present disclosure, the concentration of 1,3 BG in the liquid feed into the high boiling substance removal column C is preferably 96.7% or higher, 97% or higher, more preferably 98% or higher, and even more preferably 99% or higher. The concentration of 1,3 BG in the charged liquid into the high boiling point component removal column C can be improved by adjustment of the distillation conditions of the dehydration column A and the desalting column B. For example, increasing the reflux ratio of the dehydration column A or increasing the bottom ratio of the desalting column B can increase the concentration of 1,3 BG in the charged liquid into the high boiling point component removal column C. Note that the concentration of the 1,3 BG described above is an area proportion (area %) of a 1,3 BG peak relative to a total peak area in a gas chromatographic analysis (GC analysis) under the following conditions.

In which the Conditions for the Gas Chromatographic Analysis are as Follows:
Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
a flame ionization detector (FID), 280 °C.

A content of the high boiling point component in the charged liquid into the high boiling point component removal column C is, for example, 4% or less, preferably 3% or less, more preferably 2% or less, even more preferably 1% or less, 0.5% or less, 0.4% or less, 0.3% or less, 0.2% or less, 0.1% or less, or 0.05% or less, and particularly preferably 0.01% or less. In particular, in the manufacturing method 2 of the fifth, sixth and seventh aspects of the present disclosure, a content of the high boiling point component in the liquid feed into the high boiling substance removal column C is preferably 3% or lower, more preferably 2% or lower, even more preferably 1.5% or lower, 1% or lower, 0.5% or lower, 0.4% or lower, 0.3% or lower, 0.2% or lower, 0.1% or lower, or 0.05% or lower, and particularly preferably 0.01% or lower. The content of the high boiling point component in the charged liquid into the high boiling point component removal column C can be reduced by adjustment of the distillation conditions of the desalting column B. For example, increasing the bottom ratio of the desalting column B can reduce the content of the high boiling point component in the charged liquid into the high boiling point component removal column C. Note that the content of the high boiling point component in the charged liquid into the high boiling point component removal column C is a total area proportion (area %) of peaks longer in retention time than the 1,3 BG peaks relative to the total peak area in the gas chromatographic analysis under the above conditions.

In the manufacturing method of the present disclosure, the content of acetaldehyde in liquid feed into the high boiling substance removal column C is, for example, 500 ppm or lower, preferably 205 ppm or lower (e.g., 200 ppm or lower), more preferably 100 ppm or lower, even more preferably 90 ppm or lower, 80 ppm or lower, 70 ppm or lower, 60 ppm or lower, 50 ppm or lower, 40 ppm or lower, 30 ppm or lower, 20 ppm or lower or 10 ppm or lower, and particularly preferably 5 ppm or lower, and may be lower than 2 ppm or lower than 1 ppm. The content of crotonaldehyde in the liquid feed into the high boiling substance removal column C is, for example, 200 ppm or lower, preferably 110 ppm or lower, more preferably 100 ppm or lower, even more preferably 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, or 3 ppm or lower, and particularly preferably 2 ppm or lower, and may be lower than 1 ppm. The acetaldehyde content and the crotonaldehyde content in the liquid feed into the high boiling substance removal column C can be reduced, for example, by providing a dealcoholization column (low boiling substance removal column) and a dehydration column upstream of the high boiling substance removal column C, and adjusting the distillation conditions of the dealcoholization column (low boiling substance removal column) and the dehydration column. For example, increasing the reflux ratio and the number of plates, and the distillation rate in the dealcoholization column (low boiling substance removal column) and the dehydration column can reduce the acetaldehyde content and the crotonaldehyde content of the liquid feed into the high boiling substance removal column C. Note that the acetaldehyde content and the crotonaldehyde content of the liquid feed into the high boiling substance removal column C can be quantified by GC-MS analysis (gas mass spectrometry).

In the manufacturing method of the present disclosure, the content of water in liquid feed into the high boiling substance removal column C is, for example, 3 wt. % or lower, 2 wt. % or lower, 1.2 wt. % or lower, preferably 1.1 wt. % or lower, more preferably 1.0 wt. % or lower, even more preferably 0.95 wt. % or lower, 0.9 wt. % or lower, 0.8 wt. % or lower, 0.7 wt. % or lower, 0.6 wt. % or lower, 0.5 wt. % or lower, 0.4 wt. % or lower, 0.3 wt. % or lower, or 0.2 wt. % or lower, and particularly preferably 0.1 wt. % or lower, 0.05 wt. % or lower, 0.03 wt. % or lower. The content of water in the liquid feed into the high boiling substance removal column C can be reduced by adjusting the distillation conditions of the dehydration column A. For example, increasing the reflux ratio and the number of plates, and the distillation rate in the dehydration column A can reduce the concentration of water in the liquid feed into the high boiling substance removal column C. Note that the water content of the liquid feed into the high boiling substance removal column CF can be quantified by the Karl Fischer moisture measurement instrument.

In the manufacturing method of the present disclosure, the content of the low boiling point component (excluding water) in the liquid feed into the high boiling substance removal column C is, for example, 1.8% or lower, preferably 1.6% or lower, more preferably 1.4% or lower, even more preferably 1.2% or lower, 1.1% or lower, 1% or lower, 0.9% or lower, 0.8% or lower, 0.7% or lower, 0.6% or lower, 0.5% or lower, 0.4% or lower, 0.3% or lower, or 0.2% or lower, and particularly preferably 0.1% or lower. The content of the low boiling point component (also referred to as “low boiling point substance” or “low boiling substance”) excluding water in the liquid feed into the high boiling substance removal column C is a total area proportion (area %) of peaks of having shorter retention times than the peak of 1,3-butylene glycol relative to the total peak area in the gas chromatographic analysis under the above conditions. The content of the low boiling point component (excluding water) in the liquid feed into the high boiling substance removal column C can be reduced, for example, by providing a dealcoholization column (low boiling substance removal column) upstream of the high boiling substance removal column C, and adjusting the distillation conditions of the dealcoholization column (low boiling substance removal column). For example, increasing the reflux ratio and the number of plates, and the distillation rate in the dealcoholization column (low boiling substance removal column) can reduce the content of the low boiling point component (excluding water) in the liquid feed into the high boiling substance removal column C.

In the producing method according to the first aspect of the present disclosure, the reflux ratio in the high boiling point component removal column C [high boiling point component removal column reflux amount/high boiling point component removal column distilled amount (discharge amount to outside of distillation column)] is greater than 0.02 (e.g., 0.025 or greater) when the concentration of 1,3 BG in the charged liquid into the high boiling point component removal column C is less than 95%, and is greater than 0.01 (e.g., 0.015 or greater) when the concentration of 1,3 BG in the high boiling point component removal column C is 95% or greater. In the manufacturing method of the fifth aspect of the present disclosure, the reflux ratio in the high boiling substance removal column C [high boiling substance removal column reflux amount/high boiling substance removal column distilled amount (discharge amount to outside of distillation column)] is 0.02 or higher. In the manufacturing method 1 of the sixth aspect of the present disclosure, the reflux ratio in the high boiling substance removal column C [high boiling substance removal column reflux amount/high boiling substance removal column distilled amount (discharge amount to outside of distillation column)] is, for example, 0.02 or higher. From the perspective of lowering the dry point of 1,3-butylene glycol product, the reflux ratio is preferably greater than 0.02 (e.g., 0.05 or greater), 0.03 or greater, 0.04 or greater, 0.05 or greater, more preferably 0.1 or greater, more preferably 0.2 or greater, 0.3 or greater, 0.4 or greater, 0.5 or greater, 0.6 or greater, 0.7 or greater, 0.8 or greater, 0.9 or greater, 1 or greater, 1.2 or greater, 1.5 or greater, 2 or greater, 3 or greater, 4 or greater, 5 or greater, or 10 or greater, and particularly preferably 20 or greater. In the manufacturing method of the second, third, and fourth aspects of the present disclosure, the reflux ratio in the high boiling point component removal column C is, for example, 0.015 or greater, preferably 0.02 or greater, 0.03 or greater, 0.05 or greater, 0.07 or greater, 0.1 or greater, 0.2 or greater, 0.3 or greater, 0.4 or greater, 0.5 or greater, 0.6 or greater, 0.7 or greater, 0.8 or greater, 0.9 or greater, 1 or greater, 2 or greater, 3 or greater, 4 or greater, 5 or greater, 6 or greater, 7 or greater, 8 or greater, 9 or greater, 10 or greater, 15 or greater, or 20 or greater, and more preferably 30 or greater. In particular, in the manufacturing method 2 of the fifth and sixth aspects of the present disclosure, the reflux ratio in the high boiling substance removal column C is preferably 0.02 or higher, 0.03 or higher, 0.04 or higher, 0.05 or higher, 0.06 or higher, 0.07 or higher, 0.08 or higher, 0.09 or higher, 0.1 or higher, more preferably 0.2 or higher, 0.3 or higher, 0.4 or higher, 0.5 or higher, 0.6 or higher, 0.7 or higher, 0.8 or higher, 0.9 or higher, 1 or higher, 1.2 or higher, 1.5 or higher, 2 or higher, 3 or higher, 4 or higher, 5 or higher or 10 or higher, and particularly preferably 20 or higher. In the manufacturing method of the first, second, third, fourth, fifth, sixth, seventh, eighth, and ninth aspects of the present disclosure, note that an upper limit of the reflux ratio is, for example, 100, preferably 50, from the perspective of energy costs. In a case where the number of theoretical plates of the high boiling substance removal column C is large, sufficient separation can be performed, even when the reflux ratio in the high boiling substance removal column C is about 1 or lower (for example, 0.02).

In the producing method according to the present disclosure, the reflux ratio in the high boiling point component removal column C is within the specified range described above, and thus high-purity 1,3 BG having a very low content of a high boiling point component and a low dry point can be produced with a high recovery ratio.

In the producing method according to the present disclosure, the bottom ratio of the high boiling point component removal column C is, for example, less than 30 wt. %. It should be noted that the bottom ratio is not limited to this value when the bottom of the high boiling point component removal column is distilled in a further distillation column and subjected to high boiling point component removal and 1,3 BG after this process is made a product. When an amount ratio of the final high boiling point component-containing product extracted to the outside of the system is reduced to less than 30 wt. % with respect to the charged amount into the high boiling point component removal column C, it is possible to obtain 1,3 BG in a high yield. Note that the bottom ratio refers to a proportion (wt. %) of an amount of liquid extracted from below the charging plate (for example, bottom of the column) of the high boiling point component removal column C relative to the charged amount into the high boiling point component removal column C (also including a recycled amount of the liquid, when the liquid is recycled to the previous steps which will be described later). Note that when the liquid is recycled to the previous steps described below, the recovery ratio of 1,3 BG is improved as the discharge ratio to the outside is smaller.

From the perspective of improving the recovery ratio of 1,3 BG, the bottom ratio of the high boiling point component removal column C can also be preferably 25 wt. % or less, more preferably 20 wt. % or less, even more preferably 15 wt. % or less, 10 wt. % or less, 7 wt. % or less, 5 wt. % or less, 4 wt. % or less, 3 wt. % or less, or 2 wt. % or less, and can be 1 wt. % or less. In addition, from the perspective of lowering the dry point of the 1,3-butylene glycol product, the bottom ratio of the high boiling point component removal column is, for example, 0.01 wt. % or greater, preferably 0.1 wt. % or greater, 0.5 wt. % or greater, or 1 wt. % or greater, more preferably 2 wt. % or greater, 3 wt. % or greater, 4 wt. % or greater, 5 wt. % or greater, 6 wt. % or greater, 7 wt. % or greater, 8 wt. % or greater, 9 wt. % or greater, 10 wt. % or greater, or 15 wt. % or greater, and particularly preferably 20 wt. % or greater.

At least a portion of the liquid that has been yielded by concentrating the high boiling component extracted from below the charging plate of the high boiling point component removal column C (hereinafter, sometimes referred to as “bottom”) may be recycled to step prior to the high boiling point component removal (dashed arrow illustrated in the lower part of the high boiling point component removal column C in FIG. 1). The recovery ratio of 1,3 BG can be improved by recycling at least a portion of the bottom to the step prior to the high boiling point component removal. Note that, in the present specification, the recovery ratio of 1,3 BG in the high boiling point component removal column C is a value (%) determined by the following formula.

{1−[GC area % of 1,3 BG in bottom]×(bottom amount (part)−recycled amount (part) of bottom]/(GC area % of 1,3 BG in charged liquid×charged amount (part)}×100

Note that the low boiling point substance and the high boiling point substance may be hydrolyzed by water to produce 1,3 BG, while the high boiling point substance may be produced by polymerization of 1,3 BG. Further, due to the formation or disappearance of trace impurities, the mass balance in the high boiling point component removal column may not always be ensured. This applies to the dealcoholization column (low boiling point component removal column), the dehydration column, the product column, and other distillation columns.

The recovery rate of 1,3 BG in the high boiling substance removal column C is, for example, higher than 80%, preferably 85% or higher, more preferably 90% or higher, even more preferably 95% or higher, and particularly preferably 99% or higher.

Examples of the step prior to the removing a high boiling point component include acetaldehyde polymerization (aldol condensation of acetaldehyde), reaction (hydrogenation), dealcoholization (low boiling point component removal), dehydration, and desalting. Among these steps, the bottom is preferably recycled to the acetaldehyde polymerization (aldol condensation of acetaldehyde), since 1,3 BG is produced by hydrolysis of the high boiling point substance. In addition, the hydrogen addition reduction may produce 1,3 BG, and the bottom may be recycled to the hydrogen addition from that viewpoint.

The recycled amount of the bottom recycled to the step prior to the removing a high boiling point component can be appropriately selected within a range of the amount of the bottom. The recycled amount of the bottom recycled to the step prior to the removing a high boiling point component is, for example, less than 30 wt. %, preferably 25 wt. % or less with respect to the charged amount into the high boiling point component removal column C. Note that the recycled amount of the bottom recycled may be 20 wt. % or less, 15 wt. % or less, 10 wt. % or less, 7 wt. % or less, 5 wt. % or less, 4 wt. % or less, 3 wt. % or less, 2 wt. % or less, or 1 wt. % or less with respect to the charged amount into the high boiling point component removal column C. In addition, from the perspective of improving the recovery ratio of 1,3 BG in the high boiling point component removal column and the yield of 1,3 BG throughout the 1,3 BG producing process, the recycled amount of the bottom recycled to the steps before the removing a high boiling point component is, for example, 0.01 wt. % or greater, preferably 0.1 wt. % or greater, more preferably 2 wt. % or greater, 3 wt. % or greater, 4 wt. % or greater, 5 wt. % or greater, 7 wt. % or greater, or 10 wt. % or greater, and particularly preferably 20 wt. % or greater with respect to the charged amount into the high boiling point component removal column C. Note that, in a case where the amount of the bottom is reduced to the minimum, 1,3 BG can be recovered in a high yield, even without being recycled to the previous steps.

The crude 1,3-butylene glycol stream taken out from above the feed plate of the high boiling substance removal column C can be a 1,3-butylene glycol product as it is in the manufacturing method of the present disclosure, especially in the manufacturing method 2 of the fifth, sixth and seventh aspects of the present disclosure. Furthermore, the crude 1,3-butylene glycol stream taken out from above the feed plate of the high boiling substance removal column C is subjected to alkaline treatment in the alkaline reactor D described below, and evaporated (or distilled) with the dealkalization column E, and the distillate at the top of the dealkalization column E can be a 1,3-butylene glycol product.

According to the manufacturing method 2 of the fifth aspect of the present disclosure, the content of acetaldehyde and the content of crotonaldehyde in the liquid feed into the dehydration column A are within the predetermined ranges, and the reflux ratio in the dehydration column A is the predetermined value or higher. The reflux ratio in the high boiling substance removal column C is the predetermined value or higher. Therefore, it is possible to industrially efficiently manufacture high-purity 1,3-butylene glycol having a high potassium permanganate test value, a very low content of low boiling point components and high boiling point components, a high initial boiling point, and a low dry point.

According to the manufacturing method 2 of the sixth aspect of the present disclosure, the acetaldehyde content and the crotonaldehyde content in the liquid feed into the dehydration column A are within the predetermined ranges, and the reflux ratio in the high boiling substance removal column C is the predetermined value or higher. Therefore, it is possible to industrially efficiently manufacture high-purity 1,3-butylene glycol having a high potassium permanganate test value, a very low content of low boiling point components and high boiling point components, a high initial boiling point, and a low dry point.

According to the production method 2 of the seventh and eighth aspects of the present disclosure, the content of acetaldehyde and the content of crotonaldehyde in the charged liquid into the high boiling point component removal column are within the specific ranges, and the reflux ratio of the high boiling point component removal column is within the specific range. Therefore, it is possible to industrially efficiently yield high-purity 1,3-butylene glycol that is colorless and odorless (or almost colorless and odorless), unlikely to cause or increase coloration and odor over time, and, besides, unlikely to cause an acid concentration increase over time even in a state containing water.

According to the producing method 2 of the ninth aspect of the present disclosure, the contents of acetaldehyde, crotonaldehyde and water in the charged liquid into the high boiling point component removal column are within the specific ranges, and the reflux ratio of the high boiling point component removal column is within the specific range. Therefore, it is possible to industrially efficiently produce high-purity 1,3-butylene glycol that is colorless and odorless (or almost colorless and odorless), unlikely to cause or increase coloration and odor over time, and, in addition, unlikely to cause an acid concentration increase over time also in a state containing water.

The crude 1,3-butylene glycol stream taken out from above the charging plate of the high boiling point component removal column C is fed to the alkaline reactor (e.g., a flow-through tubular reactor) D and is treated with a base (treated with alkali).

Here, the fifth and sixth aspects of the present disclosures correspond to manufacturing method 1. The base treatment can decompose by-products contained in the crude 1,3-butylene glycol. The base is added to the alkaline reactor D or its upstream piping or the like. The base is added in an amount of, for example, from 0.05 to 10 wt. %, preferably from 0.1 to 1.0 wt. % relative to the crude 1,3-butylene glycol stream subjected to the alkaline treatment. With the added amount of the base exceeding 10 wt. %, the base would precipitate in the distillation column, piping, or the like, and this may cause blockage. In addition, the decomposition reaction of a high boiling point compound would occur, and by-products may be produced adversely. With the added amount of the base of less than 0.05 wt. %, the effect of decomposing by-products is reduced.

The base added in the alkaline reactor D or its upstream piping is not particularly limited but is, for example, preferably an alkali metal compound. Examples of the alkali metal compound include sodium hydroxide, potassium hydroxide, sodium (bi)carbonate, and potassium (bi)carbonate. A basic ion exchange resin can also be used as the base. The base is preferably sodium hydroxide or potassium hydroxide from the perspective of reducing the byproducts contained in the 1,3-butylene glycol product produced finally. The base may be added as is in the solid form but is preferably added in an aqueous solution to facilitate operation and contact with a solution to be treated. One of the bases described above may be used alone, or two or more may be used simultaneously.

The reaction temperature in the alkaline reactor D is not particularly limited but is, for example, preferably from 90 to 140 °C and more preferably from 110 to 130 °C. The reaction at a reaction temperature lower than 90 °C would require long reaction residence time and thus require a reactor with a large volume and make the process uneconomical. The reaction at a reaction temperature exceeding 140 °C would increase coloration in the 1,3-butylene glycol product produced finally. The reaction residence time is, for example, preferably from 5 to 120 minutes and more preferably from 10 to 30 minutes. A reaction residence time shorter than 5 minutes may cause an insufficient reaction and adversely affect the quality of the 1,3-butylene glycol product produced finally. A reaction residence time exceeding 120 minutes would require a large reactor and increase the cost of equipment, and thus would be disadvantageous from the economic point of view.

After exiting the alkaline reactor D, the reaction crude liquid stream is fed to the dealkalization column (e.g., thin film evaporator) E if necessary, and the base and the like are removed from the bottom of the column by evaporation. On the other hand, from the top of the dealkalization column E, a crude 1,3-butylene glycol stream after the removal of a base (which is used as the 1,3-butylene glycol product, at least in the manufacturing method 2 of the fifth, sixth and seventh aspects of the present disclosure) is yielded. The evaporator used for the dealkalization column E is suitably a natural downward flow thin-film evaporator or a forced-stirring thin-film evaporator with a short residence time for the purpose of reducing the thermal history to the process fluid. A demister may be installed in a space above the charging position of the dealkalization column (e.g., thin film evaporator) E, and droplets of a base or the like may be removed. This makes it possible to prevent the base and the high boiling point substance from being mixed into the 1,3-butylene glycol product.

Evaporation is performed in the evaporator used for the dealkalization column E. for example, under a reduced pressure at the top of the column of 20 kPa or less (absolute pressure), preferably from 0.5 to 10 kPa (absolute pressure). The temperature of the evaporator is, for example, preferably from 90 to 120 °C. The crude 1,3-butylene glycol stream containing a low boiling point substance distilled from the top of the column is fed to the product distillation column (product column) F. Note that, as described above, at least in the manufacturing method 2 of the fifth, sixth and seventh aspects of the present disclosure, the distillate (corresponding to E-1) from the top of the dealkalization column E can be a 1,3-butylene glycol product.

Note that the alkaline reactor D and the dealkalization column E may be installed between the desalting column B and the high boiling point component removal column C, between the dehydration column A and the desalting column B (in this case, the desalting column may also serve as a dealkalization column), or before the dehydration column A. In addition, without providing the alkaline reactor D or the dealkalization column E, the alkaline treatment can be performed by charging the base into a high boiling point component removal column charging line or into a dehydration column charging line, or adding the base to the reaction solution after the hydrogenation [and then charging the base into the dealcoholization column (low boiling point component removal column)].

In the production method according to the second aspect of the present disclosure, in the product column F for use in the product distillation, a charged liquid containing 1,3-butylene glycol is distilled under a condition of a reflux ratio of greater than 0.1, a liquid in which a low boiling point component is concentrated is distilled off from above the charging plate (corresponding to “X-6” in FIG. 1 of U.S. Patent Application Publication No. US2023/0035233), and 1,3-butylene glycol is extracted from below the charging plate (corresponding to “Y” in FIG. 1 of U.S. Patent Application Publication No. US2023/0035233). The 1,3-butylene glycol has a very low content of the low boiling component and a high initial boiling point, and thus can be made into a 1,3-butylene glycol product as it is.

In the manufacturing method according to the third aspect of the present disclosure, in the product column F for use in the product distillation step, a liquid feed having a 1,3-butylene glycol concentration of 97% or higher, an acetaldehyde content of 500 ppm or lower, and a crotonaldehyde content of 200 ppm or lower is distilled under a condition of a reflux ratio of higher than 0.1, and a liquid concentrated with acetaldehyde and crotonaldehyde is distilled off from above a feed plate (corresponding to “X-6” in FIG. 1 of U.S. Patent Application Publication No. US2023/0055591), and 1,3-butylene glycol is extracted from below the feed plate (corresponding to “Y” in FIG. 1 of U.S. Patent Application Publication No. US2023/0055591). The 1,3-butylene glycol has a high potassium permanganate test value, and thus can be made into a 1,3-butylene glycol product as it is.

In the manufacturing method of the fourth aspect of the present disclosure, and in the manufacturing method 1 of the fifth and sixth aspects of the present disclosure, in the product column F for use in the product distillation, a 1,3-butylene glycol liquid feed with an acetaldehyde content of 500 ppm or lower and a crotonaldehyde content of 200 ppm or lower is distilled under a condition of a reflux ratio of higher than 0.1, a liquid concentrated with a low boiling point component is distilled off from above a feed plate (corresponding to “X-6” in FIG. 1 of U.S. Patent Application Publication No. US2022/0354760 or US2023/0048638), and a 1,3-butylene glycol product is extracted from below the feed plate (corresponding to “Y” in FIG. 1 of U.S. Patent Application Publication No. US2022/0354760 or US2023/0048638). The extracted 1,3-butylene glycol can be a 1,3-butylene glycol product.

In the production method 1 of the present disclosure, in the product column F for use in the product distillation, a charged liquid having a concentration of 1,3-butylene glycol of, for example, 97.6 area % or greater determined by GC analysis is distilled, a liquid with a concentrated low boiling point component is distilled from above the charging plate (corresponding to “X-6” in FIG. 4), and 1,3-butylene glycol is extracted from below the charging plate (corresponding to “Y” in FIG. 4). The extracted 1,3-butylene glycol can be a 1,3-butylene glycol product.

The product distillation column F can be, for example, a perforated-plate column, a bubble column, or the like, but is more preferably a packed column with a low pressure loss, filled with Sulzer Packing, Melapack (trade names of Sumitomo Heavy Industries, Ltd.). This is because 1,3-butylene glycol and trace impurities would be thermally decomposed at a high temperature (e.g., 150 °C or higher) and form a low boiling point substance, which is a coloring component. Thus, the distillation temperature is to be lowered. In addition, this is also because a long thermal history (residence time) for 1,3-butylene glycol would also have a similar effect. Thus, the reboiler employed is preferably one with a short residence time of the process side fluid, for example, a thin-film evaporator, such as a natural downward flow thin-film evaporator or a forced-stirring thin-film evaporator.

A theoretical number of plates of the product column F is, for example, from 1 to 100 plates, preferably from 2 to 90 plates, from 3 to 80 plates, from 4 to 70 plates, from 5 to 60 plates, from 8 to 50 plates, or from 10 to 40 plates, and more preferably from 15 to 30 plates. In particular, in the manufacturing method of the first aspect of the present disclosure, a theoretical number of plates of the product distillation column F is, in a case where the concentration of the low boiling point substance in the charged liquid is 5 wt. % or less, for example, from 1 to 100 plates, preferably from 2 to 90 plates, from 3 to 80 plates, from 4 to 70 plates, or from 5 to 60 plates, from 8 to 50 plates, or from 10 to 40 plates, and more preferably from 15 to 30 plates. A feed position for the charged liquid is, for example, from 10 to 90%, preferably from 20 to 80%, and more preferably from 30 to 70%, and even more preferably from to 60% of the height of the column downward from the top of the column. In the distillation in the product distillation column F, a pressure (absolute pressure) at the top of the column is, for example, from 20 kPa or less, preferably from 0.1 to 10 kPa, more preferably from 0.3 to 8 kPa, even more preferably from 0.5 to 5 kPa. The reflux ratio in the product distillation column F is, for example, from 0.05 to 500, preferably from 0.1 to 300, more preferably from 0.2 to 200, more preferably from 0.5 to 100, from 1 to 50, from 2 to 40, or from 3 to 30, and particularly preferably from 4 to 25.

In FIG. 1, in charging to the product distillation column F, the column top vapor from the dealkalization column E is condensed in the condenser E-1, and the resulting condensed liquid is fed, but the column top vapor from the dealkalization column E may be directly fed to the product distillation column F. In the product distillation column F, an impurity, such as a low boiling point substance, is distilled off from the top of the column, and 1,3-butylene glycol as a product is produced from the bottom of the column of the product distillation column F (corresponding to “Y”).

The concentration of 1,3-butylene glycol in the charged liquid (1,3-butylene glycol liquid feed) into the product column F is, for example, 90% or greater, preferably 91% or greater, more preferably 92% or greater, even more preferably 93% or greater, 94% or greater, 95% or greater, 96% or greater, 97% or greater, 97.1% or greater, 97.2% or greater, 97.3% or greater, 97.4% or greater, 97.5% or greater, 97.6% or greater, 97.7% or greater, 97.8% or greater, 97.9% or greater, 98% or greater, 98.1% or greater, 98.2% or greater, 98.3% or greater, 98.4% or greater, 98.5% or greater, 98.6% or greater, 98.7% or greater, 98.8% or greater, 98.9% or greater, and particularly preferably 99% or greater (e.g., 99.1% or greater, 99.2% or greater, 99.3% or greater, 99.4% or greater, 99.5% or greater, 99.6% or greater, 99.7% or greater, 99.8% or greater, or 99.9% or greater). When the number of theoretical plates, the reflux amount, and the reflux ratio of the product column F are sufficient, 1,3-butylene glycol can be commercialized even if the concentration of 1,3-butylene glycol in the liquid feed into the product column F is lower than 90%. In the manufacturing method of the third aspect of the present disclosure, the concentration of 1,3-butylene glycol in the liquid feed into the product column F is 97% or higher, preferably 98% or higher, and more preferably 99% or higher. The concentration of 1,3-butylene glycol in the charged liquid into the product column F can be improved, for example, by adjusting the distillation condition of the dehydration column A, providing a dealcoholization column (low boiling point component removal column) before the dehydration column A, and adjusting the distillation condition thereof, or adjusting the distillation condition of the high boiling point component removal column C. For example, it is possible to increase the purity of 1,3-butylene glycol in charged liquid into the product column F by increasing the reflux ratio of the dealcoholization column (low boiling point component removal column), the dehydration column A, and/or the high boiling point component removal column C or increasing the number of plates. Note that the concentration of 1,3-butylene glycol in the liquid feed (1,3-butylene glycol liquid feed) into the product column F is an area proportion (area %) of the 1,3-butylene glycol peak relative to the total peak area in the gas chromatographic (GC) analysis of the following conditions, in which the conditions for the gas chromatographic analysis are as follows:
Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

In the production method according to the present disclosure, at least in the production method 1 according to the fifth, sixth and seventh aspects of the present disclosure, the content of water in the charged liquid into the product column F is, for example, 3 wt. % or less, preferably 2.8 wt. % or less, more preferably 2.6 wt. % or less, 2.4 wt. % or less, 2.2 wt. % or less, 2 wt. % or less, 1.8 wt. % or less, 1.6 wt. % or less, 1.4 wt. % or less, 1.4 wt. % or less, 1.2 wt. % or less, 1.1 wt. % or less, 1 wt. % or less, 0.95 wt. % or less, 0.9 wt. % or less, 0.8 wt. % or less, 0.7 wt. % or less, 0.6 wt. % or less, 0.5 wt. % or less, 0.4 wt. % or less, 0.3 wt. % or less, or 0.2 wt. % or less, and particularly preferably 0.1 wt. % or less. The content of water in the charged liquid into the product column F can be reduced by adjusting the distillation condition of the dehydration column A. For example, increasing the reflux ratio, the number of plates, and the distillation ratio in the dehydration column A can reduce the concentration of water in the charged liquid into the product column F. Note that the water content of the charged liquid into the product column F can be quantified by the Karl Fischer water content measurement instrument.

In the production method according to the present disclosure, at least in the production method 1 according to the fifth, sixth and seventh aspects of the present disclosure, the content of the low boiling point component (excluding water) in the charged liquid into the product column F is, for example, 1.8% or less, preferably 1.6% or less, 1.4% or less, 1.2% or less, 1.1% or less, 1% or less, 0.9% or less, 0.8% or less, 0.7% or less, 0.6% or less, or 0.5% or less, or 0.4% or less, or 0.3% or less, or 0.2% or less, and more preferably 0.1% or less. The content of the low boiling point component (also referred to as “low boiling point substance”) excluding water in the charged liquid into the product column F is a total area proportion (area %) of peaks shorter in retention time than the 1,3-butylene glycol peak relative to the total peak area in the gas chromatographic analysis under the above conditions. The content of the low boiling point component (excluding water) in the charged liquid into the product column F can be reduced, for example, by providing a dealcoholization column (low boiling point component removal column) upstream of the product column F, and adjusting the distillation condition of the dealcoholization column (low boiling point component removal column). For example, increasing the reflux ratio, the number of plates, and the distillation ratio in the dealcoholization column (low boiling point component removal column) can reduce the content of the low boiling point component (excluding water) in the charged liquid into the product column F.

The content of the high boiling point component (excluding water) in the liquid feed into the product column F is, for example, 1.8% or lower, preferably 1.6% or lower, more preferably 1.4% or lower, more preferably 1.2% or lower, 1.1% or lower, 1% or lower, 0.9% or lower, 0.8% or lower, 0.7% or lower, 0.6% or lower, 0.5% or lower, 0.4% or lower, 0.3% or lower or 0.2% or lower, and particularly preferably 0.1% or lower. The content of the high boiling point component (also referred to as “high boiling point substance” or “high boiling substance”) excluding water in the liquid feed into the product column F is a total area proportion (area %) of peaks having longer retention times than the peak of 1,3-butylene glycol relative to the total peak area in the gas chromatographic analysis under the above conditions. The content of the high boiling point component (excluding water) in the liquid feed into the product column F can be reduced, for example, by adjusting the distillation conditions of the high boiling substance removal column. For example, increasing the reflux ratio, the number of plates, or the bottom rate of the high boiling substance removal column can reduce the concentration of the high boiling point component (excluding water) in the liquid feed into the product column F.

In the manufacturing method according to the third and fourth aspects of the present disclosure, and in the manufacturing method 1 according to the fifth, sixth and seventh aspects of the present disclosure, the content of acetaldehyde in the liquid feed into the product column F is 500 ppm or lower, and the content of crotonaldehyde therein is 200 ppm or lower. The content of acetaldehyde in the liquid feed into the product column F is preferably 205 ppm or lower (e.g., 200 ppm or lower), more preferably 150 ppm or lower, 120 ppm or lower, 100 ppm or lower, even more preferably 90 ppm or lower, 80 ppm or lower, 70 ppm or lower, 60 ppm or lower, 50 ppm or lower, 40 ppm or lower, 30 ppm or lower, 20 ppm or lower, or 10 ppm or lower, and particularly preferably 5 ppm or lower, and may be lower than 2 ppm. The content of the crotonaldehyde in the liquid feed into the product column F is preferably 150 ppm or lower, 130 ppm or lower, 110 ppm or lower, more preferably 100 ppm or lower, even more preferably 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, or 3 ppm or lower, and particularly preferably 2 ppm or lower, and may be lower than 1 ppm. The acetaldehyde content and the crotonaldehyde content in the liquid feed into the product column F can be reduced, for example, by providing a dealcoholization column (low boiling substance removal column) and a dehydration column upstream of the product column F, and adjusting the distillation conditions of the dealcoholization column (low boiling substance removal column) and the dehydration column. For example, increasing the reflux ratio and the number of plates, and the distillation rate in the dealcoholization column (low boiling substance removal column) and the dehydration column can reduce the acetaldehyde content and the crotonaldehyde content of the liquid feed into the product column F. In addition, the acetaldehyde content and the crotonaldehyde content in the liquid feed into the product column F can be reduced by increasing the reaction temperature, increasing the residence time, or increasing the added amount of the base, in the alkaline reaction. Note that the acetaldehyde content and the crotonaldehyde content of the liquid feed into the product column F can be quantified by GC-MS analysis (gas mass spectrometry).

In the production method according to the second, third and fourth aspects of the present disclosure, and in the production method according to the fifth and sixth aspects of the present disclosure, the reflux ratio in the product column F [product column reflux amount/product column distilled amount)] is greater than 0.1 (for example, 0.15 or greater). The reflux ratio is preferably 0.2 or greater, more preferably 0.3 or greater, even more preferably 0.4 or greater, 0.5 or greater, 1 or greater, 2 or greater, 3 or greater, 4 or greater, 5 or greater, 6 or greater, 7 or greater, 8 or greater, 9 or greater, 10 or greater, 20 or greater, or 50 or greater, and particularly preferably 400 or greater (for example, 500 or greater), from the perspective of increasing the initial boiling point of the 1,3-butylene glycol product. The reflux ratio is preferably 0.2 or higher, more preferably 0.3 or higher, even more preferably 0.4 or higher, 0.5 or higher, 1 or higher, 2 or higher, 3 or higher, 4 or higher, 5 or higher, 6 or higher, 7 or higher, 8 or higher, 9 or higher, 10 or higher, 11 or higher, 12 or higher, 13 or higher, 14 or higher, 15 or higher, 16 or higher, 17 or higher, 18 or higher, 19 or higher, 20 or higher, 25 or higher, 30 or higher, 35 or higher, 40 or higher, or 50 or higher, and particularly preferably 400 or higher (e.g., 500 or higher), from the perspective of increasing the potassium permanganate test value of the 1,3-butylene glycol product. An upper limit of the reflux ratio of the product column F is, for example, 700 or 1000 from the perspective of energy cost.

In the manufacturing method of the fourth aspect of the present disclosure, the concentration of 1,3-butylene glycol, the content of acetaldehyde, the content of crotonaldehyde, and the content of water in the liquid feed into the dehydration column A are within the predetermined ranges; the reflux ratio in the dehydration column A is the predetermined value or higher; the content of water in the liquid feed into the product column F is 1.2 wt. % or lower (preferably, the content of low boiling point components other than water in the liquid feed into the product column F is 1.8 wt. % or lower); and the reflux ratio in the product column F is within the predetermined range, and thus it is possible to manufacture high-purity 1,3-butylene glycol having a high potassium permanganate test value, a very low content of low boiling point components and a high initial boiling point, with a high recovery rate.

In the production method according to the present disclosure, the reflux ratio in the product column F is within the specified range described above, and thus high-purity 1,3-butylene glycol having a very low content of the low boiling point component and a high initial boiling point can be produced, with a high recovery ratio.

In the production method according to the present disclosure, the distillation ratio in the product column F is, for example, less than 30 wt. %, less than 29 wt. %, less than 28 wt. %, less than 27 wt. %, less than 26 wt. %, less than 25 wt. %, less than 24 wt. %, less than 23 wt. %, less than 22 wt. %, less than 21 wt. %, or less than 20 wt. %, more preferably 19 wt. % or less, 18 wt. % or less, 17 wt. % or less, 16 wt. % or less, or 15 wt. % or less, even more preferably 12 wt. % or less, 10 wt. % or less, 8 wt. % or less, 5 wt. % or less, 3 wt. % or less, 2 wt. % or less, 1 wt. % or less, 0.8 wt. % or less, or 0.6 wt. % or less, and particularly preferably 0.4 wt. % or less, 0.2 wt. % or less, from the perspective of improving the recovery ratio of 1,3-butylene glycol. In the manufacturing method of the present disclosure, the distillation rate in the product column F is, for example, lower than 30 wt. %, 29 wt. % or lower, more preferably 28 wt. % or lower, even more preferably 27 wt. % or lower, 26 wt. % or lower, 25 wt. % or lower, 24 wt. % or lower, 23 wt. % or lower, 22 wt. % or lower, 21 wt. % or lower, 20 wt. % or lower, from the perspective of improving the recovery rate of 1,3-butylene glycol. Note that the distillation ratio refers to a proportion (wt. %) of an amount of liquid extracted from above the charging plate of the product column F (for example, the top of the column) to the outside of the distillation column (this amount including also the recycled amount of liquid when recycled to the previous step which will be described below) with respect to a charged amount into the product column F. At least in the manufacturing method according to the third aspect of the present disclosure, the distillation rate of the product column F is preferably lower than 20 wt. %.

At least a portion of the liquid (hereinafter, sometimes referred to as “distillate”) in which the low boiling point component is concentrated, which is extracted from above the charging plate of the product column F, may be recycled to the step prior to the performing product distillation (dashed arrow illustrated on the right side of the product column F in FIG. 1 of U.S. Patent Application Publication No. US2023/0035233). The recovery ratio of 1,3-butylene glycol can be improved by recycling at least a portion of the distillate to the step prior to the performing product distillation.

Examples of the step prior to the performing product distillation include dehydration and dealcoholization (low boiling point component removal). Note that the dealcoholization (low boiling point component removal) is preferably provided before the dehydration.

At least in the production method according to the third aspect of the present disclosure, note that the distillate from the product column F is not recycled to a high boiling substance removal step in which the distillate from the high boiling substance removal column is purified to obtain a purified product. However, the distillate from the product column F can be recycled to a high boiling substance removal step in which, as side cut of the high boiling substance removal column, a distillate containing a purified product and a low boiling point substance is taken out, and discarded or recycled to an upstream step. The dealcoholization step (low boiling substance removal step) is preferably provided before the dehydration step. Also, when the crude reaction liquid containing 1,3-butylene glycol is a crude reaction liquid obtained by hydrogen reduction of acetaldols, at least a portion of the distillate from the product column may be recycled to the hydrogen reduction step (hydrogenation step) of acetaldols or a step upstream of the hydrogen reduction. At least a portion of the distillate from the product column is recycled to a step of the hydrogen reduction (hydrogenation step) of acetaldols or a step upstream of the hydrogen reduction, and thus a reducing substance such as acetaldehyde or crotonaldehyde contained in the distillate from the product column can be made essentially harmless by hydrogen addition in the hydrogenation. Therefore, it is possible to prevent such reducing substances from accumulating in the purification system and eventually getting mixed in the 1,3-butylene glycol product. This either makes it unnecessary to discharge the distillate from the product column to the outside the system or an amount to be discharged be extremely small, even if the distillate is discharged to the outside of the system. Therefore, it is possible to achieve two effects of improving the product quality and improving the product yield at the same time.

The recycled amount of the distillate recycled to the step prior to performing the product distillation can be appropriately selected within the range of the amount of distillate. The recycled amount of the distillate recycle to the step prior to performing the product distillation is, for example, less than 30 wt. % with respect to the charged amount into the product column F. Also, from the perspective of improving the 1,3 BG recovery ratio in the product column and the yield throughout the entire process, the recycled amount of the distillate recycled to the step prior to the performing product distillation is, for example, 0.01 wt. % or greater, preferably 0.05 wt. % or greater, more preferably 0.1 wt. % or greater, 0.5 wt. % or greater, 1 wt. % or greater, 1.5 wt. % or greater, 2 wt. % or greater, 3 wt. % or greater, 4 wt. % or greater, 5 wt. % or greater, 7 wt. % or greater, or 10 wt. % or greater, and particularly preferably 20 wt. % or greater with respect to the charged amount into the product column F.

At least in the production method according to the third aspect of the present disclosure, the upper limit is preferably 90 wt. % or lower, more preferably 80 wt. % or lower, and even more preferably 70 wt. % or lower, 60 wt. % or lower, 50 wt. % or lower, or 40 wt. % or lower, and may be 30 wt. % or lower, with respect to a charged amount into the product column F.

Thus, according to the producing method of the first aspect of the present disclosure, it is possible to produce high-purity 1,3-butylene glycol having a very low content of a high boiling point component and a low dry point, with a high recovery ratio.

Moreover, thus, according to the production method of the second aspect of the present disclosure, it is possible to produce high-purity 1,3-butylene glycol having a very low content of the low boiling point component and a high initial boiling point, with a high recovery ratio.

Moreover, thus, according to the manufacturing method of the third aspect of the present disclosure, 1,3-butylene glycol having a very low content of acetaldehyde and crotonaldehyde and a high potassium permanganate test value can be generally manufactured with a high recovery rate.

Moreover, thus, according to the manufacturing method of the fourth aspect of the present disclosure, it is possible to manufacture high-purity 1,3-butylene glycol having a high potassium permanganate test value, a very low content of low boiling point components, and a high initial boiling point with a high recovery rate.

Moreover, in the manufacturing method 1 of the fifth aspect of the present disclosure, the content of acetaldehyde and the content of crotonaldehyde in the liquid feed into the dehydration column A are within the predetermined ranges, and the reflux ratio in the dehydration column A is the predetermined value or higher; the reflux ratio of the high boiling substance removal column C is the predetermined value or higher; and further the content of acetaldehyde and the content of crotonaldehyde in the liquid feed into product column F are the predetermined values or lower, and the reflux ratio of the product column F is within the predetermined range. Therefore, it is possible to industrially efficiently manufacture high-purity 1,3-butylene glycol having a high potassium permanganate test value, a very low content of low boiling point components and high boiling point components, a high initial boiling point, and a low dry point, generally with a high recovery rate.

Moreover, in the manufacturing method 1 of the sixth aspect of the present disclosure, the acetaldehyde content and the crotonaldehyde content in the liquid feed into the dehydration column A are within the predetermined ranges, the acetaldehyde content and the crotonaldehyde content in the liquid feed into product column F are the predetermined values or lower, and the reflux ratio of the product column F is within the predetermined range. Therefore, it is possible to industrially efficiently manufacture high-purity 1,3-butylene glycol having a high potassium permanganate test value, a very low content of low boiling point components and high boiling point components, a high initial boiling point, and a low dry point, generally with a high recovery rate.

In the manufacturing method of the fourth, fifth and sixth aspects of the present disclosure, The recovery rate of 1,3 BG in the product column F is, for example, higher than 80%, preferably 85% or higher, more preferably 90% or higher, even more preferably 95% or higher, and particularly preferably 99% or higher.

In the production method 1 of the seventh and eighth aspects of the present disclosure, the content of acetaldehyde and the content of crotonaldehyde in the charged liquid into product column F are within the specific ranges, and the reflux ratio of the product column F is within the specific range. Therefore, it is possible to industrially efficiently produce high-purity 1,3-butylene glycol that is colorless and odorless (or almost colorless and odorless), unlikely to cause or increase coloration and odor over time, and, besides, unlikely to cause an acid concentration increase over time even in a state containing water.

In the producing method 1 of the ninth aspect of the present disclosure, the contents of acetaldehyde, crotonaldehyde and water in the charged liquid into product column F are within the specific ranges, and the reflux ratio of the product column F is within the specific range. Therefore, it is possible to industrially efficiently produce high-purity 1,3-butylene glycol that is colorless and odorless (or almost colorless and odorless), unlikely to cause or increase coloration and odor over time, and, in addition, unlikely to cause an acid concentration increase over time also in a state containing water.

The recovery ratio of 1,3 BG in the product column F is, for example, greater than 80%, preferably 85% or greater, more preferably 90% or greater, even more preferably 95% or greater, and particularly preferably 99% or greater.

Note that, in the present specification, the recovery ratio of 1,3 BG in the product column F is a value (%) determined by the following formula.

{1−[GC area % of 1,3 BG in distillate]×(distilled amount (part)−recycled amount (part) of distillate]/(GC area % of 1,3 BG in charged liquid×charged amount (part)}×100

Note that the low boiling point substance and the high boiling point substance may be hydrolyzed by water to produce 1,3 BG, while the high boiling point substance may be produced by polymerization of 1,3 BG. Further, due to the formation or disappearance of trace impurities, the mass balance in the product column may not always be ensured. This applies to the dealcoholization column (low boiling point component removal column), the dehydration column, the high boiling point component removal column, and other distillation columns.

### 1,3-Butylene Glycol Product

The 1,3-butylene glycol product according to the present disclosure can be produced by the producing method according to the present disclosure described above. The 1,3-butylene glycol product according to the first aspect of the present disclosure has a dry point of 209 °C or lower and an area ratio of a 1,3-butylene glycol peak of greater than 98.5% and a total area ratio of peaks longer in retention time than the 1,3-butylene glycol peak of less than 1.2%, according to a gas chromatographic analysis (GC analysis) performed under conditions set forth below:

### The Conditions for the Gas Chromatographic Analysis are as Follows:

Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

The 1,3-butylene glycol product according to the second aspect of the present disclosure can be produced by the producing method according to the present disclosure described above. The 1,3-butylene glycol product according to the present disclosure has an initial boiling point higher than 203 °C and a water content of less than 0.4 wt. %. The initial boiling point is preferably 204 °C or higher, more preferably 205 °C or higher, even more preferably 206 °C or higher or 207 °C, and particularly preferably 208 °C or higher. The content of water is preferably 0.3 wt. % or less, more preferably 0.2 wt. % or less, even more preferably 0.1 wt. % or less, 0.07 wt. % or less, 0.05 wt. % or less, 0.03 wt. % or less, 0.02 wt. % or less, or 0.01 wt. % or less, and particularly preferably 0.005 wt. % or less. Note that the content of water can be quantified by a Karl Fischer water content analyzer.

The 1,3-butylene glycol product of the third aspect of the present disclosure can be obtained by the manufacturing method of the present disclosure. The 1,3-butylene glycol product of the present disclosure has a high potassium permanganate test value (PMT) of higher than 10 minutes. The potassium permanganate test value (PMT) is preferably 15 minutes or longer, more preferably 30 minutes or longer, even more preferably 40 minutes or longer, and particularly preferably 50 minutes or longer (particularly, 60 minutes or longer).

The 1,3-butylene glycol product of the fourth aspect of the present disclosure can be obtained by the manufacturing method of the present disclosure. The 1,3-butylene glycol product of the present disclosure has an initial boiling point of higher than 203 °C and a potassium permanganate test value (PMT) of 30 minutes or longer. The initial boiling point is preferably 204 °C or higher, more preferably 205 °C or higher, even more preferably 206 °C or higher or 207 °C or higher, and particularly preferably 208 °C or higher. The potassium permanganate test value (PMT) is preferably longer than 30 minutes (e.g., 32 minutes or longer), more preferably 35 minutes or longer, even more preferably 40 minutes or longer, and particularly preferably 50 minutes or longer (especially, 60 minutes or longer).

The 1,3-butylene glycol product of the fifth aspect of the present disclosure can be obtained by the manufacturing method of the present disclosure. The 1,3-butylene glycol product of the present disclosure has an initial boiling point of higher than 203 °C and a dry point of 209 °C or lower. Also, preferably, the 1,3-butylene glycol product has a potassium permanganate test value (PMT) of 30 minutes or longer. The initial boiling point is preferably 204 °C or higher, more preferably 205 °C or higher, even more preferably 206 °C or higher or 207 °C, and particularly preferably 208 °C or higher. The potassium permanganate test value (PMT) is more preferably longer than 30 minutes (e.g., 32 minutes or longer), even more preferably 35 minutes or longer (e.g., 40 minutes or longer), and particularly preferably 50 minutes or longer (especially, 60 minutes or longer).

According to the method for manufacturing 1,3-butylene glycol of the sixth aspect of the present disclosure, a 1,3-butylene glycol product having an initial boiling point of higher than 203 °C and a potassium permanganate test value (PMT) of 30 minutes or longer, a 1,3-butylene glycol product having an initial boiling point of higher than 203 °C and a dry point of 209 °C or lower, and further, a 1,3-butylene glycol product having an initial boiling point of higher than 203 °C, a dry point of 209 °C or lower, and a potassium permanganate test value (PMT) of 30 minutes or longer can be obtained. The initial boiling point is preferably 204 °C or higher, more preferably 205 °C or higher, even more preferably 206 °C or higher or 207 °C or higher, and particularly preferably 208 °C or higher. The potassium permanganate test value (PMT) is more preferably longer than 30 minutes (e.g., 32 minutes or longer), even more preferably 35 minutes or longer (e.g., 40 minutes or longer), and particularly preferably 50 minutes or longer (especially, 60 minutes or longer).

The 1,3-butylene glycol product according to the second aspect of the present disclosure preferably has, in a gas chromatographic analysis (GC analysis) performed under conditions set forth below:
an area ratio of a 1,3-butylene glycol peak of greater than 98.5% and a total area ratio of peaks shorter in retention time than the 1,3-butylene glycol peak of less than 0.3%,
in which the conditions for the gas chromatographic analysis are as follows:
Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

Additionally, in the 1,3-butylene glycol product of the third aspect of the present disclosure, the peak area ratio of 1,3-butylene glycol is preferably higher than 98.5%, according to a gas chromatographic analysis (GC analysis) performed under conditions set forth below.

Conditions for the gas chromatographic analysis are the same as above. Therefore, the conditions for gas chromatographic analysis are omitted here.

Additionally, in the 1,3-butylene glycol product of the fourth and fifth aspects of the present disclosure, the peak area ratio of 1,3-butylene glycol is preferably higher than 98.7% in the gas chromatographic analysis (GC analysis) under the following conditions.

Furthermore, the total area ratio of peaks having shorter retention times than the 1,3-butylene glycol peak is preferably lower than 0.3%.

Conditions for the gas chromatographic analysis are the same as above. Therefore, the conditions for gas chromatographic analysis are omitted here.

Furthermore, in the 1,3-butylene glycol product of the fifth aspect of the present disclosure, the total area ratio of peaks having longer retention times than the peak of 1,3-butylene glycol is preferably lower than 1.2%.

Additionally, according to the method for manufacturing 1,3-butylene glycol of the sixth aspect of the present disclosure, a 1,3-butylene glycol product further having an area ratio of a peak of 1,3-butylene glycol of higher than 98.7% in the gas chromatographic analysis (GC analysis) under the following conditions, in addition to the above characteristics, can be obtained. Additionally, according to the method for manufacturing 1,3-butylene glycol of the present disclosure, a 1,3-butylene glycol product further having a total area ratio of peaks having shorter retention times than the peak of 1,3-butylene glycol of lower than 0.3%, in addition to the above characteristics, can be obtained. Additionally, according to the method for manufacturing 1,3-butylene glycol of the present disclosure, a 1,3-butylene glycol product further having a total area ratio of peaks having longer retention times than the peak of 1,3-butylene glycol of lower than 1.2%, in addition to the above characteristics, can be obtained.

The conditions for the gas chromatographic analysis are the same as above. Therefore, the conditions for gas chromatographic analysis are omitted here.

The area ratio of the 1,3-butylene glycol peak is preferably 98.6% or greater, 98.7% or greater, 98.8% or greater, 98.9% or greater, 99% or greater, 99.1% or greater, 99.2% or greater, or 99.3% or greater, more preferably 99.4% or greater, even more preferably 99.5% or greater or 99.6% or greater or 99.7% or greater, and particularly preferably 99.8% or greater.

The total area ratio of the peaks longer in retention time than the 1,3-butylene glycol peak is preferably 1.1% or less, 1% or less, 0.9% or less, 0.8% or less, 0.7% or less, or 0.6% or less, more preferably 0.5% or less, even more preferably 0.4% or less, 0.3% or less, 0.2% or less, 0.1% or less, or 0.05% or less, and particularly preferably 0.03% or less.

The total area ratio of the peaks shorter in retention time than the 1,3-butylene glycol peak is preferably, 0.28% or less, 0.25% or less, more preferably 0.23% or less, even more preferably 0.2% or less, 0.17% or less, 0.15% or less, 0.12% or less, 0.1% or less, 0.07% or less, 0.04% or less, 0.03% or less, 0.02% or less, 0.01% or less, or 0.007% or less, and particularly preferably 0.005% or less (for example, 0.002% or less).

In the present disclosure, the “(peak) area ratio” means an area proportion (area %) of a specific peak relative to the sum of the areas of all peaks appearing in the chromatogram. In addition, all peaks mean, for example, all of the peaks appearing in the analysis continued until and discontinued at a relative retention time of 7.8, provided that the relative retention time of 1,3-butylene glycol is 1.0.

Also, in the 1,3-butylene glycol product of the present disclosure, a content of water is preferably lower than 0.4 wt. %. According to the method for manufacturing 1,3-butylene glycol of the present disclosure, a 1,3-butylene glycol product having a water content of lower than 0.4 wt. % can be obtained. The content of water is more preferably 0.3 wt. % or lower, even more preferably 0.2 wt. % or lower, 0.1 wt. % or lower, 0.07 wt. % or lower, 0.05 wt. % or lower, 0.03 wt. % or lower, 0.02 wt. % or lower, or 0.01 wt. % or lower, and particularly preferably 0.005 wt. % or lower. Note that the content of water can be quantified by a Karl Fischer moisture.

In addition, in the 1,3-butylene glycol product of the present disclosure, the content of acetaldehyde is preferably 2 ppm or lower. Furthermore, the content of the crotonaldehyde is preferably 1.2 ppm or lower.

Further, in the 1,3-butylene glycol product of the present disclosure, a content of acetaldehyde is preferably lower than 2 ppm. Furthermore, the content of the crotonaldehyde is preferably lower than 1.2 ppm.

According to the method for manufacturing 1,3-butylene glycol of the present disclosure, a 1,3-butylene glycol product having an acetaldehyde content of lower than 2 ppm can be obtained. According to the method for manufacturing 1,3-butylene glycol of the present disclosure, a 1,3-butylene glycol product having a crotonaldehyde content of lower than 1.2 ppm can be obtained.

The acetaldehyde content and the crotonaldehyde content of the 1,3-butylene glycol product can be quantified by GC-MS analysis (gas mass analysis), for example GC-MS analysis under the following conditions. In GC-MS analysis, even very small peaks are all subjected to mass spectrometry, and each component is quantified. Since the analysis is performed for a specific mass, a substance different in mass is not detected even when another impurity overlaps the peak. Therefore, the analysis is more sensitive than GC analysis which will be described below. In the present specification, the unit “ppm” of the content of each component by GC-MS analysis [content of each compound or the like represented by Formula (A) or (B)] means “ppm by weight”.

### Conditions for GC-MS Analysis

Analytical Column: a column with dimethylpolysiloxane as a stationary phase, having a length of 30 m, an inner diameter of 0.25 mm, and a film thickness of 1.0 μm
Heating Conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Ion source temperature: EI 230 °C, CI 250 °C
Q Pole temperature: 150 °C
Sample: subjected to analysis as it was

When the relative retention time of the peak of 1,3-butylene glycol is 1.0 in the GC-MS analysis conditions, a relative retention time of a peak of acetaldehyde is from 0.3 to 0.5, and a relative retention time of a peak of crotonaldehyde is from 0.3 to 0.5.

Furthermore, the acetaldehyde content of the 1,3-butylene glycol product is more preferably 1.8 ppm or lower, 1.7 ppm or lower, even more preferably 1.5 ppm or lower, 1.4 ppm or lower, 1.3 ppm or lower, and particularly preferably 1.2 ppm or lower, 1.1 ppm or lower, 1.0 ppm or lower, 0.9 ppm or lower, 0.8 ppm or lower, 0.7 ppm or lower, 0.6 ppm or lower, 0.5 ppm or lower, or 0.3 ppm or lower (for example, 0.2 ppm or lower). Furthermore, the crotonaldehyde content of the 1,3-butylene glycol product is more preferably 1.0 ppm or lower, 0.9 ppm or lower, 0.8 ppm or lower, even more preferably 0.7 ppm or lower, 0.6 ppm or lower, and particularly preferably 0.5 ppm or lower, 0.4 ppm or lower, 0.3 ppm or lower, or 0.2 ppm or lower (for example, 0.1 ppm or lower).

In the production method according to the first aspect of the present disclosure, the area ratio of the 1,3-butylene glycol peak and the total area ratio of the peaks longer in retention time than the 1,3-butylene glycol peak are within the ranges described above, and thus high-purity and high-quality 1,3-butylene glycol products are provided.

In the production method according to the second aspect of the present disclosure, the area ratio of the 1,3-butylene glycol peak and the total area ratio of the peaks shorter in retention time than the 1,3-butylene glycol peak are within the ranges described above, and thus high-purity and high-quality 1,3-butylene glycol products are provided.

In the production method according to the third aspect of the present disclosure, with the peak area ratio of 1,3-butylene glycol, the acetaldehyde content, and the crotonaldehyde content in the 1,3-butylene glycol product being within the above ranges, a high-quality and high-quality 1,3-butylene glycol product is provided.

In the production method according to the fourth aspect of the present disclosure, with the initial boiling point and potassium permanganate test value within the above ranges, and further, preferably with the area ratio of the 1,3-butylene glycol peak, the total area ratio of the peaks having shorter retention times than the 1,3-butylene glycol peak, the acetaldehyde content, and the crotonaldehyde content also within the above ranges, a high-quality and high-quality 1,3-butylene glycol product is provided.

In the production method according to the fifth aspect of the present disclosure, the initial boiling point and dry point are within the above ranges, and, further, the potassium permanganate test value, the peak area ratio of 1,3-butylene glycol, the total area ratio of the peaks having shorter retention times than the peak of 1,3-butylene glycol, the total area ratio of the peaks having longer retention times than the peak of the 1,3-butylene glycol, and the content of acetaldehyde, and the content of crotonaldehyde are within the above ranges, and thus a high-quality and high-quality 1,3-butylene glycol product is provided.

In the production method according to the sixth aspect of the present disclosure, according to the method for manufacturing 1,3-butylene glycol of the present disclosure, it is possible to obtain a high-purity and high-quality 1,3-butylene glycol product having excellent initial boiling point and dry point, and, further, a large potassium permanganate test value, a high area ratio of the peak of 1,3-butylene glycol, a low total area ratio of the peaks having shorter retention times than the peak of 1,3-butylene glycol, a low total area ratio of the peaks having longer retention times than the peak of the 1,3-butylene glycol, a low content of acetaldehyde, and a low content of crotonaldehyde.

### 1,3-Butylene Glycol Product—Seventh Aspect of the Present Disclosure

In the 1,3-butylene glycol product according to the seventh aspect of the present disclosure, at least one of a content of methyl vinyl ketone, a content of acetone, a content of butylaldehyde, a content of acetaldol, a content of a compound represented by Formula (1) below, a content of a compound represented by Formula (2) below, and a content of a compound represented by Formula (3) below, and a total content of a compound represented by Formula (4) below and a compound represented by Formula (5) below, is less than 8 ppm.

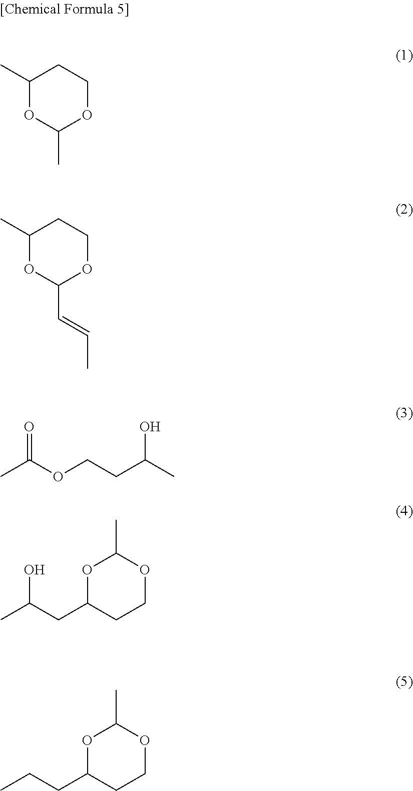

Each of the contents of methyl vinyl ketone, acetone, butylaldehyde, acetaldol, the compound represented by Formula (1), the compound represented by Formula (2), the compound represented by Formula (3), the compound represented by Formula (4), and the compound represented by Formula (5) can be quantified by GC-MS analysis performed under the following conditions. In GC-MS analysis, even very small peaks are all subjected to mass spectrometry, and each component is quantified. Since the analysis is performed for a specific mass, a substance different in mass is not detected even when another impurity overlaps the peak. Therefore, the analysis is more sensitive than GC analysis which will be described below. In the present specification, the unit “ppm” of the content of each component by GC-MS analysis means “ppm by weight”.

### Conditions for GC-MS Analysis

Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Ion source temperature: EI 230 °C, CI 250 °C
Q Pole temperature: 150 °C
Sample: subjected to analysis as it is

Retention times of peaks of methyl vinyl ketone, acetone, butylaldehyde, acetaldol [CH₃CH(OH)CH₂CH(═O)], and the compound represented by Formula (1) below are usually shorter than a retention time of a peak of 1,3-butylene glycol. In the above analytical conditions, the retention time of a peak of 1,3-butylene glycol is usually from 5.5 minutes to 7 minutes. When a relative retention time of the peak of 1,3-butylene glycol is defined as 1.0 under the above analysis conditions, a relative retention time of the peak of methyl vinyl ketone is from 0.3 to 0.5, a relative retention time of the peak of acetone is from 0.3 to 0.5, a relative retention time of the peak of butylaldehyde is from 0.3 to 0.5, a relative retention time of the peak of acetaldol is from 0.4 to 0.6, and a relative retention time of the peak of the compound represented by Formula (1) above is from 0.6 to 0.8. The compound represented by Formula (1) is an acetal compound produced by a reaction of acetaldehyde with 1,3-butylene glycol.

Under the above analysis conditions, retention times of peaks of the compound represented by Formula (2), the compound represented by Formula (3), the compound represented by Formula (4), and the compound represented by Formula (5) are usually longer than the retention time of the peak of 1,3-butylene glycol. When the relative retention time of the peak of 1,3-butylene glycol is defined as 1.0 under the above analysis conditions, a relative retention time of the peak of the compound represented by Formula (2) is from 1.3 to 1.7, a relative retention time of the peak of the compound represented by Formula (3) is from 1.0 to 1.2, a relative retention time of the peak of the compound represented by Formula (4) is from 1.6 to 2.0, and a relative retention time of the peak of the compound represented by Formula (5) is from 1.3 to 1.7. The compound represented by Formula (2) is an acetal compound produced by a reaction of crotonaldehyde with 1,3-butylene glycol. The compound represented by Formula (3) is 1,3-butanediol monoacetate produced by a reaction of acetic acid with 1,3-butylene glycol. The compound represented by Formula (4) and the compound represented by Formula (5) are acetaldehyde multimeric acetals [the compound represented by Formula (4) has a hydroxy group, and the compound represented by Formula (5) does not have a hydroxy group).

In the 1,3-butylene glycol product according to the present disclosure, at least one of eight contents: the content of methyl vinyl ketone, the content of acetone, the content of butylaldehyde, the content of acetaldol, the content of the compound represented by Formula (1) below, the content of the compound represented by Formula (2) below, the content of the compound represented by Formula (3) below, and the total content of the compound represented by Formula (4) below and the compound represented by Formula (5) below, is less than 8 ppm (for example, 7 ppm or less, preferably 6 ppm or less, more preferably 5 ppm or less, even more preferably 4 ppm or less, 3 ppm or less, 2 ppm or less, 1 ppm or less, or 0.5 ppm or less). Thus, among the eight contents, one content may be less than 8 ppm (for example, 7 ppm or less, preferably 6 ppm or less, more preferably 5 ppm or less, even more preferably 4 ppm or less, 3 ppm or less, 2 ppm or less, 1 ppm or less, or 0.5 ppm or less); two contents may be less than 8 ppm (for example, 7 ppm or less, preferably 6 ppm or less, more preferably 5 ppm or less, even more preferably 4 ppm or less, 3 ppm or less, 2 ppm or less, 1 ppm or less, or 0.5 ppm or less); three contents may be less than 8 ppm (for example, 7 ppm or less, preferably 6 ppm or less, more preferably 5 ppm or less, even more preferably 4 ppm or less, 3 ppm or less, 2 ppm or less, 1 ppm or less, or 0.5 ppm or less); four contents may be less than 8 ppm (for example, 7 ppm or less, preferably 6 ppm or less, more preferably 5 ppm or less, even more preferably 4 ppm or less, 3 ppm or less, 2 ppm or less, 1 ppm or less, or 0.5 ppm or less); five contents may be less than 8 ppm (for example, 7 ppm or less, preferably 6 ppm or less, more preferably 5 ppm or less, even more preferably 4 ppm or less, 3 ppm or less, 2 ppm or less, 1 ppm or less, or 0.5 ppm or less); six contents may be less than 8 ppm (for example, 7 ppm or less, preferably 6 ppm or less, more preferably 5 ppm or less, even more preferably 4 ppm or less, 3 ppm or less, 2 ppm or less, 1 ppm or less, or 0.5 ppm or less); seven contents may be less than 8 ppm (for example, 7 ppm or less, preferably 6 ppm or less, more preferably 5 ppm or less, even more preferably 4 ppm or less, 3 ppm or less, 2 ppm or less, 1 ppm or less, or 0.5 ppm or less); and all of the eight contents may be less than 8 ppm (for example, 7 ppm or less, preferably 6 ppm or less, more preferably 5 ppm or less, even more preferably 4 ppm or less, 3 ppm or less, 2 ppm or less, 1 ppm or less, or 0.5 ppm or less).

In the 1,3-butylene glycol product, among the eight contents: the content of methyl vinyl ketone, the content of acetone, the content of butylaldehyde, the content of acetaldol, the content of the compound represented by Formula (1), the content of the compound represented by Formula (2), the content of the compound represented by Formula (3), and the total content of the compound represented by Formula (4) and the compound represented by Formula (5), at least four (four, five, six, seven or eight) contents are preferably less than 8 ppm (for example, 7 ppm or less, preferably 6 ppm or less, more preferably 5 ppm or less, even more preferably 4 ppm or less, 3 ppm or less, 2 ppm or less, 1 ppm or less, or 0.5 ppm or less). Particularly, all of the eight contents are preferably less than 8 ppm (for example, 7 ppm or less, preferably 6 ppm or less, more preferably 5 ppm or less, and even more preferably 4 ppm or less, 3 ppm or less, 2 ppm or less, 1 ppm or less, or 0.5 ppm or less).

In the 1,3-butylene glycol product, among the eight contents: the content of methyl vinyl ketone, the content of acetone, the content of butylaldehyde, the content of acetaldol, the content of the compound represented by Formula (1), the content of the compound represented by Formula (2), the content of the compound represented by Formula (3), and the total content of the compound represented by Formula (4), and the compound represented by Formula (4), at least one (for example, one, two, three, four, . . . or all) of sums of two contents (for example, a sum of the content of methyl vinyl ketone and the content of acetone, a sum of the content of methyl vinyl ketone and the content of butyl aldehyde, a sum of the content of methyl vinyl ketone and the content of acetaldole, a sum of the content of methyl vinyl ketone and the content of the compound represented by Formula (1), a sum of the content of methyl vinyl ketone and the content of the compound represented by Formula (2), a sum of the content of methyl vinyl ketone and the content of the compound represented by Formula (3), a sum of the content of methyl vinyl ketone and the total content of the compound represented by Formula (4) and the compound represented by Formula (5), and a sum of the content of acetone and the content of butyl aldehyde) may be less than 16 ppm (for example, 15 ppm or less, preferably 14 ppm or less, more preferably 13 ppm or less, even more preferably 12 ppm or less, and particularly preferably 11 ppm or less, 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, or 0.5 ppm or less).

Further, in the 1,3-butylene glycol product, among the eight contents: the content of methyl vinyl ketone, the content of acetone, the content of butylaldehyde, the content of acetaldol, the content of the compound represented by Formula (1), the content of the compound represented by Formula (2), the content of the compound represented by Formula (3), and the total content of the compound represented by Formula (4), and the compound represented by Formula (5), at least one (for example, one, two, three, four, . . . or all) of sums of three contents (for example, a sum of the content of methyl vinyl ketone, the content of acetone, and the content of butylaldehyde; a sum of the content of methyl vinyl ketone, the content of acetaldol, and the content of the compound represented by the formula (1); and a sum of the content of acetone, the content of butylaldehyde, and the content of acetaldol) may be less than 24 ppm (for example, 20 ppm or less, preferably 18 ppm or less, more preferably 16 ppm or less, even more preferably 14 ppm or less, and particularly preferably 12 ppm or less, 11 ppm or less, 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, or 0.5 ppm or less).

Also, in the 1,3-butylene glycol product, among the content of methyl vinyl ketone, the content of acetone, the content of butylaldehyde, the content of acetaldol, the content of the compound represented by Formula (1), the content of the compound represented by Formula (2), the content of the compound represented by Formula (3), and the total content of the compound represented by Formula (4), and the compound represented by Formula (5), at least one (for example, one, two . . . or all) of sums of four contents (for example, a sum of the content of methyl vinyl ketone, the content of acetone, the content of butylaldehyde, and the content of acetaldol; and a sum of the content of acetone, the content of butylaldehyde, the content of acetaldol, and the content of the compound represented by Formula (1)) may be less than 32 ppm (for example, 30 ppm or less, preferably 25 ppm or less, more preferably 20 ppm or less, even more preferably 18 ppm or less, and particularly preferably 16 ppm or less, 14 ppm or less, 12 ppm or less, 10 ppm or less, 8 ppm or less, 7 ppm or less, 6 ppm or less, 5 ppm or less, 4 ppm or less, 3 ppm or less, 2 ppm or less, 1 ppm or less, or 0.5 ppm or less).

Further, in the 1,3-butylene glycol product, a sum of the content of methyl vinyl ketone, the content of acetone, the content of butylaldehyde, the content of acetaldol, the content of the compound represented by Formula (1), the content of the compound represented by Formula (2), the content of the compound represented by Formula (3), and the content of the compound represented by Formula (4) and the content of the compound represented by Formula (5) may be less than 71 ppm (for example, 60 ppm or less, preferably 50 ppm or less, more preferably 40 ppm or less, even more preferably 30 ppm or less, and particularly preferably 20 ppm or less, 18 ppm or less, 16 ppm or less, 14 ppm or less, 12 ppm or less, 10 ppm or less, 8 ppm or less, 7 ppm or less, 6 ppm or less, 5 ppm or less, 4 ppm or less, 3 ppm or less, 2 ppm or less, 1 ppm or less, or 0.5 ppm or less).

In addition, in the 1,3-butylene glycol product, a sum of the content of methyl vinyl ketone, the content of acetone, the content of butylaldehyde, the content of acetaldol, and the content of the compound represented by Formula (1), which are contents of impurities generally shorter in GC retention time than 1,3-butylene glycol, is preferably less than 47 ppm (for example, 40 ppm or less, preferably 30 ppm or less, more preferably 25 ppm or less, even more preferably 20 ppm or less, and particularly preferably 18 ppm or less, 16 ppm or less, 14 ppm or less, 12 ppm or less, 10 ppm or less, 8 ppm or less, 7 ppm or less, 6 ppm or less, 5 ppm or less, 4 ppm or less, 3 ppm or less, 2 ppm or less, 1 ppm or less, or 0.5 ppm or less), and a sum of the content of the compound represented by Formula (2), the content of the compound represented by Formula (3), the content of the compound represented by Formula (4), and the content of the compound represented by Formula (5), which are contents of impurities generally longer in GC retention time than 1,3-butylene glycol, is preferably less than 24 ppm (for example, 20 ppm or less, preferably 18 ppm or less, more preferably 16 ppm or less, even more preferably 14 ppm or less, and particularly preferably 13 ppm or less, 12 ppm or less, 11 μm or less, 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, or 0.5 ppm or less).

It is preferable that, in the 1,3-butylene glycol product according to the present disclosure, at least the content of acetaldol be less than 8 ppm. The acetaldol generates crotonaldehyde by heat. The crotonaldehyde can be a substance that causes coloration, a substance that causes odor, or a substance that causes acidity. The content of acetaldol is more preferably 7 ppm or less, even more preferably 6 ppm or less, 5 ppm or less, 4 ppm or less, 3 ppm or less, 2 ppm or less, 1 ppm or less, or 0.5 ppm or less.

It is preferable that, in the 1,3-butylene glycol product according to the present disclosure, at least the content of the compound represented by Formula (3) is less than 8 ppm. The compound represented by Formula (3) generates acetic acid by hydrolysis, which causes acetic acid odor. Also, it increases an acid concentration (acid content) of the product. The content of the compound represented by Formula (3) is more preferably 7 ppm or less, and even more preferably 6 ppm or less, 5 ppm or less, 4 ppm or less, 3 ppm or less, 2 ppm or less, 1 ppm or less, or 0.5 ppm or less.

In the 1,3-butylene glycol product according to the present disclosure, the total content of methyl vinyl ketone, acetone, and butylaldehyde is preferably 24 ppm or less. All of these compounds have a carbonyl group and can be a substance that causes coloration, a substance that causes odor, or a substance that causes acidity. The total content of methyl vinyl ketone, acetone and butylaldehyde is more preferably 20 ppm or less, and even more preferably 18 ppm or less, 16 ppm or less, 14 ppm or less, 12 ppm or less, 11 ppm or less, 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, or 0.5 ppm or less.

In the 1,3-butylene glycol product according to the present disclosure, the total content of the compound represented by Formula (1), the compound represented by Formula (2), the compound represented by Formula (4), and the compound represented by Formula (5) is preferably 24 ppm or less. All of these compounds are acetal compounds, which generate acetaldehyde by hydrolysis. The acetaldehyde can be a substance that causes coloration, a substance that causes odor, or a substance that causes acidity.

In the 1,3-butylene glycol product according to the present disclosure, the content of acetaldehyde is preferably less than 4 ppm (in particular, less than 2 ppm). Furthermore, the content of crotonaldehyde is preferably less than 2 ppm (particularly less than 1.2 ppm). The acetaldehyde and the crotonaldehyde can be substances that cause coloration, substances that cause odor, or substances that cause acidity. They also reduce the potassium permanganate test value of the product. Note that the acetaldehyde content and the crotonaldehyde content of the 1,3-butylene glycol product can be quantified by GC-MS analysis (gas mass spectrometry) described above.

Under the GC-MS analysis conditions, when the relative retention time of the peak of 1,3-butylene glycol is defined as 1.0, a relative retention time of a peak of acetaldehyde is from 0.3 to 0.5, and a relative retention time of a peak of crotonaldehyde is from 0.3 to 0.5.

The content of acetaldehyde in the 1,3-butylene glycol product is more preferably 1.8 ppm or less, even more preferably 1.7 ppm or less, 1.5 ppm or less, 1.4 ppm or less, 1.3 ppm or less, 1.2 ppm or less, 1.1 ppm or less, 1.0 ppm or less, 0.9 ppm or less, 0.8 ppm or less, 0.7 ppm or less, 0.6 ppm or less, or 0.5 ppm or less, and particularly preferably 0.3 ppm or less (for example, 0.2 ppm or less). Furthermore, the content of crotonaldehyde in the 1,3-butylene glycol product is more preferably 1.0 ppm or less, even more preferably 0.9 ppm or less, 0.8 ppm or less, 0.7 ppm or less, 0.6 ppm or less, 0.5 ppm or less, 0.4 ppm or less, or 0.3 ppm or less, and particularly preferably 0.2 ppm or less (e.g., 0.1 ppm or less).

The 1,3-butylene glycol product preferably has an acid concentration of less than 11 ppm in terms of acetic acid, and an acid concentration of less than 23 ppm in terms of acetic acid after a 90 wt. % aqueous solution has been kept at 100 °C for 1 week.

Desirably, the 1,3-butylene glycol product according to the present disclosure preferably has an acid concentration of, for example, 10 ppm or less (preferably 9 ppm or less, more preferably 8 ppm or less, even preferably 7 ppm or less, particularly preferably 6 ppm or less, and most preferably 5 ppm or less, 4 ppm or less, or 3 ppm or less) in terms of acetic acid, and an acid concentration of, for example, 20 ppm or less (preferably 19 ppm or less, 18 ppm or less, 17 ppm or less, or 16 ppm or less, more preferably 15 ppm or less, even more preferably 14 ppm or less, 13 ppm or less, 12 ppm or less, 11 ppm or less, and particularly preferably 10 ppm or less, 9 ppm or less, 8 ppm or less, 7 ppm or less, 6 ppm or less, 5 ppm or less, 4 ppm or less, 3 ppm or less, or 2 ppm or less) in terms of acetic acid after a 90 wt. % aqueous solution has been kept at 100 °C for 1 week. The 90 wt. % aqueous solution means an aqueous solution prepared by mixing the 1,3-butylene glycol product and water (e.g., pure water) to adjust the 1,3-butylene glycol product to 90 wt. %.

For the acid concentration in terms of acetic acid of the 90 wt. % aqueous solution of the 1,3-butylene glycol product according to the present disclosure, a ratio of the acid concentration after the solution has been kept at 100 °C for 1 week to the acid concentration before the solution has kept at 100 °C for 1 week, that is [(acid concentration after the solution has been kept at 100 °C for 1 week)/(acid concentration before the solution has been kept at 100 °C for 1 week)×100(%)], is preferably 200% or less, more preferably 150% or less, and even more preferably 120% or less.

Desirably, in the 1,3-butylene glycol product according to the present disclosure, an APHA (Hazen color number) is, for example, 6 or less (preferably 5 or less, more preferably 4 or less, even more preferably 3 or less, and particularly preferably 2 or less). And after the 1,3-butylene glycol product has been kept at 180 °C for 3 hours in air atmosphere, an APHA is, for example, 78 or less (preferably 65 or less, more preferably 60 or less, even more preferably 55 or less, 50 or less, 45 or less, 40 or less, 35 or less, 30 or less, 25 or less, 20 or less, and particularly preferably 18 or less, 15 or less, 14 or less, 13 or less, 12 or less, 11 or less, 10 or less, 9 or less, 8 or less, or 7 or less). Furthermore, an APHA, after the 1,3-butylene glycol product has been kept at 100 °C for 75 days in air atmosphere, is, for example, 42 or less (preferably 35 or less, 30 or less, 25 or less, 20 or less, 18 or less, 16 or less, 15 or less, 14 or less, or 13 or less, more preferably 12 or less, 11 or less, 10 or less, 9 or less, 8 or less, 7 or less, or 6 or less, and even more preferably 5 or less, 4 or less, 3 or less, or 2 or less).

For the APHA of the 1,3-butylene glycol product according to the present disclosure, a ratio of the APHA after the 1,3-butylene glycol product has been kept at 100 °C for 75 days to the APHA before the 1,3-butylene glycol product has been kept at 100 °C for 75 days, [(APHA after the 1,3-butylene glycol product has been kept at 100 °C for 75 days)/(APHA before the 1,3-butylene glycol product has been kept at 100 °C for 75 days)], is not particularly limited, but is preferably 10 or less, more preferably 8 or less, even more preferably 7 or less, and particularly preferably 6 or less (for example, 5 or less, 4 or less, or 3 or less). In addition, the ratio is 1 or greater, or may be 2 or greater.

Additionally, the 1,3-butylene glycol product according to the present disclosure preferably has an initial boiling point of higher than 203 °C. The initial boiling point is preferably 204 °C or higher, more preferably 205 °C or higher, even more preferably 206 °C or higher or 207 °C, and particularly preferably 208 °C or higher.

Additionally, the 1,3-butylene glycol product according to the present disclosure preferably has a dry point of 209 °C or lower.

The 1,3-butylene glycol product according to the present disclosure preferably has a potassium permanganate test value (PMT) of 30 minutes or longer. The potassium permanganate test value (PMT) is more preferably longer than 30 minutes (e.g., 32 minutes or longer), even more preferably 35 minutes or longer (e.g., 40 minutes or longer), and particularly preferably 50 minutes or longer (especially, 60 minutes or longer).

In the 1,3-butylene glycol product according to the present disclosure, an area ratio (GC area ratio) of the peak of 1,3-butylene glycol is preferably greater than 98.7%, in the gas chromatographic analysis (GC analysis) performed under conditions set forth below. Furthermore, a total area ratio of peaks shorter in retention time than the peak of 1,3-butylene glycol is preferably less than 0.3%. Furthermore, a total area ratio of peaks longer in retention time than the peak of 1,3-butylene glycol is preferably less than 1.2%.

### The Conditions for the Gas Chromatographic Analysis are as Follows:

Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

In the present disclosure, the “(peak) area ratio” means a ratio of the area of a specific peak relative to the sum of the areas of all peaks appearing in the chromatogram. In addition, all peaks mean, for example, all of the peaks appearing in the analysis continued until and discontinued at a relative retention time of 7.8, provided that the relative retention time of 1,3-butylene glycol is 1.0. When the area ratios of the peaks are within the ranges described above, the occurrence of odor, the increase in acid concentration over time in a state containing water, and the coloration over time are reduced.

The area ratio of the peak of 1,3-butylene glycol is 98.8% or greater, more preferably 98.9% or greater, even more preferably 99% or greater, 99.1% or greater, 99.2% or greater, 99.3% or greater, 99.4% or greater, 99.5% or greater, 99.6% or greater, or 99.7% or greater, and particularly preferably 99.8% or greater.

The total area ratio of the peaks shorter in retention time than the peak of 1,3-butylene glycol is preferably 0.28% or less, more preferably 0.25% or less, even more preferably 0.23% or less, 0.2% or less, 0.17% or less, 0.15% or less, 0.12% or less, 0.1% or less, 0.07% or less, 0.04% or less, 0.03% or less, 0.02% or less, 0.01% or less, or 0.007% or less, and particularly preferably 0.005% or less (for example, 0.002% or less).

The total area ratio of the peaks longer in retention time than the peak of 1,3-butylene glycol is preferably 1% or less, more preferably 0.9% or less, more preferably 0.8% or less, 0.7% or less, 0.6% or less, 0.5% or less, 0.4% or less, 0.3% or less, or 0.2% or less, and particularly preferably 0.1% or less.

In the 1,3-butylene glycol product according to the present disclosure, a content of water is preferably less than 0.4 wt. %. The content of water is preferably 0.3 wt. % or less, more preferably 0.2 wt. % or less, even more preferably 0.1 wt. % or less, 0.07 wt. % or less, 0.05 wt. % or less, 0.03 wt. % or less, 0.02 wt. % or less, or 0.01 wt. % or less, and particularly preferably 0.005 wt. % or less. Note that the content of water can be quantified by a Karl Fischer water content measurement instrument.

A high-purity and high-quality 1,3-butylene glycol product with little deterioration in quality over time is provided by suppressing the contents of the specific nine compounds, and, additionally, by setting the content of acetaldehyde and the content of crotonaldehyde to within the above ranges, by setting the initial boiling point and the dry point to within the above ranges, and further by setting the potassium permanganate test value, the area ratio of the peak of 1,3-butylene glycol, the total area ratio of the peaks shorter in retention time than the peak of 1,3-butylene glycol, and the total area ratio of the peaks longer in retention time than the peak of 1,3-butylene glycol to within the above ranges.

### 1,3-Butylene Glycol Product—Eighth Aspect of the Present Disclosure

The 1,3-butylene glycol product according to the eighth aspect of the present disclosure is a 1,3-butylene glycol product containing 1,3-butylene glycol, in which, after the 1,3-butylene glycol product has been kept at 180 °C for 3 hours in air atmosphere, at least one of contents of compounds represented by the following Formula (A) or (B) is less than 8 ppm:

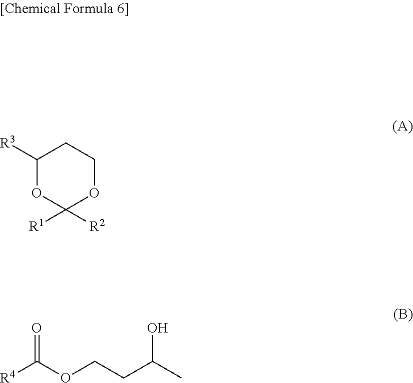

where R¹ to R⁴ are the same as or different from each other, and each of R¹ to R⁴ is a hydrogen atom, an alkyl group with from 1 to 4 carbon atoms which may be substituted with a hydroxy group, or an alkenyl group with 2 to 4 carbon atoms which may be substituted with a hydroxy group. The compound represented by Formula (A) is a cyclic acetal compound, and the compound represented by Formula (B) is a 3-hydroxybutyl carboxylate compound. These compounds are also present as impurities during production of 1,3-butylene glycol, but are impurities whose content increases over time, when the 1,3-butylene glycol product is stored for a long period of time. The compound represented by Formula (A) is hydrolyzed in the presence of water to produce a corresponding carbonyl compound. The thus-produced carbonyl compound is itself a reducing substance, not only diminishes the potassium permanganate test value, but also is highly reactive and likely to produce a variety of complex compounds by heat or oxygen, and may become a substance that causes coloration, a substance that causes odor or a substance that causes acidity. Also, the compound represented by Formula (B) is hydrolyzed in the presence of water to produce a corresponding carboxylic acid. The thus-produced carboxylic acid may be a substance that causes odor or a substance that causes acidity.

In Formulas (A) and (B), R¹ to R⁴ are the same as or different from each other, and each of R¹ to R⁴ is a hydrogen atom, an alkyl group with from 1 to 4 carbon atoms which may be substituted with a hydroxy group, or an alkenyl group with 2 to 4 carbon atoms which may be substituted with a hydroxy group. Examples of the alkyl group with from 1 to 4 carbons which may be substituted with a hydroxy group include methyl, ethyl, propyl, butyl, 2-hydroxyethyl, and 2-hydroxypropyl groups. Examples of the alkenyl group with 2 to 4 carbon atoms which may be substituted with a hydroxy group include vinyl and 1-propenyl groups. Examples of R¹ include a hydrogen atom and methyl group. Examples of R² include a hydrogen atom, methyl, vinyl, 2-hydroxyethyl, 2-hydroxypropyl, 1-propenyl, and butyl group. Examples of R³ include a methyl group. Examples of R⁴ include a hydrogen atom, methyl, 2-hydroxypropyl, and 1-propenyl group.

Representative examples of the compound represented by Formula (A) include the compounds represented by the following formulas (1) to (7). Representative examples of the compound represented by Formula (B) include the compounds represented by the following formulas (8) to (10).

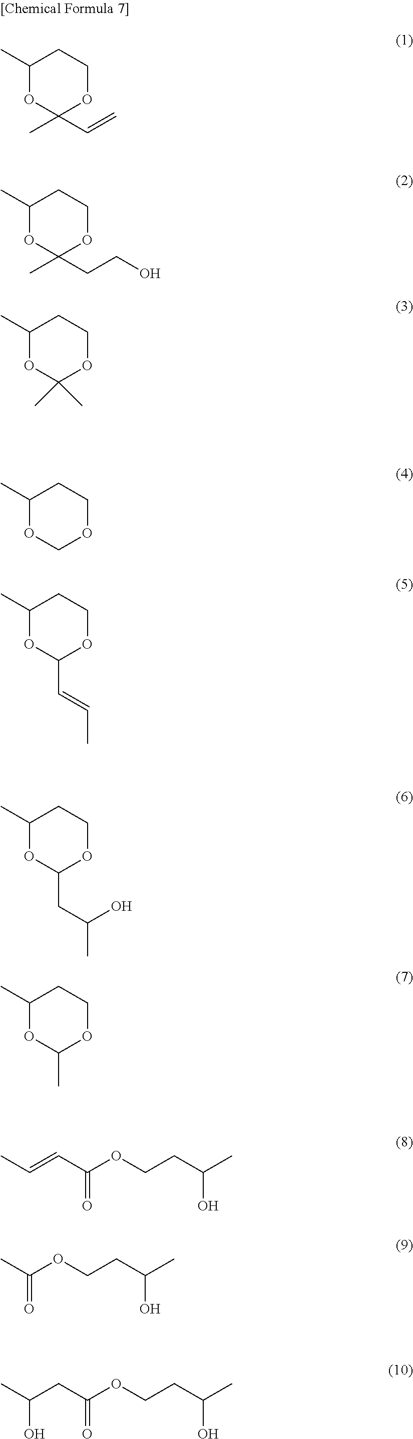

A content of each of the compounds represented by Formula (A) or (B) [e.g., compounds represented by Formulas (1) to (10) above] can be quantified by GC-MS analysis under the following conditions. In GC-MS analysis, even very small peaks are all subjected to mass spectrometry, and each component is quantified. Since the analysis is performed for a specific mass, a substance different in mass is not detected even when another impurity overlaps the peak. Therefore, the analysis is more sensitive than GC analysis which will be described below. In the present specification, the unit “ppm” of the content of each component determined by GC-MS analysis [content of each compound or the like represented by Formula (A) or (B)] means “ppm by weight”.

### Conditions for GC-MS Analysis

Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Ion source temperature: EI 230 °C, CI 250 °C
Q Pole temperature: 150 °C
Sample: subjected to analysis as it is

In the above analytical conditions, the retention time of a peak of 1,3-butylene glycol is usually from 5.5 minutes to 7 minutes. Under the analysis conditions described above, when the relative retention time of the peak of 1,3-butylene glycol is defined as 1.0, usually, a relative retention time of a peak of the compound represented by Formula (1) is from 1.3 to 1.7, and a relative retention time of a peak of the compound represented by Formula (2) is from 1.6 to 2.0, a relative retention time of a peak of the compound represented by Formula (3) is between 0.7 and 1.0, a relative retention time of a peak of the compound represented by Formula (4) is between 0.4 to 0.6, a relative retention time of a peak of the compound represented by Formula (5) is from 1.3 to 1.7, a relative retention time of a peak of the compound represented by Formula (6) is from 1.6 to 2.0, a relative retention time of a peak of the compound represented by Formula (7) is from 0.6 to 0.8, a relative retention time of a peak of the compound represented by Formula (8) is from 1.6 to 2.0, a relative retention time of a peak of the compound represented by Formula (9) is from 1.0 to 1.2, and a relative retention time of a peak of the compound represented by Formula (10) is from 1.6 to 2.0.

Note that the compound represented by Formula (1) is a compound produced by a reaction (an acetalization reaction) of methyl vinyl ketone with 1,3-butylene glycol. Methyl vinyl ketone is produced by a dehydration reaction of 1-hydroxy-3-butanone. 1-hydroxy-3-butanone is produced by oxidation of 1,3-butylene glycol. The compound represented by Formula (2) is a compound produced by a reaction (an acetalization reaction) of 1-hydroxy-3-butanone with 1,3-butylene glycol. The compound represented by Formula (3) is a compound produced by a reaction (an acetalization reaction) of acetone with 1,3-butylene glycol. The compound represented by Formula (4) is a compound produced by a reaction (an acetalization reaction) of formaldehyde with 1,3-butylene glycol. Acetone and formaldehyde are produced by decomposition of 1-hydroxy-3-butanone. The compound represented by Formula (5) is a compound produced by a reaction (an acetalization reaction) of crotonaldehyde with 1,3-butylene glycol. Crotonaldehyde is produced by a dehydration reaction of acetaldol. Acetaldol is produced by oxidation of 1,3-butylene glycol. Acetaldol is also produced by dimerization of acetaldehyde. The compound represented by Formula (6) is produced by a reaction (an acetalization reaction) of acetaldol with 1,3-butylene glycol. The compound represented by Formula (7) is produced by a reaction (an acetalization reaction) of acetaldehyde with 1,3-butylene glycol. Acetaldehyde is produced by decomposition of acetaldol. The compound represented by Formula (8) is produced by a reaction (an esterification reaction) of crotonic acid with 1,3-butylene glycol. Crotonic acid is produced by oxidation of crotonaldehyde. The compound represented by Formula (9) is produced by a reaction (an esterification reaction) of acetic acid with 1,3-butylene glycol. Acetic acid is produced by oxidation of acetaldehyde. The compound represented by Formula (10) is produced by a reaction (an esterification reaction) of 3-hydroxybutanoic acid with 1,3-butylene glycol. 3-hydroxybutanoic acid is produced by oxidation of acetaldol. In addition, the oxidation of formaldehyde produces formic acid, and a reaction between the formic acid and 1,3-butylene glycol (an esterification reaction) produces 2-hydroxypropyl formate. Decarbonation of the 2-hydroxypropyl formate produces 2-butanol. An estimated formation pathway for each compound (impurity) (sometimes referred to as “impurity formation pathway diagram”) is illustrated below. In the following formula, “1,3 BG” represents 1,3-butylene glycol.

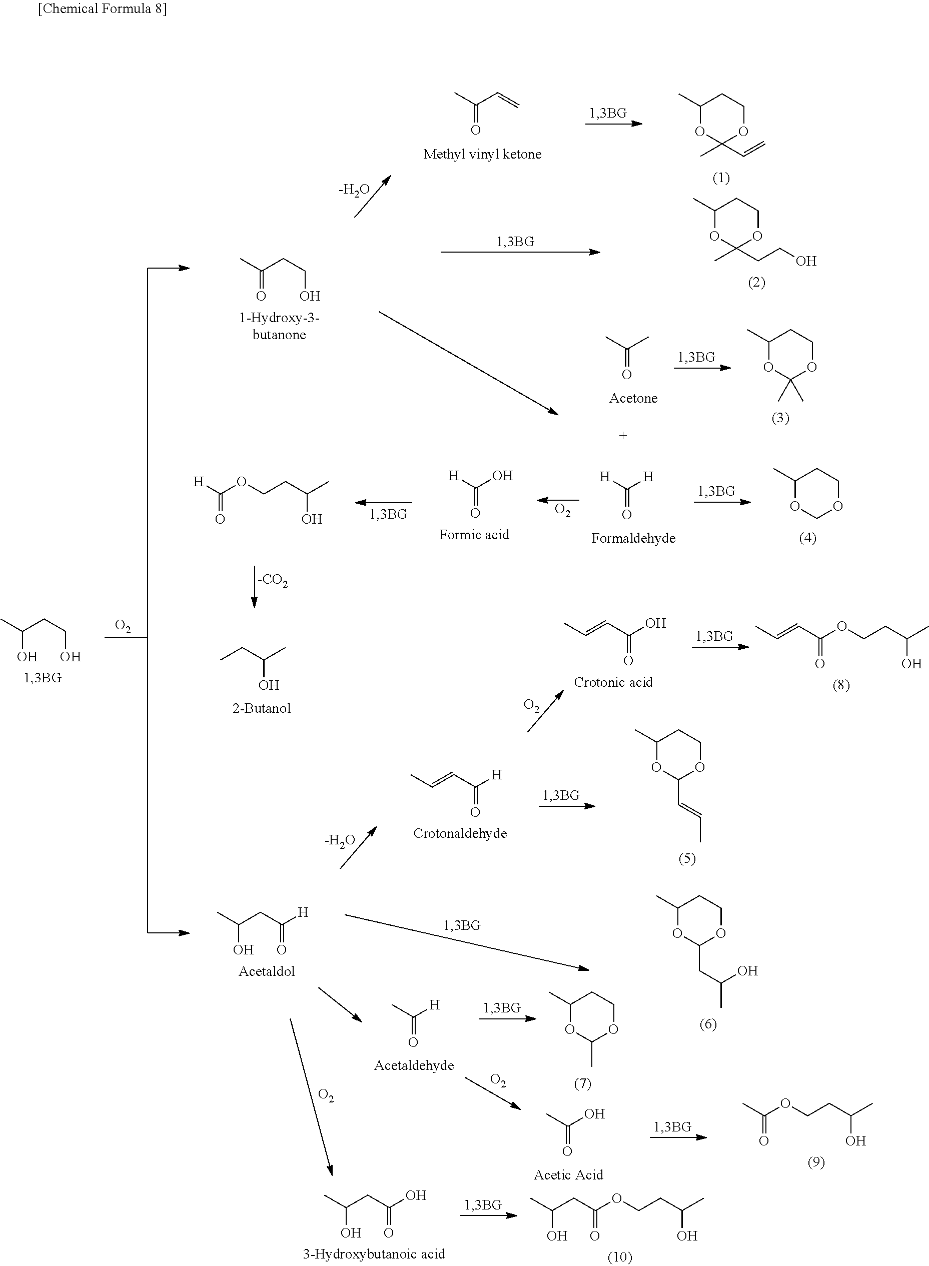

In the 1,3-butylene glycol product according to the present disclosure, after the 1,3-butylene glycol product has been kept at 180 °C for 3 hours in air atmosphere, at least one of contents of compounds represented by Formula (A) or (B) [for example, compounds represented by Formulas (1) to (10)] is less than 8 ppm (e.g., 7 ppm or less, preferably 6 ppm or less, more preferably 5 ppm or less, even more preferably 4 ppm or less, 3 ppm or less, 2 ppm or less, 1 ppm or less, 0.5 ppm or less, 0.4 ppm or less, 0.3 ppm or less, or less than 0.2 ppm). Thus, of the contents of the compounds represented by Formula (A) or (B) [for example, the compounds represented by Formulas (1) to (10)], a content of one compound may be less than 8 ppm (e.g., 7 ppm or less, preferably 6 ppm or less, more preferably 5 ppm or less, even more preferably 4 ppm or less, 3 ppm or less, 2 ppm or less, 1 ppm or less, 0.5 ppm or less, 0.4 ppm or less, 0.3 ppm or less, or less than 0.2 ppm), and contents of two compounds (or three compounds, four compounds, five compounds, six compounds, seven compounds, eight compounds, nine compounds, ten compounds, etc.) may be each less than 8 ppm (e.g., 7 ppm or less, preferably 6 ppm or less, more preferably 5 ppm or less, even more preferably 4 ppm or less, 3 ppm or less, 2 ppm or less, 1 ppm or less, 0.5 ppm or less, 0.4 ppm or less, 0.3 ppm or less, or less than 0.2 ppm).

In the 1,3-butylene glycol product according to the present disclosure, after the 1,3-butylene glycol product has been kept at 180 °C for 3 hours in air atmosphere, contents of at least four (four, five, six, seven, eight, nine, or ten) compounds, of the contents of the compounds represented by Formula (A) or (B) [for example, the compounds represented by Formulas (1) to (10)], are each less than 8 ppm (e.g., 7 ppm or less, preferably 6 ppm or less, more preferably 5 ppm or less, even more preferably 4 ppm or less, 3 ppm or less, 2 ppm or less, 1 ppm or less, 0.5 ppm or less, 0.4 ppm or less, 0.3 ppm or less, or less than 0.2 ppm).

Particularly, all of the contents of the compounds represented by Formulas (1) to (10) are preferably less than 8 ppm (e.g., 7 ppm or less, preferably 6 ppm or less, more preferably 5 ppm or less, and even more preferably 4 ppm or less, 3 ppm or less, 2 ppm or less, 1 ppm or less, 0.5 ppm or less, 0.4 ppm or less, 0.3 ppm or less, or less than 0.2 ppm).

In the 1,3-butylene glycol product according to the present disclosure, after the 1,3-butylene glycol product has been kept at 180 °C for 3 hours in air atmosphere, at least one (e.g., one, two, three, four, . . . or ten) of sums of contents of two compounds, of the contents of the compounds represented by Formula (A) or (B) [for example, the compounds represented by Formulas (1) to (10)], is less than 16 ppm (e.g., 15 ppm or less, preferably 14 ppm or less, more preferably 13 ppm or less, even more preferably 12 ppm or less, and particularly preferably 11 ppm or less, 10 ppm or less, 9 ppm or less, 8 ppm or less, 7 ppm or less, 6 ppm or less, 5 ppm or less, 4 ppm or less, 3 ppm 2 ppm or less, 1 ppm or less, 0.5 ppm or less, 0.4 ppm or less, 0.3 ppm or less, or less than 0.2 ppm).

In the 1,3-butylene glycol product according to the present disclosure, after the 1,3-butylene glycol product has been kept at 180 °C for 3 hours in air atmosphere, at least one (e.g., one, two, three, four, . . . ) of sums of contents of three compounds, of the contents of the compounds represented by Formula (A) or (B) [for example, the compounds represented by Formulas (1) to (10)], is less than 24 ppm (e.g., 20 ppm or less, preferably 18 ppm or less, more preferably 16 ppm or less, even more preferably 14 ppm or less, and particularly preferably 12 ppm or less, 11 ppm or less, 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

In the 1,3-butylene glycol product according to the present disclosure, after the 1,3-butylene glycol product has been kept at 180 °C for 3 hours in air atmosphere, at least one (e.g., one, two, . . . ) of sums of contents of four compounds, of the contents of the compounds represented by Formula (A) or (B) [for example, the compounds represented by Formulas (1) to (10)], is less than 32 ppm (e.g., 30 ppm or less, preferably 25 ppm or less, more preferably ppm or less, even more preferably 18 ppm or less, and particularly preferably 16 ppm or less, 14 ppm or less, 12 ppm or less, 10 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

Also, in the 1,3-butylene glycol product according to the present disclosure, after the 1,3-butylene glycol product has been kept at 180 °C for 3 hours in air atmosphere, a sum of the contents of the compounds represented by Formula (A), in particular, a sum of contents of compounds represented by Formulas (1) to (7), is less than 66 ppm (e.g., 60 ppm or less, preferably 50 ppm or less, more preferably 40 ppm or less, even more preferably 30 ppm or less, and particularly preferably 20 ppm or less, 18 ppm or less, 16 ppm or less, 14 ppm or less, 12 ppm or less, 10 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

Also, in the 1,3-butylene glycol product according to the present disclosure, after the 1,3-butylene glycol product has been kept at 180 °C for 3 hours in air atmosphere, a sum of the contents of the compounds represented by Formula (B), in particular, a sum of contents of compounds represented by Formulas (8) to (10), is less than 31 ppm (e.g., 25 ppm or less, preferably 20 ppm or less, more preferably 18 ppm or less, even more preferably 16 ppm or less, and particularly preferably 14 ppm or less, 13 ppm or less, 12 ppm or less, 11 μm or less, ppm or less, 9 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

Further, in the 1,3-butylene glycol product according to the present disclosure, after the 1,3-butylene glycol product has been kept at 180 °C for 3 hours in air atmosphere, a total content of the compounds represented by Formulas (1) to (4) is less than 34 ppm (e.g., 30 ppm or less, preferably 25 ppm or less, more preferably 20 ppm or less, even more preferably 18 ppm or less, and particularly preferably 16 ppm or less, 15 ppm or less, 14 ppm or less, 13 ppm or less, 12 ppm or less, 11 μm or less, 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

Also, in the 1,3-butylene glycol product according to the present disclosure, after the 1,3-butylene glycol product has been kept at 180 °C for 3 hours in air atmosphere, a total content of the compounds represented by Formulas (5) to (10) is less than 63 ppm (e.g., 60 ppm or less, preferably 50 ppm or less, more preferably 40 ppm or less, even more preferably ppm or less, and particularly preferably 20 ppm or less, 18 ppm or less, 16 ppm or less, 14 ppm or less, 12 ppm or less, 10 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

Further, in the 1,3-butylene glycol product according to the present disclosure, after the 1,3-butylene glycol product has been kept at 180 °C for 3 hours in air atmosphere, a total content of the compounds represented by Formulas (1) to (10) is less than 97 ppm (e.g., 80 ppm or less, preferably 70 ppm or less, more preferably 60 ppm or less, even more preferably 50 ppm or less, and particularly preferably 40 ppm or less, 30 ppm or less, 20 ppm or less, 18 ppm or less, 16 ppm or less, 14 ppm or less, 12 ppm or less, 10 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

Methyl vinyl ketone, 1-hydroxy-3-butanone, acetone, formaldehyde, crotonaldehyde, acetaldol, and acetaldehyde, as shown in the impurity formation pathway diagram, are precursors of the compounds represented by Formula (A) or (B). Butylaldehyde can also be a precursor of the compound represented by Formula (A) due to its structure. In addition, these compounds are carbonyl compounds, and are prone to produce various complex compounds by heat or oxygen, and may be a substance that causes coloration, a substance that causes odor or a substance that causes acidity. Therefore, the 1,3-butylene glycol product desirably has low contents of these compounds after being kept at 180 °C for 3 hours in air atmosphere. For example, the 1,3-butylene glycol product of the present disclosure preferably has a content of methyl vinyl ketone of less than 8 ppm (e.g., 7 ppm or less, preferably 6 ppm or less, more preferably 5 ppm or less, even more preferably 4 ppm or less, 3 ppm or less, 2 ppm or less, 1 ppm or less, 0.5 ppm or less, 0.4 ppm or less, 0.3 ppm or less, or less than 0.2 ppm) and/or a content of 1-hydroxy-3-butanone of less than 1 ppm (preferably 0.5 ppm or less), and/or a content of acetone of less than 6 ppm (e.g., 5 ppm or less, preferably 4 ppm or less, more preferably 3 ppm or less, even more preferably 2 ppm or less, 1 ppm or less, 0.5 ppm or less, 0.4 ppm or less, 0.3 ppm or less, or less than 0.2 ppm), and/or a content of formaldehyde of less than 2 ppm (preferably 1 ppm or less, more preferably 0.5 ppm or less, 0.4 ppm or less, 0.3 ppm or less, or less than 0.2 ppm), and/or a content of crotonaldehyde of less than 1 ppm (preferably 0.5 ppm or less), and/or a content of acetaldol of less than 1 ppm (preferably 0.5 ppm or less, 0.4 ppm or less, 0.3 ppm or less, or less than 0.2 ppm), and/or a content of acetaldehyde of less than 5 ppm (e.g., 4 ppm or less, preferably 3 ppm or less, more preferably 2 ppm or less, even more preferably 1 ppm or less, 0.5 ppm or less, 0.4 ppm or less, 0.3 ppm or less, or less than 0.2 ppm), and/or a content of butylaldehyde of less than 4 ppm (e.g., 3 ppm or less, preferably 2 ppm or less, more preferably 1 ppm or less, even more preferably 0.5 ppm or less, 0.4 ppm or less, 0.3 ppm or less, or less than 0.2 ppm), after being kept at 180 °C for 3 hours in air atmosphere.

Each of the contents of methyl vinyl ketone, 1-hydroxy-3-butanone, acetone, formaldehyde, crotonaldehyde, acetaldol, acetaldehyde, and butylaldehyde in the 1,3-butylene glycol product can be quantified by GC-MS analysis (gas mass analysis) under the conditions described above. When the relative retention time of the peak of 1,3-butylene glycol is defined as 1.0 under the above analysis conditions, usually, a relative retention time of a peak of methyl vinyl ketone is from 0.3 to 0.5, a relative retention time of a peak of 1-hydroxy-3-butanone is from 0.4 to 0.6, a relative retention time of a peak of acetone is from 0.3 to 0.5, a relative retention time of a peak of formaldehyde is from 0.3 to 0.5, a relative retention time of a peak of crotonaldehyde is from 0.3 to 0.5, a relative retention time of a peak of acetaldol is from 0.4 to 0.6, a relative retention time of a peak of acetaldehyde is from 0.3 to 0.5, and a relative retention time of a peak of butylaldehyde is from 0.3 to 0.5.

Also, the 1,3-butylene glycol product according to the present disclosure preferably has a total content of methyl vinyl ketone, 1-hydroxy-3-butanone, acetone, formaldehyde, crotonaldehyde, acetaldol, and acetaldehyde of less than 24 ppm (e.g., 20 ppm or less, preferably 18 ppm or less, more preferably 16 ppm or less, even more preferably 14 ppm or less, and particularly preferably 12 ppm or less, 11 ppm or less, 10 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm), after being kept at 180 °C for 3 hours in air atmosphere.

Further, the 1,3-butylene glycol product according to the present disclosure preferably has a total content of methyl vinyl ketone, acetone, and butylaldehyde of less than 18 ppm (e.g., 16 ppm or less, preferably 15 ppm or less, more preferably 14 ppm or less, even more preferably 13 ppm or less, and particularly preferably 12 ppm or less, 11 ppm or less, 10 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm), after being kept at 180 °C for 3 hours in air atmosphere. Methyl vinyl ketone, acetone and butylaldehyde are impurities present in relatively large amounts in the 1,3-butylene glycol product after the heating test.

Furthermore, in the 1,3-butylene glycol product according to the present disclosure, after the 1,3-butylene glycol product has been kept at 180 °C for 3 hours in air atmosphere, an APHA is less than 79 (preferably 65 or less, more preferably 60 or less, even more preferably 55 or less, 50 or less, 45 or less, 40 or less, 35 or less, 30 or less, 25 or less, 20 or less, and particularly preferably 18 or less, 15 or less, 14 or less, 13 or less, 12 or less, 11 or less, 10 or less, 9 or less, 8 or less, or 7 or less).

Also, the 1,3-butylene glycol product according to the present disclosure preferably has an initial boiling point of 203 °C or higher and preferably has a dry point of 209 °C or lower, after the 1,3-butylene glycol product has been kept at 180 °C for 3 hours in air atmosphere. The initial boiling point is preferably 204 °C or higher, more preferably 205 °C or higher, even more preferably 206 °C or higher or 207 °C, and particularly preferably 208 °C or higher.

In the 1,3-butylene glycol product according to the present disclosure, after the 1,3-butylene glycol product has been kept at 180 °C for 3 hours in air atmosphere, a potassium permanganate test value (PMT) is 30 minutes or longer. The potassium permanganate test value is more preferably longer than 30 minutes (e.g., 32 minutes or longer), even more preferably 35 minutes or longer (e.g., 40 minutes or longer), and particularly preferably 50 minutes or longer (especially, 60 minutes or longer).

In the 1,3-butylene glycol product according to the present disclosure, after the 1,3-butylene glycol product has been kept at 180 °C for 3 hours in air atmosphere, an acid concentration (in terms of acetic acid) is less than 16 ppm. The acid concentration (in terms of acetic acid) is more preferably 14 ppm or less, even more preferably 12 ppm or less, 11 ppm or less, 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm.

As described above, methyl vinyl ketone, 1-hydroxy-3-butanone, acetone, formaldehyde, crotonaldehyde, acetaldol, acetaldehyde, and butylaldehyde are precursors of the compounds represented by Formula (A) or (B). In particular, 1-hydroxy-3-butanone and acetaldol are precursors of various impurities. Therefore, these impurities are desirably as less as possible in the 1,3-butylene glycol product before the heating test. From such a viewpoint, the 1,3-butylene glycol product according to the present disclosure preferably has a total content of 1-hydroxy-3-butanone and acetaldol of less than 19 ppm (e.g., 18 ppm or less, preferably 16 ppm or less, more preferably 14 ppm or less, even more preferably 12 ppm or less, and particularly preferably 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm), in a state where the 1,3-butylene glycol product has not undergone a test in which 1,3-butylene glycol product is kept at 180 °C for 3 hours in air atmosphere (before the heating test).

Further, the 1,3-butylene glycol product according to the present disclosure preferably has a content of methyl vinyl ketone of less than 10 ppm (e.g., 8 ppm or less, preferably 6 ppm or less, more preferably 5 ppm or less, even more preferably 4 ppm or less, 3 ppm or less, 2 ppm or less, 1 ppm or less, 0.5 ppm or less, 0.4 ppm or less, 0.3 ppm or less, or less than 0.2 ppm), and/or a content of 1-hydroxy-3-butanone of less than 9 ppm (e.g., 7 ppm or less, preferably 6 ppm or less, more preferably 5 ppm or less, even more preferably 4 ppm or less, 3 ppm or less, 2 ppm or less, 1 ppm or less, 0.5 ppm or less, 0.4 ppm or less, 0.3 ppm or less, or less than 0.2 ppm), and/or a content of acetone of less than 9 ppm (e.g., 7 ppm or less, preferably 6 ppm or less, more preferably 5 ppm or less, even more preferably. 4 ppm or less, 3 ppm or less, 2 ppm or less, 1 ppm or less, 0.5 ppm or less, 0.4 ppm or less, 0.3 ppm or less, or less than 0.2 ppm), and/or a content of formaldehyde of less than 3 ppm (e.g., 2 ppm or less, preferably 1 ppm or less, more preferably 0.5 ppm or less, 0.4 ppm or less, 0.3 ppm or less, or less than 0.2 ppm), and/or a content of crotonaldehyde of less than 2 ppm (e.g., 1.5 ppm or less, preferably 1 ppm or less, more preferably 0.5 ppm or less, 0.4 ppm or less, 0.3 ppm or less, or less than 0.2 ppm), and/or a content of acetaldol of less than 10 ppm (e.g., 8 ppm or less, preferably 6 ppm or less, more preferably 5 ppm or less, more preferably 4 ppm or less, 3 ppm or less, 2 ppm or less, 1 ppm or less, 0.5 ppm or less, 0.4 ppm or less, 0.3 ppm or less, or less than 0.2 ppm), and/or a content of acetaldehyde of or less 4 ppm (e.g., 3 ppm or less, preferably 2 ppm or less, more preferably 1 ppm or less, even more preferably 0.5 ppm or less, 0.4 ppm or less, 0.3 ppm or less, or less than 0.2 ppm) and/or a content of butylaldehyde of less than 8 ppm (e.g., 7 ppm or less, preferably 6 ppm or less, more preferably 5 ppm or less, even more preferably 4 ppm or less, 3 ppm or less, 2 ppm or less, 1 ppm or less, 0.5 ppm or less, 0.4 ppm or less, 0.3 ppm or less, or less than 0.2 ppm), in a state where the 1,3-butylene glycol product has not undergone a test in which 1,3-butylene glycol product is kept at 180 °C for 3 hours in air atmosphere (before the heating test).

Furthermore, the 1,3-butylene glycol product according to the present disclosure preferably has a total content of methyl vinyl ketone, 1-hydroxy-3-butanone, acetone, formaldehyde, crotonaldehyde, acetaldol, and acetaldehyde of less than 47 ppm (e.g., 40 ppm or less, preferably 30 ppm or less, more preferably 25 ppm or less, even more preferably 20 ppm or less, and particularly preferably 18 ppm or less, 16 ppm or less, 14 ppm or less, 13 ppm or less, 12 ppm or less, 11 μm or less, 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm), in a state where the 1,3-butylene glycol product has not undergone a test in which 1,3-butylene glycol product is kept at 180 °C for 3 hours in air atmosphere (before the heating test).

Furthermore, the 1,3-butylene glycol product according to the present disclosure preferably has an acid concentration (in terms of acetic acid) of less than 11 ppm (e.g., 10 ppm or less, preferably 9 ppm or less, more preferably 8 ppm or less, even more preferably 7 ppm or less, particularly preferably 6 ppm or less, and most preferably 5 ppm or less, 4 ppm or less, 3 ppm or less, 2 ppm or less, 1 ppm or less, 0.5 ppm or less, 0.4 ppm or less, 0.3 ppm or less, or less than 0.2 ppm), in a state where the 1,3-butylene glycol product has not undergone a test in which 1,3-butylene glycol product is kept at 180 °C for 3 hours in air atmosphere (before the heating test). Reduction of the acid concentration in the 1,3-butylene glycol product can suppress an increase of impurities represented by Formula (B) over time.

With the 1,3-butylene glycol product according to the present disclosure, an acid concentration (in terms of acetic acid) of a 90 wt. % aqueous solution of the 1,3-butylene glycol product kept at 100 °C for 1 week in air atmosphere is less than 23 ppm (e.g., 20 ppm or less, preferably 19 ppm or less, 18 ppm or less, 17 ppm or less, or 16 ppm or less, more preferably 15 ppm or less, even more preferably 14 ppm or less, 13 ppm or less, 12 ppm or less, 11 ppm or less, and particularly preferably 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm), in a state where the 1,3-butylene glycol product has not undergone a test in which 1,3-butylene glycol product is kept at 180 °C for 3 hours in air atmosphere (before the heating test). The 90 wt. % aqueous solution means an aqueous solution prepared by mixing the 1,3-butylene glycol product and water (e.g., pure water) to adjust the 1,3-butylene glycol product to 90 wt. %. Reduction of a concentration of an acid precursor in the 1,3-butylene glycol product can suppress an increase of impurities represented by Formula (B) over time.

For the acid concentration (in terms of acetic acid) of the 90 wt. % aqueous solution of the 1,3-butylene glycol product of the present disclosure, a ratio of the acid concentration after keeping the aqueous solution at 100 °C for 1 week to the acid concentration before keeping the solution at 100 °C for 1 week, [(acid concentration after keeping the solution at 100 °C for 1 week)/(acid concentration before keeping the solution at 100 °C for 1 week)×100(%) ], is preferably 200% or less, more preferably 150% or less, and even more preferably 120% or less, in a state where the 1,3-butylene glycol product has not undergone a test in which 1,3-butylene glycol product is kept at 180 °C for 3 hours in air atmosphere (before the heating test).

The 1,3-butylene glycol product according to the present disclosure has an APHA (Hazen color number) of, for example, 6 or less (preferably 5 or less, more preferably 4 or less, even more preferably 3 or less, and particularly preferably 2 or less), in a state where the 1,3-butylene glycol product has not undergone a test in which 1,3-butylene glycol product is kept at 180 °C for 3 hours in air atmosphere (before the heating test). Furthermore, an APHA, after the 1,3-butylene glycol product has been kept at 100 °C for 75 days in air atmosphere, is, for example, 42 or less (preferably 35 or less, 30 or less, 25 or less, 20 or less, 18 or less, 16 or less, 15 or less, 14 or less, or 13 or less, more preferably 12 or less, 11 or less, 10 or less, 9 or less, 8 or less, 7 or less, or 6 or less, and more preferably 5 or less, 4 or less, 3 or less, or 2 or less.

For the APHA of the 1,3-butylene glycol product according to the present disclosure, a ratio of the APHA after the 1,3-butylene glycol product has been kept at 100 °C for 75 days to the APHA before the 1,3-butylene glycol product has been kept at 100 °C for 75 days, [(APHA after the 1,3-butylene glycol product has been kept at 100 °C for 75 days)/(APHA before the 1,3-butylene glycol product has been kept at 100 °C for 75 days)], is not particularly limited, but is preferably 10 or less, more preferably 8 or less, even more preferably 7 or less, and particularly preferably 6 or less (for example, 5 or less, 4 or less, or 3 or less). In addition, the ratio is 1 or greater, or may be 2 or greater.

Additionally, the 1,3-butylene glycol product according to the present disclosure preferably has an initial boiling point of higher than 203 °C, in a state where the 1,3-butylene glycol product has not undergone a test in which 1,3-butylene glycol product is kept at 180 °C for 3 hours in air atmosphere (before the heating test). The initial boiling point is preferably 204 °C or higher, more preferably 205 °C or higher, even more preferably 206 °C or higher or 207 °C, and particularly preferably 208 °C or higher.

Additionally, the 1,3-butylene glycol product according to the present disclosure preferably has a dry point of 209 °C or lower, in a state where the 1,3-butylene glycol product has not undergone a test in which 1,3-butylene glycol product is kept at 180 °C for 3 hours in air atmosphere (before the heating test).

Furthermore, the 1,3-butylene glycol product according to the present disclosure preferably has a potassium permanganate test value (PMT) of 30 minutes or longer, in a state where the 1,3-butylene glycol product has not undergone a test in which 1,3-butylene glycol product is kept at 180 °C for 3 hours in air atmosphere (before the heating test). The potassium permanganate test value (PMT) is more preferably longer than 30 minutes (e.g., 32 minutes or longer), even more preferably 35 minutes or longer (e.g., 40 minutes or longer), and particularly preferably 50 minutes or longer (especially, 60 minutes or longer).

In the 1,3-butylene glycol product according to the present disclosure, a content of 1,3-butylene glycol is, for example, 98.6% or greater. The content of the 1,3-butylene glycol corresponds to an area ratio (GC area ratio) of the peak of 1,3-butylene glycol, in the gas chromatographic analysis (GC analysis) performed under the following conditions.

### The Conditions for the Gas Chromatographic Analysis are as Follows:

Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

In the present disclosure, the “(peak) area ratio” means a ratio of the area of a specific peak relative to the sum of the areas of all peaks appearing in the chromatogram. In addition, all peaks mean, for example, all of the peaks appearing in the analysis continued until and discontinued at a relative retention time of 7.8, provided that the relative retention time of 1,3-butylene glycol is 1.0. The content of 1,3-butylene glycol in the 1,3-butylene glycol product is, for example, 98.6% or greater, and thus basic properties inherent in 1,3-butylene glycol are guaranteed.

The content (GC area ratio) of the 1,3-butylene glycol is preferably 98.7% or greater, more preferably 98.8% or greater, even more preferably 98.9% or greater, and particularly preferably 99.0% or greater. Furthermore, the content (GC area ratio) of 1,3-butylene glycol is especially 99.1% or greater, 99.2% or greater, 99.3% or greater, 99.4% or greater, 99.5% or greater, 99.6% or greater, 99.7% or greater, or 99.8% or greater.

In the GC analysis, a total area ratio of peaks shorter in retention time than the peak of 1,3-butylene glycol is preferably 0.28% or less, more preferably 0.25% or less, even more preferably 0.23% or less, 0.2% or less, 0.17% or less, 0.15% or less, 0.12% or less, 0.1% or less, 0.07% or less, 0.04% or less, 0.03% or less, 0.02% or less, 0.01% or less, or 0.007% or less, and particularly preferably 0.005% or less (for example, 0.002% or less).

In the GC analysis, the total area ratio of peaks longer in retention time than the peak of 1,3-butylene glycol is preferably 1% or less, more preferably 0.9% or less, more preferably 0.8% or less, 0.7% or less, 0.6% or less, 0.5% or less, 0.4% or less, 0.3% or less, or 0.2% or less, and particularly preferably 0.1% or less.

In the 1,3-butylene glycol product according to the present disclosure, a content of water is preferably less than 0.4 wt. %. The content of water is preferably 0.3 wt. % or less, more preferably 0.2 wt. % or less, even more preferably 0.1 wt. % or less, 0.07 wt. % or less, 0.05 wt. % or less, 0.03 wt. % or less, 0.02 wt. % or less, or 0.01 wt. % or less, and particularly preferably 0.005 wt. % or less. Note that the content of water can be quantified by a Karl Fischer water content measurement instrument.

A 1,3-butylene glycol product having a content of 1,3-butylene glycol of a specific value or greater and a content of a specific substance after the heating test under the specific conditions has high purity and high quality, and is hardly deteriorated in quality over time. Therefore, it can be suitably used as a raw material for moisturizers or cosmetic products.

### 1,3-Butylene Glycol Product—Ninth Aspect of the Present Disclosure

The 1,3-butylene glycol product according to the ninth aspect of the present disclosure is a 1,3-butylene glycol product containing 1,3-butylene glycol, wherein a sum of contents of acetaldehyde, crotonaldehyde, methyl vinyl ketone, acetone, formaldehyde, butylaldehyde, acetaldol, 1-hydroxy-3-butanone, 2-butanol, a compound represented by Formula (1) below, a compound represented by Formula (2) below, a compound represented by Formula (3) below, a compound represented by Formula (4) below, a compound represented by Formula (5) below, a compound represented by Formula (6) below, a compound represented by Formula (7) below, a compound represented by Formula (8) below, a compound represented by Formula (9) below, and a compound represented by Formula (10) below is less than 65 ppm.

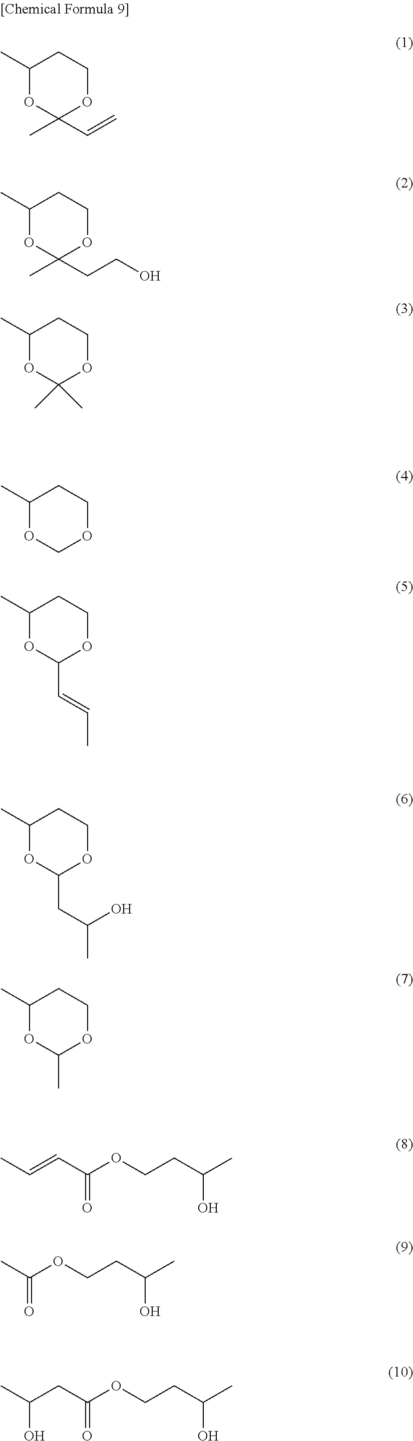

The compounds represented by Formulas (1) to (7) above are cyclic acetal compounds, and the compounds represented by Formulas (8) to (10) are 3-hydroxybutyl carboxylate compounds. These compounds are also present as impurities during production of 1,3-butylene glycol, but are impurities whose content increases over time, when the 1,3-butylene glycol product is stored for a long period of time. The cyclic acetal compounds are hydrolyzed in the presence of water to produce corresponding carbonyl compounds. The thus-produced carbonyl compound is itself a reducing substance, not only diminishes the potassium permanganate test value, but also is highly reactive and likely to produce a variety of complex compounds by heat or oxygen, and may become a substance that causes coloration, a substance that causes odor or a substance that causes acidity. Also, the 3-hydroxybutyl carboxylate compounds are hydrolyzed in the presence of water to produce corresponding carboxylic acids. The thus-produced carboxylic acid may be a substance that causes odor or a substance that causes acidity.

Acetaldehyde, crotonaldehyde, methyl vinyl ketone, acetone, formaldehyde, acetaldol, and 1-hydroxy-3-butanone are precursors of the cyclic acetal compounds or 3-hydroxybutyl carboxylate compounds. Butylaldehyde can also be precursors of the cyclic acetal compounds considering its structure. In addition, these compounds are carbonyl compounds, and are prone to produce various complex compounds by heat or oxygen, and may be a substance that causes coloration, a substance that causes odor or a substance that causes acidity.

In a 1,3-butylene glycol product, having high contents of acetaldehyde, crotonaldehyde, methyl vinyl ketone, acetone, formaldehyde, butylaldehyde, acetaldol, 1-hydroxy-3-butanone, 2-butanol, and the compounds represented by Formulas (1) to (10) below, odor increases over time. Even when it is odorless at the time of manufacture, odor is likely to be caused by long-term storage, for example. Thus, in the 1,3-butylene glycol product of the present disclosure, a sum of contents of acetaldehyde, crotonaldehyde, methyl vinyl ketone, acetone, formaldehyde, butylaldehyde, acetaldol, 1-hydroxy-3-butanone, 2-butanol, and the compounds represented by or Formulas (1) to (10) is less than 65 ppm, preferably 50 ppm or less, more preferably 45 ppm or less, 40 ppm or less, 35 ppm or less, 30 ppm or less, 25 ppm or less, or 20 ppm or less, and even more preferably 15 ppm or less, 13 ppm or less, 10 ppm or less, 8 ppm or less, 5 ppm or less, 4 ppm or less, 3 ppm or less, or 2 ppm or less.

In addition, in the 1,3-butylene glycol product of the present disclosure, a sum of the contents of the compounds (cyclic acetal compounds) represented by Formulas (1) to (7) above is preferably less than 28 ppm, more preferably 25 ppm or less, 20 ppm or less, or 15 ppm or less, and even more preferably 12 ppm or less, 10 ppm or less, 8 ppm or less, 6 ppm or less, 4 ppm or less, 2 ppm or less, or 1.4 ppm or less.

In addition, in the 1,3-butylene glycol product of the present disclosure, a sum of the contents of the compounds (3-hydroxybutyl carboxylate compounds) represented by Formulas (8) to (10) above is preferably less than 6 ppm, more preferably 5 ppm or less, and still more preferably 4 ppm or less, 3 ppm or less, 2 ppm or less, 1 ppm or less, 0.8 ppm or less, or 0.6 ppm or less.

Also, the 1,3-butylene glycol product of the present disclosure preferably has a sum of the contents of the compounds represented by Formulas (1) to (10) above of preferably less than 34 ppm, more preferably 30 ppm or less, and still more preferably 25 ppm or less, 20 ppm or less, 18 ppm or less, 16 ppm or less, 14 ppm or less, 12 ppm or less, 10 ppm or less, 8 ppm or less, 7 ppm or less, 6 ppm or less, 5 ppm or less, 4 ppm or less, 3 ppm or less, or 2 ppm or less.

In addition, in the 1,3-butylene glycol product of the present disclosure, the content of acetaldehyde is preferably less than 1.6 ppm, and more preferably 1.5 ppm or less, 1.4 ppm or less, 1.2 ppm or less, 1.0 ppm or less, 0.8 ppm or less, 0.6 ppm or less, 0.4 ppm or less, or 0.2 ppm or less. The content of the crotonaldehyde is preferably less than 1 ppm, and more preferably 0.8 ppm or less, 0.6 ppm or less, 0.4 ppm or less, or 0.2 ppm or less. The content of methyl vinyl ketone is preferably less than 6 ppm, and more preferably 5 ppm or less, 4 ppm or less, 3 ppm or less, 2 ppm or less, 1 ppm or less, 0.8 ppm or less, 0.6 ppm or less, 0.4 ppm or less, or 0.2 ppm or less. The content of acetone is preferably less than 5 ppm, and more preferably 4 ppm or less, 3 ppm or less, 2 ppm or less, 1 ppm or less, 0.8 ppm or less, 0.6 ppm or less, 0.4 ppm or less, or 0.2 ppm or less. The content of formaldehyde is preferably less than 1 ppm, and more preferably 0.8 ppm or less, 0.6 ppm or less, 0.4 ppm or less, 0.3 ppm or less, or 0.2 ppm or less. The content of butylaldehyde is preferably less than 5 ppm, and more preferably 4 ppm or less, 3 ppm or less, 2 ppm or less, 1 ppm or less, 0.8 ppm or less, 0.6 ppm or less, 0.4 ppm or less, or 0.2 ppm or less. The content of acetaldol is preferably 6 ppm or less, and more preferably 5 ppm or less, 4 ppm or less, 3 ppm or less, 2 ppm or less, 1 ppm or less, 0.8 ppm or less, 0.4 ppm or less, or 0.2 ppm or less. The content of 1-hydroxy-3-butanone is preferably less than 6 ppm, and more preferably 5 ppm or less, 4 ppm or less, 3 ppm or less, 2 ppm or less, 1 ppm or less, 0.8 ppm or less, 0.6 ppm or less, 0.4 ppm or less, or 0.2 ppm or less. The content of 2-butanol is preferably 0.3 ppm or less, and more preferably less than 0.2 ppm. The content of the compound represented by Formula (1) is preferably less than 2 ppm, and more preferably 1.8 ppm or less, 1.6 ppm or less, 1.4 ppm or less, 1.2 ppm or less, 0.8 ppm or less, 0.6 ppm or less, 0.4 ppm or less, or 0.2 ppm or less. The content of the compound represented by Formula (2) is preferably less than 1 ppm, and more preferably 0.8 ppm or less, 0.6 ppm or less, 0.4 ppm or less, 0.3 ppm or less, or 0.2 ppm or less. The content of the compound represented by Formula (3) is preferably less than 4 ppm, and more preferably 3 ppm or less, 2 ppm or less, 1 ppm or less, 0.8 ppm or less, 0.6 ppm or less, 0.4 ppm or less, or 0.2 ppm or less. The content of the compound represented by Formula (4) is preferably less than 3 ppm, and more preferably 2 ppm or less, 1 ppm or less, 0.8 ppm or less, 0.6 ppm or less, 0.4 ppm or less, 0.3 ppm or less, or 0.2 ppm or less. The content of the compound represented by Formula (5) is preferably less than 6 ppm, and more preferably 5 ppm or less, 4 ppm or less, 3 ppm or less, 2 ppm or less, 1 ppm or less, 0.8 ppm or less, 0.6 ppm or less, 0.4 ppm or less, or 0.2 ppm or less. The content of the compound represented by Formula (6) is preferably less than 5 ppm, and more preferably 4 ppm or less, 3 ppm or less, 2 ppm or less, 1 ppm or less, 0.8 ppm or less, 0.6 ppm or less, 0.4 ppm or less, or 0.2 ppm or less. The content of the compound represented by Formula (7) is preferably less than 7 ppm, and more preferably 6 ppm or less, 5 ppm or less, 4 ppm or less, 3 ppm or less, 2 ppm or less, 1 ppm or less, 0.8 ppm or less, 0.6 ppm or less, 0.4 ppm or less, or 0.2 ppm or less. The content of the compound represented by Formula (8) is preferably less than 1 ppm, and more preferably 0.8 ppm or less, 0.6 ppm or less, 0.4 ppm or less, 0.3 ppm or less, or 0.2 ppm or less. The content of the compound represented by Formula (9) is preferably less than 4 ppm, and more preferably 3 ppm or less, 2 ppm or less, 1 ppm or less, 0.8 ppm or less, 0.6 ppm or less, 0.4 ppm or less, or 0.2 ppm or less. The content of the compound represented by Formula (10) is preferably less than 1 ppm, and more preferably 0.8 ppm or less, 0.6 ppm or less, 0.4 ppm or less, 0.3 ppm or less, or 0.2 ppm or less.

Each of the contents of acetaldehyde, crotonaldehyde, methyl vinyl ketone, acetone, formaldehyde, butylaldehyde, acetaldol, 1-hydroxy-3-butanone, 2-butanol, and the compounds represented by the following formulas (1) to (10) can be quantified by GC-MS analysis under the conditions described above. In GC-MS analysis, even very small peaks are all subjected to mass spectrometry, and each component is quantified. Since the analysis is performed for a specific mass, a substance different in mass is not detected even when another impurity overlaps the peak. Therefore, the analysis is more sensitive than GC analysis which will be described below. In the present specification, the unit “ppm” of the content of each component by GC-MS analysis means “ppm by weight”.

### Conditions for GC-MS Analysis

Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Ion source temperature: EI 230 °C, CI 250 °C
Q Pole temperature: 150 °C
Sample: subjected to analysis as it is

In the above analytical conditions, the retention time of a peak of 1,3-butylene glycol is usually from 5.5 minutes to 7 minutes. When a relative retention time of the peak of 1,3-butylene glycol under the above analysis conditions is defined as 1.0, a relative retention time of the peak of acetaldehyde is from 0.3 to 0.5, a relative retention time of the peak of crotonaldehyde is from 0.3 to 0.5, a relative retention time of the peak of methyl vinyl ketone is from 0.3 to 0.5, a relative retention time of the peak of acetone is from 0.3 to 0.5, a relative retention time of the peak of formaldehyde is from 0.3 to 0.5, a relative retention time of the peak of butylaldehyde is from 0.3 to 0.5, and a relative retention time of the peak of acetaldol is from 0.4 to 0.6, a relative retention time of the peak of 1-hydroxy-3-butanone is from 0.4 to 0.6, and a relative retention time of the peak of 2-butanol is from 0.3 to 0.5, a relative retention time of the peak of the compound represented by Formula (1) above is from 1.3 to 1.7, a relative retention time of the peak of the compound represented by Formula (2) is from 1.6 to 2.0, a relative retention time of the peak of the compound represented by Formula (3) is from 0.7 to 1.0, a relative retention time of the peak of the compound represented by Formula (4) is from 0.4 to 0.6, a relative retention time of the peak of the compound represented by Formula (5) is from 1.3 to 1.7, a relative retention time of the peak of the compound represented by Formula (6) is from 1.6 to 2.0, a relative retention time of the peak of the compound represented by Formula (7) is from 0.6 to 0.8, a relative retention time of the peak of the compound represented by Formula (8) is from 1.6 to 2.0, and a relative retention time of the peak of the compound represented by Formula (9) is from 1.0 to 1.2, and a relative retention time of the peak of the compound represented by Formula (10) is from 1.6 to 2.0.

Note that the compound represented by Formula (1) is a compound produced by a reaction (an acetalization reaction) of methyl vinyl ketone with 1,3-butylene glycol. Methyl vinyl ketone is produced by a dehydration reaction of 1-hydroxy-3-butanone. 1-hydroxy-3-butanone is produced by oxidation of 1,3-butylene glycol. The compound represented by Formula (2) is a compound produced by a reaction (an acetalization reaction) of 1-hydroxy-3-butanone with 1,3-butylene glycol. The compound represented by Formula (3) is a compound produced by a reaction (an acetalization reaction) of acetone with 1,3-butylene glycol. The compound represented by Formula (4) is a compound produced by a reaction (an acetalization reaction) of formaldehyde with 1,3-butylene glycol. Acetone and formaldehyde are produced by decomposition of 1-hydroxy-3-butanone. The compound represented by Formula (5) is a compound produced by a reaction (an acetalization reaction) of crotonaldehyde with 1,3-butylene glycol. Crotonaldehyde is produced by a dehydration reaction of acetaldol. Acetaldol is produced by oxidation of 1,3-butylene glycol. Acetaldol is also produced by dimerization of acetaldehyde. The compound represented by Formula (6) is produced by a reaction (an acetalization reaction) of acetaldol with 1,3-butylene glycol. The compound represented by Formula (7) is produced by a reaction (an acetalization reaction) of acetaldehyde with 1,3-butylene glycol. Acetaldehyde is produced by decomposition of acetaldol. The compound represented by Formula (8) is produced by a reaction (an esterification reaction) of crotonic acid with 1,3-butylene glycol. Crotonic acid is produced by oxidation of crotonaldehyde. The compound represented by Formula (9) is produced by a reaction (an esterification reaction) of acetic acid with 1,3-butylene glycol. Acetic acid is produced by oxidation of acetaldehyde. The compound represented by Formula (10) is produced by a reaction (an esterification reaction) of 3-hydroxybutanoic acid with 1,3-butylene glycol. 3-hydroxybutanoic acid is produced by oxidation of acetaldol. In addition, the oxidation of formaldehyde produces formic acid, and a reaction between the formic acid and 1,3-butylene glycol (an esterification reaction) produces 2-hydroxypropyl formate. Decarbonation of the 2-hydroxypropyl formate produces 2-butanol. An estimated formation pathway for each compound (impurity) (sometimes referred to as “impurity formation pathway diagram”) is illustrated below. In the following formula, “1,3 BG” represents 1,3-butylene glycol. [Chemical Formula 10]

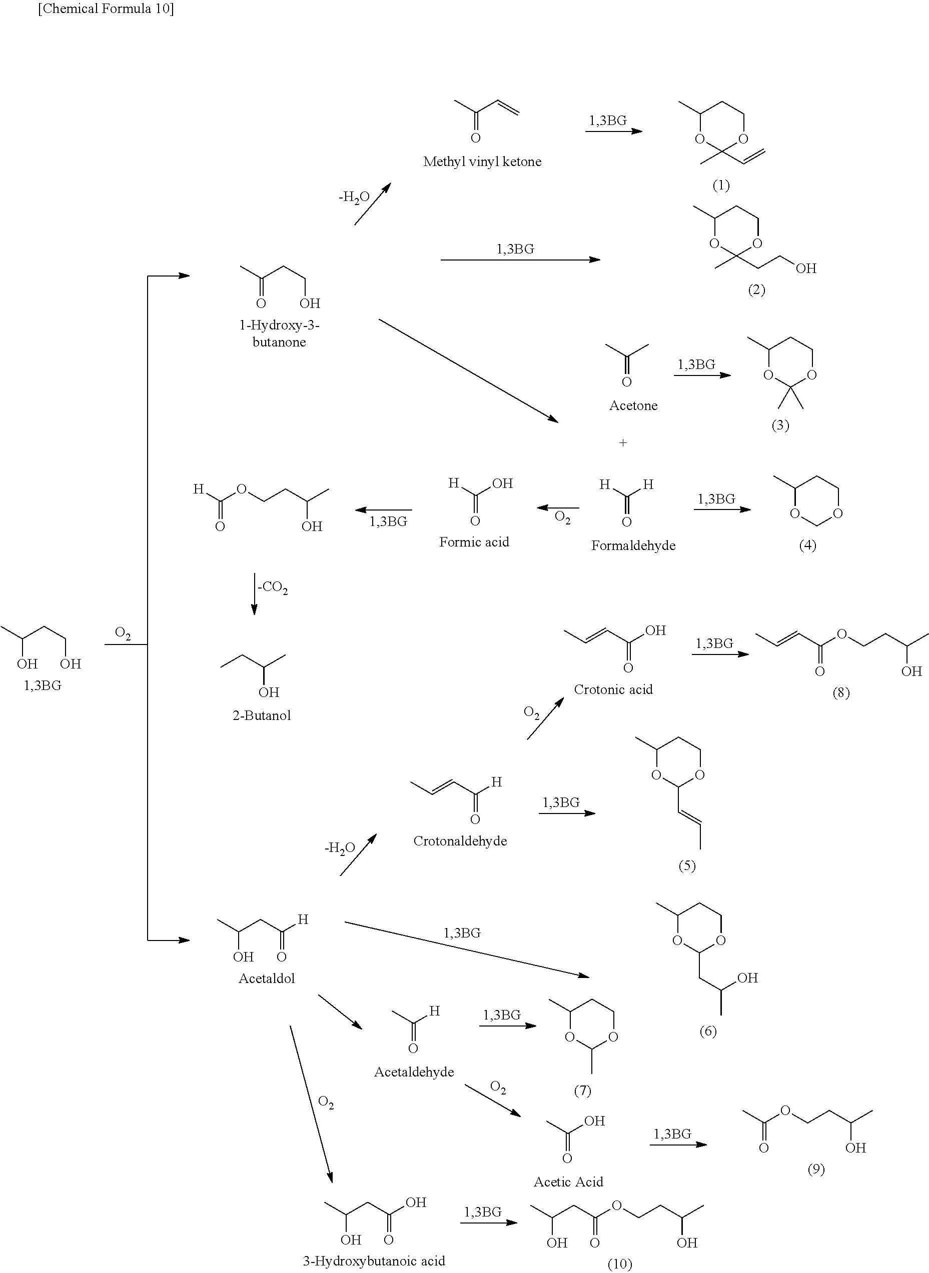

As described above, acetaldehyde, crotonaldehyde, methyl vinyl ketone, acetone, formaldehyde, butylaldehyde, acetaldol, and 1-hydroxy-3-butanone are precursors of the cyclic acetal compounds or 3-hydroxybutyl carboxylate compounds. In particular, 1-hydroxy-3-butanone and acetaldol are precursors of various impurities. Therefore, these impurities are present desirably as little as possible in the 1,3-butylene glycol product of the present disclosure. From such a perspective, in the 1,3-butylene glycol product of the present disclosure, a total content of acetaldehyde, crotonaldehyde, methyl vinyl ketone, acetone, formaldehyde, butylaldehyde, acetaldol, and 1-hydroxy-3-butanone is desirably 31 ppm or less (preferably, 30 ppm or less, 25 ppm or less, 20 ppm or less, 18 ppm or less, 16 ppm or less, 14 ppm or less, 13 ppm or less, 12 ppm or less, 11 μm or less, 10 ppm or less, 9 ppm or less, 8 ppm or less, 7 ppm or less, 6 ppm or less, 5 ppm or less, 4 ppm or less, 3 ppm or less, 2 ppm or less, or 1.5 ppm or less). In the 1,3-butylene glycol product of the present disclosure, a content of acetaldol and 1-hydroxy-3-butanone is less than 12 ppm (preferably, 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, or 0.4 ppm or less).

Furthermore, in the 1,3-butylene glycol product of the present disclosure, an acid concentration (in terms of acetic acid) is less than 6 ppm (e.g., 5 ppm or less, preferably 4 ppm or less, more preferably 3 ppm or less, even more preferably 2 ppm or less, and particularly preferably 1 ppm or less). Lowering the acid concentration in the 1,3-butylene glycol product can suppress increases, over time, of 3-hydroxybutyl carboxylate compounds such as the compounds represented by Formulas (8) to (10) above. The 3-hydroxybutyl carboxylate compounds are hydrolyzed in the presence of water to produce corresponding carboxylic acids. The thus-produced carboxylic acid may be a substance that causes odor or a substance that causes acidity.

The 1,3-butylene glycol product of the present disclosure preferably has an acid concentration (in terms of acetic acid) of preferably less than 9 ppm (for example, 8 ppm or less, preferably 7 ppm or less, more preferably 6 ppm or less, still more preferably 5 ppm or less, and particularly preferably 4 ppm or less, 3 ppm or less, 2 ppm or less, or 1 ppm or less), after a 90 wt. % aqueous solution thereof has been kept at 100 °C for 1 week. The 90 wt. % aqueous solution means an aqueous solution prepared by mixing the 1,3-butylene glycol product and water (e.g., pure water) to adjust the 1,3-butylene glycol product to 90 wt. %. Reduction in the concentrations of acid precursors in the 1,3-butylene glycol product can suppress increases, over time, of 3-hydroxybutyl carboxylate compounds such as the compounds represented by Formulas (8) to (10) above.

For the acid concentration (in terms of acetic acid) of the 90 wt. % aqueous solution of the 1,3-butylene glycol product according to the present disclosure, a ratio of the acid concentration after keeping the aqueous solution at 100 °C for 1 week to the acid concentration before keeping the solution at 100 °C for 1 week, [(acid concentration after keeping the solution at 100 °C for 1 week)/(acid concentration before keeping the solution at 100 °C for 1 week)×100(%)], is preferably 150% or less, more preferably 120% or less, and even more preferably 110% or less.

The 1,3-butylene glycol product of the present disclosure has an APHA (Hazen color number) of, for example, 3 or less (preferably 2 or less, and more preferably 1 or less). Furthermore, an APHA, after the 1,3-butylene glycol product has been kept at 100 °C for 75 days in air atmosphere, is 11 or less (preferably 10 or less, more preferably 8 or less, 7 or less, 6 or less, and even more preferably 5 or less, 4 or less, 3 or less, or 2 or less).

For the APHA of the 1,3-butylene glycol product according to the present disclosure, a ratio of the APHA after the 1,3-butylene glycol product has been kept at 100 °C for 75 days to the APHA before the 1,3-butylene glycol product has been kept at 100 °C for 75 days, [(APHA after the 1,3-butylene glycol product has been kept at 100 °C for 75 days)/(APHA before the 1,3-butylene glycol product has been kept at 100 °C for 75 days)], is not particularly limited, but is preferably less than 3, more preferably 2.5 or less, even more preferably 2 or less, and particularly preferably 1.5 or less (for example, 1.2 or less).

Additionally, the 1,3-butylene glycol product according to the present disclosure preferably has an initial boiling point of 204 °C or higher. The initial boiling point is preferably 204.5 °C or higher, more preferably 205 °C or higher, even more preferably 206 °C or higher or 207 °C, and particularly preferably 208 °C or higher.

In addition, the 1,3-butylene glycol product of the present disclosure preferably has a dry point of lower than 209 °C (e.g., 208.8 °C or lower).

The 1,3-butylene glycol product of the present disclosure preferably has a potassium permanganate test value (PMT) of 36 minutes or longer. The potassium permanganate test value (PMT) is more preferably 38 minutes or longer, even more preferably 40 minutes or longer, and particularly preferably 50 minutes or longer (especially 60 minutes or longer).

The 1,3-butylene glycol product of the present disclosure has a content of 1,3-butylene glycol of, for example, 99.3% or greater. The content of the 1,3-butylene glycol corresponds to an area ratio (GC area ratio) of the peak of 1,3-butylene glycol, in the gas chromatographic analysis (GC analysis) performed under the following conditions.

### The Conditions for the Gas Chromatographic Analysis are as Follows:

Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

In the present disclosure, the “(peak) area ratio” means a ratio of the area of a specific peak relative to the sum of the areas of all peaks appearing in the chromatogram. In addition, all peaks mean, for example, all of the peaks appearing in the analysis continued until and discontinued at a relative retention time of 7.8, provided that the relative retention time of 1,3-butylene glycol is 1.0. The content of 1,3-butylene glycol in the 1,3-butylene glycol product is, for example, 98.6% or greater, and thus basic properties inherent in 1,3-butylene glycol are guaranteed.

The content (GC area ratio) of the 1,3-butylene glycol is preferably 99.4% or greater, more preferably 99.5% or greater, even more preferably 99.6% or greater, and particularly preferably 99.7% or greater, and especially 99.8% or greater.

In the GC analysis, a total area ratio of peaks shorter in retention time than a peak of 1,3-butylene glycol is preferably 0.09% or less, more preferably 0.08% or less, even more preferably 0.07% or less, 0.04% or less, 0.03% or less, 0.02% or less, 0.01% or less, 0.007% or less, and particularly preferably 0.005% or less (e.g., 0.003% or less).

In the GC analysis, a total area ratio of peaks longer in retention time than the peak of 1,3-butylene glycol is preferably 0.7% or less, more preferably 0.6% or less, even more preferably 0.5% or less, 0.4% or less, 0.3% or less, or 0.2% or less, and particularly preferably 0.1% or less.

In the 1,3-butylene glycol product according to the present disclosure, a content of water is preferably 0.2 wt. % or less. The content of water is preferably 0.15 wt. % or less, more preferably 0.1 wt. % or less, even more preferably 0.07 wt. % or less, 0.05 wt. % or less, 0.03 wt. % or less, 0.02 wt. % or less, or 0.01 wt. % or less, and particularly preferably 0.005 wt. % or less. Note that the content of water can be quantified by a Karl Fischer water content measurement instrument.

The 1,3-butylene glycol product of the present disclosure has no or very little coloration or odor even after storage for a long period of time. In addition, the 1,3-butylene glycol product of the present disclosure has extremely low contents of impurities that may serve as a substance that causes coloration or a substance that causes odor even after storage for a long period of time. For example, even after an acceleration test assuming long-term storage of the 1,3-butylene glycol product (test in which the 1,3-butylene glycol product has been kept at 180 °C for 3 hours in air atmosphere), the contents of impurities that may serve as a substance that causes coloration or a substance that causes odor in the product are extremely low.

In the 1,3-butylene glycol product of the present disclosure, a total content of acetaldehyde, crotonaldehyde, methyl vinyl ketone, acetone, formaldehyde, butylaldehyde, acetaldol, 1-hydroxy-3-butanone, 2-butanol and the compounds represented by Formulas (1) to (10) is, for example, less than 70 ppm, preferably 65 ppm or less, 60 ppm or less, 55 ppm or less, 50 ppm or less, 45 ppm or less, 40 ppm or less, 35 ppm or less, 30 ppm or less, 25 ppm or less, or 20 ppm or less, and even more preferably 18 ppm or less, 15 ppm or less, 13 ppm or less, 10 ppm or less, 8 ppm or less, 5 ppm or less, 4 ppm or less, or 3.5 ppm or less, after the 1,3-butylene glycol product has been kept at 180 °C for 3 hours in air atmosphere.

Also, the 1,3-butylene glycol product of the present disclosure preferably has a sum of the contents of the compounds represented by Formulas (1) to (7) above (cyclic acetal compounds) of, for example, less than 40 ppm, preferably 35 ppm or less, and more preferably 30 ppm or less, 25 ppm or less, 20 ppm or less, 15 ppm or less, 12 ppm or less, 10 ppm or less, 8 ppm or less, 6 ppm or less, 5 ppm or less, 4 ppm or less, 3 ppm or less, 2 ppm or less, or 1.4 ppm or less, after the product has been kept at 180 °C for 3 hours in air atmosphere.

Also, the 1,3-butylene glycol product of the present disclosure has a sum of the contents of the compounds represented by Formulas (8) to (10) above (3-hydroxybutyl carboxylate compounds) of, for example, less than 18 ppm, preferably 16 ppm or less, more preferably 14 ppm or less, 13 ppm or less, 12 ppm or less, 11 μm or less, 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, or 0.6 ppm or less, after the product has been kept at 180 °C for 3 hours in air atmosphere.

Further, the 1,3-butylene glycol product of the present disclosure preferably has a sum of the contents of the compounds represented by Formulas (1) to (10) above of, for example, less than 59 ppm, preferably 55 ppm or less, more preferably 50 ppm or less, 40 ppm or less, 30 ppm or less, 20 ppm or less, 18 ppm or less, 16 ppm or less, 14 ppm or less, 12 ppm or less, 10 ppm or less, 8 ppm or less, 7 ppm or less, 6 ppm or less, 5 ppm or less, 4 ppm or less, 3 ppm or less, or 2 ppm or less, after the product has been kept at 180 °C for 3 hours in air atmosphere.

In addition, in the 1,3-butylene glycol product of the present disclosure, the content of acetaldehyde is preferably less than 1 ppm, and more preferably 0.9 ppm or less, 0.8 ppm or less, 0.7 ppm or less, 0.6 ppm or less, 0.5 ppm or less, 0.4 ppm or less, 0.3 ppm or less, or 0.2 ppm or less, after the 1,3-butylene glycol product has been kept at 180 °C for 3 hours in air atmosphere. The content of the crotonaldehyde is preferably less than 0.5 ppm, and more preferably 0.4 ppm or less, 0.3 ppm or less, 0.2 ppm or less, or 0.1 ppm or less. The content of methyl vinyl ketone is preferably less than 4 ppm, and more preferably 3 ppm or less, 2 ppm or less, 1 ppm or less, 0.8 ppm or less, 0.6 ppm or less, 0.4 ppm or less, 0.3 ppm or less, or 0.2 ppm or less. The content of acetone is preferably less than 2 ppm, and more preferably 1 ppm or less, 0.8 ppm or less, 0.6 ppm or less, 0.4 ppm or less, 0.3 ppm or less, or 0.2 ppm or less. The content of formaldehyde is preferably less than 0.5 ppm, and more preferably 0.4 ppm or less, 0.3 ppm or less, or 0.2 ppm or less. The content of butylaldehyde is preferably less than 3 ppm, and more preferably 2 ppm or less, 1 ppm or less, 0.8 ppm or less, 0.6 ppm or less, 0.4 ppm or less, 0.3 ppm or less, or 0.2 ppm or less. The content of acetaldol is preferably less than 0.5 ppm, and more preferably 0.4 ppm or less, 0.3 ppm or less, or 0.2 ppm or less. The content of 1-hydroxy-3-butanone is preferably less than 0.5 ppm, and more preferably 0.4 ppm or less, 0.3 ppm or less, or 0.2 ppm or less. The content of 2-butanol is preferably less than 0.2 ppm. The content of the compound represented by Formula (1) is preferably less than 6 ppm, and more preferably 5 ppm or less, 4 ppm or less, 3 ppm or less, 2 ppm or less, 1 ppm or less, 0.8 ppm or less, 0.6 ppm or less, 0.4 ppm or less, 0.3 ppm or less, or 0.2 ppm or less. The content of the compound represented by Formula (2) is preferably less than 5 ppm, and more preferably 4 ppm or less, 3 ppm or less, 2 ppm or less, 1 ppm or less, 0.8 ppm or less, 0.6 ppm or less, 0.4 ppm or less, 0.3 ppm or less, or 0.2 ppm or less. The content of the compound represented by Formula (3) is preferably less than 4 ppm, and more preferably 3 ppm or less, 2 ppm or less, 1 ppm or less, 0.8 ppm or less, 0.6 ppm or less, 0.4 ppm or less, 0.3 ppm or less, or 0.2 ppm or less. The content of the compound represented by Formula (4) is preferably less than 5 ppm, and more preferably 4 ppm or less, 3 ppm or less, 2 ppm or less, 1 ppm or less, 0.8 ppm or less, 0.6 ppm or less, 0.4 ppm or less, 0.3 ppm or less, or 0.2 ppm or less. The content of the compound represented by Formula (5) is preferably less than 7 ppm, and more preferably 6 ppm or less, 5 ppm or less, 4 ppm or less, 3 ppm or less, 2 ppm or less, 1 ppm or less, 0.8 ppm or less, 0.6 ppm or less, 0.4 ppm or less, 0.3 ppm or less, or 0.2 ppm or less. The content of the compound represented by Formula (6) is preferably less than 6 ppm, and more preferably 5 ppm or less, 4 ppm or less, 3 ppm or less, 2 ppm or less, 1 ppm or less, 0.8 ppm or less, 0.6 ppm or less, 0.4 ppm or less, 0.3 ppm or less, or 0.2 ppm or less. The content of the compound represented by Formula (7) is preferably less than 8 ppm, and more preferably 7 ppm or less, 6 ppm or less, 5 ppm or less, 4 ppm or less, 3 ppm or less, 2 ppm or less, 1 ppm or less, 0.8 ppm or less, 0.6 ppm or less, 0.4 ppm or less, 0.3 ppm or less, or 0.2 ppm or less. The content of the compound represented by Formula (8) is preferably less than 6 ppm, and more preferably 5 ppm or less, 4 ppm or less, 3 ppm or less, 2 ppm or less, 1 ppm or less, 0.8 ppm or less, 0.6 ppm or less, 0.4 ppm or less, 0.3 ppm or less, or 0.2 ppm or less. The content of the compound represented by Formula (9) is preferably less than 5 ppm, and more preferably 4 ppm or less, 3 ppm or less, 2 ppm or less, 1 ppm or less, 0.8 ppm or less, 0.6 ppm or less, 0.4 ppm or less, 0.3 ppm or less, or 0.2 ppm or less. The content of the compound represented by Formula (10) is preferably less than 7 ppm, and more preferably 6 ppm or less, 5 ppm or less, 4 ppm or less, 3 ppm or less, 2 ppm or less, 1 ppm or less, 0.8 ppm or less, 0.6 ppm or less, 0.4 ppm or less, 0.3 ppm or less, or 0.2 ppm or less.

In the 1,3-butylene glycol product, a sum of the contents of acetaldehyde, crotonaldehyde, methyl vinyl ketone, acetone, formaldehyde, butylaldehyde, acetaldol, and 1-hydroxy-3-butanone is, for example, less than 12 ppm, preferably 10 ppm or less, and more preferably 8 ppm or less, 7 ppm or less, 6 ppm or less, 5 ppm or less, 4 ppm or less, 3 ppm or less, 2 ppm or less, or 1.6 ppm or less, after the 1,3-butylene glycol product has been kept at 180 °C for 3 hours in air atmosphere.

In addition, in the 1,3-butylene glycol product of the present disclosure, a sum of the contents of acetaldol and 1-hydroxy-3-butanone is, for example, less than 1 ppm, preferably 0.9 ppm or less, and more preferably 0.8 ppm or less, 0.7 ppm or less, 0.6 ppm or less, 0.5 ppm or less, or 0.4 ppm or less, after the 1,3-butylene glycol product has been kept at 180 °C for 3 hours in air atmosphere.

Furthermore, in the 1,3-butylene glycol product according to the present disclosure, an APHA is less than 25 (preferably 20 or less, and more preferably 18 or less, 15 or less, 14 or less, 13 or less, 12 or less, 11 or less, 10 or less, 9 or less, 8 or less, or 7 or less), after the 1,3-butylene glycol product has been kept at 180 °C for 3 hours in air atmosphere.

Also, the 1,3-butylene glycol product according to the present disclosure preferably has an initial boiling point of 204 °C or greater and preferably has a dry point of less than 209 °C, after the 1,3-butylene glycol product has been kept at 180 °C for 3 hours in air atmosphere. The initial boiling point is preferably 205 °C or greater, more preferably 206 °C or greater, and even more preferably 207 °C or greater.

The 1,3-butylene glycol product according to the present disclosure preferably has a potassium permanganate test value (PMT) of longer than 30 minutes, after the 1,3-butylene glycol product has been kept at 180 °C for 3 hours in air atmosphere. The potassium permanganate test value is more preferably longer than 32 minutes or longer, even more preferably 35 minutes or longer (e.g., 40 minutes or longer), and particularly preferably 50 minutes or longer (especially, 60 minutes or longer).

The 1,3-butylene glycol product according to the present disclosure preferably has an acid concentration (in terms of acetic acid) of less than 8 ppm, after the 1,3-butylene glycol product has been kept at 180 °C for 3 hours in air atmosphere. The acid concentration (in terms of acetic acid) is more preferably 7 ppm or less, even more preferably 6 ppm or less, 5 ppm or less, 4 ppm or less, 3 ppm or less, 2 ppm or less, or 1 ppm or less.

A 1,3-butylene glycol product having a content of 1,3-butylene glycol of a specific value or greater and a content of a specific substance after the heating test under the specific conditions has high purity and high quality, and is hardly deteriorated in quality over time. In particular, the 1,3-butylene glycol product according to the present disclosure is colorless and odorless (or almost colorless and odorless), and hardly causes or increases odor over time.

Therefore, it can be suitably used as a raw material for moisturizers or cosmetic products.

### Moisturizer and Cosmetic Product

A moisturizer of the present disclosure contains the 1,3-butylene glycol product described above. Therefore, the moisturizer has excellent moisturizing performance. Therefore, the 1,3-butylene glycol product obtained by the manufacturing method of the present disclosure can be suitably used as a raw material for moisturizers and cosmetic products. The moisturizer of the present disclosure may contain a component besides the 1,3-butylene glycol product described above, such as a moisturizer component besides the 1,3-butylene glycol product described above. In the moisturizer of the present disclosure, the content of the 1,3-butylene glycol product described above is, for example, 10 wt. % or greater, preferably 30 wt. % or greater, more preferably 50 wt. % or greater, even more preferably 80 wt. % or greater, and particularly preferably 90 wt. % or greater, and the moisturizer may be composed of only the 1,3-butylene glycol product described above.

A cosmetic of the present disclosure contains the moisturizer described above. The blending amount of the 1,3-butylene glycol product in the cosmetic product of the present disclosure is any amount in which the moisturizing performance can be exhibited according to the type and form of cosmetic. The blending amount of the 1,3-butylene glycol product in the cosmetic product of the present disclosure is, for example, from 0.01 to 40 wt. %, preferably from 0.1 to 30 wt. %, more preferably from 0.2 to 20 wt. %, even more preferably from 0.5 to 15 wt. %, and particularly preferably from 1 to 10 wt. %.

The cosmetic product of the present disclosure may contain, in addition to the 1,3-butylene glycol product, for example, another moisturizer; an oil, such as a vegetable oil, a hydrocarbon oil, a higher fatty acid, a higher alcohol, or a silicone; a surfactant, such as an anionic surfactant, a cationic surfactant, an amphoteric surfactant, or a nonionic surfactant; a preservative, a sequestrant, a thickener, a powder, an ultraviolet absorber, an ultraviolet blocker, a fragrance, or a pH adjuster; or a medicinal ingredient or bioactive component, such as a vitamin preparation, a skin activator, a blood circulation promoter, a skin-lightening preparation, an antibacterial agent, or an anti-inflammatory agent.

The cosmetic product of the present disclosure can be a skin cosmetic product, such as a lotion, an emulsion, a cream, a gel, a pack, or a mask; or a hair cosmetic product, such as a shampoo, a rinse, or a hair restorer. In addition, the cosmetic product may be a sunscreen cosmetic product, a make-up cosmetic product or the like. Furthermore, the cosmetic product can be a pharmaceutical product or quasi drug containing a medical component.

The cosmetic product of the present disclosure can be produced by utilizing a method known per se.

Each aspect disclosed in the present specification can be combined with any other feature disclosed herein. Note that each of the configurations, combinations thereof, and the like in each of the embodiments are an example, and various additions, omissions, and other changes may be made as appropriate without departing from the spirit of the present disclosure. The present disclosure is not limited by the embodiments and is limited only by the claims.

### EXAMPLES

Hereinafter, the present disclosure will be described more specifically with reference to examples, but the present disclosure is not limited by these examples. “Parts” used in the examples means “parts by weight” unless otherwise specified. Gas chromatographic analysis (GC analysis), measurement of an initial boiling point, measurement of dry point, measurement of water content, GC-MS analysis (gas mass spectrometry), and potassium permanganate test were performed by the methods which will be described below.

The following examples, comparative examples, and reference examples primarily relate to the first aspect of the present disclosure.

### Example 1—Primarily Related to the First Aspect of the Present Disclosure

The method of manufacturing 1,3-butylene glycol will be described using FIG. 1.

Relative to 100 parts of an acetaldol solution containing 30 wt. % of water (mixed solution of 70 parts of acetaldol and 30 parts of water) as a raw material, 10 parts of hydrogen were charged in a reactor for liquid-phase hydrogenation reduction, and 15 parts of Raney nickel were added as a catalyst. The reactor was kept at 120 °C and 10 MPa (gauge pressure), and liquid-phase hydrogenation reduction was performed. After the catalyst was separated, the liquid after the reaction was neutralized with sodium hydroxide, alcohols were removed, and crude 1,3-butylene glycol (1) containing a substance having a higher boiling point than that of 1,3-butylene glycol was produced.

Note that the acetaldol solution containing 30 wt. % of water, which was used as the raw material, had been produced by stirring acetaldehyde and water in the presence of 100 ppm by weight NaOH at 30 °C for a residence time of 10 hours, and dimerizing the acetaldehyde [acetaldehyde polymerization (aldol condensation of acetaldehyde)].

The crude 1,3-butylene glycol (1) (corresponding to “X-1” in FIG. 1) was charged in the dehydration column A. In the dehydration column A, water was extracted from the top of the column, and 15 parts of fresh water was added as reflux water relative to 100 parts of the charged liquid amount. The pressure at the top of the column was adjusted to 7 kPa (absolute pressure), and crude 1,3-butylene glycol (2) was produced from the bottom of the column, the crude 1,3-butylene glycol (2) having a total area ratio of impurity peaks shorter in retention time (RT) than 1,3-butylene glycol of 1.8% in the GC analysis which will be described later. The water extracted from the top of the column was discharged (corresponding to “X-2” in FIG. 1).

The crude 1,3-butylene glycol (2) was then charged in the desalting column B. In the desalting column B, a salt, a high boiling point substance, and a portion of 1,3-butylene glycol were discharged as the evaporation residue from the bottom of the column (corresponding to “X-3” in FIG. 1). The discharge amount of the evaporation residue was 5 parts relative to 100 parts of the charged liquid amount. On the other hand, from the top of the column, the crude 1,3-butylene glycol (3) containing 1,3-butylene glycol, a low boiling point substance, and a portion of a high boiling point substance was produced. As a result of the GC analysis of the crude 1,3-butylene glycol (3), the total area ratio of the impurity peaks longer in retention time than 1,3-butylene glycol was 3%, and the area ratio of the 1,3-butylene glycol peak was 95%.

The crude 1,3-butylene glycol (3) was then charged in the high boiling point component removal column C. In the high boiling point component removal column C, distillation was performed under the condition of a pressure at the top of the column of 5 kPa (absolute pressure), and a high boiling point substance and a portion of 1,3-butylene glycol were discharged from the bottom of the column (corresponding to “X-4” in FIG. 1). The discharge amount from the bottom of the column was 20 parts relative to 100 parts of the charged liquid amount, and the discharge was incinerated as it was. On the other hand, at the top of the column, 80 parts of crude 1,3-butylene glycol (4) containing a low boiling point substance was produced, as a distillate, in a reflux ratio (reflux amount/distilled amount) of 0.05.

The crude 1,3-butylene glycol (4) was then charged in the alkaline reactor D. At this time, a 20 wt. % sodium hydroxide aqueous solution was added to give a concentration of sodium hydroxide of 0.1 wt. % relative to the charged liquid. The reaction temperature was maintained at 120 °C in the alkaline reactor D, and a reaction was performed at a residence time of 20 minutes.

A reaction crude liquid exiting the alkaline reactor D was then charged in the dealkalization column E. In the dealkalization column E, sodium hydroxide, a high boiling point substance, and a portion of 1,3-butylene glycol were discharged from the bottom of the column (corresponding to “X-5” in FIG. 1). The discharge amount from the bottom of the column was 5 parts relative to 100 parts of the charged liquid amount. On the other hand, from the top of the column were produced 95 parts of crude 1,3-butylene glycol (5) containing 1,3-butylene glycol and a low boiling point substance.

The crude 1,3-butylene glycol (5) was then charged in the product distillation column F. In the product distillation column F, 20 parts of a low boiling point substance and a portion of 1,3-butylene glycol relative to 100 parts of the charged liquid amount were distilled off from the top of the column (corresponding to “X-6” in FIG. 1), and the entire amount was discharged to the outside of the system. The operation was performed at a reflux ratio (reflux amount/distilled amount) of 1, and 80 parts of a 1,3-butylene glycol product was produced from the bottom of the column (corresponding to “Y” in FIG. 1).

As a result of the dry point and GC analysis for the above-described 1,3-butylene glycol product, the dry point was 208.9 °C, the total area ratio of the impurity peaks longer in retention time (RT) than 1,3-butylene glycol was 0.6%, and the area ratio of the 1,3-butylene glycol peak was 99.1%. Furthermore, the 1,3-butylene glycol recovery ratio at the high boiling point component removal column C was 83%.

### Example 2—Primarily Related to the First Aspect of the Present Disclosure

A 1,3-butylene glycol product was produced from the bottom of the product column F by the same method as in Example 1 except that the distilled amount from the top of the high boiling point component removal column C was changed to 85 parts. As a result of the dry point and GC analysis for the 1,3-butylene glycol product, the dry point was 209.0 °C, the total area ratio of the impurity peaks longer in retention time than 1,3-butylene glycol was 0.7%, and the area ratio of the 1,3-butylene glycol peak was 99.0%. Additionally, the 1,3-butylene glycol recovery ratio at the high boiling point component removal column C was 88%.

### Example 3—Primarily Related to the First Aspect of the Present Disclosure

A 1,3-butylene glycol product was produced from the bottom of the product column F by the same method as in Example 1 except that the reflux ratio of the high boiling point component removal column C was changed to 0.2. As a result of the dry point and GC analysis for the 1,3-butylene glycol product, the dry point was 208.7 °C, the total area ratio of the impurity peaks longer in retention time than 1,3-butylene glycol was 0.2%, and the area ratio of the 1,3-butylene glycol peak was 99.5%. Furthermore, the 1,3-butylene glycol recovery ratio at the high boiling point component removal column C was 84%.

### Example 4—Primarily Related to the First Aspect of the Present Disclosure

A 1,3-butylene glycol product was produced from the bottom of the product column F by the same method as in Example 1 except that the reflux ratio of the high boiling point component removal column C was changed to 0.6. As a result of the dry point and GC analysis for the 1,3-butylene glycol product, the dry point was 208.6 °C, the total area ratio of the impurity peaks longer in retention time than 1,3-butylene glycol was 0.05%, and the area ratio of the 1,3-butylene glycol peak was 99.7%. Furthermore, the 1,3-butylene glycol recovery ratio at the high boiling point component removal column C was 84%.

### Example 5—Primarily Related to the First Aspect of the Present Disclosure

A 1,3-butylene glycol product was produced from the bottom of the product column F by the same method as in Example 1 except that the reflux ratio of the high boiling point component removal column C was changed to 0.6, and that the distilled amount from the top of the column was changed to 90 parts. As a result of the dry point and GC analysis for the 1,3-butylene glycol product, the dry point was 208.7 °C, the total area ratio of the impurity peaks longer in retention time than 1,3-butylene glycol was 0.2%, and the area ratio of the 1,3-butylene glycol peak was 99.5%.

Furthermore, the 1,3-butylene glycol recovery ratio at the high boiling point component removal column C was 94%.

### Example 6—Primarily Related to the First Aspect of the Present Disclosure

A 1,3-butylene glycol product was produced from the bottom of the product column F by the same method as in Example 1 except that the reflux ratio of the high boiling point component removal column C was changed to 1, and that the distilled amount form the top of the column was changed to 90 parts. As a result of the dry point and GC analysis for the 1,3-butylene glycol product, the dry point was 208.6 °C, the total area ratio of the impurity peaks longer in retention time than 1,3-butylene glycol was 0.1%, and the area ratio of the 1,3-butylene glycol peak was 99.6%. Furthermore, the 1,3-butylene glycol recovery ratio at the high boiling point component removal column C was 94%.

### Example 7—Primarily Related to the First Aspect of the Present Disclosure

A 1,3-butylene glycol product was produced from the bottom of the product column F by the same method as in Example 1 except that the distilled amount from the top of the high boiling point component removal column C was 90 parts, that 5 parts of 10 parts of the bottom of the high boiling point component removal column C was discarded, that 5 parts thereof was recycled to the acetaldol production (aldol condensation of acetaldehyde), and that the reflux ratio of the high boiling point component removal column C was 1. As a result of the dry point and GC analysis for the 1,3-butylene glycol product, the dry point was 208.7 °C, the total area ratio of the impurity peaks longer in retention time than 1,3-butylene glycol was 0.2%, and the area ratio of the 1,3-butylene glycol peak was 99.5%. Furthermore, the 1,3-butylene glycol recovery ratio at the high boiling point component removal column C was 99% or greater.

### Example 8—Primarily Related to the First Aspect of the Present Disclosure

A 1,3-butylene glycol product was produced from the bottom of the product column F by the same method as in Example 1 except that the distilled amount from the top of the high boiling point component removal column C was 90 parts, that 5 parts of 10 parts of the bottom of the high boiling point component removal column C was discarded, that 5 parts thereof was recycled to the acetaldol production, and that the reflux ratio of the high boiling point component removal column C was 10. As a result of the dry point and GC analysis for the 1,3-butylene glycol product, the dry point was 208.7 °C, the total area ratio of the impurity peaks longer in retention time than 1,3-butylene glycol was 0.1%, and the area ratio of the 1,3-butylene glycol peak was 99.6%. Furthermore, the 1,3-butylene glycol recovery ratio at the high boiling point component removal column C was 99% or greater.

### Example 9—Primarily Related to the First Aspect of the Present Disclosure

A 1,3-butylene glycol product was produced from the bottom of the product column F by the same method as in Example 1 except that the distilled amount from the top of the high boiling point component removal column C was 90 parts, that 5 parts of the bottom of the high boiling point component removal column C was discarded, that 5 parts thereof was recycled to the acetaldol production, and that the reflux ratio was 20. As a result of the dry point and GC analysis for the 1,3-butylene glycol product, the dry point was 208.6 °C, the total area ratio of the impurity peaks longer in retention time than 1,3-butylene glycol was 0.08%, and the area ratio of the 1,3-butylene glycol peak was 99.6%. Furthermore, the 1,3-butylene glycol recovery ratio at the high boiling point component removal column C was 99% or greater.

### Example 10—Primarily Related to the First Aspect of the Present Disclosure

The procedure up to the dehydration was carried out in the same manner as in Example 1.

The crude 1,3-butylene glycol (2) produced from the bottom of the dehydration column A was charged in the desalting column B. In the desalting column B, a salt, a high boiling point substance, and a portion of 1,3-butylene glycol were discharged as the evaporation residue from the bottom of the column (corresponding to “X-3” in FIG. 1). The discharge amount of the evaporation residue was 10 parts relative to 100 parts of the charged liquid amount. On the other hand, from the top of the column, the crude 1,3-butylene glycol (3) containing 1,3-butylene glycol, a low boiling point substance, and a portion of a high boiling point substance was produced. As a result of the GC analysis of the crude 1,3-butylene glycol (3), the total area ratio of the impurity peaks longer in retention time than 1,3-butylene glycol was 2%, and the area ratio of the 1,3-butylene glycol peak was 97%.

The crude 1,3-butylene glycol (3) was then charged in the high boiling point component removal column C. In the high boiling point component removal column C, a high boiling point substance and a portion of 1,3-butylene glycol were discharged from the bottom of the column (corresponding to “X-4” in FIG. 1). The discharge amount at the bottom of the column was 5 parts relative to 100 parts of the charged liquid amount, and the discharge was incinerated as it was. On the other hand, at the top of the column, 95 parts of crude 1,3-butylene glycol (4) containing a low boiling point substance was produced, as a distillate, in a reflux ratio (reflux amount/distilled amount) of 0.02.

Alkaline reaction, dealkalization, and product distillation were performed in the same manner as in Example 1 using this crude 1,3-butylene glycol (4). As a result of the dry point and GC analysis for the above-described 1,3-butylene glycol product produced from the bottom of the product distillation column, the dry point was 208.9 °C, the total area ratio of the impurity peaks longer in retention time (RT) than 1,3-butylene glycol was 0.5%, and the area ratio of the 1,3-butylene glycol peak was 99.2%. Additionally, the 1,3-butylene glycol recovery ratio at the high boiling point component removal column C was 97%.

### Example 11—Primarily Related to the First Aspect of the Present Disclosure

A 1,3-butylene glycol product was produced from the bottom of the product column F by the same method as in Example 10 except that the reflux ratio of the high boiling point component removal column C was 0.05. As a result of the dry point and GC analysis for the 1,3-butylene glycol product, the dry point was 208.7 °C, the total area ratio of the impurity peaks longer in retention time than 1,3-butylene glycol was 0.3%, and the area ratio of the 1,3-butylene glycol peak was 99.4%. Additionally, the 1,3-butylene glycol recovery ratio at the high boiling point component removal column C was 97%.

### Example 12—Primarily Related to the First Aspect of the Present Disclosure

A 1,3-butylene glycol product was produced from the bottom of the product column F by the same method as in Example 10 except that the reflux ratio of the high boiling point component removal column C was 1. As a result of the dry point and GC analysis for the 1,3-butylene glycol product, the dry point was 208.4 °C, the total area ratio of the impurity peaks longer in retention time than 1,3-butylene glycol was 0.03%, and the area ratio of the 1,3-butylene glycol peak was 99.7%. Additionally, the 1,3-butylene glycol recovery ratio at the high boiling point component removal column C was 97%.

### Example 13—Primarily Related to the First Aspect of the Present Disclosure

The procedure up to the dehydration was carried out in the same manner as in Example 1.

The crude 1,3-butylene glycol (2) produced from the bottom of the dehydration column A was charged in the desalting column B. In the desalting column B, a salt, a high boiling point substance, and a portion of 1,3-butylene glycol were discharged as the evaporation residue from the bottom of the column (corresponding to “X-3” in FIG. 1). The discharge amount of the evaporation residue was 15 parts relative to 100 parts of the charged liquid amount. On the other hand, from the top of the column, the crude 1,3-butylene glycol (3) containing 1,3-butylene glycol, a low boiling point substance, and a portion of a high boiling point substance was produced. As a result of the GC analysis of the crude 1,3-butylene glycol (3), the total area ratio of the impurity peaks longer in retention time than 1,3-butylene glycol was 0.5%, and the area ratio of the 1,3-butylene glycol peak was 99%.

The crude 1,3-butylene glycol (3) was then charged in the high boiling point component removal column C. In the high boiling point component removal column C, a high boiling point substance and a portion of 1,3-butylene glycol were discharged from the bottom of the column (corresponding to “X-4” in FIG. 1). The discharge amount at the bottom of the column was 5 parts relative to 100 parts of the charged liquid amount, and the discharge was incinerated as it was. On the other hand, at the top of the column, 95 parts of crude 1,3-butylene glycol (4) containing a low boiling point substance was produced, as a distillate, in a reflux ratio (reflux amount/distilled amount) of 0.02.

Alkaline reaction, dealkalization, and product distillation were performed in the same manner as in Example 1 using this crude 1,3-butylene glycol (4). As a result of the dry point and GC analysis for the above-described 1,3-butylene glycol product produced from the bottom of the product distillation column, the dry point was 208.6 °C, the total area ratio of the impurity peaks longer in retention time (RT) than 1,3-butylene glycol was 0.2%, and the area ratio of the 1,3-butylene glycol peak was 99.7%. Additionally, the 1,3-butylene glycol recovery ratio at the high boiling point component removal column C was 95%.

### Comparative Example 1—Primarily Related to the First Aspect of the Present Disclosure

A 1,3-butylene glycol product was produced from the bottom of the product column F by the same method as in Example 1 except that the reflux ratio of the high boiling point component removal column C was changed to 0.01, and that the reflux ratio of the product column was changed to 0.1. As a result of the dry point and GC analysis for the 1,3-butylene glycol product, the dry point was 210.0 °C, the total area ratio of the impurity peaks longer in retention time than 1,3-butylene glycol was 1.4%, and the area ratio of the 1,3-butylene glycol peak was 98.3%. Furthermore, the 1,3-butylene glycol recovery ratio at the high boiling point component removal column C was 83%.

### Comparative Example 2—Primarily Related to the First Aspect of the Present Disclosure

A 1,3-butylene glycol product was produced from the bottom of the product column F by the same method as in Example 1 except that the reflux ratio of the high boiling point component removal column C was changed to 0.02, and that the reflux ratio of the product column was changed to 0.1. As a result of the dry point and GC analysis for the 1,3-butylene glycol product, the dry point was 209.8 °C, the total area ratio of the impurity peaks longer in retention time than 1,3-butylene glycol was 1.2%, and the area ratio of the 1,3-butylene glycol peak was 98.6%. Furthermore, the 1,3-butylene glycol recovery ratio at the high boiling point component removal column C was 83%.

### Reference Example 1—Primarily Related to the First Aspect of the Present Disclosure

A 1,3-butylene glycol product was produced from the bottom of the product column F by the same method as in Example 1 except that the reflux ratio of the high boiling point component removal column C was 0.02, that the reflux ratio of the product column was 0.1, and that the distilled amount from the top of the high boiling point component removal column C was 70 parts. As a result of the dry point and GC analysis for the 1,3-butylene glycol product, the dry point was 209.0 °C, the total area ratio of the impurity peaks longer in retention time than 1,3-butylene glycol was 0.7%, and the area ratio of the 1,3-butylene glycol peak was 99.0%. Furthermore, the 1,3-butylene glycol recovery ratio at the high boiling point component removal column C was 73%.

The following examples, comparative examples, and reference examples primarily relate to the seventh aspect of the present disclosure.

### Example 1—Primarily Related to the Seventh Aspect of the Present Disclosure

The method of producing 1,3-butylene glycol will be described using FIG. 4.

Relative to 100 parts of an acetaldol solution containing 30 wt. % of water (mixed solution of 69 parts of acetaldol and 29 parts of water, containing a total of 2 parts of low boiling and high boiling impurities, Na salt: less than 0.1 parts) as a raw material, 10 parts of hydrogen were charged in a reactor for liquid-phase hydrogen reduction, and 15 parts of Raney nickel were added as a catalyst. The reactor was kept at 120 °C and 10 MPa (gauge pressure), and liquid-phase hydrogen reduction was performed. After the catalyst was separated, the liquid after the reaction was neutralized with sodium hydroxide, and crude 1,3-butylene glycol (1) containing water was produced.

Note that the acetaldol solution containing 30 wt. % of water used as the raw material was produced by stirring acetaldehyde and water in the presence of 100 ppm by weight NaOH at 30 °C at a residence time of 10 hours and dimerizing the acetaldehyde [acetaldehyde polymerization (aldol condensation of acetaldehyde)].

The crude 1,3-butylene glycol (1) (corresponding to “X-1” in FIG. 4) was charged in the dehydration column A. The concentration of 1,3-butylene glycol in the charged liquid into the dehydration column A was 56 wt. %, the water concentration was 40 wt. %, the content of acetaldehyde (AD) was 130 ppm, the content of crotonaldehyde (CR) was 89 ppm, the total area ratio of impurity peaks shorter in retention time (RT) than 1,3-butylene glycol in GC analysis which will be described later was 3%, and the total area ratio of impurity peaks longer in retention time than 1,3-butylene glycol was 1%. In the dehydration column A, the charged liquid containing 1,3-butylene glycol was distilled under conditions of a pressure at the top of 10 kPa (absolute pressure) and a reflux ratio of 1, water was extracted from the top of the column, and 43 parts (distilled amount) relative to 100 parts of the charged liquid amount was discharged and removed to the outside of the system (corresponding to “X-2” in FIG. 4). From the bottom of the column, crude 1,3-butylene glycol (2) having a 1,3-butylene glycol concentration of 96.9 GC area %, a water content of 0.9 wt. %, a total area ratio of impurity peaks shorter in retention time than 1,3-butylene glycol in the GC analysis which will be described later of 0.8%, a total area ratio of peaks longer in retention time than peak of 1,3-butylene glycols of 2.3%, an acetaldehyde content of 18 ppm, and a crotonaldehyde content of 17 ppm was produced.

The crude 1,3-butylene glycol (2) was then charged in the desalting column B. In the desalting column B, a salt, a high boiling point substance, and a portion of 1,3-butylene glycol were discharged as the evaporation residue from the bottom of the column (corresponding to “X-3” in FIG. 4). The discharge amount of the evaporation residue was 5 parts relative to 100 parts of the charged liquid amount. On the other hand, from the top of the column, crude 1,3-butylene glycol (3) containing 1,3-butylene glycol, a low boiling point substance, and a portion of a high boiling point substance was produced.

The crude 1,3-butylene glycol (3) was then charged in the high boiling point component removal column C. In the high boiling point component removal column C, distillation was performed under the conditions of a pressure at the top of the column of 5 kPa (absolute pressure) and a reflux ratio of 0.05, and a high boiling point substance and a portion of 1,3-butylene glycol were discharged from the bottom of the column (corresponding to “X-4” in FIG. 4). The discharge amount from the bottom of the column was 20 parts relative to 100 parts of the charged liquid amount. On the other hand, 80 parts of crude 1,3-butylene glycol (4) containing a low boiling point substance was produced, as a distillate, from the top of the column.

The crude 1,3-butylene glycol (4) was then charged in the alkaline reactor D. At this time, a 20 wt. % sodium hydroxide aqueous solution was added to give a concentration of sodium hydroxide of 0.1 wt. % relative to the charged liquid. The reaction temperature was maintained at 120 °C in the alkaline reactor D, and a reaction was performed at a residence time of 20 minutes.

A reaction crude liquid exiting the alkaline reactor D was then charged in the dealkalization column E. In the dealkalization column E, sodium hydroxide, a high boiling point substance, and a portion of 1,3-butylene glycol were discharged from the bottom of the column (corresponding to “X-5” in FIG. 4). The discharge amount from the bottom of the column was 10 parts relative to 100 parts of the charged liquid amount. On the other hand, from the top of the column 90 parts of crude 1,3-butylene glycol (5) containing 1,3-butylene glycol and a low boiling point substance were produced. The crude 1,3-butylene glycol (5) containing 1,3-butylene glycol and a low boiling point substance was measured for water content, and subjected to GC analysis and GC-MS analysis. As a result, the water concentration was 1 wt. %, the area ratio of 1,3-butylene glycol was 99%, the total area ratio of impurity peaks shorter in retention time than 1,3-butylene glycol was 0.4%, the total area ratio of impurity peaks longer in retention time than 1,3-butylene glycol was 0.6%, the content of acetaldehyde was 20 ppm, and the content of crotonaldehyde was 9 ppm.

The crude 1,3-butylene glycol (5) was then charged in the product column F. In the product column F, 10 parts of the low boiling point substance and a portion of 1,3-butylene glycol relative to 100 parts of the charged liquid amount were distilled off from the top of the column (corresponding to “X-6” in FIG. 4), and the entire amount was discharged to the outside of the system. The operation was performed at a reflux ratio (reflux amount/distilled amount) of 0.5 at that time, and 90 parts of a 1,3-butylene glycol product was produced from the bottom of the column (distilled amount: 10 parts) (corresponding to “Y” in FIG. 4).

The produced 1,3-butylene glycol product was measured for initial boiling point and water content, and subjected to GC analysis and GC-MS analysis. As a result, the 1,3-butylene glycol product had an initial boiling point of 203.3 °C, a dry point of 209 °C, a water concentration of 0.2 wt. %, an area ratio of 1,3-butylene glycol of 99.2%, a total area ratio of impurity peaks shorter in retention time than 1,3-butylene glycol of 0.08%, a total area ratio of impurity peaks longer in retention time than 1,3-butylene glycol of 0.7%, an acetaldehyde content of 1.5 ppm, and a crotonaldehyde content of 0.9 ppm. The potassium permanganate test value was 35 minutes. The 1,3-butylene glycol recovery ratio in the product column F was 90%.

### Example 2

The same operation as in Example 1 was performed except that the reflux ratio of the dehydration column A was changed to 50. A 1,3-butylene glycol product was produced from the bottom of the product column F. Note that, due to the changes in conditions employed in the dehydration column A, the dehydration column bottom composition changed, and the charged liquid compositions in the high boiling point component removal column C and the product column F changed. As a result, the product qualities changed.

The produced 1,3-butylene glycol product was measured for initial boiling point and water content, and subjected to GC analysis and GC-MS analysis. As a result, the 1,3-butylene glycol product had an initial boiling point of 206.7 °C, a dry point of 208.9 °C, a water concentration of 0.1 wt. %, an area ratio of 1,3-butylene glycol of 99.3%, a total area ratio of impurity peaks shorter in retention time than 1,3-butylene glycol of 0.05%, a total area ratio of impurity peaks longer in retention time than 1,3-butylene glycol of 0.7%, an acetaldehyde content of 0.7 ppm, and a crotonaldehyde content of 0.7 ppm. The potassium permanganate test value was 45 minutes. The 1,3-butylene glycol recovery ratio in the product column F was 90%.

### Examples 3 to 27—Primarily Related to the Seventh Aspect of the Present Disclosure

Under the conditions shown in Table 3 and Table 4, the dehydration column A, the high boiling point component removal column C, and the product column F were operated. In Examples 4 to 22 and 24 to 27, the entire amount of the distillate from the product column F was recycled into hydrogen reduction reactor. In Example 23, the product column F was not used, and a distillate at the top of the dealkalization column E (demister was installed in a space above the charging position) was a 1,3-butylene glycol product. At this time, the concentration of the sodium hydroxide aqueous solution in the alkaline reactor D was set to 1.5 times, and the added amount of the sodium hydroxide aqueous solution was set to the half of the amount in Example 1. Thus, the increase in water content due to alkaline treatment was avoided as much as possible. Note that, when the alkali concentration of the sodium hydroxide aqueous solution is too high and crystals precipitate. Therefore, heating is preferably performed to 40 °C or higher. In Table 4, the column “Bottom from product column F” of Example 23 shows the composition and physical properties of the distillate at the top of the dealkalization column E. Note that, in Example 16, 8 parts of 10 parts of the bottom from the high boiling point component removal column was recycled into the hydrogenation, and 2 parts thereof was discharged to the outside of the system. In addition, in Example 25, the pressure of the hydrogen addition reaction was reduced to 7 MPaG (gauge pressure).

Therefore, the acetaldehyde content and crotonaldehyde content of the charged liquid into the dehydration column are high. In Example 26, the reflux ratio of the dehydration column was reduced to 0.3, and the purity of 1,3-butylene glycol in the charged liquid into the product column was reduced, and the reflux ratio of the product column was increased to 20. In Example 27, the pressure of the hydrogen addition reaction was increased to 40 MPaG (gauge pressure) (the remaining conditions are the same as in Example 18).

### Comparative Example 1—Primarily Related to the Seventh Aspect of the Present Disclosure

Eighty (80) parts of a 1,3-butylene glycol product was produced from the bottom of the product column F by the same method as in Example 1 except that the reflux ratio of the dehydration column A was changed to 0.5, that the distilled amount was changed to 42 parts, that the reflux ratio of the high boiling point component removal column C was changed to 0.02, that the reflux ratio of the product column F was changed to 0.05, and that the distilled amount was changed to 20 parts. The produced 1,3-butylene glycol product had an initial boiling point of 193.2 °C, a dry point of 210.3 °C, a water concentration of 0.6 wt. %, an area ratio of 1,3-butylene glycol of 98.3%, a total area ratio of impurity peaks shorter in retention time than 1,3-butylene glycol of 0.2%, a total area ratio of impurity peaks longer in retention time than 1,3-butylene glycol of 1.5%, an acetaldehyde content of 5 ppm, and a crotonaldehyde content of 4 ppm. The potassium permanganate test value was 0 minutes. The 1,3-butylene glycol recovery ratio in the product column F was 80%.

### Comparative Example 2—Primarily Related to the Seventh Aspect of the Present Disclosure

Eighty (80) parts of a 1,3-butylene glycol product was produced from the bottom of the product column F by the same method as in Example 1 except that the charged composition in the dehydration column A was changed, the reflux ratio and the distilled amount were changed to 0.5 and 32 parts, respectively, that the reflux ratio of the high boiling point component removal column C was changed to 0.02, that the reflux ratio of the product column F was changed to 0.05, and that the distilled amount was changed to 20 parts. The produced 1,3-butylene glycol product had an initial boiling point of 199.0 °C, a dry point of 210.1 °C, a water concentration of 0.4 wt. %, an area ratio of 1,3-butylene glycol of 98.5%, a total area ratio of impurity peaks shorter in retention time than 1,3-butylene glycol of 0.1%, a total area ratio of impurity peaks longer in retention time than 1,3-butylene glycol of 1.4%, an acetaldehyde content of 4 ppm, and a crotonaldehyde content of 2 ppm. The potassium permanganate test value was 5 minutes. The 1,3-butylene glycol recovery ratio in the product column F was 80%.

### Comparative Example 3—Primarily Related to the Seventh Aspect of the Present Disclosure

Seventy (70) parts of a 1,3-butylene glycol product was produced from the bottom of the product column F by the same method as in Example 1 except that the charged composition in the dehydration column A was changed, the reflux ratio and the distilled amount were changed to 0.5 and 32 parts, respectively, that the reflux ratio of the high boiling point component removal column C was changed to 0.02, that the reflux ratio of the product column F was changed to 0.05, and that the distilled amount was changed to 30 parts. The produced 1,3-butylene glycol product had an initial boiling point of 203.0 °C, a dry point of 210.2 °C, a water concentration of 0.2 wt. %, an area ratio of 1,3-butylene glycol of 98.4%, a total area ratio of impurity peaks shorter in retention time than 1,3-butylene glycol of 0.1%, a total area ratio of impurity peaks longer in retention time than 1,3-butylene glycol of 1.5%, an acetaldehyde content of 2 ppm, and a crotonaldehyde content of 1.3 ppm. The potassium permanganate test value was 30 minutes. The 1,3-butylene glycol recovery ratio in the product column F was 70%.

### Comparative Example 4—Primarily Related to the Seventh Aspect of the Present Disclosure

Eighty (80) parts of a 1,3-butylene glycol product was produced from the bottom of the product column F by the same method as in Example 1 except that the charged composition in the dehydration column A was changed, the distilled amount was changed to 23 parts, that the reflux ratio of the high boiling point component removal column C was changed to 0.02, that the reflux ratio of the product column F was changed to 0.1, and that the distilled amount was changed to 20 parts. The produced 1,3-butylene glycol product had an initial boiling point of 203.1 °C, a dry point of 209.5 °C, a water concentration of 0.2 wt. %, an area ratio of 1,3-butylene glycol of 98.8%, a total area ratio of impurity peaks shorter in retention time than 1,3-butylene glycol of 0.1%, a total area ratio of impurity peaks longer in retention time than 1,3-butylene glycol of 1.1%, an acetaldehyde content of 2 ppm, and a crotonaldehyde content of 1.3 ppm. The potassium permanganate test value was 30 minutes. The 1,3-butylene glycol recovery ratio in the product column F was 80%.

### Gas Chromatographic Analysis

A gas chromatographic analysis of the target 1,3-butylene glycol product was performed under the conditions below.

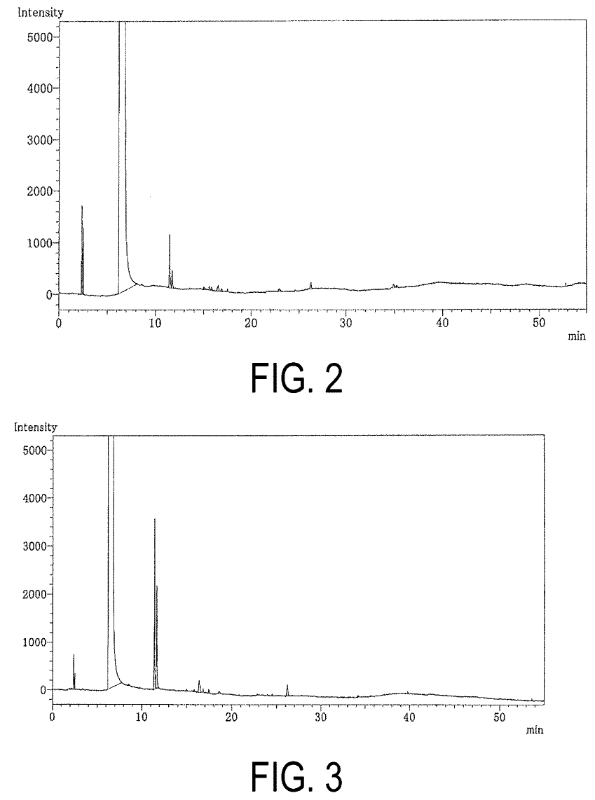

A chromatogram of the gas chromatographic analysis of the 1,3-butylene glycol product in Example 10 primarily related to the first aspect of the present disclosure is illustrated in FIG. 2. In addition, a chromatogram of the gas chromatographic analysis of the 1,3-butylene glycol product in Comparative Example 2 primarily related to the first aspect of the present disclosure is illustrated in FIG. 3.

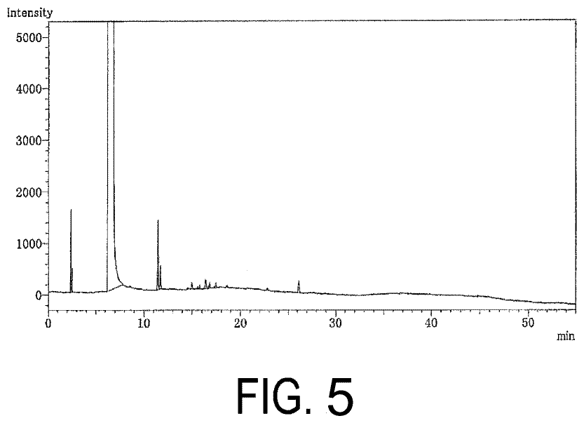

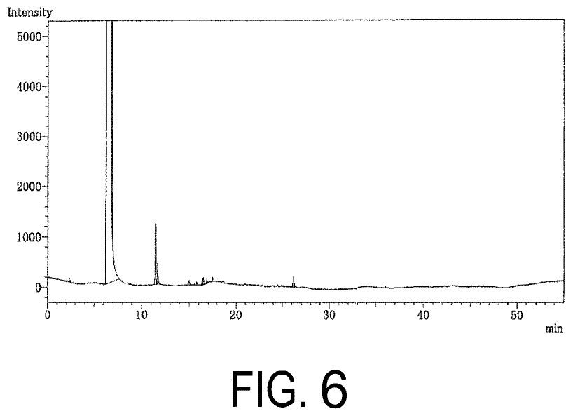

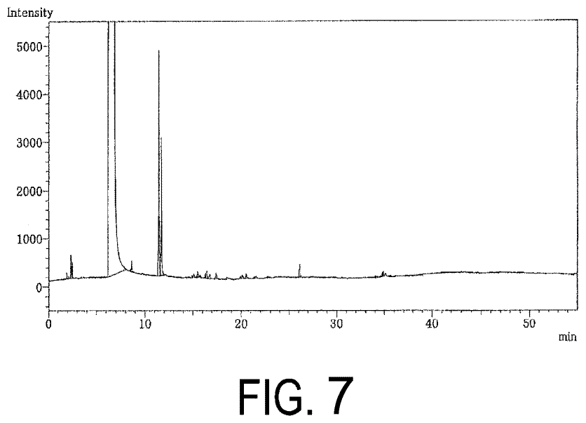

A gas chromatographic analysis of the target 1,3-butylene glycol product was performed under the conditions below. A chromatogram of the gas chromatographic analysis of the 1,3-butylene glycol product in Example 1 primarily related to the seventh aspect of the present disclosure is shown in FIG. 5. A gas chromatographic analysis of the target 1,3-butylene glycol product was performed under the conditions below. A chromatogram of the gas chromatographic analysis of the 1,3-butylene glycol product in Example 12 primarily related to the seventh aspect of the present disclosure is shown in FIG. 6. In addition, a chromatogram of the gas chromatographic analysis of the 1,3-butylene glycol product in Comparative Example 2 primarily related to the seventh aspect of the present disclosure is shown in FIG. 7.

### Conditions for the Gas Chromatographic Analysis are as Follows:

Analytical Instrument: Shimadzu GC 2010
Analytical Column: column with dimethylpolysiloxane as a stationary phase having a film thickness of 1.0 μm, a length of 30 m, and an inner diameter of 0.25 mm (Agilent J&W GC column—DB-1 available from Agilent Technologies Japan, Ltd.)
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction and Temperature: split sample introduction, 250 °C
Gas Flow Rate of Split and Carrier Gas: 23 mL/min, helium
Column Gas Flow Rate and Carrier Gas: 1 mL/min, helium
Detector and Temperature: a flame ionization detector (FID), 280 °C.

Injection Sample: 0.2 μL of a 80 wt. % 1,3-butylene glycol product aqueous solution

### [Measurement of an Initial Boiling Point and Measurement of Dry Point]

The test was conducted according to the test method specified in the normal pressure distillation test method of JIS K2254 “Petroleum products—distillation test method”.

### [Measurement of Water Content]

Water content was measured by a Karl Fischer water content measurement instrument.

### [GC-MS Analysis]

Analytical Instrument: Agilent 6890A-GC/5973A-MSD
Analytical Column: a column with dimethylpolysiloxane as a stationary phase, having a length of 30 m, an inner diameter of 0.25 mm, and a film thickness of 1.0 μm
Heating Conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Ion source temperature: EI 230 °C, CI 250 °C
Q Pole temperature: 150 °C
Sample: subjected to analysis as it was

### [Potassium Permanganate Test]

In the present specification, the potassium permanganate test value (PMT) is a value measured in accordance with the visual colorimetric procedure of JIS K1351 (1993).

### Color Aging Test 1

The target 1,3-butylene glycol product was placed in a wide-mouth bottle, the bottle was stoppered tightly and kept in a constant temperature oven set at 180 °C for 3 hours. A Hazen color number (APHA) of the 1,3-butylene glycol product after the 1,3-butylene glycol product had been kept at 180 °C for 3 hours was measured using a color difference meter (“ZE6000” available from Nippon Denshoku Industries Co., Ltd.) and a quartz cell with an optical path length of 10 mm. The Hazen color number (APHA) of the 1,3-butylene glycol product prior to the test was similarly measured. Furthermore, each impurity content of the 1,3-butylene glycol product before and after the coloration-over-time test 1 was measured by GC-MS analysis. Also, the initial boiling point, dry point, potassium permanganate test value, odor and acid content of the 1,3-butylene glycol product before and after the coloration-over-time test 1 were measured.

### Color Aging Test 2

The target 1,3-butylene glycol product was placed in a wide-mouth bottle, and the bottle was stoppered tightly and kept in a constant temperature oven set at 100 °C for 75 days. A Hazen color number (APHA) of the 1,3-butylene glycol product after the 1,3-butylene glycol product had been kept at 100 °C for 75 days was measured using a color difference meter (“ZE6000” available from Nippon Denshoku Industries Co., Ltd.) and a quartz cell with an optical path length of 10 mm.

### Water Added Heating Test (Acid Concentration Analysis)

The target 1,3-butylene glycol product adjusted to a 90 wt. % aqueous solution and then kept at 100 °C for 1 week was used as a sample for an acid concentration analysis performed according to the technique below. In addition, the 1,3-butylene glycol product prior to the test was also subjected to acid concentration analysis by the following technique.

### Acid Concentration Analysis

The sample was measured using an automatic potentiometric titrator (AT-510 available from Kyoto Electronics Manufacturing Co., Ltd.). First, 50 g of the sample was diluted with 50 g of distilled water and titrated with 0.01 N aqueous sodium hydroxide solution from a burette with stirring until the titration was automatically stopped at the endpoint. Then, the acid concentration (acid content) in terms of acetic acid was calculated based on the following equation.

Acid concentration (wt. %)=titration volume (mL)×F×A×(100/sample amount (g))
F: 1.0 (factor of the 0.01 N aqueous sodium hydroxide solution)
A: 0.0006 (grams of acetic acid corresponding to 1 mL of the aqueous sodium hydroxide solution)

### Odor Test (by Person)

The target 1,3-butylene glycol product (100 mL) was placed in a wide-mouth reagent bottle (internal volume: 100 mL), and the bottle was stoppered tightly and allowed to stand at room temperature for a while (about 120 minutes). Then, the stopper was opened, the content was transferred to a 300-mL wide-mouth beaker, and 100 mL of pure water was added into the beaker to make a total of 200 mL. The wide-mouth beaker was shaken manually to stir the content, and the odor was then immediately smelled and scored according to the following evaluation. The same odor test was performed also on the sample after the coloration-over-time test 1, and the sample after the water added heating test.
1: Odor is not sensed
2: Slightly odorous
3: Odor is clearly sensed

### Odor Test (Using Odor Sensor)

The target 1,3-butylene glycol product was measured for the fluorescence intensity change rate (fluorescent brightness change amount) correlated with odor described in WO 2019/035476. That is, the fluorescent brightness change amount was measured using an odor measurement device (odor sensor, the flavor fermentation manufactured by Komi Hakko Corp.), and the fluorescent brightness amount of the obtained Comparative Example 1 was evaluated as odor rank 4, and odor was evaluated as the following six ranks (0, 1, 2, 3, 4, or 5). The same odor test was performed also on the sample after the coloration-over-time test 1.

| Fluorescent brightness change amount | odor rank |
|---|---|
| less than 3.4 | 0 |
| 3.4 or greater and less than 3.5 | 1 |
| 3.5 or greater and less than 3.7 | 2 |
| 3.7 or greater and less than 3.9 | 3 |
| 3.9 or greater and less than 4.1 | 4 |
| 4.1 or greater | 5 |

The coloration-over-time tests 1 and 2 and the water added heating test are accelerated tests assuming long-term storage of 1,3 BG products.

### Considerations of Results

The results of the above comparative examples, reference examples and examples are shown in Table 1 to 5.

Table 1 and Table 2 primarily relate to the first aspect of the present disclosure.

Table 3, 4 and 5 primarily relate to the seventh aspect of the present disclosure.

TABLE 1

| | | Comparative | Comparative | Reference | Example | Example | Example | Example | Example |
|---|---|---|---|---|---|---|---|---|---|
| | | Example 1 | Example 2 | Example 1 | 1 | 2 | 3 | 4 | 5 |
| Charge | Part | 100 | ← | ← | ← | ← | ← | ← | ← |
| into high | 1,3 BG GC | 95 | ← | ← | ← | ← | ← | ← | ← |
| boiling | area % | | | | | | | | |
| point | High boiling | 3 | ← | ← | ← | ← | ← | ← | ← |
| component | product GC | | | | | | | | |
| removal | area % | | | | | | | | |
| column C | | | | | | | | | |
| High | Reflux ratio* | 0.01 | 0.02 | 0.02 | 0.05 | 0.05 | 0.2 | 0.6 | 0.6 |
| boiling | Distilled amount | 80 | 80 | 70 | 80 | 80 | 80 | 80 | 90 |
| point | part | | | | | | | | |
| component | Amount of | 20 | 20 | 30 | 20 | 15 | 20 | 20 | 10 |
| removal | bottom part** | | | | | | | | |
| column C | Recycled | Absent | Absent | Absent | Absent | Absent | Absent | Absent | Absent |
| | amount of | | | | | | | | |
| | bottom part | | | | | | | | |
| | 1,3 BG recovery | 83 | 83 | 73 | 83 | 88 | 84 | 84 | 94 |
| | ratio % | | | | | | | | |
| Bottom | Part | 80 | 80 | 80 | 80 | 80 | 80 | 80 | 80 |
| (product) | 1,3 BG GC | 98.3 | 98.6 | 99 | 99.1 | 99 | 99.5 | 99.7 | 99.5 |
| from | area % | | | | | | | | |
| product | High boiling | 1.4 | 1.2 | 0.7 | 0.6 | 0.7 | 0.2 | 0.05 | 0.2 |
| column F | product GC | | | | | | | | |
| | area % | | | | | | | | |
| | Dry point ° C. | 210 | 209.8 | 209 | 208.9 | 209 | 208.7 | 208.6 | 208.7 |

*Reflux ratio = high boiling point component removal column reflux amount/high boiling point component removal column distilled amount
**The numerical values put in parentheses in Examples 7 to 9 are discharge amounts to the outside of the system.

TABLE 2

| | | Example | Example | Example | Example | Example | Example | Example | Example |
|---|---|---|---|---|---|---|---|---|---|
| | | 6 | 7 | 8 | 9 | 10 | 11 | 12 | 13 |
| Charge into | Part | 100 | ← | ← | ← | 100 | ← | ← | 100 |
| high boiling | 1,3 BG GC area % | 95 | ← | ← | ← | 97 | ← | ← | 99 |
| point | High boiling product | 3 | ← | ← | ← | 2 | ← | ← | 0.5 |
| component | GC area % | | | | | | | | |
| removal | | | | | | | | | |
| column C | | | | | | | | | |
| High | Reflux ratio* | 1 | 1 | 10 | 20 | 0.02 | 0.05 | 1 | 0.02 |
| boiling | Distilled amount part | 90 | 90 | 90 | 90 | 95 | 95 | 95 | 95 |
| point | Amount of bottom | 10 | 10(5) | 10(5) | 10(5) | 5 | 5 | 5 | 5 |
| component | part** | | | | | | | | |
| removal | Recycled amount of | Absent | 5 | 5 | 5 | Absent | Absent | Absent | Absent |
| column C | bottom part | | | | | | | | |
| | 1,3 BG recovery | 94 | 99 or | 99 or | 99 or | 97 | 97 | 97 | 95 |
| | ratio % | | greater | greater | greater | | | | |
| Bottom | Part | 80 | 80 | 80 | 80 | 80 | 80 | 80 | 80 |
| (product) | 1,3 BG GC area % | 99.6 | 99.5 | 99.6 | 99.6 | 99.2 | 99.4 | 99.7 | 99.7 |
| from | High boiling product | 0.1 | 0.2 | 0.1 | 0.08 | 0.5 | 0.3 | 0.03 | 0.2 |
| product | GC area % | | | | | | | | |
| column F | Dry point ° C. | 208.6 | 208.7 | 208.7 | 208.6 | 209 | 208.7 | 208.4 | 208.6 |

*Reflux ratio = high boiling point component removal column reflux amount/high boiling point component removal column distilled amount
**The numerical values put in parentheses in Examples 7 to 9 are discharge amounts to the outside of the system.

TABLE 1

| | | Comparative | Comparative | Comparative | Comparative | Example | Example | Example |
|---|---|---|---|---|---|---|---|---|
| | | Example 1 | Example 2 | Example 3 | Example 4 | 1 | 2 | 3 |
| Charge into | Part | 100 | 100 | ← | 100 | 100 | ← | ← |
| dehydration | 1,3 BG GC area % | 56 | 66 | ← | 76 | 56 | ← | ← |
| column A | Water wt. % | 40 | 30 | ← | 20 | 40 | ← | ← |
| | AD ppm | 130 | 145 | ← | 155 | 130 | ← | ← |
| | CR ppm | 89 | 110 | ← | 117 | 89 | ← | ← |
| | Low boiling substance | 3 | 3 | ← | 3 | 3 | ← | ← |
| | GC area % | | | | | | | |
| | High boiling substance | 1 | 1 | ← | 1 | 1 | ← | ← |
| | GC area % | | | | | | | |
| Reflux ratio of | | 0.5 | 0.5 | ← | 1 | 1 | 50 | 50 |
| dehydration | | | | | | | | |
| columu A | | | | | | | | |
| Distillate from | Part | 42 | 32 | ← | 23 | 43 | 43 | 43 |
| dehydration | | | | | | | | |
| column A | | | | | | | | |
| 1,3 BG | % | 99.3 | 99.8 | ← | 99.8 | 99.6 | 99.9 or | 99.6 |
| recovery ratio | | | | | | | greater | |
| Charge into | Part | 100 | ← | ← | ← | ← | ← | ← |
| high boiling | 1,3 BG GC area % | 96.6 | 97.4 | ← | 97.9 | 97.4 | 97.9 | ← |
| point | Water wt.% | 1.2 | 1.1 | ← | 0.7 | 0.9 | 0.2 | ← |
| component | AD ppm | 63 | 48 | ← | 39 | 20 | 9 | 9 |
| removal | CR ppm | 45 | 37 | ← | 23 | 19 | 12 | 9 |
| column C | Low boding substance | 1.6 | 1.1 | ← | 0.8 | 0.8 | 0.3 | ← |
| | GC area % | | | | | | | |
| | High boiling substance | 1.8 | 1.5 | ← | 1.3 | 1.8 | 1.8 | ← |
| | GC area % | | | | | | | |
| High boiling | Reflux ratio | 0.02 | 0.02 | 0.02 | 0.02 | 0.05 | 0.05 | 0.05 |
| point | Distillation extraction | 80 | 80 | 80 | 80 | 80 | 80 | 80 |
| component | part | | | | | | | |
| removal | Bottom extraction part | 2020 | | 20 | 20 | 20 | 20 | 20 |
| column C | Recycle | Absent | Absent | Absent | Absent | Absent | Absent | Absent |
| | 1,3 BG recovery | 81 | 81 | 81 | 81 | 82 | 81 | 81 |
| | ratio % | | | | | | | |
| Charge into | Part | 100 | 100 | ← | 100 | 100 | ← | 100 |
| product | 1,3 BG GC area % | 97.8 | 98.3 | ← | 98.6 | 99.0 | 99.1 | 99.2 |
| column F | Water wt.% | 1.3 | 1.2 | ← | 0.8 | 1 | 0.3 | 0.3 |
| | AD ppm | 45 | 39 | ← | 38 | 20 | 9 | 9 |
| | CR ppm | 20 | 12 | ← | 139 | | 6 | 6 |
| | Low boiling substance | 1.0 | 0.6 | ← | 0.6 | 0.4 | 0.3 | 0.3 |
| | GC area % | | | | | | | |
| | High boiling substance | 1.2 | 1.1 | ← | 0.8 | 0.6 | 0.6 | 0.5 |
| | GC area % | | | | | | | |
| Reflux ratio of | | 0.05 | 0.05 | 0.05 | 0.1 | 0.5 | 0.5 | 10 |
| product | | | | | | | | |
| column F | | | | | | | | |
| Distillation | Part | 20 | 20 | 30 | 20 | 10 | 10 | 1 |
| column F | | | | | | | | |
| from product | Recycle | Absent | Absent | Absent | Absent | Absent | Absent | Absent |
| Bottom | Part | 80 | 80 | 70 | 80 | 90 | 90 | 99 |
| (product) from | 1,3 BG GC area % | 98.3 | 98.5 | 98.4 | 98.8 | 99.2 | 99.3 | 99.5 |
| product | Water wt. % | 0.6 | 0.4 | 0.2 | 0.2 | 0.2 | 0.1 | 0.1 |
| column F | AD ppm | 5 | 4 | 2 | 2 | 1.5 | 0.7 | 1 |
| | CR ppm | 4 | 2 | 1.3 | 1.3 | 0.9 | 0.7 | 0.7 |
| | Low boiling substance | 0.2 | 0.1 | 0.1 | 0.1 | 0.08 | 0.05 | 0.05 |
| | GC area % | | | | | | | |
| | High boiling substance | 1.5 | 1.4 | 1.5 | 1.1 | 0.7 | 0.7 | 0.5 |
| | GC area % | | | | | | | |
| | Initial boiling point ° C. | 193.2 | 199.0 | 203.0 | 203.1 | 203.3 | 206.7 | 206.7 |
| | Dry point ° C. | 210.3 | 210.1 | 210.2 | 209.5 | 209 | 208.9 | 208.8 |
| | PMT min | 0 | 5 | 30 | 30 | 35 | 45 | 40 |
| | 1,3 BG recovery | 81 | 80 | 70 | 80 | 90 | 90 | 99 or |
| | ratio % | | | | | | | greater |
| | | Example | Example | Example | Example | Example | Example | Example |
| | | 4 | 5 | 6 | 7 | 8 | 9 | 10 |
| Charge into | Part | 100 | ← | ← | ← | ← | ← | ← |
| dehydration | 1,3 BG GC area % | 66 | ← | ← | ← | ← | ← | ← |
| column A | Water wt. % | 30 | ← | ← | ← | ← | ← | ← |
| | AD ppm | 145 | ← | ← | ← | ← | ← | ← |
| | CR ppm | 110 | ← | ← | ← | ← | ← | ← |
| | Low boiling substance | 3 | ← | ← | ← | ← | ← | ← |
| | GC area % | | | | | | | |
| | High boiling substance | 1 | ← | ← | ← | ← | ← | ← |
| | GC area % | | | | | | | |
| Reflux ratio of | | 2 | 10 | 20 | 20 | 20 | 50 | 100 |
| dehydration | | | | | | | | |
| columu A | | | | | | | | |
| Distillate from | Part | 33 | 33 | 33 | 33 | 33 | 33 | 33 |
| dehydration | | | | | | | | |
| column A | | | | | | | | |
| 1,3 BG | % | 99.9 | 99.9 or | 99.9 or | 99.9 or | 99.9 or | 99.9 or | 99.9 or |
| recovery ratio | | | greater | greater | greater | greater | greater | greater |
| Charge into | Part | ← | ← | ← | ← | ← | ← | ← |
| high boiling | 1,3 BG GC area % | 98.2 | 98.3 | 98.3 | 98.3 | 98.3 | 98.3 | 98.4 |
| point | Water wt. % | 0.5 | 0.1 | 0.08 | 0.08 | 0.08 | 0.03 | 0.02 |
| component | AD ppm | 38 | 18 | 13 | 13 | 13 | 5 | 4 |
| removal | CR ppm | 30 | 12 | 11 | 11 | 11 | 5 | 3 |
| column C | Low boding substance | 0.3 | 0.2 | 0.2 | 0.2 | 0.2 | 0.2 | 0.1 |
| | GC area % | | | | | | | |
| | High boiling substance | 1.5 | 1.5 | 1.5 | 1.5 | 1.5 | 1.5 | 1.5 |
| | GC area % | | | | | | | |
| High boiling | Reflux ratio | 0.05 | 0.05 | 0.05 | 0.05 | 0.05 | 0.05 | 0.05 |
| point | Distillation extraction | 80 | 80 | 80 | 80 | 80 | 80 | 80 |
| component | part | | | | | | | |
| removal | Bottom extraction part | 20 | 20 | 20 | 20 | 20 | 20 | 20 |
| column C | Recycle | Absent | Absent | Absent | Absent | Absent | Absent | Absent |
| | 1,3 BG recovery | 81 | 81 | 81 | 81 | 81 | 81 | 81 |
| | ratio % | | | | | | | |
| Charge into | Part | 100 | ← | ← | ← | ← | ← | ← |
| product | 1,3 BG GC area % | 99.2 | 99.3 | 99.3 | 99.3 | 99.3 | 99.3 | 99.4 |
| column F | Water wt.% | 0.6 | 0.3 | 0.2 | 0.2 | 0.2 | 0.1 | 0.08 |
| | AD ppm | 29 | 16 | 10 | 11 | 9 | 3 | 2 |
| | CR ppm | 13 | 5 | 3 | 3 | 3 | 2 | 1 |
| | Low boiling substance | 0.3 | 0.2 | 0.2 | 0.2 | 0.2 | 0.2 | 0.1 |
| | GC area % | | | | | | | |
| | High boiling substance | 0.5 | 0.5 | 0.5 | 0.5 | 0.5 | 0.5 | 0.5 |
| | GC area % | | | | | | | |
| Reflux ratio of | | 0.5 | 0.5 | 2 | 100 | 500 | 2 | 2 |
| product | | | | | | | | |
| column F | | | | | | | | |
| Distillation | Part | 10 | 10 | 1 | 1 | 1 | 1 | 1 |
| column F | | | | | | | | |
| from product | Recycle | Present | Present | Present | Present | Present | Present | Present |
| Bottom | Part | 90 | 90 | 99 | 99 | 99 | 99 | 99 |
| (product) from | 1,3 BG GC area % | 99.4 | 99.4 | 99.4 | 99.5 | 99.5 | 99.4 | 99.5 |
| product | Water wt. % | 0.2 | 0.1 | 0.1 | 0.003 | 0.002 | 0.05 | 0.04 |
| column F | AD ppm | 2 | 1.1 | 1 | 0.2 | less than | 0.3 | less than |
| | | | | | | 0.2 | | 0.2 |
| | CR ppm | 1 | 0.7 | 0.6 | 0.1 | less than | 0.2 | less than |
| | | | | | | 0.1 | | 0.1 |
| | Low boiling substance | 0.04 | 0.03 | 0.07 | 0.004 | 0.003 | 0.06 | 0.03 |
| | GC area % | | | | | | | |
| | High boiling substance | 0.6 | 0.6 | 0.5 | 0.5 | 0.5 | 0.5 | 0.5 |
| | GC area % | | | | | | | |
| | Initial boiling point ° C. | 203.8 | 206.7 | 206.8 | 208.4 | 208.4 | 207.5 | 207.2 |
| | Dry point ° C. | 208.9 | 208 9 | 208.8 | 208.8 | 208.8 | 208.8 | 208.8 |
| | PMT min | 30 | 40 | 40 | 55 | 60 | 50 | 55 |
| | 1,3 BG recovery | 99 or | 99 or | 99 or | 99 or | 99 or | 99 or | 99 or |
| | ratio % | greater | greater | greater | greater | greater | greater | greater |

TABLE 2

| | | Example | Example | Example | Example | Example | Example | Example | Example | Example |
|---|---|---|---|---|---|---|---|---|---|---|
| | | 11 | 12 | 13 | 14 | 15 | 16 | 17 | 18 | 19 |
| Charge into | Part | 100 | ← | ← | 100 | ← | ← | ← | ← | ← |
| dehydration | 1,3 BG GC | 76 | ← | ← | 66 | ← | ← | ← | ← | ← |
| column A | area % | | | | | | | | | |
| | Water wt. % | 20 | ← | ← | 30 | ← | ← | ← | ← | ← |
| | AD ppm | 155 | ← | ← | 145 | ← | ← | ← | ← | ← |
| | CR ppm | 117 | ← | ← | 110 | ← | ← | ← | ← | ← |
| | Low boiling | 3 | ← | ← | 3 | ← | ← | ← | ← | ← |
| | substance | | | | | | | | | |
| | GC area % | | | | | | | | | |
| | High boiling | 1 | ← | ← | ← | ← | 12 | ← | ← | ← |
| | substance | | | | | | | | | |
| | GC area % | | | | | | | | | |
| Reflux ratio of | | 2 | 10 | 100 | 10 | 10 | 10 | to | to | 10 |
| dehydration | | | | | | | | | | |
| column A | | | | | | | | | | |
| Distillate from | Part | 23 | 23 | 23 | 33 | 33 | 33 | 33 | 33 | 23 |
| dehydration | | | | | | | | | | |
| column A | | | | | | | | | | |
| 1,3 BG | % | 99.8 | 99.9 or | 99.9 or | 99.8 | 99.8 | 99.8 | 99.8 | 99.8 | 99.8 |
| recovery ratio | | | greater | greater | | | | | | |
| Charge into | Part | 100 | ← | ← | ← | ← | ← | ← | ← | ← |
| high boiling | 1,3 BG GC | 98.4 | 98.5 | 98.6 | 98.3 | ← | 98.1 | 98.3 | ← | ← |
| point | area % | | | | | | | | | |
| component | Water wt. % | 0.1 | 0.08 | 0.01 | 0.1 | ← | 0.1 | 0.1 | ← | ← |
| removal | AD ppm | 29 | 15 | 2 | 18 | ← | 18 | 18 | ← | ← |
| column C | CR ppm | 21 | 8 | 1 | 12 | | 12 | 12 | ← | ← |
| | Low boiling | 0.3 | 0.2 | 0.1 | 0.2 | ← | 0.2 | 0.2 | ← | ← |
| | substance | | | | | | | | | |
| | GC area % | | | | | | | | | |
| | High boiling | 1.3 | 1.3 | 1.3 | 1.5 | | 1.7 | 1.5 | ← | ← |
| | substance | | | | | | | | | |
| | GC area % | | | | | | | | | |
| High boiling | Reflux ratio | 0.05 | 0.05 | 0.05 | 0.1 | 0.5 | 0.5 | 0.5 | 1 | 2 |
| point | Distillation | 80 | 80 | 80 | 90 | 90 | 90 | 95 | 95 | 95 |
| component | extraction part | | | | | | | | | |
| removal | Bottom | 20 | 20 | 20 | 10 | 10 | 10 | 5 | 5 | 5 |
| column C | extraction part | | | | | | | | | |
| | Recycle | Absent | Absent | Absent | Absent | Absent | Present | Absent | Absent | Absent |
| | 1,3 BG recovery | 81 | 81 | 81 | 91 | 91 | 99.9 or | 96 | 96 | 96 |
| | ratio % | | | | | | greater | | | |
| Charge into | Part | 100 | ← | ← | 100 | 100 | 100 | 100 | 100 | 100 |
| product | 1,3 BG GC | 99.3 | 99.4 | 99.5 | 99.4 | 99.6 | 99.5 | 99.5 | 99.7 | 99.7 |
| column F | area % | | | | | | | | | |
| | Water wt. % | 0.3 | 0.1 | 0.09 | 0.3 | 0.3 | 0.3 | 0.3 | 0.3 | 0.3 |
| | AD ppm | 22 | 12 | 1 | 10 | 9 | 10 | 10 | 9 | 8 |
| | CR ppm | 14 | 5 | 0.7 | 5 | 6 | 6 | 5 | 5 | 5 |
| | Low boiling | 0.3 | 0.2 | 0.1 | 0.2 | 0.2 | 0.2 | 0.2 | 0.2 | 0.2 |
| | substance | | | | | | | | | |
| | GC area % | | | | | | | | | |
| | High boiling | 0.4 | 0.4 | 0.4 | 0.4 | 0.3 | 0.3 | 0.3 | 0.1 | 0.09 |
| | substance | | | | | | | | | |
| | GC area % | | | | | | | | | |
| Reflux ratio of | | 1 | 2 | 2 | 1 | 1 | 1 | 1 | 1 | 1 |
| product | | | | | | | | | | |
| column F | | | | | | | | | | |
| Distillate from | Part | 10 | 1 | 1 | 10 | 10 | 10 | 10 | 10 | 10 |
| product | Recycle | Present | Present | Present | Present | Present | Present | Present | Present | Present |
| column F | | | | | | | | | | |
| Bottom | Part | 90 | 99 | 99 | 90 | 90 | 90 | 90 | 90 | 90 |
| (product) from | 1,3 BG GC | 99.4 | 99.4 | 99.5 | 99.5 | 99.6 | 99.6 | 99.6 | 99.9 | 99.9 |
| product | area % | | | | | | | | | |
| column F | Water wt. % | 0.06 | 0.05 | 0.04 | 0.05 | 0.05 | 0.05 | 0.05 | 0.05 | 0.05 |
| | AD ppm | 1.6 | 1 | less than | 0.9 | 0.8 | 0.8 | 0.9 | 0.8 | 0.9 |
| | | | | 0.2 | | | | | | |
| | CR ppm | 1 | 0.8 | less than | 0.7 | 0.6 | 0.6 | 0.5 | 0.5 | 0.4 |
| | | | | 0.1 | | | | | | |
| | Low boiling | 0.09 | 0.07 | 0.05 | 0.02 | 0.02 | 0.02 | 0.02 | 0.02 | 0.02 |
| | substance | | | | | | | | | |
| | GC area % | | | | | | | | | |
| | High boiling | 0.5 | 0.5 | 0.5 | 0.5 | 0.2 | 0.4 | 0.4 | 0.1 | 0.09 |
| | substance | | | | | | | | | |
| | GC area % | | | | | | | | | |
| | Initial boiling | 207.3 | 207.5 | 207.2 | 207.5 | 207.5 | 207.5 | 207.5 | 207.5 | 207.5 |
| | point ° C. | | | | | | | | | |
| | Dry point ° C. | 208.9 | 208.9 | 208.9 | 208.8 | 208.6 | 208.8 | 208.8 | 208.6 | 208.5 |
| | PMT min | 35 | 40 | 60 | 45 | 45 | 45 | 45 | 45 | 45 |
| | 1,3 BG recovery | 90 | 99 or | 99 or | 99.9 or | 99.9 or | 99.9 or | 99.9 or | 99.9 or | 99.9 or |
| | ratio % | | greater | greater | greater | greater | greater | greater | greater | greater |
| | | | Example | Example | Example | Example | Example | Example | Example | Example |
| | | | 20 | 21 | 22 | 23 | 24 | 25 | 26 | 27 |
| | Charge into | Part | ← | ← | 100 | 100 | 100 | 100 | 100 | 100 |
| | dehydration | 1,3 BG GC | ← | ← | 56 | 76 | 9 | 66 | 67 | 69 |
| | column A | area % | | | | | | | | |
| | | Water wt. % | ← | ← | 40 | 20 | 90 | 30 | 30 | 30 |
| | | AD ppm | ← | ← | 130 | 155 | 85 | 970 | 145 | 3 |
| | | CR ppm | ← | ← | 89 | 117 | 71 | 390 | 110 | 1 |
| | | Low boiling | ← | ← | 3 | 3 | 0.4 | 3 | 3 | 1 |
| | | substance | | | | | | | | |
| | | GC area % | | | | | | | | |
| | | High boiling | ← | ← | 1 | 1 | 0.1 | 1 | 1 | 0.5 |
| | | substance | | | | | | | | |
| | | GC area % | | | | | | | | |
| | Reflux ratio of | | 10 | 10 | 0.5 | 100 | 20 | 2 | 0.3 | 10 |
| | dehydration | | | | | | | | | |
| | column A | | | | | | | | | |
| | Distillate from | Part | 33 | 33 | 42 | 23 | 91 | 33 | 32 | 32 |
| | dehydration | | | | | | | | | |
| | column A | | | | | | | | | |
| | 1,3 BG | % | 99.8 | 99.8 | 99.3 | 99.9 or | 99.0 | 99.9 or | 99.8 | 99.9 or |
| | recovery ratio | | | | | greater | | greater | | greater |
| | Charge into | Part | ← | ← | 100 | | 100 | 100 | 100 | 4 |
| | high boiling | 1,3 BG GC | ← | ← | 96.6 | 98.6 | 97.5 | 98.2 | 96.7 | 99.1 |
| | point | area % | | | | | | | | |
| | component | Water wt. % | ← | ← | 1.2 | 0.01 | 3 | 0.5 | 1.3 | 0.1 |
| | removal | AD ppm | ← | ← | 63 | 2 | 9 | 22.1 | 61 | 0.3 |
| | column C | CRppm | ← | ← | 45 | 1 | 4 | 128 | 48 | 0.2 |
| | | Low boiling | ← | ← | 1.6 | 0.1 | 1.1 | 0.3 | 1.8 | 0.1 |
| | | substance | | | | | | | | |
| | | GC area % | | | | | | | | |
| | | High boiling | ← | ← | 1.8 | 1.3 | 1.4 | 1.5 | 1.5 | 0.8 |
| | | substance | | | | | | | | |
| | | GC area % | | | | | | | | |
| | High boiling | Reflux ratio | 5 | 20 | 0.1 | 1 | 1 | 0.05 | 0.05 | 1 |
| | point | Distillation | 95 | 95 | 90 | 90 | 90 | 80 | 80 | 95 |
| | component | extraction part | | | | | | | | |
| | removal | Bottom | 5 | 5 | 10 | 10 | 10 | 20 | 20 | 5 |
| | column C | extraction part | | | | | | | | |
| | | Recycle | Absent | Absent | Absent | Absent | Absent | Absent | Absent | Absent |
| | | 1,3 BG recovery | 96 | 96 | 92 | 91 | 91 | 81 | 81 | 96 |
| | | ratio % | | | | | | | | |
| | Charge into | Part | too | 100 | 100 | — | 100 | 100 | 100 | too |
| | product | 1,3 BG GC | 99.7 | 99.7 | 98.7 | — | 98.7 | 99.2 | 97.6 | 99.7 |
| | column F | area % | | | | | | | | |
| | | Water wt. % | 0.3 | 0.3 | 1.2 | — | 3 | 0.7 | 1.4 | 0.3 |
| | | AD ppm | 8 | 9 | 43 | — | 7 | 205 | 54 | 0.3 |
| | | CR ppm | 4 | 5 | 17 | — | 3 | 110 | 38 | 0.2 |
| | | Low boiling | 0.2 | 0.2 | 0.9 | — | 0.8 | 0.3 | 1.8 | 0.1 |
| | | substance | | | | | | | | |
| | | GC area % | | | | | | | | |
| | | High boiling | 0.07 | 0.06 | 0.4 | — | 0.5 | 0.5 | 0.6 | 0.2 |
| | | substance | | | | | | | | |
| | | GC area % | | | | | | | | |
| | Reflux ratio of | | 1 | 1 | 5 | — | 10 | 10 | 20 | 1 |
| | product | | | | | | | | | |
| | column F | | | | | | | | | |
| | Distillate from | Part | 10 | 10 | 10 | — | 10 | 10 | 10 | 10 |
| | product | Recycle | Present | Present | Present | — | Present | Present | Present | Present |
| | column F | | | | | | | | | |
| | Bottom | Part | 90 | 90 | 96 | — | 90 | 96 | 90 | 90 |
| | (product) from | 1,3 BG GC | 99.9 | 99.9 | 99.4 | 99.5 | 99.4 | 99.4 | 99.3 | 99.8 |
| | product | area % | | | | | | | | |
| | column F | Water wt. % | 0.05 | 0.05 | 0.1 | 0.09 | 0.1 | 6.62 | 0.01 | 6.65 |
| | | AD ppm | 0.9 | 0.8 | 1.1 | 1.5 | 0.3 | 1.2 | 0.5 | less than |
| | | | | | | | | | | 0.2 |
| | | CR ppm | 0.4 | 0.3 | 0.4 | 1 | 0.2 | 1.1 | 0.4 | less than |
| | | | | | | | | | | 6.1 |
| | | Low boiling | 0.02 | 0.02 | 0.1 | 0.1 | 0.02 | 0.004 | 0.03 | 6.01 |
| | | substance | | | | | | | | |
| | | GC area % | | | | | | | | |
| | | High boiling | 0.08 | 0.07 | 0.5 | 0.4 | 0.6 | 0.6 | 0.7 | 0.2 |
| | | substance | | | | | | | | |
| | | GC area % | | | | | | | | |
| | | Initial boiling | 207.6 | 207.6 | 206.8 | 206.9 | 206.7 | 208.0 | 207.8 | 207.6 |
| | | point ° C. | | | | | | | | |
| | | Dry point ° C. | 208.5 | 208.4 | 208.8 | 208.8 | 208.9 | 208.9 | 208.9 | 208.6 |
| | | PMT min | 45 | 45 | 45 | 35 | 50 | 35 | 45 | 65 |
| | | 1,3 BG recovery | 99.9 or | 99.9 or | 99.9 or | — | 99.9 or | 99 or | 99 or | 99.9 or |
| | | ratio % | greater | greater | greater | | greater | greater | greater | greater |

TABLE 3

| | Compar- | Compar- | | | |
|---|---|---|---|---|---|
| | ative | ative | | | |
| | Exam- | Exam- | Exam- | Exam- | Exam- |
| | ple 1 | ple 2 | ple 1 | ple 5 | ple 7 |
| 1,3 BG GC area % | 98.3 | 98.5 | 99.2 | 99.4 | 99 5 |
| Water wt. % | 0.6 | 0.4 | 0.2 | 0.1 | 0.003 |
| AD ppm | 5 | 4 | 1.5 | 1.1 | 0.2 |
| CR ppm | 4 | 2 | 0.9 | 0.7 | 0.1 |
| Low boiling substance GC area % | 0.2 | 0.1 | 0.08 | 0.03 | 0.004 |
| High boiling substance GC area % | 15 | 1.4 | 0.7 | 0.6 | 0.5 |
| Initial boiling point ° C. | 193.2 | 199 | 203.3 | 206.7 | 208.4 |
| Dry point ° C. | 210.3 | 210.1 | 209 | 208.9 | 208.9 |
| PMT min | 0 | 5 | 35 | 40 | 55 |
| Methyl vinyl ketone ppm | 11 | 10 | 6 | 2 | less than 0.2 |
| Acetone ppm | 10 | 9 | 5 | 2 | less than 0.2 |
| Butylaldehyde ppm | 9 | 8 | 5 | 2 | less than 0.2 |
| Acetaldol ppm | 10 | 10 | 6 | 2 | less than 0.2 |
| Compound ppm of Formula (1) | 11 | 10 | 6 | 1.4 | less than 0.2 |
| Compound ppm of Formula (2) | 9 | 8 | 5 | 1.1 | 0.5 |
| Compound ppm of Formula (3) | 9 | 8 | 4 | 1 | 0.4 |
| Compounds ppm of Formula (4) + | 8 | 8 | 4 | 0.9 | 0.3 |
| Formula (5) | | | | | |
| APHA before coloration test | 7 | 7 | 4 | 2 | 1 |
| Odor before coloration test | 2 | 2 | 1 | 1 | l |
| Odor after coloration test 1 | 3 | 2 | 2 | 1 | 1 |
| APHA of coloration test 1 | 85 | 79 | 25 | 17 | 10 |
| APHA of coloration test 2 | 51 | 43 | 12 | 7 | 3 |
| Acid content (ppm) before water | 11 | 11 | 6 | 4 | 2 |
| added heating test | | | | | |
| Odor before water added heating test | 2 | 2 | 1 | 1 | 1 |
| Acid content (ppm) after water added | 27 | 23 | 9 | 5 | 2 |
| heating test | | | | | |
| Odor after water added heating test | 3 | 3 | 2 | 1 | 1 |
| | Exam- | Exam- | Exam- | Exam- | Exam- |
| | ple 12 | ple 18 | ple 24 | ple 26 | ple 27 |
| 1,3 BG GC area % | 99.4 | 99.9 | 99.3 | 99.3 | 99.8 |
| Water wt. % | 0.05 | 0.05 | 0.03 | 0.03 | 0.05 |
| AD ppm | 1 | 0.8 | 0.5 | 0.5 | less than 0.2 |
| CR ppm | 0.8 | 0.5 | 0.4 | 0.4 | less than 0.1 |
| Low boiling substance GC area % | 0.07 | 0.02 | 0.2 | 0.03 | 0.01 |
| High boiling substance GC area % | 0.5 | 0.1 | 0.6 | 0.7 | 0.2 |
| Initial boiling point ° C. | 207.5 | 207.5 | 206.7 | 207.8 | 207 6 |
| Dry point ° C. | 208.9 | 208.6 | 208.9 | 208.9 | 208.6 |
| PMT min | 40 | 45 | 50 | 45 | 65 |
| Methyl vinyl ketone ppm | 0.3 | 0.9 | less than 0.2 | 3 | less than 0.2 |
| Acetone ppm | 0.2 | 0.8 | less than 0.2 | 2 | less than 0.2 |
| Butylaldehyde ppm | 0.3 | 07 | less than 0.2 | 2 | less than 0 2 |
| Acetaldol ppm | less than 0.2 | 0.5 | less dian 0.2 | 2 | less than 0.2 |
| Compound ppm of Formula (1) | less than 0 2 | 0.8 | less than 0.2 | 2 | less than 0.2 |
| Compound ppm of Formula (2) | 0.4 | less than 0.2 | 0.6 | 0.3 | less than 0.2 |
| Compound ppm of Formula (3) | 0.5 | less than 0.2 | 0.7 | 0.5 | less than 0 2 |
| Compounds ppm of Formula (4) + | 0.3 | less than 0.2 | 0.4 | 0.2 | less than 0.2 |
| Formula (5) | | | | | |
| APHA before coloration test | 2 | 2 | 1 | 2 | 1 |
| Odor before coloration test | 1 | 1 | 1 | 1 | 1 |
| Odor after coloration test 1 | 1 | ] | 1 | 1 | 1 |
| APHA of coloration test 1 | 12 | 13 | 11 | 18 | 7 |
| APHA of coloration test 2 | 4 | 5 | 3 | 8 | 2 |
| Acid content (ppm) before water | 3 | 3 | 2 | 4 | 1 |
| added heating test | | | | | |
| Odor before water added heating test | 3 | 1 | 1 | 1 | 1 |
| Acid content (ppm) after water added | 3 | 3 | 2 | 4 | 1 |
| heating test | | | | | |
| Odor after water added heating test | 1 | 1 | 1 | 1 | 1 |

The following examples, comparative examples, and reference examples primarily relate to the first aspect of the present disclosure.

From Comparative Examples 1 and 2 of Table 1, in the case where the 1,3 BG purity in charged liquid into the high boiling point component removal column C was 95 GC area %, when the distilled amount was 20 parts relative to 100 parts of the charged liquid into the high boiling point component removal column C, the effect of removing the high boiling point substance was low and the product dry point was not satisfactory, even though the reflux ratio was 0.02. As illustrated in Reference Example 1 of Table 1, when the distilled amount was 30 parts with respect to 100 parts of the charged liquid in the high boiling point component removal column C, the reflux ratio was 0.02, and the dry point satisfied 209 °C or lower. However, the 1,3 BG recovery ratio in the high boiling point component removal column C was very bad, i.e., 73%. Thus, this was economically disadvantageous.

From Comparative Examples 1 and 2 of Table 1 and Examples 1, 3, and 4 of Table 1, it can be understood that, when the reflux ratio of the high boiling point component removal column was increased to 0.05 or greater, it was possible to further concentrate and remove the high boiling impurities in the high boiling point component removal column C, that the GC purity of the product was improved, and that the dry point of the product was 209 °C or lower, which was not higher than the standard value (quasi-drug raw material standard).

It can be seen that the 1,3 BG recovery ratio can be improved by reducing the bottom ratio (the bottom cut ratio) of the high boiling point component removal column C, from Examples 1 and 2 of Table 1 and Examples 4 to 6 of Table 1 or Table 2. Therefore, when the bottom ratio was reduced, the high boiling impurities that could be removed decreased, the high boiling impurities in the product increased, and thus the dry point lowered. Therefore, the product dry point needs to be adjusted to 209 °C or lower.

According to Examples 6 and 7 of Table 2, when a portion of the bottom of the high boiling point component removal column C was recycled to the acetaldehyde polymerization (acetaldol production by dimerization of acetaldehyde), the high boiling impurities were decomposed and/or removed. Therefore, the 1,3 BG recovery ratio was greatly improved, while the decrease in product purity and the increase in dry point could be suppressed to the minimum.

It can be seen that, in Examples 7 to 9 of Table 2, even when the reflux ratio of the high boiling point component removal column C was increased to a certain value or greater, the product quality was not improved any more. According to Comparative Example 2 of Table 1, Reference Example 1 of Table 1, and Examples 10 and 13 of Table 2, either the amount of the bottom extracted from the high boiling point component removal column C was increased or the 1,3 BG purity of the charged liquid into the high boiling point component removal column C was increased, and the quality of the 1,3 BG product was sufficiently improved even when the reflux ratio was somewhat small. The quality of the 1,3 BG product (purity, dry point, etc.) was improved by increasing the reflux ratio of the high boiling point component removal column C, according to Examples 10 to 12 of Table 2.

Example 13 of Table 2 was a case in which the concentration of 1,3 BG in the charged liquid in the high boiling point component removal column was greater than that in Examples 1 and 10 of Table 1 or Table 2. It can be seen that, when the 1,3 BG concentration was high and the high boiling point impurity concentration was low, a good product was produced even with a small reflux ratio of the high boiling point component removal column.

Note that, among 1,3-butylene glycol products produced by known methods, there was no high-quality product having a dry point of 209 °C or lower, a purity (GC area %) of 1,3-butylene glycol of greater than 98.5%, and a content of the high boiling point substance (GC area %) of less than 1.2%.

The following examples, comparative examples, and reference examples primarily relate to the seventh aspect of the present disclosure.

From Comparative Example 1, Comparative Example 2, Example 1, Example 5, and Example 7 of Table 3 and Table 5, it can be seen that, for acetaldehyde, crotonaldehyde, and other impurities detected by the gas chromatographic analysis (quantification limit: 10 ppm), and methyl vinyl ketone, acetone, butylaldehyde, acetaldol, the compound represented by Formula (1), the compound represented by Formula (2), the compound represented by Formula (3), the compound represented by Formula (4), and the compound represented by Formula (5) detected by the GC-MS analysis, the lower contents resulted in better long-term storage stability in terms of coloration, acid concentration and odor.

The same applies to Example 7 of Table 3 and 5, and Examples 12, and 18 of Table 4. and Table 5. From Examples 12 and 18 of Table 4 and Table 5, the reduction in contents of methyl vinyl ketone, acetone, butylaldehyde, acetaldol, and the compound represented by Formula (1) having a boiling point less than that of 1,3 BG and the reduction in contents of the compound represented by Formula (2), the compound represented by Formula (3), the compound represented by Formula (4), and the compound represented by Formula (5) have a boiling point higher than that of 1,3 BG were both effective for long-term stability. However, total reduction in contents of these impurities as well as in contents of acetaldehyde, crotonaldehyde, and other impurities detected by the gas chromatographic analysis (quantification limit: 10 ppm) is most effective for long-term stability of the quality of the 1,3 BG product, and the stability of the product quality is significantly better in Example 7 of Table 3 and Table 5. Note that there was no clear correlation between the contents of the impurities detected by the gas chromatographic analysis other than 1,3 BG (the quantification limit of around 10 ppm) and contents of impurities, which were less than 10 ppm, ranging from methyl vinyl ketone to the compound represented by Formula (5) detected by the GC-MS analysis. This is thought to result from slight variation in concentration of each component depending on the reaction conditions and the individual distillation conditions.

Example 24 of Table 4 and Table 5 was a case where the water content of the charged liquid into the dehydration column was extremely high, but shows the same tendency as described above. The relationship between the overall behavior of the impurities and the APHA and acid concentration (acid content) showed the same tendency as in Example 7 of Table 3 and Table 5.

In Example 26 of Table 4 and Table 5, it can be seen that, while the concentration of 1,3-butylene glycol in the charged liquid into the product column was reduced, the quality of the 1,3 BG product could be maintained by maintaining a relatively high reflux ratio of the product column.

It can be seen, from Examples 18 and 27 of Table 4 and Table 5, that the quality of the 1,3 BG product was further improved by increasing the hydrogen addition pressure, totally reacting the unsaturated compounds such as aldehyde and olefin and acetals in the reaction system, and adjusting the distillation conditions in each distillation column.

As a summary of the above, configurations and variations of the present disclosure are described below.

[1]A method for producing 1,3-butylene glycol to yield purified 1,3-butylene glycol from a reaction crude liquid containing 1,3-butylene glycol,
the method including: performing dehydration including removing water by distillation, removing high boiling point component including removing a high boiling point component by distillation, and performing product distillation to yield purified 1,3-butylene glycol,
wherein, in a high boiling point component removal column for use in the removing the high boiling point component, a charged liquid containing 1,3-butylene glycol is distilled under a condition that a reflux ratio is greater than 0.02 (or 0.025 or greater, 0.05 or greater, 0.1 or greater, 0.2 or greater, 0.3 or greater, 0.4 or greater, 0.5 or greater, 0.6 or greater, 0.7 or greater, 0.8 or greater, 0.9 or greater, 1 or greater, 1.2 or greater, 1.5 or greater, 2 or greater, 3 or greater, 4 or greater, 5 or greater, 10 or greater, or 20 or greater) in a case where a concentration of 1,3-butylene glycol in the charged liquid is 95% or less, or a reflux ratio is greater than 0.01 (or 0.015 or greater, 0.05 or greater, 0.1 or greater, 0.2 or greater, 0.3 or greater, 0.4 or greater, 0.5 or greater, 0.6 or greater, 0.7 or greater, 0.8 or greater, 0.9 or greater, 1 or greater, 1.2 or greater, 1.5 or greater, 2 or greater, 3 or greater, 4 or greater, 5 or greater, 10 or greater, or 20 or greater) in a case where the concentration of 1,3-butylene glycol in the charged liquid is greater than 95%, high-purity 1,3-butylene glycol is distilled off from above a charging plate, and a liquid in which a high boiling point component is concentrated is extracted from below the charging plate.

[2] The method for producing 1,3-butylene glycol according to [1], wherein an upper limit of the reflux ratio is 100 (or 50).

[3] The method for producing 1,3-butylene glycol according to [1] or [2], wherein the reaction crude liquid containing 1,3-butylene glycol is a reaction crude liquid produced by hydrogen reduction of acetaldols (or a reaction crude liquid produced by hydrolysis of 1,3-butylene oxide, a reaction crude liquid produced by selective hydrocracking of erythritol, a reaction crude liquid produced by selective hydration to butadiene, a reaction crude liquid produced by hydrogen addition to n-butanal-3-one, a reaction crude liquid produced by hydrogen addition to 1-butanol-3-one, a reaction crude liquid produced by hydrogen addition to 3-hydroxy-1-butanoic acid, a reaction crude liquid produced by hydrogen addition to $-butyrolactone, or a reaction crude liquid produced by hydrogen addition to diketene.

[4] The method for producing 1,3-butylene glycol according to [3], wherein the acetaldols used as a raw material in reduction (hydrogenation) of the acetaldols is any of acetaldol; its cyclic dimer paraldol; aldoxane as a cyclic trimer of acetaldehyde; and mixtures of these.

[5] The method for producing 1,3-butylene glycol according to [4], wherein the acetaldols is produced by an aldol condensation reaction of acetaldehyde in the presence of a basic catalyst or produced by pyrolysis of aldoxane.

[6] The method for producing 1,3-butylene glycol according to any one of [3] to [5], wherein the reaction crude liquid containing the acetaldols is neutralized with an acid and used in the production of 1,3-butylene glycol.

[7] The method for producing 1,3-butylene glycol according to any one of [3] to [6], wherein the reaction crude liquid containing the acetaldols is subjected to simple evaporation, distillation, or hydrogen addition to remove aldehydes, followed by the hydrogenation.

[8] The method for producing 1,3-butylene glycol according to any one of [3] to [7], wherein a content of the acetaldols used as the raw material in reduction (hydrogenation) of the acetaldols is 30 wt. % or greater (e.g., from 30 to 99 wt. %) (or 40 wt. % or greater (e.g., from 40 to 98 wt. %), 50 wt. % or greater (e.g., from 50 to 97 wt. %), 60 wt. % or greater (e.g., from 60 to 95 wt. %), from 65 to 90 wt. %, from 70 to 90 wt. %, or 75 to 90 wt. %).

[9] The method for producing 1,3-butylene glycol according to any one of [3] to [8], wherein the raw material for reduction (hydrogenation) of the acetaldols contains water, and a content of the water is 2 wt. % or greater (or 5 wt. % or greater, 10 wt. % or greater, or 15 wt. % or greater), an upper limit of the water content is 90 wt. % (or 80 wt. %, 70 wt. %, 60 wt. %, 50 wt. %, 40 wt. %, 30 wt. %, or 20 wt. %).

[10] The method for producing 1,3-butylene glycol according to any one of [3] to [9], wherein an amount of a hydrogenation catalyst for reduction (hydrogenation) of the acetaldols is from 1 to 30 parts by weight (or from 4 to 25 parts by weight, from 8 to 20 parts by weight, or from 12 to 18 parts by weight) relative to 100 parts by weight of a raw material for hydrogenation.

[11] The method for producing 1,3-butylene glycol according to any one of [3] to [10], wherein an amount of hydrogen used in a reduction (hydrogenation) reaction of the acetaldols is from 0.5 to 40 parts by weight (or from 1 to 30 parts by weight, from 4 to 20 parts by weight, or from 8 to 12 parts by weight) relative to 100 parts by weight of the raw material for hydrogenation.

[12] The method for producing 1,3-butylene glycol according to any one of [3] to [11], wherein a pressure (total pressure, gauge pressure) in a reaction system in the reduction (hydrogenation) reaction of the acetaldols is from 9 to 70 MPa (or from 10 to 40 MPa).

[13] The method for producing 1,3-butylene glycol according to any one of [3] to [12], wherein a hydrogen pressure (partial pressure of hydrogen) in the reaction system in the reduction (hydrogenation) reaction of the acetaldols is from 7 to 60 MPa (or 10 to 30 MPa).

[14] The method for producing 1,3-butylene glycol according to any one of [3] to [13], wherein a reaction temperature in the reduction (hydrogenation) reaction of the acetaldols is from 40 to 150 °C (or from 50 to 140 °C, or from 60 to 130 °C).

[15] The method for producing 1,3-butylene glycol according to any one of [4] to [14], wherein a reaction time (residence time) in the reduction (hydrogenation) reaction of the acetaldols is from to 500 minutes (or from 20 to 400 minutes, from 30 to 300 minutes, from 50 to 280 minutes, or from 80 to 250 minutes).

[16] The method for producing 1,3-butylene glycol according to any one of [1] to [15], further including dealcoholization including removing a low boiling point component containing an alcohol in a process stream containing 1,3-butylene glycol.

[17] The method for producing 1,3-butylene glycol according to any one of [1] to [16], further including desalting including removing a salt in the process stream containing 1,3-butylene glycol.

[18] The method for producing 1,3-butylene glycol according to [17], wherein a bottom ratio (%) in the desalting [(desalting column bottom amount (part)/desalting column charged amount (part)·100] is from 0.1 to 40 wt. % (or from 1 to 35 wt. %, from 2 to 30 wt. %, from 3 to 25 wt. %, from 5 to 20 wt. %, or from 7 to 15 wt. %).

[19] The method for producing 1,3-butylene glycol according to any one of [1] to [18], wherein the high boiling point component removal column is a packed column (or perforated-plate column or a bubble column), and a reboiler employed is a natural downward flow thin-film evaporator or a forced-stirring thin-film evaporator.

[20] The method for producing 1,3-butylene glycol according to any one of [1] to [19], wherein a reflux ratio of the high boiling point component removal column is greater than 0.02.

[21] The method for producing 1,3-butylene glycol according to any one of [1] to [20], wherein a concentration of 1,3-butylene glycol in the charged liquid into the high boiling point component removal column is 95% or greater (or 96% or greater, 97% or greater, 98% or greater, or 99% or greater).

[22] The method for producing 1,3-butylene glycol according to any one of [1] to [21], wherein a content of the high boiling point component in the charged liquid into the high boiling point component removal column is 4% or less (or 3% or less, 2% or less, 1% or less, 0.5% or less, 0.4% or less, 0.3% or less, or 0.2% or less).

[23] The method for producing 1,3-butylene glycol according to any one of [1] to [22], wherein a content of the high boiling point component in the charged liquid into the high boiling point component removal column is 0.1% or less (or 0.05% or less, or 0.01% or less).

[24] The method for producing 1,3-butylene glycol according to any one of [1] to [23], wherein a bottom ratio in the high boiling point component removal column is less than 30 wt. % (or 25 wt. % or less, 20 wt. % or less, 15 wt. % or less, 10 wt. % or less, 7 wt. % or less, 5 wt. % or less, 4 wt. % or less, 3 wt. % or less, 2 wt. % or less, or 1 wt. % or less).

[25] The method for producing 1,3-butylene glycol according to any one of [1] to [24], wherein a bottom ratio in the high boiling point component removal column is 0.01 wt. % or greater (or 0.1 wt. % or greater, 0.5 wt. % or greater, 1 wt. % or greater, 2 wt. % or greater, 3 wt. % or greater, 4 wt. % or greater, 5 wt. % or greater, 6 wt. % or greater, 7 wt. % or greater, 8 wt. % or greater, 9 wt. % or greater, 10 wt. % or greater, 15 wt. % or greater, or 20 wt. % or greater).

[26] The method for producing 1,3-butylene glycol according to any one of the above [1] to [25], wherein a theoretical number of plates of the high boiling point component removal column is from 1 to 100 plates (or from 2 to 90 plates, from 3 to 80 plates, from 4 to 70 plates, from 5 to 60 plates, from 8 to 50 plates, from 10 to 40 plates, or from 15 to 30 plates).

[27] The method for producing 1,3-butylene glycol according to any one of [1] to [26], wherein a feed position of the charged liquid is from 10 to 90% (or from 20 to 80%, from 30 to 70%, or from 40 to 60%) of a height of the column downward from the top of the high boiling point component removal column.

[28] The method for producing 1,3-butylene glycol according to any one of [1] to [27], wherein, in the distillation in the high boiling point component removal column, a pressure (absolute pressure) at the top of the column is from 0.01 to 50 kPa (or from 0.1 to 30 kPa, or from 0.3 to 20 kPa, or from 0.5 to 10 kPa).

[29] The method for producing 1,3-butylene glycol according to any one of the items [1] to [28], wherein at least a portion of the bottom from the high boiling point component removal column is recycled to the step prior to the removing a high boiling point component.

[30] The method for producing 1,3-butylene glycol according to [29], wherein the step prior to the removing a high boiling point component is an acetaldehyde polymerization (or reaction (hydrogenation), dealcoholization (low boiling point component removal), dehydration, or desalting).

[31] The method for producing 1,3-butylene glycol according to [29] or [30], wherein a recycled amount of the bottom in the high boiling point component removal column recycled to the step prior to the removing a high boiling point component is, in a range not greater than an amount of the bottom in the high boiling point component removal column, less than 30 wt. % (or 25 wt. % or less, 20 wt. % or less, 15 wt. % or less, 10 wt. % or less, 7 wt. % or less, 5 wt. % or less, 4 wt. % or less, 3 wt. % or less, 2 wt. % or less, or 1 wt. % or less), with respect to a charged amount into the high boiling point component removal column.

[32] The method for producing 1,3-butylene glycol according to any one of [29] to [31], wherein the recycled amount of the bottom in the high boiling point component removal column recycled to the step prior to the removing a high boiling point component is, in a range not greater than an amount of the bottom in the high boiling point component removal column, 0.01 wt. % or greater (or 0.1 wt. % or greater, 2 wt. % or greater, 3 wt. % or greater, 4 wt. % or greater, 5 wt. % or greater, 7 wt. % or greater, 10 wt. % or greater, or 20 wt. % or greater), with respect to the charged amount into the high boiling point component removal column.

[33] The method for producing 1,3-butylene glycol according to any one of [1] to [32], further including performing alkaline treatment including treating a process stream comprising 1,3-butylene glycol with a base.

[34] The method for producing 1,3-butylene glycol according to [33], wherein the base is added in an amount of from 0.05 to 10 wt. % (or from 0.1 to 1.0 wt. %) relative to a crude 1,3-butylene glycol stream subjected to the alkaline treatment.

[35] The method for producing 1,3-butylene glycol according to [34], wherein the base added in the alkaline treatment is an alkali metal compound.

[36] The method for producing 1,3-butylene glycol according to [35], wherein the alkali metal compound is sodium hydroxide (or potassium hydroxide, sodium (bi)carbonate, or potassium (bi)carbonate).

[37] The method for producing 1,3-butylene glycol according to any one of [33] to [36], wherein a reaction temperature of the alkaline treatment is from 90 to 140 °C (or from 110 to 130 °C), and a reaction residence time is from 5 to 120 minutes (or from 10 to 30 minutes).

[38] The method for producing 1,3-butylene glycol according to any one of [33] to [37], wherein evaporation is performed in an evaporator used in the alkaline treatment under a reduced pressure at the top of the column of 20 kPa or less (absolute pressure) (or from 0.5 to 10 kPa (absolute pressure)) at from 90 to 120 °C.

[39] The method for producing 1,3-butylene glycol according to any one of [1] to [38], wherein the product distillation column used in the product distillation is a packed column (or perforated-plate column or a bubble column), and a reboiler employed is a natural downward flow thin-film evaporator or a forced-stirring thin-film evaporator.

[40] The method for producing 1,3-butylene glycol according to [39], wherein a theoretical number of plates of the product distillation column is from 1 to 100 plates (or from 2 to 90 steps, from 3 to 80 plates, from 4 to 70 plates, from 5 to 60 plates, from 8 to 50 plates, from 10 to 40 plates, or from 15 to 30 plates), in a case where the concentration of the low boiling point substance in the charged liquid is 5 wt. % or less.

[41] The method for producing 1,3-butylene glycol according to [39] or [40], wherein a feed position of the charged liquid in the product distillation column is from 10 to 90% (or from 20 to 80%, from 30 to 70%, from 40 to 60%) of a height of the column downward from the top of the column, and a pressure (absolute pressure) at the top of the column is from 20 kPa or less (or from 0.1 to 10 kPa, from 0.3 to 8 kPa, or from 0.5 to 5 kPa).

[42] The method for producing 1,3-butylene glycol according to any one of [39] to [41], wherein a reflux ratio in the product distillation column is from 0.05 to 500 (or from 0.1 to 300, from 0.2 to 200, from 0.5 to 100, from 1 to 50, from 2 to 40, from 3 to 30, or from 4 to 25).

[43]A 1,3-butylene glycol product, having a dry point of 209 °C or lower and, according to a gas chromatographic analysis performed under conditions set forth below:
an area ratio of a 1,3-butylene glycol peak of greater than 98.5% (or 98.6% or greater, 98.7% or greater, 98.8% or greater, 98.9% or greater, 99% or greater, 99.1% or greater, 99.2% or greater, 99.3%, 99.4% or greater, 99.5% or greater, 99.6% or greater, or 99.7% or greater) and a total area ratio of peaks longer in retention time than the 1,3-butylene glycol peak of less than 1.2% (or 1.1% or less, 1% or less, 0.9% or less, 0.8% or less, 0.7% or less, 0.6% or less, 0.5% or less, 0.4% or less, 0.3% or less, 0.2% or less, 0.1% or less, 0.05% or less, or 0.03% or less),
in which the conditions for the gas chromatographic analysis are as follows:
Analytical Column: a column with dimethylpolysiloxane as a stationary phase, having a length of m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

[44] The 1,3-butylene glycol product according to [43], wherein the area ratio of the 1,3-butylene glycol peak is 99.0% or greater.

[45] The 1,3-butylene glycol product according to [43] or [44], wherein the total area ratio of the peaks longer in retention time than the 1,3-butylene glycol peak is 0.6% or less.

[46]A moisturizer containing the 1,3-butylene glycol product described in any one of [43] to [45].

[47]A cosmetic product containing the moisturizer described in [46].

[201]A method for producing 1,3-butylene glycol to yield purified 1,3-butylene glycol from a reaction crude liquid containing 1,3-butylene glycol,
the method including: performing dehydration including removing water by distillation, removing a high boiling point component including removing a high boiling point component by distillation, and performing product distillation to yield purified 1,3-butylene glycol,
wherein, in a product column for use in the product distillation, a charged liquid containing 1,3-butylene glycol is distilled off under a condition that a reflux ratio is greater than 0.1 (or 0.15 or greater), a liquid in which a low boiling point component is concentrated is distilled off from above a charging plate, and 1,3-butylene glycol is extracted from below the charging plate.

[202] The method for producing 1,3-butylene glycol according to [201], wherein the reaction crude liquid containing 1,3-butylene glycol is a reaction crude liquid produced by hydrogen reduction of acetaldols (or a reaction crude liquid produced by hydrolysis of 1,3-butylene oxide, a reaction crude liquid produced by selective hydrocracking of erythritol, a reaction crude liquid produced by selective hydrogen addition to butadiene, a reaction crude liquid produced by hydrogen addition to n-butanal-3-one, a reaction crude liquid produced by hydrogen addition to 1-butanol-3-one, a reaction crude liquid produced by hydrogen addition to 3-hydroxy-1-butanoic acid, a reaction crude liquid produced by hydrogen addition to β-butyrolactone, or a reaction crude liquid produced by hydrogen addition to diketene.

[203] The method for producing 1,3-butylene glycol according to [202], wherein the acetaldols used as a raw material in reduction (hydrogenation) of the acetaldols is any of acetaldol; its cyclic dimer paraldol; aldoxane as a cyclic trimer of acetaldehyde; and mixtures of these.

[204] The method for producing 1,3-butylene glycol according to [202] or [203], wherein the reaction crude liquid containing acetaldols is subjected to simple evaporation, distillation, or hydrogen addition to remove aldehydes, followed by the hydrogenation.

[205] The method for producing 1,3-butylene glycol according to any one of [202] to [204], wherein a content of the acetaldols used as the raw material in reduction (hydrogenation) of the acetaldols is 30 wt. % or greater (e.g., from 30 to 99 wt. %) (or 40 wt. % or greater (e.g., from 40 to 98 wt. %), 50 wt. % or greater (e.g., from 50 to 97 wt. %), 60 wt. % or greater (e.g., from 60 to 95 wt. %), from 65 to 90 wt. %, from 70 to 90 wt. %, or 75 to 90 wt. %).

[206] The method for producing 1,3-butylene glycol according to any one of [202] to [205], wherein the raw material for reduction (hydrogenation) of the acetaldols contains water, and a content of the water is 2 wt. % or greater (or 5 wt. % or greater, 10 wt. % or greater, or 15 wt. % or greater), an upper limit of the water content is 90 wt. % (or 80 wt. %, 70 wt. %, 60 wt. %, 50 wt. %, 40 wt. %, 30 wt. %, or 20 wt. %).

[207] The method for producing 1,3-butylene glycol according to any one of [202] to [206], wherein an amount of a hydrogenation catalyst for reduction (hydrogenation) of the acetaldols is from 1 to 30 parts by weight (or from 4 to 25 parts by weight, from 8 to 20 parts by weight, or from 12 to 18 parts by weight) relative to 100 parts by weight of a raw material for hydrogenation.

[208] The method for producing 1,3-butylene glycol according to any one of [202] to [207], wherein an amount of hydrogen used in a reduction (hydrogenation) reaction of the acetaldols is from 0.5 to 40 parts by weight (or from 1 to 30 parts by weight, from 4 to 20 parts by weight, or from 8 to 12 parts by weight) relative to 100 parts by weight of the raw material for hydrogenation.

[209] The method for producing 1,3-butylene glycol according to any one of [202] to [208], wherein a pressure (total pressure, gauge pressure) in a reaction system in the reduction (hydrogenation) reaction of the acetaldols is from 9 to 70 MPa (or from 10 to 40 MPa).

[210] The method for producing 1,3-butylene glycol according to any one of [202] to [209], wherein a hydrogen pressure (partial pressure of hydrogen) in the reaction system in the reduction (hydrogenation) reaction of the acetaldols is from 7 to 60 MPa (or 10 to 30 MPa).

[211] The method for producing 1,3-butylene glycol according to any one of [202] to [210], wherein a reaction temperature in the reduction (hydrogenation) reaction of the acetaldols is from 40 to 150 °C (or from 50 to 140 °C, or from 60 to 130 °C).

[212] The method for producing 1,3-butylene glycol according to any one of [202] to [211], wherein a reaction time (residence time) in the reduction (hydrogenation) reaction of the acetaldols is from 10 to 500 minutes (or from 20 to 400 minutes, from 30 to 300 minutes, from 50 to 280 minutes, or from 80 to 250 minutes).

[213] The method for producing 1,3-butylene glycol according to any one of [201] to [212], further including dealcoholization for removing a low boiling point substance containing an alcohol in a process stream containing 1,3-butylene glycol.

[214] The method for producing 1,3-butylene glycol according to any one of [201] to [213], further including desalting including removing a salt in the process stream containing 1,3-butylene glycol.

[215] The method for producing 1,3-butylene glycol according to [214], wherein a bottom ratio (%) in the desalting [(desalting column bottom amount (part)/desalting column charged amount (part)·100] is from 0.1 to 40 wt. % (or from 1 to 35 wt. %, from 2 to 30 wt. %, from 3 to 25 wt. %, from 5 to 20 wt. %, or from 7 to 15 wt. %).

[216] The method for producing 1,3-butylene glycol according to any one of [201] to [215], wherein the high boiling point component removal column used in the removing a high boiling point component is a packed column (or perforated-plate column or a bubble column), and a reboiler employed is a natural downward flow thin-film evaporator or a forced-stirring thin-film evaporator.

[217] The method for producing 1,3-butylene glycol according to [216], wherein a theoretical number of plates of the high boiling point component removal column is from 1 to 100 plates (or from 2 to 90 plates, from 3 to 80 plates, from 4 to 70 plates, from 5 to 60 plates, from 8 to 50 plates, from 10 to 40 plates, or from 15 to 30 plates).

[218] The method for producing 1,3-butylene glycol according to any one of [216] or [217], wherein a feed position of the charged liquid is from 10 to 90% (or from 20 to 80%, from 30 to 70%, or from 40 to 60%) of a height of the column downward from the top of the high boiling point component removal column.

[219] The method for producing 1,3-butylene glycol according to any one of [216] to [218], wherein, in the distillation in the high boiling point component removal column, a pressure (absolute pressure) at the top of the column is from 0.01 to 50 kPa (or from 0.1 to 30 kPa, or from 0.3 to 20 kPa, or from 0.5 to 10 kPa).

[220] The method for producing 1,3-butylene glycol according to any one of the items [216] to [219], wherein at least a portion of the bottom from the high boiling point component removal column is recycled to a step prior to the removing a high boiling point component.

[221] The method for producing 1,3-butylene glycol according to any one of [216] to [220], wherein the reflux ratio of the high boiling point component removal column is 0.015 or greater (or 0.02 or greater, 0.03 or greater, 0.05 or greater, 0.07 or greater, 0.1 or greater, 0.2 or greater, 0.3 or greater, 0.4 or greater. 0.5 or greater, 0.6 or greater, 0.7 or greater, 0.8 or greater, 0.9 or greater, 1 or greater, 2 or greater, 3 or greater, 4 or greater, 5 or greater, 6 or greater, 7 or greater, 8 or greater, 9 or greater, 10 or greater, 15 or greater, 20 or greater, or 30 or greater).

[222] The method for producing 1,3-butylene glycol according to [221], wherein an upper limit of the reflux ratio is 100 (or 50).

[223] The method for producing 1,3-butylene glycol according to any one of [201] to [222], further including alkaline treatment for treating a process stream including 1,3-butylene glycol with a base.

[224] The method for producing 1,3-butylene glycol according to [223], wherein the base is added in an amount of from 0.05 to 10 wt. % (or from 0.1 to 1.0 wt. %) relative to a crude 1,3-butylene glycol stream subjected to the alkaline treatment.

[225] The method for producing 1,3-butylene glycol according to [223] or [224], wherein the base added in the alkaline treatment is an alkali metal compound.

[226] The method for producing 1,3-butylene glycol according to [225], wherein the alkali metal compound is sodium hydroxide (or potassium hydroxide, sodium (bi)carbonate, or potassium (bi)carbonate).

[227] The method for producing 1,3-butylene glycol according to any one of [223] to [226], wherein a reaction temperature of the alkaline treatment is from 90 to 140 °C (or from 110 to 130 °C), and a reaction residence time is from 5 to 120 minutes (or from 10 to 30 minutes).

[228] The method for producing 1,3-butylene glycol according to any one of [223] to [227], wherein evaporation is performed in an evaporator used in the alkaline treatment under a reduced pressure at the top of the column of 20 kPa or less (absolute pressure) (or from 0.5 to 10 kPa (absolute pressure)) at from 90 to 120 °C.

[229] The method for producing 1,3-butylene glycol according to any one of [201] to [228], wherein the product column used in the product distillation is a packed column (or perforated-plate column or a bubble column), and a reboiler employed is a natural downward flow thin-film evaporator or a forced-stirring thin-film evaporator.

[230] The method for producing 1,3-butylene glycol according to [229], wherein a theoretical number of plates of the product column is from 1 to 100 plates (or from 2 to 90 plates, from 3 to 80 plates, from 4 to 70 plates, from 5 to 60 plates, from 8 to 50 plates, from 10 to 40 plates, or from 15 to 30 plates).

[231] The method for producing 1,3-butylene glycol according to [229] or [230], wherein a feed position of the charged liquid in the product column is from 10 to 90% (or from 20 to 80%, from 30 to 70%, from to 60%) of a height of the column downward from the top of the column, and a pressure (absolute pressure) at the top of the column is from 20 kPa or less (or from 0.1 to 10 kPa, from 0.3 to 8 kPa, or from 0.5 to 5 kPa).

[232] The method for producing 1,3-butylene glycol according to any one of [229] to [231], wherein the reflux ratio in the product column is 0.2 or greater (or 0.3 or greater, 0.4 or greater, 0.5 or greater, 1 or greater, 2 or greater, 3 or greater, 4 or greater, 5 or greater, 6 or greater, 7 or greater, 8 or greater, 9 or greater, 10 or greater, 20 or greater, 50 or greater, 400 or greater, or 500 or greater).

[233] The method for producing 1,3-butylene glycol according to any one of [229] to [232], wherein a concentration of 1,3-butylene glycol in the charged liquid into the product column is 90% or greater (or 91% or greater, 92% or greater, 93% or greater, 94% or greater, 95% or greater, 96% or greater, 97% or greater, 98% or greater, or 99% or greater).

[234] The method for producing 1,3-butylene glycol according to any one of [229] to [233], wherein a content of water in the charged liquid into the product column is, for example, 3 wt. % or less, (2.8 wt. % or less, 2.6 wt. % or less, 2.4 wt. % or less, 2.2 wt. % or less, 2 wt. % or less, 1.8 wt. % or less, 1.6 wt. % or less, 1.4 wt. % or less, 1.4 wt. % or less, 1.2 wt. % or less, 1 wt. % or less, 0.95 wt. % or less, 0.9 wt. % or less, 0.8 wt. % or less, 0.7 wt. % or less, 0.6 wt. % or less, 0.5 wt. % or less, 0.4 wt. % or less, 0.3 wt. % or less, 0.2 wt. % or less, or 0.1 wt. % or less).

[235] The method for producing 1,3-butylene glycol according to any one of [229] to [234], wherein a content of the low boiling point component other than water in the charged liquid into the product column is 1.8% or less (or 1.6% or less, 1.4% or less, 1.2% or less, 1.1% or less, 1% or less, 0.9% or less, 0.8% or less, 0.7% or less, 0.6% or less, 0.5% or less, or 0.4%).

[236] The method for producing 1,3-butylene glycol according to any one of [229] to [235], wherein a distillation ratio in the product column is less than 30 wt. % (or less than 29 wt. %, less than 28 wt. %, less than 27 wt. %, less than 26 wt. %, less than 25 wt. %, less than 24 wt. %, less than 23 wt. %, less than 22 wt. %, less than 21 wt. %, or less than 20 wt. %, 19 wt. % or less, 18 wt. % or less, 17 wt. % or less, 16 wt. % or less or 15 wt. % or less, 12 wt. % or less, 10 wt. % or less, 8 wt. % or less, 5 wt. % or less, 3 wt. % or less, 2 wt. % or less, 1 wt. % or less, 0.8 wt. % or less, 0.6 wt. % or less, or 0.4 wt. % or less).

[237] The method for producing 1,3-butylene glycol according to any one of [229] to [236], wherein at least a portion of a distillate from the product column is recycled to the dehydration, dealcoholization, low boiling point component removal prior to the performing product distillation, or a step prior to the steps.

[238] The method for producing 1,3-butylene glycol according to [237], wherein a recycled amount of the distillate from the product column recycled to the step prior to the performing product distillation is less than 30 wt. % with respect to a charged amount into the product column, in a range of not greater than a distilled amount in the product column.

[239] The method for producing 1,3-butylene glycol according to[237] or [238], wherein the recycled amount of the distillate from the product column recycled to the step prior to the performing product distillation is 0.01 wt. % or greater (or 0.05 wt. % or greater, 0.1 wt. % or greater, 0.5 wt. % or greater, 1 wt. % or greater, 1.5 wt. % or greater, 2 wt. % or greater, 3 wt. % or greater, 4 wt. % or greater 5 wt. % or greater, 7 wt. % or greater, 10 wt. % or greater, or 20 wt. % or greater) with respect to the charged amount into the product column, in the range of not greater than the distilled amount in the product column.

[240]A 1,3-butylene glycol product, having an initial boiling point of higher than 203 °C (or 204 °C or higher, 05 °C or higher, 206 °C or higher, 207 °C, or 208 °C or higher) and a water content of less than 0.4 wt. % (or 0.3 wt. % or less, 0.2 wt. % or less, 0.1 wt. % or less, 0.07 wt. % or less, 0.05 wt. % or less, 0.03 wt. % or less, 0.02 wt. % or less, 0.01 wt. % or less, or 0.005 wt. % or less).

[241] The 1,3-butylene glycol product according to [240], having, in a gas chromatographic analysis performed under conditions set forth below:
an area ratio of a 1,3-butylene glycol peak of greater than 98.5% (or 98.6% or greater, 98.7% or greater, 98.8% or greater, 98.9% or greater, 99% or greater, 99.1% or greater, 99.2% or greater, 99.3%, 99.4% or greater, 99.5% or greater, 99.6% or greater, 99.7% or greater, or 99.8% or greater) and a total area ratio of peaks shorter in retention time than the 1,3-butylene glycol peak of less than 0.3% (or 0.25% or less, 0.23% or less, 0.2% or less, 0.17% or less, 0.15% or less, 0.12% or less, 0.1% or less, 0.07% or less, 0.04% or less, 0.03% or less, 0.02% or less, 0.01% or less, 0.007% or less, 0.005% or less, or 0.002% or less),
the conditions for the gas chromatographic analysis are as follows:
Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

[242]A moisturizer containing the 1,3-butylene glycol product described in any one of [240] or [241]. [243] The moisturizer according to [242], wherein a content of the 1,3-butylene glycol product is 10 wt. % or greater (or 30 wt. % or greater, 50 wt. % or greater, 80 wt. % or greater, 90 wt. % or greater, or 100 wt. %).

[244]A cosmetic product containing the moisturizer described in [242] or [243]. [245] The cosmetic product according to [244], wherein a blending amount of the 1,3-butylene glycol product is from 0.01 to 40 wt. % (or from 0.1 to 30 wt. %, from 0.2 to 20 wt. %, from 0.5 to 15 wt. %, or from 1 to 10 wt. %).

[301]A method for manufacturing 1,3-butylene glycol, which is a method for obtaining purified 1,3-butylene glycol from a crude reaction liquid containing 1,3-butylene glycol, the method including:
a dehydration step of removing water by distillation; a high boiling substance removal step of removing a high boiling point component by distillation; and a product distillation step of obtaining purified 1,3-butylene glycol,
wherein, in the product distillation step, a product column is used in which a liquid feed having a 1,3-butylene glycol concentration of 97% or higher (or 98% or higher, or 99% or higher), an acetaldehyde content of 500 ppm or lower (or 200 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, or lower than 2 ppm), and a crotonaldehyde content of 200 ppm or lower (or 110 ppm or lower, 100 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, 3 ppm or lower, 2 ppm or lower, or lower than 1 ppm) is distilled under a condition of a reflux ratio of higher than 0.1 (or 0.15 or higher), and a liquid concentrated with acetaldehyde and crotonaldehyde is distilled off from above a feed plate, and 1,3-butylene glycol is extracted from below the feed plate.

[302] The method for manufacturing 1,3-butylene glycol according to [301], wherein the crude reaction liquid containing 1,3-butylene glycol is: a crude reaction liquid obtained by hydrogen reduction of an acetaldol (or a crude reaction liquid obtained by hydrolyzation of 1,3-butylene oxide; a crude reaction liquid obtained by selective hydrocracking of erythritol; a crude reaction liquid obtained by selective water addition to butadiene; a crude reaction liquid obtained by hydrogen addition to n-butanal-3-one; a crude reaction liquid obtained by hydrogen addition to 1-butanol-3-one; a crude reaction liquid obtained by hydrogen addition to 3-hydroxy-1-butanoic acid; a crude reaction liquid obtained by hydrogen addition to P-butyrolactone; or a crude reaction liquid obtained by hydrogen addition to diketene.

[303] The method for manufacturing 1,3-butylene glycol according to [302], wherein the acetaldol used as a raw material in reduction (hydrogenation) of the acetaldol is any of acetaldol; its cyclic dimer paraldol; aldoxane as a cyclic trimer of acetaldehyde; and mixtures of these.

[304] The method for manufacturing 1,3-butylene glycol according to [302] or [303], wherein the crude reaction liquid containing an acetaldol is subjected to simple evaporation, distillation, or hydrogen addition to remove aldehydes, followed by a hydrogenation step.

[305] The method for manufacturing 1,3-butylene glycol according to any one of [302] to [304], wherein a content of the acetaldol used as the raw material in reduction (hydrogenation) of the acetaldol is 30 wt. % or higher (e.g., from 30 to 99 wt. %) (or 40 wt. % or higher (e.g., from 40 to 98 wt. %), 50 wt. % or higher (e.g., from 50 to 97 wt. %), 60 wt. % or higher (e.g., from 60 to 95 wt. %), from 65 to 90 wt. %, from 70 to 90 wt. %, or 75 to 90 wt. %).

[306] The method for manufacturing 1,3-butylene glycol according to any one of [302] to [305], wherein the raw material for the reduction (hydrogenation) of the acetaldol contains water, a content of the water is 2 wt. % or higher (or 5 wt. % or higher, 10 wt. % or higher, or 15 wt. % or higher), and an upper limit of the content of the water is 90 wt. % (or 80 wt. %, 70 wt. %, 60 wt. %, 50 wt. %, 40 wt. %, 30 wt. %, or 20 wt. %).

[307] The method for manufacturing 1,3-butylene glycol according to any one of [302] to [306], wherein an amount of a hydrogenation catalyst for the reduction (hydrogenation) of the acetaldol is from 1 to 30 parts by weight (or from 4 to 25 parts by weight, from 8 to 20 parts by weight, or from 12 to 18 parts by weight) relative to 100 parts by weight of the raw material for hydrogenation.

[308] The method for manufacturing 1,3-butylene glycol according to any one of [302] to [307], wherein an amount of hydrogen used in the reduction (hydrogenation) reaction of the acetaldol is from 0.5 to 40 parts by weight (or from 1 to 30 parts by weight, from 4 to 20 parts by weight, or from 8 to 12 parts by weight) relative to 100 parts by weight of the raw material for hydrogenation.

[309] The method for manufacturing 1,3-butylene glycol according to any one of [302] to [308], wherein a pressure (total pressure, gauge pressure) in a reaction system in the reduction (hydrogenation) reaction of the acetaldol is from 9 to 70 MPa (or from 10 to 40 MPa).

[310] The method for manufacturing 1,3-butylene glycol according to any one of [302] to [309], wherein a hydrogen pressure (partial pressure of hydrogen) in the reaction system in the reduction (hydrogenation) reaction of the acetaldol is from 7 to 60 MPa (or 10 to 30 MPa).

[311] The method for manufacturing 1,3-butylene glycol according to any one of [302] to [310], wherein a reaction temperature in the reduction (hydrogenation) reaction of the acetaldol is from to 150 °C (or from 50 to 140 °C, or from 60 to 130 °C).

[312] The method for manufacturing 1,3-butylene glycol according to any one of [302] to [311], wherein a reaction time (residence time) in the reduction (hydrogenation) reaction of the acetaldol is from 10 to 500 minutes (or from 20 to 400 minutes, from 30 to 300 minutes, from 50 to 280 minutes, or from 80 to 250 minutes).

[313] The method for manufacturing 1,3-butylene glycol according to any one of [301] to [312], further including a dealcoholization step of removing a low boiling substance containing alcohols in a process stream containing 1,3-butylene glycol.

[314] The method for manufacturing 1,3-butylene glycol according to any one of [301] to [313], further including a desalting step of removing a salt in a process stream containing 1,3-butylene glycol.

[315] The method for manufacturing 1,3-butylene glycol according to [314], wherein a bottom rate (%) in the a desalting step [(desalting column bottom amount (part)/desalting column charged amount (part)·100] is from 0.1 to 40 wt. % (or from 1 to 35 wt. %, from 2 to 30 wt. %, from 3 to 25 wt. %, from 5 to 20 wt. %, or from 7 to 15 wt. %).

[316] The method for manufacturing 1,3-butylene glycol according to any one of [301] to [315], wherein the high boiling substance removal column used in the high boiling substance removal is a packed column (or perforated-plate column or a bubble column), and a reboiler employed is a natural downward flow thin-film evaporator or a forced-stirring thin-film evaporator.

[317] The method for manufacturing 1,3-butylene glycol according to [316], wherein the number of theoretical plates of the high boiling substance removal column is from 1 to 100 plates (or from 2 to 90 plates, from 3 to 80 plates, from 4 to 70 plates, from 5 to 60 plates, from 8 to 50 plates, from 10 to 40 plates, or from 15 to 30 plates).

[318] The method for manufacturing 1,3-butylene glycol according to any one of [316] or [317], wherein a feed position of the liquid feed is from 10 to 90% (or from 20 to 80%, from 30 to 70%, or from 40 to 60%) of a height of the column facing downward from the top of the high boiling substance removal column.

[319] The method for manufacturing 1,3-butylene glycol according to any one of [316] to [318], wherein, in the distillation in the high boiling substance removal column, a pressure (absolute pressure) at the top of the column is from 0.01 to 50 kPa (or from 0.1 to 30 kPa, or from 0.3 to 20 kPa, or from 0.5 to 10 kPa).

[320] The method for manufacturing 1,3-butylene glycol according to any one of the items [316] to [319], wherein at least a portion of a bottom from the high boiling substance removal column is recycled to a step prior to the high boiling substance removal step.

[321] The method for manufacturing 1,3-butylene glycol according to any one of [316] to [320], wherein a reflux ratio of the high boiling substance removal column is 0.015 or higher (or 0.02 or higher, 0.03 or higher, 0.05 or higher, 0.07 or higher, 0.1 or higher, 0.2 or higher, 0.3 or higher, 0.4 or higher, 0.5 or higher, 0.6 or higher, 0.7 or higher, 0.8 or higher, 0.9 or higher, 1 or higher, 2 or higher, 3 or higher, 4 or higher, 5 or higher, 6 or higher, 7 or higher, 8 or higher, 9 or higher, 10 or higher, 15 or higher, 20 or higher, or 30 or higher).

[322] The method for manufacturing 1,3-butylene glycol according to [321], wherein an upper limit of the reflux ratio is 100 (or 50).

[323] The method for manufacturing 1,3-butylene glycol according to any one of [301] to [322], further including an alkaline treatment step of treating a process stream containing 1,3-butylene glycol with a base.

[324] The method for manufacturing 1,3-butylene glycol according to [323], wherein, in the alkaline treatment, the base is added in an amount of from 0.05 to 10 wt. % (or from 0.1 to 1.0 wt. %) relative to a crude 1,3-butylene glycol stream subjected to the alkaline treatment.

[325] The method for manufacturing 1,3-butylene glycol according to [323] or [324], wherein the base added in the alkaline treatment is an alkali metal compound.

[326] The method for manufacturing 1,3-butylene glycol according to [325], wherein the alkali metal compound is sodium hydroxide (or potassium hydroxide, sodium (bi)carbonate, or potassium (bi)carbonate).

[327] The method for manufacturing 1,3-butylene glycol according to any one of [323] to [326], wherein a reaction temperature of the alkaline treatment is from 90 to 140 °C (or from 110 to 130 °C), and a reaction residence time is from 5 to 120 minutes (or from 10 to 30 minutes).

[328] The method for manufacturing 1,3-butylene glycol according to any one of [323] to [327], wherein evaporation is performed in an evaporator used in the alkaline treatment under a reduced pressure at the top of the column of 20 kPa or lower (absolute pressure) (or from 0.5 to 10 kPa (absolute pressure)) at from 90 to 120 °C.

[329] The method for manufacturing 1,3-butylene glycol according to any one of [301] to [328], wherein the product column is a packed column (or perforated-plate column or a bubble column), and a reboiler employed is a natural downward flow thin-film evaporator or a forced-stirring thin-film evaporator.

[330] The method for manufacturing 1,3-butylene glycol according to any one of [301] to [329], wherein the number of theoretical plates of the product column is from 1 to 100 plates (or from 2 to 90 plates, from 3 to 80 plates, from 4 to 70 plates, from 5 to 60 plates, from 8 to 50 plates, from 10 to 40 plates, or from 15 to 30 plates).

[331] The method for manufacturing 1,3-butylene glycol according to any one of [301] to [330], wherein a feed position of the liquid feed in the product column is from 10 to 90% (or from 20 to 80%, from 30 to 70%, from 40 to 60%) of the height of the column downward from the top of the column, and the pressure (absolute pressure) at the top of the column is from 20 kPa or less (or from 0.1 to 10 kPa, from 0.3 to 8 kPa, or from 0.5 to 5 kPa).

[332] The method for manufacturing 1,3-butylene glycol according to any one of [301] to [331], wherein the reflux ratio in the product column is 0.2 or higher (or 0.3 or higher, 0.4 or higher, 0.5 or higher, 1 or higher, 2 or higher, 3 or higher, 4 or higher, 5 or higher, 6 or higher, 7 or higher, 8 or higher, 9 or higher, 10 or higher, 11 or higher, 12 or higher, 13 or higher, 14 or higher, 15 or higher, 16 or higher, 17 or higher, 18 or higher, 19 or higher, 20 or higher, 25 or higher, 30 or higher, 35 or higher, 40 or higher, 50 or higher, 400 or higher, or 500 or higher).

[333] The method for manufacturing 1,3-butylene glycol according to any one of [301] to [332], wherein the content of acetaldehyde in the liquid feed into the product column is 205 ppm or lower (or 200 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, or lower than 2 ppm).

[334] The method for manufacturing 1,3-butylene glycol according to any one of [301] to [333], wherein the content of crotonaldehyde in the liquid feed into the product column is 110 ppm or lower (or 100 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, 3 ppm or lower, 2 ppm or lower, or lower than 1 ppm).

[335] The method for manufacturing 1,3-butylene glycol according to any one of [301] to [334], wherein a distillation rate in the product column is lower than 20 wt. % (or 15 wt. % or lower, 12 wt. % or lower, 10 wt. % or lower, 8 wt. % or lower, 5 wt. % or lower, 3 wt. % or lower, 2 wt. % or lower, 1 wt. % or lower, 0.8 wt. % or lower, 0.6 wt. % or lower, 0.4 wt. % or lower, or 0.2 wt. % or lower).

[336] The method for manufacturing 1,3-butylene glycol according to any one of [301] to [335], wherein at least a portion of a distillate from the product column is recycled to a step prior to the product distillation step, namely the dehydration step, a dealcoholization step, a low boiling substance removal step, or another step prior to these step.

[337] The method for manufacturing 1,3-butylene glycol according to [336], wherein the crude reaction liquid containing 1,3-butylene glycol is a crude reaction liquid obtained by hydrogen reduction of an acetaldol, and at least a portion of the distillate from the product column is recycled to a step of the hydrogen reduction of an acetaldol or a step upstream of the hydrogen reduction.

[338] The method for manufacturing 1,3-butylene glycol according to[336] or [337], wherein an amount of the distillate from the product column being recycled to a step prior to the product distillation is lower than 30 wt. % (or 90 wt. % or lower, 80 wt. % or lower, 70 wt. % or lower, 60 wt. % or lower, 50 wt. % or lower, 40 wt. % or lower, or 30 wt. % or lower) with respect to a charged amount into the product column within a range not higher than a distilled amount in the product column.

[339] The method for manufacturing 1,3-butylene glycol according to any one of [336] to [338], wherein the amount of the distillate from the product column being recycled to a step prior to the product distillation is 0.01 wt. % or higher (or 0.05 wt. % or higher, 0.1 wt. % or higher, 0.5 wt. % or higher, 1 wt. % or higher, 1.5 wt. % or higher, 2 wt. % or higher, 3 wt. % or higher, 4 wt. % or higher, 5 wt. % or higher, 7 wt. % or higher, 10 wt. % or higher, or 20 wt. % or higher) with respect to the charged amount into the product column within a range not higher than the distilled amount in the product column.

[340]A 1,3-butylene glycol product, having a potassium permanganate test value of longer than 10 minutes (or 5 minutes or longer, 30 minutes or longer, 40 minutes or longer, 50 minutes or longer, or 60 minutes or longer).

[341] The 1,3-butylene glycol product according to [340], having a peak area ratio of 1,3-butylene glycol of higher than 98.5% (or 98.6% or higher, 98.7% or higher, 98.8% or higher, 98.9% or higher, 99% or higher, 99.1% or higher, 99.2% or higher, 99.3%, 99.5% or higher, or 99.8% or higher), according to a gas chromatographic analysis performed under conditions set forth below,
in which the conditions for the gas chromatographic analysis are as follows:
Analytical Column: a column with dimethylpolysiloxane as a stationary phase, having a length of m, an inner diameter of 0.25 mm, and a film thickness of 1.0 μm
Heating Conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

[342]

The 1,3-butylene glycol product according to [340] or [341], wherein a content of acetaldehyde is 2 ppm or lower (or 1.7 ppm or lower, 1.5 ppm or lower, 1.2 ppm or lower, 1.0 ppm or lower, 0.7 ppm or lower, 0.5 ppm or lower, 0.3 ppm or lower, or 0.2 ppm or lower).

[343] The 1,3-butylene glycol product according to any one of [340] to [342], wherein a content of crotonaldehyde is 1.2 ppm or lower (or 1.0 ppm or lower, 0.7 ppm or lower, 0.5 ppm or lower, 0.3 ppm or lower, 0.2 ppm or lower, or 0.1 ppm or lower).

[344]A moisturizer containing the 1,3-butylene glycol product described in any one of [340] to [343]. [345] The moisturizer according to [344], wherein a content of the 1,3-butylene glycol product is 10 wt. % or higher (or 30 wt. % or higher, 50 wt. % or higher, 80 wt. % or higher, 90 wt. % or higher, or 100 wt. % or higher).

[346]A cosmetic product containing the moisturizer described in [344] or [345].

[347] The cosmetic product according to [346], wherein an amount of the 1,3-butylene glycol product blended is from 0.01 to 40 wt. % (or from 0.1 to 30 wt. %, from 0.2 to 20 wt. %, from 0.5 to 15 wt. %, or from 1 to 10 wt. %).

[401]A method for manufacturing 1,3-butylene glycol, which is a method for obtaining purified 1,3-butylene glycol from a crude reaction liquid containing 1,3-butylene glycol, the method including:
a dehydration step of removing water by distillation; a high boiling substance removal step of removing a high boiling point component by distillation; and a product distillation step of obtaining purified 1,3-butylene glycol,
wherein in the dehydration step, a dehydration column is used in which a liquid feed containing 1,3-butylene glycol and water with an acetaldehyde content of 1000 ppm or lower (or 900 ppm or lower, 800 ppm or lower, 700 ppm or lower, 600 ppm or lower, 500 ppm or lower, 400 ppm or lower, 300 ppm or lower, 200 ppm or lower, 155 ppm or lower, 140 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, 3 ppm or lower, 2 ppm or lower, or 1 ppm or lower), and a crotonaldehyde content of 400 ppm or lower (or 200 ppm or lower, 150 ppm or lower, 130 ppm or lower, 117 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, 3 ppm or lower, 2 ppm or lower, or 1 ppm or lower) is distilled under a condition of a reflux ratio of higher than 0.3 (or 0.4 or higher, 0.5 or higher, 0.6 or higher, 0.7 or higher, 0.8 or higher, 0.9 or higher, 1 or higher, 1.1 or higher, 1.2 or higher, 1.3 or higher, 1.4 or higher, 1.5 or higher, 1.6 or higher, 1.7 or higher, 1.8 or higher, 1.9 or higher, 2 or higher, 3 or higher, 4 or higher, 5 or higher, 6 or higher, 7 or higher, 8 9 or higher, 10 or higher, 15 or higher, 20 or higher, 25 or higher, 30 or higher, or 40 or higher), and a liquid concentrated with a low boiling point component containing water is distilled off from above a feed plate, and
in the product distillation step, a product column is used in which a 1,3-butylene glycol liquid feed with an acetaldehyde content of 500 ppm or lower (or 205 ppm or lower, 200 ppm or lower, 150 ppm or lower, 120 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, or lower than 2 ppm) and a crotonaldehyde content of 200 ppm or lower (or 150 ppm or lower, 130 ppm or lower, 110 ppm or lower, 100 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, 3 ppm or lower, 2 ppm or lower, or lower than 1 ppm) is distilled under a condition of a reflux ratio of higher than 0.1 (or 0.15 or higher, 0.2 or higher, 0.3 or higher, 0.4 or higher, 0.5 or higher, 1 or higher, 2 or higher, 3 or higher, 4 or higher, 5 or higher, 6 or higher, 7 or higher, 8 or higher, 9 or higher, 10 or higher, 20 or higher, 50 or higher, 400 or higher, or 500 or higher), a liquid concentrated with a low boiling point component is distilled off from above a feed plate, and 1,3-butylene glycol is extracted from below the feed plate.

[402] The method for manufacturing 1,3-butylene glycol according to [401], wherein the crude reaction liquid containing 1,3-butylene glycol is a crude reaction liquid obtained by hydrogen reduction of an acetaldol.

[403] The method for manufacturing 1,3-butylene glycol according to [401] or [402], further including an alkaline treatment step of treating a process stream containing 1,3-butylene glycol with a base.

[404] The method for manufacturing 1,3-butylene glycol according to any one of [401] to [403], further including a desalting step of removing a salt in a process stream containing 1,3-butylene glycol.

[405] The method for manufacturing 1,3-butylene glycol according to any one of [401] to [404], further including a dealcoholization step of removing a low boiling substance containing alcohols in a process stream containing 1,3-butylene glycol.

[406] The method for manufacturing 1,3-butylene glycol according to any one of [401] to [405], wherein the liquid feed into the dehydration column has an acetaldehyde content of 155 ppm or lower (or 140 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, 3 ppm or lower, 2 ppm or lower, or 1 ppm or lower), and a crotonaldehyde content of 117 ppm or lower (or 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, 3 ppm or lower, 2 ppm or lower, or 1 ppm or lower).

[407] The method for manufacturing 1,3-butylene glycol according to any one of [401] to [406], wherein the liquid feed into the dehydration column has a water content of 90 wt. % or lower (or 85 wt. % or lower, 80 wt. % or lower, 70 wt. % or lower, 60 wt. % or lower, 50 wt. % or lower, 40 wt. % or lower, 35 wt. % or lower, 30 wt. % or lower, or 25 wt. % or lower).

[408] The method for manufacturing 1,3-butylene glycol according to any one of [401] to [407], wherein the dehydration column has the number of theoretical plates from 1 to 100.

[409] The method for manufacturing 1,3-butylene glycol according to any one of [401] to [408], wherein the liquid feed into the product column has a 1,3-butylene glycol concentration of 90 GC area % or higher (or 92 GC area % or higher, 95 GC area % or higher, more preferably 97 GC area % or higher, 97.1 GC area % or higher, 97.2 GC area % or higher, 97.3 GC area % or higher, 97.4 GC area % or higher, 97 0.5 GC area % or higher, 97.6 GC area % or higher, 97.7 GC area % or higher, 97.8 GC area % or higher, 97.9 GC area % or higher, 98 GC area % or higher, 98.1 GC area % or higher, 98.2 GC area % or higher, 98.3 GC area % or higher, 98.4 GC area % or higher, 98.5 GC area % or higher, 98.6 GC area % or higher, 98.7 GC area % or higher, 98.8 GC area % or higher, 98.9 GC area % or higher, or 99 GC area % or higher), and has a water content of 3 wt. % or lower (or 2.8 wt. % or lower, 2.6 wt. % or lower, 2.4 wt. % or lower, 2.2 wt. % or lower, 2 wt. % or lower, 1.8 wt. % or lower, 1.6 wt. % or lower, 1.4 wt. % or lower, 1.2 wt. % or lower, 1.1 wt. % or lower, 1.0 wt. % or lower, 0.95 wt. % or lower, 0.9 wt. % or lower, 0.8 wt. % or lower, 0.7 wt. % or lower, 0.6 wt. % or lower, 0.5 wt. % or lower, 0.4 wt. % or lower, 0.3 wt. % or lower, 0.2 wt. % or lower, or 0.1 wt. % or lower).

[410] The method for manufacturing 1,3-butylene glycol according to any one of [401] to [409], wherein the liquid feed into the product column has a content of the low boiling point component other than water of 1.8 GC area % or lower (or 1.6 GC area % or lower, 1.4 GC area % or lower, 1.2 GC area % or lower, 1.1 GC area % or lower, 1 GC area % or lower, 0.9 GC area % or lower, 0.8 GC area % or lower, 0.7 GC area % or lower, 0.6 GC area % or lower, 0.5 GC area % or lower, 0.4 GC area % or lower, 0.3 GC area % or lower, 0.2 GC area % or lower, or 0.1 GC area % or lower).

[411] The method for manufacturing 1,3-butylene glycol according to any one of [401] to [410], wherein the liquid feed into the product column has the acetaldehyde content of 205 ppm or lower (or 200 ppm or lower, 150 ppm or lower, 120 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, or lower than 2 ppm), and the crotonaldehyde content of 110 ppm or lower (or 100 ppm or lower, 80 ppm or lower, 70 ppm or lower, 60 ppm or lower, 50 ppm or lower, 40 ppm or lower, ppm or lower, 20 ppm or lower, 10 ppm or lower, 5 ppm or lower, 3 ppm or lower, 2 ppm or lower, or lower than 1 ppm).

[412] The method for manufacturing 1,3-butylene glycol according to any one of [401] to [411], wherein the product column has a distillation rate of lower than 30 wt. %, (or 29 wt. % or lower, 28 wt. % or lower, 27 wt. % or lower, 26 wt. % or lower, 25 wt. % or lower, 24 wt. % or lower, 23 wt. % or lower, 22 wt. % or lower, 21 wt. % or lower, 20 wt. % or lower, 19 wt. % or lower, 18 wt. % or lower, 17 wt. % or lower, 16 wt. % or lower, 15 wt. % or lower, 12 wt. % or lower, 10 wt. % or lower, 8 wt. % or lower, 5 wt. % or lower, 3 wt. % or lower, 2 wt. % or lower, 1 wt. % or lower, 0.8 wt. % or lower, 0.6 wt. % or lower, or 0.4 wt. % or lower).

[413] The method for manufacturing 1,3-butylene glycol according to any one of [401] to [412], wherein the product columns has the number of theoretical plates from 1 to 100.

[414] The method for manufacturing 1,3-butylene glycol according to any one of [401] to [413], wherein at least a portion of a distillate from the product column is recycled to a step prior to the product distillation step, namely the dehydration step, a dealcoholization step, a low boiling substance removal step, or another step prior to these steps.

[415] The method for manufacturing 1,3-butylene glycol according to [414], wherein the crude reaction liquid containing 1,3-butylene glycol is a crude reaction liquid obtained by hydrogen reduction of an acetaldol, and at least a portion of the distillate from the product column is recycled to the hydrogen reduction of an acetaldol or a step upstream of the hydrogen reduction.

[416] The method for manufacturing 1,3-butylene glycol according to [414] or [415], wherein an amount of the distillate from the product column being recycled to a step prior to the product distillation is lower than 30 wt. % with respect to a charged amount into the product column within a range not higher than a distilled amount in the product column.

[417]A 1,3-butylene glycol product, having an initial boiling point of higher than 203 °C (or 204 °C or higher, 205 °C or higher, 206 °C or higher, 207 °C or higher, or 208 °C or higher) and a potassium permanganate test value of 30 minutes or longer (or longer than 30 minutes, 32 minutes or longer, 35 minutes or longer, 40 minutes or longer, 50 minutes or longer, or 60 minutes or longer).

[418] The 1,3-butylene glycol product according to [417],
having, according to a gas chromatographic analysis performed under conditions set forth below:
an area ratio of a 1,3-butylene glycol peak of higher than 98.7% (or 98.8% or higher, 98.9% or higher, 99% or higher, 99.1% or higher, 99.2% or higher, 99.3% or higher, 99.4% or higher, 99.5% or higher, 99.6% or higher, 99.7% or higher, or 99.8% or higher), and
a total area ratio of peaks having shorter retention times than the 1,3-butylene glycol peak of lower than 0.3% (or 0.28% or lower, 0.25% or lower, 0.23% or lower, 0.2% or lower, 0.17% or lower, 0.15% or lower. 0.12% or lower, 0.1% or lower, 0.07% or lower, 0.04% or lower, 0.03% or lower, 0.02% or lower, 0.01% or lower, 0.007% or lower, 0.005% or lower, or 0.002% or lower), and having: a water content of lower than 0.4 wt. % (or 0.3 wt. % or lower, 0.2 wt. % or lower, 0.1 wt. % or lower, 0.07 wt. % or lower, 0.05 wt. % or lower, 0.03 wt. % or lower, 0.02 wt. % or lower, 0.01 wt. % or lower, or 0.005 wt. % or lower);
an acetaldehyde content of lower than 2 ppm (or 1.8 ppm or lower, 1.7 ppm or lower, 1.5 ppm or lower, 1.4 ppm or lower, 1.3 ppm or lower, 1.2 ppm or lower, 1.0 ppm or lower, 1.1 ppm or lower, 0.9 ppm or lower, 0.8 ppm or lower, 0.7 ppm or lower, 0.6 ppm or lower, 0.5 ppm or lower, 0.3 ppm or lower, or 0.2 ppm or lower); and
a crotonaldehyde content of lower than 1.2 ppm (or 1.0 ppm or lower, 0.9 ppm or lower, 0.8 ppm or lower, 0.7 ppm or lower, 0.6 ppm or lower, 0.5 ppm or lower, 0.4 ppm or lower, 0.3 ppm or lower, 0.2 ppm or lower, or 0.1 ppm or lower),
wherein the conditions for the gas chromatographic analysis are as follows:
Analytical Column: a column with dimethylpolysiloxane as a stationary phase, having a length of 30 m, an inner diameter of 0.25 mm, and a film thickness of 1.0 μm
Heating Conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

[419]A moisturizer containing the 1,3-butylene glycol product described in [417] or [418].

[420] The moisturizer according to [419], wherein a content of the 1,3-butylene glycol product described in [417] or [418] is 10 wt. % or higher (or 30 wt. % or higher, 50 wt. % or higher, 80 wt. % or higher, or 90 wt. % or higher).

[421]A cosmetic product containing the moisturizer described in [419] or [420].

[422] The cosmetic product according to [421], wherein a content of the 1,3-butylene glycol product described in [417] or [418] is from 0.01 to 40 wt. % (or from 0.1 to 30 wt. %, from 0.2 to 20 wt. %, from 0.5 to 15 wt. %, or from 1 to 10 wt. %).

[423] The cosmetic product according to [421] or [422], which is a skin cosmetic product, a hair cosmetic product, a sunscreen cosmetic product, or a make-up cosmetic product.

[501]A method for manufacturing 1,3-butylene glycol, which is a method for obtaining purified 1,3-butylene glycol from a crude reaction liquid containing 1,3-butylene glycol, the method including:
a dehydration step of removing water by distillation; a high boiling point substance removal step of removing a high boiling point component by distillation; and a product distillation step of obtaining purified 1,3-butylene glycol,
wherein in the dehydration step, a dehydration column is used in which a liquid feed containing 1,3 butylene glycol and water with an acetaldehyde content of 1000 ppm or lower (or 900 ppm or lower, 800 ppm or lower, 700 ppm or lower, 600 ppm or lower, 500 ppm or lower, 400 ppm or lower, 300 ppm or lower, 200 ppm or lower, 155 ppm or lower, 140 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, 3 ppm or lower, 2 ppm or lower, or 1 ppm or lower) and a crotonaldehyde content of 400 ppm or lower (or 200 ppm or lower, 150 ppm or lower, 130 ppm or lower, 117 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, 3 ppm or lower, 2 ppm or lower, or 1 ppm or lower) is distilled under a condition of a reflux ratio of higher than 0.3 (or 0.4 or higher, 0.5 or higher, 0.6 or higher, 0.7 or higher, 0.8 or higher, 0.9 or higher, 1 or higher, 1.1 or higher, 1.2 or higher, 1.3 or higher, 1.4 or higher, 1.5 or higher, 1.6 or higher, 1.7 or higher, 1.8 or higher, 1.9 or higher, 2 or higher, 3 or higher, 4 or higher, 5 or higher, 6 or higher, 7 or higher, 8 9 or higher, 10 or higher, 15 or higher, 20 or higher, 25 or higher, 30 or higher, or 40 or higher), and a liquid concentrated with a low boiling point component containing water is distilled off from above a feed plate,
in the high boiling point substance removal step, a high boiling substance removal column is used in which a liquid feed containing 1,3-butylene glycol is distilled under a condition of a reflux ratio of higher than 0.02 (or 0.05 or higher, 0.1 or higher, 0.2 or higher, 0.3 or higher, 0.4 or higher, 0.5 or higher, 0.6 or higher, 0.7 or higher, 0.8 or higher, 0.9 or higher, 1 or higher, 1.2 or higher, 1.5 or higher, 2 or higher, 3 or higher, 4 or higher, 5 or higher, 10 or higher, or 20 or higher), higher-purity 1,3-butylene glycol is distilled off from above a feed plate, and a liquid concentrated with a high boiling point component is extracted from below the feed plate, and
in the product distillation step, a product column is used in which a 1,3-butylene glycol liquid feed with an acetaldehyde content of 500 ppm or lower (or 205 ppm or lower, 200 ppm or lower, 150 ppm or lower, 120 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, or lower than 2 ppm) and a crotonaldehyde content of 200 ppm or lower (or 205 ppm or lower, 200 ppm or lower, 150 ppm or lower, 120 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, ppm or lower, 5 ppm or lower, or lower than 2 ppm) is distilled under a condition of a reflux ratio of higher than 0.1 (or 0.2 or higher, 0.3 or higher, 0.4 or higher, 0.5 or higher, 1 or higher, 2 or higher, 3 or higher, 4 or higher, 5 or higher, 6 or higher, 7 or higher, 8 or higher, 9 or higher, 10 or higher, 20 or higher, 50 or higher, 400 or higher, or 500 or higher), a liquid concentrated with a low boiling point component is distilled off from above a feed plate, and 1,3-butylene glycol is extracted from below the feed plate.

[502] The method for manufacturing 1,3-butylene glycol according to [501], wherein the liquid feed into the product column has a 1,3-butylene glycol concentration of 90 GC area % or higher (or 92 GC area % or higher, 95 GC area % or higher, more preferably 97 GC area % or higher, 97.1 GC area % or higher, 97.2 GC area % or higher, 97.3 GC area % or higher, 97.4 GC area % or higher, 97.5 GC area % or higher, 97.6 GC area % or higher, 97.7 GC area % or higher, 97.8 GC area % or higher, 97.9 GC area % or higher, 98 GC area % or higher, 98.1 GC area % or higher, 98.2 GC area % or higher, 98.3 GC area % or higher, 98.4 GC area % or higher, 98.5 GC area % or higher, 98.6 GC area % or higher, 98.7 GC area % or higher, 98.8 GC area % or higher, 98.9 GC area % or higher, or 99 GC area % or higher), and the liquid feed has a water content of 3 wt. % or lower (or 1.2 wt. % or lower, 1.1 wt. % or lower, 1.0 wt. % or lower, 0.95 wt. % or lower, 0.9 wt. % or lower, 0.8 wt. % or lower, 0.7 wt. % or lower, 0.6 wt. % or lower, 0.5 wt. % or lower, 0.4 wt. % or lower, 0.3 wt. % or lower, 0.2 wt. % or lower, or 0.1 wt. % or lower).

[503] The method for manufacturing 1,3-butylene glycol according to [501] or [502], wherein the liquid feed into the product column has a content of the low boiling point component other than water of 1.8 GC area % or lower (or 1.6 GC area % or lower, 1.4 GC area % or lower, 1.2 GC area % or lower, 1.1 GC area % or lower, 1 GC area % or lower, 0.9 GC area % or lower, 0.8 GC area % or lower, 0.7 GC area % or lower, 0.6 GC area % or lower, 0.5 GC area % or lower, 0.4 GC area % or lower, 0.3 GC area % or lower, 0.2 GC area % or lower, or 0.1 GC area % or lower).

[504] The method for manufacturing 1,3-butylene glycol according to any one of [501] to [503], wherein the liquid feed into the product column has an acetaldehyde content of 205 ppm or lower (or 200 ppm or lower, 150 ppm or lower, 120 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, or lower than 2 ppm), and the liquid feed has a crotonaldehyde content of 110 ppm or lower (100 ppm or lower, 80 ppm or lower, 70 ppm or lower, 60 ppm or lower, 50 ppm or lower, ppm or lower, 30 ppm or lower, 20 ppm or lower, 10 ppm or lower, 5 ppm or lower, 3 ppm or lower, 2 ppm or lower, or lower than 1 ppm).

[505] The method for manufacturing 1,3-butylene glycol according to any one of [501] to [504], wherein the product columns has the number of theoretical plates of from 1 to 100.

[506] The method for manufacturing 1,3-butylene glycol according to any one of [501] to [505], wherein at least a portion of a distillate from the product column is recycled to a step prior to the product distillation step, namely the dehydration step, a dealcoholization step, a low boiling point substance removal step, or another step prior to these steps.

[507]A method for manufacturing 1,3-butylene glycol, which is a method for obtaining purified 1,3-butylene glycol from a crude reaction liquid containing 1,3-butylene glycol, the method including:
a dehydration step of removing water by distillation; and a high boiling point substance removal step of removing a high boiling point component by distillation,
wherein in the dehydration step, a dehydration column is used in which a liquid feed containing 1,3 butylene glycol and water with an acetaldehyde content of 1000 ppm or lower (or 900 ppm or lower, 800 ppm or lower, 700 ppm or lower, 600 ppm or lower, 500 ppm or lower, 400 ppm or lower, 300 ppm or lower, 200 ppm or lower, 155 ppm or lower, 140 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, 3 ppm or lower, 2 ppm or lower, or 1 ppm or lower) and a crotonaldehyde content of 400 ppm or lower (or 200 ppm or lower, 150 ppm or lower, 130 ppm or lower, 117 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, 3 ppm or lower, 2 ppm or lower, or 1 ppm or lower) is distilled under a condition of a reflux ratio of higher than 0.3 (or 0.4 or higher, 0.5 or higher, 0.6 or higher, 0.7 or higher, 0.8 or higher, 0.9 or higher, 1 or higher, 1.1 or higher, 1.2 or higher, 1.3 or higher, 1.4 or higher, 1.5 or higher, 1.6 or higher, 1.7 or higher, 1.8 or higher, 1.9 or higher, 2 or higher, 3 or higher, 4 or higher, 5 or higher, 6 or higher, 7 or higher, 8 or higher, 9 or higher, 10 or higher, 15 or higher, 20 or higher, 25 or higher, 30 or higher, or 40 or higher), and a liquid concentrated with a low boiling point component containing water is distilled off from above a feed plate, and
in the high boiling point substance removal step, a high boiling substance removal column is used in which a liquid feed containing 1,3-butylene glycol is distilled under a condition of a reflux ratio of higher than 0.02 (or 0.1 or higher, 0.2 or higher, 0.3 or higher, 0.4 or higher, 0.5 or higher, 0.6 or higher, 0.7 or higher, 0.8 or higher, 0.9 or higher, 1 or higher, 1.2 or higher, 1.5 or higher, 2 or higher, 3 or higher, 4 or higher, 5 or higher, 10 or higher, or 20 or higher), higher-purity 1,3-butylene glycol is distilled off from above a feed plate, and a liquid concentrated with the high boiling point component is extracted from below the feed plate.

[508] The method for manufacturing 1,3-butylene glycol according to any one of [501] to [507], wherein the crude reaction liquid containing 1,3-butylene glycol is a crude reaction liquid obtained by hydrogen reduction of an acetaldol.

[509] The method for manufacturing 1,3-butylene glycol according to any one of [501] to [508], further including an alkaline treatment step of treating a process stream containing 1,3-butylene glycol with a base.

[510] The method for manufacturing 1,3-butylene glycol according to any one of [501] to [509], further including a desalting step of removing a salt in a process stream containing 1,3-butylene glycol.

[511] The method for manufacturing 1,3-butylene glycol according to any one of [501] to [510], further including a dealcoholization step of removing a low boiling substance containing an alcohol in a process stream containing 1,3-butylene glycol.

[512] The method for manufacturing 1,3-butylene glycol according to any one of [501] to [511], wherein the liquid feed into the dehydration column has a water content of 90 wt. % or lower (or 85 wt. % or lower, 80 wt. % or lower, 70 wt. % or lower, 60 wt. % or lower, 50 wt. % or lower, 40 wt. % or lower, 35 wt. % or lower, 30 wt. % or lower, or 25 wt. % or lower).

[513] The method for manufacturing 1,3-butylene glycol according to any one of [501] to [512], wherein in the dehydration column and/or the high boiling substance removal column, the number of theoretical plates in at least one of these distillation columns is from 1 to 100.

[514] The method for manufacturing 1,3-butylene glycol according to any one of [501] to [513], wherein the liquid feed into the high boiling substance removal column has a 1,3-butylene glycol concentration of 95% or higher (or 96% or higher, 97% or higher, 98% or higher, or 99% or higher).

[515] The method for manufacturing 1,3-butylene glycol according to any one of [501] to [514], wherein the liquid feed into the high boiling substance removal column has a high boiling point component content of 4% or lower (or 3% or lower, 2% or lower, 1% or lower, 0.5% or lower, 0.4% or lower, 0.3% or lower, 0.2% or lower, 0.1% or lower, 0.05% or lower, or 0.01% or lower).

[516]A 1,3-butylene glycol product having an initial boiling point of higher than 203 °C (or 204 °C or higher, 205 °C or higher, 206 °C or higher, 207 °C, or 208 °C or higher), and a dry point of 209 °C or lower.

[517] The 1,3-butylene glycol product according to [516], having a potassium permanganate test value of 30 minutes or longer (or longer than 30 minutes, 32 minutes or longer, 35 minutes or longer, 40 minutes or longer, 50 minutes or longer, or 60 minutes or longer).

[518] The 1,3-butylene glycol product according to [516] or [517],
having, according to a gas chromatographic analysis performed under conditions set forth below:
an area ratio of a 1,3-butylene glycol peak of higher than 98.7% (or 98.8% or higher, 98.9% or higher, 99% or higher, 99.1% or higher, 99.2% or higher, 99.3% or higher, 99.4% or higher, 99.5% or higher, 99 6.6% or higher, 99.7% or higher, or 99.8% or higher),
a total area ratio of peaks having shorter retention times than the 1,3-butylene glycol peak of lower than 0.3% (or 0.28% or lower, 0.25% or lower, 0.23% or lower, 0.2% or lower, 0.17% or lower, 0.15% or lower, 0.12% or lower, 0.1% or lower, 0.07% or lower, 0.04% or lower, 0.03% or lower, 0.02% or lower, 0.01% or lower, 0.007% or lower, 0.005% or lower, or 0.002% or lower), and
a total area ratio of peaks having longer retention times than the 1,3-butylene glycol peak of lower than 1.2% (or 1% or lower, 0.9% or lower, 0.8% or lower, 0.7% or lower, 0.6% or lower, 0.5% or lower, 0.4% or lower, 0.3% or lower, 0.2% or lower, or 0.1% or lower), and
having: a water content of lower than 0.4 wt. % (or 0.3 wt. % or lower, 0.2 wt. % or lower, 0.1 wt. % or lower, 0.07 wt. % or lower, 0.05 wt. % or lower, 0.03 wt. % or lower, 0.02 wt. % or lower, 0.01 wt. % or lower, or 0.005 wt. % or lower);
an acetaldehyde content of lower than 2 ppm (or 1.8 ppm or lower, 1.7 ppm or lower, 1.5 ppm or lower, 1.4 ppm or lower, 1.3 ppm or lower, 1.2 ppm or lower, 1.1 ppm or lower, 1.0 ppm or lower, 0.9 ppm or lower, 0.8 ppm or lower, 0.7 ppm or lower, 0.6 ppm or lower, 0.5 ppm or lower, 0.3 ppm or lower, or 0.2 ppm or lower); and
a crotonaldehyde content of lower than 1.2 ppm (or 1.0 ppm or lower, 0.9 ppm or lower, 0.8 ppm or lower, 0.7 ppm or lower, 0.6 ppm or lower, 0.5 ppm or lower, 0.4 ppm or lower, 0.3 ppm or lower, 0.2 ppm or lower, or 0.1 ppm or lower),
in which the conditions for the gas chromatographic analysis are as follows:
Analytical Column: a column with dimethylpolysiloxane as a stationary phase, having a length of 30 m, an inner diameter of 0.25 mm, and a film thickness of 1.0 μm
Heating Conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

[519]A moisturizer containing the 1,3-butylene glycol product described in any one of [516] to [518].

[520] The moisturizer according to [519], wherein a content of the 1,3-butylene glycol product described in any one of [516] to [518] is 10 wt. % or higher (or 30 wt. % or higher, 50 wt. % or higher, 80 wt. % or higher, or 90 wt. % or higher).

[521]A cosmetic product containing the moisturizer described in [519] or [520].

[522] The cosmetic product according to [521], wherein a content of the 1,3-butylene glycol product described in any one of [516] to [518] is from 0.01 to 40 wt. % (or from 0.1 to 30 wt. %, from 0.2 to 20 wt. %, from 0.5 to 15 wt. %, or from 1 to 10 wt. %).

[523] The cosmetic product according to [521] or [522], which is a skin cosmetic product, a hair cosmetic product, a sunscreen cosmetic product, or a make-up cosmetic product.

[601]A method for manufacturing 1,3-butylene glycol, which is a method for obtaining purified 1,3-butylene glycol from a crude reaction liquid containing 1,3-butylene glycol,
the method including: a dehydration step of removing water by distillation; a high boiling substance removal step of removing a high boiling point component by distillation; and a product distillation step of obtaining purified 1,3-butylene glycol,
wherein in the dehydration step, a dehydration column is used in which a liquid feed containing 1,3-butylene glycol and water with an acetaldehyde content of 1000 ppm or lower (or 900 ppm or lower, 800 ppm or lower, 700 ppm or lower, 600 ppm or lower, 500 ppm or lower, 400 ppm or lower, 300 ppm or lower, 200 ppm or lower, 155 ppm or lower, 140 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, 3 ppm or lower, 2 ppm or lower, or 1 ppm or lower) and a crotonaldehyde content of 400 ppm or lower (or 200 ppm or lower, 150 ppm or lower, 130 ppm or lower, 117 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, 3 ppm or lower, 2 ppm or lower, or 1 ppm or lower) is distilled, and a liquid concentrated with a low boiling point component containing water is distilled off from above a feed plate, and
in the product distillation step, a product column is used in which a 1,3-butylene glycol liquid feed having an acetaldehyde content of 500 ppm or lower (or 205 ppm or lower, 200 ppm or lower, 150 ppm or lower, 120 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, or lower than 2 ppm) and a crotonaldehyde content of 200 ppm or lower (or 205 ppm or lower, 200 ppm or lower, 150 ppm or lower, 120 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, or lower than 2 ppm) is distilled under a condition of a reflux ratio of higher than 0.1 (or 0.2 or higher, 0.3 or higher, 0.4 or higher, 0.5 or higher, 1 or higher, 2 or higher, 3 or higher, 4 or higher, or higher, 6 or higher, 7 or higher, 8 or higher, 9 or higher, 10 or higher, 20 or higher, 50 or higher, 400 or higher, or 500 or higher), a liquid concentrated with a low boiling point component is distilled off from above a feed plate, and 1,3-butylene glycol is extracted from below the feed plate.

[602] The method for manufacturing 1,3-butylene glycol according to [601], wherein in the high boiling substance removal step, a high boiling substance removal column is used in which a liquid feed containing 1,3-butylene glycol is distilled under a condition of a reflux ratio of 0.02 or higher (or 0.03 or higher, 0.04 or higher, 0.05 or higher, 0.1 or higher, 0.2 or higher, 0.3 or higher, 0.4 or higher, 0.5 or higher, 0.6 or higher, 0.7 or higher, 0.8 or higher, 0.9 or higher, 1 or higher, 1.2 or higher, 1.5 or higher, 2 or higher, 3 or higher, 4 or higher, 5 or higher, 10 or higher, or 20 or higher), 1,3-butylene glycol having further improved purity is distilled off from above a feed plate, and a liquid concentrated with a high boiling point component is extracted from below the feed plate.

[603] The method for manufacturing 1,3-butylene glycol according to [601] or [602], wherein the liquid feed into the product column has a 1,3-butylene glycol concentration of 90 GC area % or higher (or 92 GC area % or higher, 95 GC area % or higher, more preferably 97 GC area % or higher, 97.1 GC area % or higher, 97.2 GC area % or higher, 97.3 GC area % or higher, 97.4 GC area % or higher, 97.5 GC area % or higher, 97.6 GC area % or higher, 97.7 GC area % or higher, 97.8 GC area % or higher, 97.9 GC area % or higher, 98 GC area % or higher, 98.1 GC area % or higher, 98.2 GC area % or higher, 98.3 GC area % or higher, 98.4 GC area % or higher, 98.5 GC area % or higher, 98.6 GC area % or higher, 98.7 GC area % or higher, 98.8 GC area % or higher, 98.9 GC area % or higher, or 99 GC area % or higher), and a content of water in the liquid feed is 3 wt. % or lower (or 1.2 wt. % or lower, 1.1 wt. % or lower, 1.0 wt. % or lower, 0.95 wt. % or lower, 0.9 wt. % or lower, 0.8 wt. % or lower, 0.7 wt. % or lower, 0.6 wt. % or lower, 0.5 wt. % or lower, 0.4 wt. % or lower, 0.3 wt. % or lower, 0.2 wt. % or lower, or 0.1 wt. % or lower).

[604] The method for manufacturing 1,3-butylene glycol according to any one of [601] to [603], wherein the liquid feed into the product column has a content of the low boiling point component other than water of 1.8 GC area % or lower (or 1.6 GC area % or lower, 1.4 GC area % or lower, 1.2 GC area % or lower, 1.1 GC area % or lower, 1 GC area % or lower, 0.9 GC area % or lower, 0.8 GC area % or lower, 0.7 GC area % or lower, 0.6 GC area % or lower, 0.5 GC area % or lower, 0.4 GC area % or lower, 0.3 GC area % or lower, 0.2 GC area % or lower, or 0.1 GC area % or lower).

[605] The method for manufacturing 1,3-butylene glycol according to any one of [601] to [604], wherein the liquid feed into the product column the acetaldehyde content of 205 ppm or lower (or 200 ppm or lower, 150 ppm or lower, 120 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, or lower than 2 ppm), and the content of crotonaldehyde in the liquid feed is 110 ppm or lower (100 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, 3 ppm or lower, 2 ppm or lower, or lower than 1 ppm).

[606] The method for manufacturing 1,3-butylene glycol according to any one of [601] to [605], wherein the product columns have the number of theoretical plates of from 1 to 100.

[607] The method for manufacturing 1,3-butylene glycol according to any one of [601] to [606], wherein at least a portion of a distillate from the product column is recycled to a step prior to the product distillation step, namely the dehydration step, the dealcoholization step, the low boiling substance removal step, or another step prior to these steps.

[608]A method for manufacturing 1,3-butylene glycol, which is a method for obtaining purified 1,3-butylene glycol from a crude reaction liquid containing 1,3-butylene glycol, the method including:
a dehydration step of removing water by distillation; and a high boiling substance removal step of removing a high boiling point component by distillation,
wherein in the dehydration step, a dehydration column is used in which a liquid feed containing 1,3-butylene glycol and water with an acetaldehyde content of 1000 ppm or lower (or 900 ppm or lower, 800 ppm or lower, 700 ppm or lower, 600 ppm or lower, 500 ppm or lower, 400 ppm or lower, 300 ppm or lower, 200 ppm or lower, 155 ppm or lower, 140 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, 3 ppm or lower, 2 ppm or lower, or 1 ppm or lower) and a crotonaldehyde content of 400 ppm or lower (or 200 ppm or lower, 150 ppm or lower, 130 ppm or lower, 117 ppm or lower, 100 ppm or lower, 90 ppm or lower, 80 ppm or lower, 70 ppm or lower, 60 ppm or lower, 50 ppm or lower, 40 ppm or lower, 30 ppm or lower, 20 ppm or lower, 10 ppm or lower, 5 ppm or lower, 3 ppm or lower, 2 ppm or lower, or 1 ppm or lower) is distilled, and a liquid concentrated with a low boiling point component containing water is distilled off from above a feed plate, and
in the high boiling substance removal step, a high boiling substance removal column is used in which a liquid feed containing 1,3-butylene glycol is distilled under a condition of a reflux ratio of higher than 0.02 (or 0.1 or higher, 0.2 or higher, 0.3 or higher, 0.4 or higher, 0.5 or higher, 0.6 or higher, 0.7 or higher, 0.8 or higher, 0.9 or higher, 1 or higher, 1.2 or higher, 1.5 or higher, 2 or higher, 3 or higher, 4 or higher, 5 or higher, 10 or higher, or 20 or higher), higher-purity 1,3-butylene glycol is distilled off from above a feed plate, and a liquid concentrated with the high boiling point component is extracted from below the feed plate.

[609] The method for manufacturing 1,3-butylene glycol according to any one of [601] to [608], wherein the dehydration column has a reflux ratio of 0.03 or higher (or 0.04 or higher, 0.05 or higher, 0.06 or higher, 0.07 or higher, 0.08 or higher, 0.09 or higher, 0.1 or higher, 0.12 or higher, 0.14 or higher, 0.16 or higher, 0.18 or higher, 0.2 or higher, 0.22 or higher, 0.24 or higher, 0.26 or higher, 0.28 or higher, 0.3 or higher, 0.33 or higher, 0.36 or higher, 0.4 or higher, 0.45 or higher, 0.5 or higher, 0.6 or higher, 0.7 or higher, 0.8 or higher, 0.9 or higher, 1 or higher, 1.1 or higher, 1.2 or higher, 1.3 or higher, 1.4 or higher, 1.5 or higher, 1.6 or higher, 1.7 or higher, 1.8 or higher, 1.9 or higher, 2 or higher, 3 or higher, 4 or higher, 5 or higher, 6 or higher, 7 or higher, 8 or higher, 9 or higher, 10 or higher, 15 or higher, 20 or higher, 25 or higher, 30 or higher, or 40 or higher).

[610] The method for manufacturing 1,3-butylene glycol according to any one of [601] to [609], wherein the crude reaction liquid containing 1,3-butylene glycol is a crude reaction liquid obtained by hydrogen reduction of an acetaldol.

[611] The method for manufacturing 1,3-butylene glycol according to any one of [601] to [610], further including an alkaline treatment step of treating a process stream containing 1,3-butylene glycol with a base.

[612] The method for manufacturing 1,3-butylene glycol according to any one of [601] to [611], further including a desalting step of removing a salt in a process stream containing 1,3-butylene glycol.

[613] The method for manufacturing 1,3-butylene glycol according to any one of [601] to [612], further including a dealcoholization step of removing a low boiling substance containing alcohols in a process stream containing 1,3-butylene glycol.

[614] The method for manufacturing 1,3-butylene glycol according to any one of [601] to [613], wherein the liquid feed into the dehydration column has a water content of 90 wt. % or lower (or 85 wt. % or lower, 80 wt. % or lower, 70 wt. % or lower, 60 wt. % or lower, 50 wt. % or lower, 40 wt. % or lower, 35 wt. % or lower, 30 wt. % or lower, or 25 wt. % or lower).

[615] The method for manufacturing 1,3-butylene glycol according to any one of [601] to [614], wherein in the dehydration column and/or the high boiling substance removal column, the number of theoretical plates in at least one of these distillation columns is 1 to 100.

[616] The method for manufacturing 1,3-butylene glycol according to any one of [601] to [615], wherein the liquid feed into the high boiling substance removal column has a 1,3-butylene glycol concentration of 95% or higher (or 96% or higher, 97% or higher, 98% or higher, or 99% or higher).

[617] The method for manufacturing 1,3-butylene glycol according to any one of [601] to [616], wherein the liquid feed into the high boiling substance removal column has a content of the high boiling point component of 4% or lower (or 3% or lower, 2% or lower, 1% or lower, 0.5% or lower, 0.4% or lower, 0.3% or lower, 0.2% or lower, 0.1% or lower, 0.05% or lower, or 0.01% or lower).

[618]A 1,3-butylene glycol product having an initial boiling point of higher than 203 °C (or 204 °C or higher, 205 °C or higher, 206 °C or higher, 207 °C, or 208 °C or higher) and a potassium permanganate test value of 30 minutes or longer (or longer than 30 minutes, 32 minutes or longer, 35 minutes or longer, 40 minutes or longer, 50 minutes or longer, or 60 minutes or longer).

[619]A 1,3-butylene glycol product having an initial boiling point higher than 203 °C (or 204 °C or higher, 205 °C or higher, 206 °C or higher, 207 °C, or 208 °C or higher), and a dry point of 209 °C or lower.

[620]A 1,3-butylene glycol product having an initial boiling point of higher than 203 °C (or 204 °C or higher, 205 °C or higher, 206 °C or higher, 207 °C, or 208 °C or higher), a dry point of 209 °C or lower, and a potassium permanganate test value of 30 minutes or longer (or longer than 30 minutes, 32 minutes or longer, 35 minutes or longer, 40 minutes or longer, 50 minutes or longer, or 60 minutes or longer).

[621] The 1,3-butylene glycol product according to any one of [618] to [620], which satisfies at least one (preferably 2 or more, more preferably 3 or more, even more preferably 4 or more, particularly preferably 5 or more, and especially all) condition(s) selected from the following six conditions:
(a) an area ratio of a 1,3-butylene glycol peak of higher than 98.7% (or 98.8% or higher, 98.9% or higher, 99% or higher, 99.1% or higher, 99.2% or higher, 99.3% or higher, 99.4% or higher, 99.5% or higher, 99.6% or higher, 99.7% or higher, or 99.8% or higher) according to a gas chromatographic analysis performed under conditions set forth below,
(b) a total area ratio of peaks having shorter retention times than the 1,3-butylene glycol peak of lower than 0.3% (or 0.28% or lower, 0.25% or lower, 0.23% or lower, 0.2% or lower, 0.17% or lower, 0.15% or lower, 0.12% or lower, 0.1% or lower, 0.07% or lower, 0.04% or lower, 0.03% or lower, 0.02% or lower, 0.01% or lower, 0.007% or lower, 0.005% or lower, or 0.002% or lower) according to a gas chromatographic analysis performed under conditions set forth below,
(c) a total area ratio of peaks having longer retention times than the 1,3-butylene glycol peak of lower than 1.2% (or 1% or lower, 0.9% or lower, 0.8% or lower, 0.7% or lower, 0.6% or lower, 0.5% or lower, 0.4% or lower, 0.3% or lower, 0.2% or lower, or 0.1% or lower) according to a gas chromatographic analysis performed under conditions set forth below,
(d) a water content of lower than 0.4 wt. % (or 0.3 wt. % or lower, 0.2 wt. % or lower, 0.1 wt. % or lower, 0.07 wt. % or lower, 0.05 wt. % or lower, 0.03 wt. % or lower, 0.02 wt. % or lower, 0.01 wt. % or lower, or 0.005 wt. % or lower),
(e) an acetaldehyde content of lower than 2 ppm (or 1.8 ppm or lower, 1.7 ppm or lower, 1.5 ppm or lower, 1.4 ppm or lower, 1.3 ppm or lower, 1.2 ppm or lower, 1.1 ppm or lower, 1.0 ppm or lower, 0.9 ppm or lower, 0.8 ppm or lower, 0.7 ppm or lower, 0.6 ppm or lower, 0.5 ppm or lower, 0.3 ppm or lower, or 0.2 ppm or lower), and
(f) a crotonaldehyde content of lower than 1.2 ppm (or 1.0 ppm or lower, 0.9 ppm or lower, 0.8 ppm or lower, 0.7 ppm or lower, 0.6 ppm or lower, 0.5 ppm or lower, 0.4 ppm or lower, 0.3 ppm or lower, 0.2 ppm or lower, or 0.1 ppm or lower),
in which the conditions for the gas chromatographic analysis are as follows:
Analytical Column: a column with dimethylpolysiloxane as a stationary phase, having a length of m, an inner diameter of 0.25 mm, and a film thickness of 1.0 μm
Heating Conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

[622]A moisturizer comprising the 1,3-butylene glycol product described in any one of [618] to [621].

[623] The moisturizer according to [622], wherein a content of the 1,3-butylene glycol product described in any one of [618] to [621] is 10 wt. % or higher (or 30 wt. % or higher, 50 wt. % or higher, 80 wt. % or higher, or 90 wt. % or higher).

[624]A cosmetic product comprising the moisturizer described in [622] or [623].

[625] The cosmetic product according to [624], wherein a content of the 1,3-butylene glycol product described in any one of [618] to [621] is from 0.01 to 40 wt. % (or from 0.1 to 30 wt. %, from 0.2 to 20 wt. %, from 0.5 to 15 wt. %, or from 1 to 10 wt. %).

[626] The cosmetic product according to [624] or [625], which is a skin cosmetic product, a hair cosmetic product, a sunscreen cosmetic product, or a make-up cosmetic product.

[701]A 1,3-butylene glycol product in which at least one of eight contents: a content of methyl vinyl ketone, a content of acetone, a content of butylaldehyde, a content of acetaldol, a content of a compound represented by Formula (1) below, a content of a compound represented by Formula (2) below, a content of a compound represented by Formula (3) below, and a total content of a compound represented by Formula (4) below and a compound represented by Formula (5) below, is less than 8 ppm (or 7 ppm or less, 6 ppm or less, 5 ppm or less, 4 ppm or less, 3 ppm or less, 2 ppm or less, 1 ppm or less, or 0.5 ppm or less).

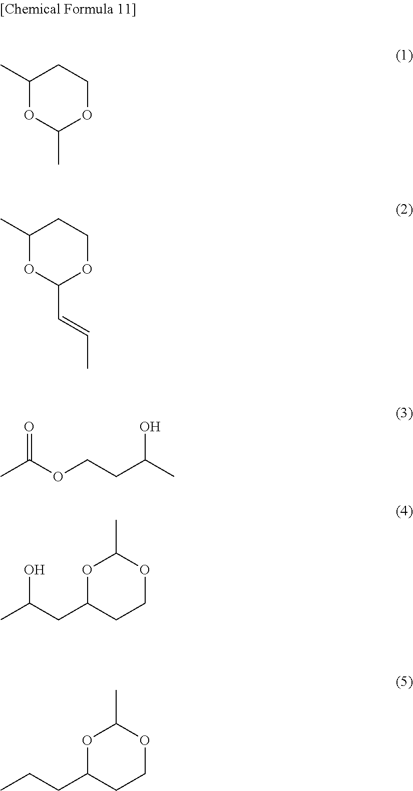

[702] The 1,3-butylene glycol product according to [701], wherein at least two (or three, four, five, six, seven, or eight) contents of the eight contents: the content of methyl vinyl ketone, the content of acetone, the content of butylaldehyde, the content of acetaldol, the content of the compound represented by Formula (1) below, the content of the compound represented by Formula (2) below, the content of the compound represented by Formula (3) below, and the total content of the compound represented by Formula (4) below and the compound represented by Formula (5) below, are each less than 8 ppm (or 7 ppm or less, 6 ppm or less, 5 ppm or less, 4 ppm or less, 3 ppm or less, 2 ppm or less, 1 ppm or less, or 0.5 ppm or less).

[703] The 1,3-butylene glycol product according to [701] or [702], wherein at least four (four, five, six, seven, or eight) contents of the eight contents: the content of methyl vinyl ketone, the content of acetone, the content of butylaldehyde, the content of acetaldol, the content of the compound represented by Formula (1) below, the content of the compound represented by Formula (2) below, the content of the compound represented by Formula (3) below, and the total content of the compound represented by Formula (4) below and the compound represented by Formula (5) below, are each less than 8 ppm (or 7 ppm or less, 6 ppm or less, 5 ppm or less, 4 ppm or less, 3 ppm or less, 2 ppm or less, 1 ppm or less, or 0.5 ppm or less).

[704] The 1,3-butylene glycol product according to any one of [701] to [703], wherein a sum of the content of methyl vinyl ketone, the content of acetone, the content of butylaldehyde, the content of acetaldol, the content of the compound represented by Formula (1), the content of the compound represented by Formula (2), the content of the compound represented by Formula (3), and the content of the compound represented by Formula (4) and the content of the compound represented by Formula (5) is less than 71 ppm (or 60 ppm or less, 50 ppm or less, 40 ppm or less, 30 ppm or less, 20 ppm or less, 18 ppm or less, 16 ppm or less, 14 ppm or less, 12 ppm or less, 10 ppm or less, 8 ppm or less, 7 ppm or less, 6 ppm or less, 5 ppm or less, 4 ppm or less, 3 ppm or less, 2 ppm or less, 1 ppm or less, or 0.5 ppm or less).

[705] The 1,3-butylene glycol product according to any one of [701] to [704], wherein a sum of the content of methyl vinyl ketone, the content of acetone, the content of butylaldehyde, the content of acetaldol, and the content of the compound represented by Formula (1) is less than 47 ppm (or, 40 ppm or less, 30 ppm or less, 25 ppm or less, 20 ppm or less, 18 ppm or less, 16 ppm or less, 14 ppm or less, 12 ppm or less, 10 ppm or less, 8 ppm or less, 7 ppm or less, 6 ppm or less, 5 ppm or less, 4 ppm or less, 3 ppm or less, 2 ppm or less, 1 ppm or less, or 0.5 ppm or less), and a sum of the content of the compound represented by Formula (2), the content of the compound represented by Formula (3), the content of the compound represented by Formula (4), and the content of the compound represented by Formula (5) is less than 24 ppm (or 20 ppm or less, 18 ppm or less, 16 ppm or less, 14 ppm or less, 13 ppm or less, 12 ppm or less, 11 μm or less, 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, or 0.5 ppm or less).

[706] The 1,3-butylene glycol product according to any one of [701] to [705], wherein at least the content of acetaldol is less than 8 ppm (or 7 ppm or less, 6 ppm or less, 5 ppm or less, 4 ppm or less, 3 ppm or less, 2 ppm or less, 1 ppm or less, or 0.5 ppm or less).

[707] The 1,3-butylene glycol product according to any one of [701] to [706], wherein at least the content of the compound represented by Formula (3) is less than 8 ppm (or 7 ppm or less, 6 ppm or less, 5 ppm or less, 4 ppm or less, 3 ppm or less, 2 ppm or less, 1 ppm or less, or 0.5 ppm or less).

[708] The 1,3-butylene glycol product according to any one of [701] to [707], wherein a total content of methyl vinyl ketone, acetone, and butylaldehyde is 24 ppm or less (or 20 ppm or less, 18 ppm or less, 16 ppm or less, 14 ppm or less, 12 ppm or less, 11 ppm or less, 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, or 0.5 ppm or less).

[709] The 1,3-butylene glycol product according to any one of [701] to [708], wherein a total content of the compound represented by Formula (1), the compound represented by Formula (2), the compound represented by Formula (4), and the compound represented by Formula (5) is 24 ppm or less.

[710] The 1,3-butylene glycol product according to any one of [701] to [709], which has a content of acetaldehyde of less than 4 ppm (or less than 2 ppm, 1.8 ppm or less, 1.7 ppm or less, 1.5 ppm or less, 1.4 ppm or less, 1.3 ppm or less, 1.2 ppm or less, 1.1 ppm or less, 1.0 ppm or less, 0.9 ppm or less, 0.8 ppm or less, 0.7 ppm or less, 0.6 ppm or less, 0.5 ppm or less, 0.3 ppm or less, or 0.2 ppm or less).

[711] The 1,3-butylene glycol product according to any one of [701] to [710], which has a content of crotonaldehyde of less than 2 ppm (or less than 1.2 ppm, 1.0 ppm or less, 0.9 ppm or less, 0.8 ppm or less, 0.7 ppm or less, 0.6 ppm or less, 0.5 ppm or less, 0.4 ppm or less, 0.3 ppm or less, 0.2 ppm or less, or 0.1 ppm or less).

[712] The 1,3-butylene glycol product according to any one of [701] to [711], which has an acid concentration of less than 11 ppm (or 10 ppm or less, 9 ppm or less, 8 ppm or less, 7 ppm or less, 6 ppm or less, 5 ppm or less, 4 ppm or less, or 3 ppm or less) in terms of acetic acid, and an acid concentration of less than 23 ppm (or 20 ppm or less, 19 ppm or less, 18 ppm or less, 17 ppm or less, 16 ppm or less, 15 ppm or less, 14 ppm or less, 13 ppm or less, 12 ppm or less, 11 ppm or less, 10 ppm or less, 9 ppm or less, 8 ppm or less, 7 ppm or less, 6 ppm or less, 5 ppm or less, 4 ppm or less, 3 ppm or less, or 2 ppm or less) in terms of acetic acid after a 90 wt. % aqueous solution has been kept at 100 °C for 1 week.

[713] The 1,3-butylene glycol product according to any one of [701] to [712], in which an APHA is 6 or less (or 5 or less, 4 or less, 3 or less, or 2 or less), and, after the 1,3-butylene glycol product has been kept at 180 °C for 3 hours in air atmosphere, an APHA is 78 or less (or 65 or less, 60 or less, 55 or less, 50 or less, 45 or less, 40 or less, 35 or less, 30 or less, 25 or less, 20 or less, 18 or less, 15 or less, 14 or less, 13 or less, 12 or less, 11 or less, 10 or less, 9 or less, 8 or less, or 7 or less).

[714] The 1,3-butylene glycol product according to any one of [701] to [713], in which, after the 1,3-butylene glycol product has been kept at 100 °C for 75 days in air atmosphere, an APHA is 42 or less (35 or less, 30 or less, 25 or less, 20 or less, 18 or less, 16 or less, 15 or less, 14 or less, 13 or less, 12 or less, 11 or less, 10 or less, 9 or less, 8 or less, 7 or less, 6 or less, 5 or less, 4 or less, 3 or less, or 2 or less).

[715] The 1,3-butylene glycol product according to any one of [701] to [714], which has an initial boiling point of higher than 203 °C (or 204 °C or higher, 205 °C or higher, 206 °C or higher, 207 °C or higher, or 208 °C or higher), and/or a dry point of 209 °C or lower.

[716] The 1,3-butylene glycol product according to any one of [701] to [715], which has a potassium permanganate test value of 30 minutes or longer (or longer than 30 minutes, 32 minutes or longer, 35 minutes or longer, 40 minutes or longer, 50 minutes or longer, or 60 minutes or longer).

[717] The 1,3-butylene glycol product according to any one of [701] to [716], which has an area ratio (GC area ratio) of a peak of 1,3-butylene glycol of greater than 98.7% (or 98.8% or greater, 98.9% or greater, 99% or greater, 99.1% or greater, 99.2% or greater, 99.3% or greater, 99.4% or greater, 99.5% or greater, 99.6% or greater, 99.7% or greater, or 99.8% or greater), in a gas chromatographic analysis (GC analysis) performed under conditions set forth below,

The conditions for the gas chromatographic analysis are as follows:
Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

[718] The 1,3-butylene glycol product according to any one of [701] to [717], wherein a total area ratio of peaks shorter in retention time than the peak of 1,3-butylene glycol is less than 0.3% (or 0.28% or less, 0.25% or less, 0.23% or less, 0.2% or less, 0.17% or less, 0.15% or less, 0.12% or less, 0.1% or less, 0.07% or less, 0.04% or less, 0.03% or less, 0.02% or less, 0.01% or less, 0.007% or less, 0.005% or less, or 0.002% or less) in the gas chromatographic analysis (GC analysis) performed under conditions set forth above.

[719] The 1,3-butylene glycol product according to any one of [701] to [718], wherein a total area ratio of peaks longer in retention time than the peak of 1,3-butylene glycol is less than 1.2% (or 1% or less, 0.9% or less, 0.8% or less, 0.7% or less, 0.6% or less, 0.5% or less, 0.4% or less, 0.3% or less, 0.2% or less, or 0.1% or less).

[720] The 1,3-butylene glycol product according to any one of [701] to [719], which has a content of water of less than 0.4 wt. % (or 0.3 wt. % or less, 0.2 wt. % or less, 0.1 wt. % or less, 0.07 wt. % or less, 0.05 wt. % or less, 0.03 wt. % or less, 0.02 wt. % or less, 0.01 wt. % or less, or 0.005 wt. % or less).

[721]A moisturizer containing the 1,3-butylene glycol product described in any one of [701] to [720]. [722] The moisturizer according to [721], which has a content of the 1,3-butylene glycol product described in any one of [701] to [720] of 10 wt. % or greater (or 30 wt. % or greater, 50 wt. % or greater, 80 wt. % or greater, or 90 wt. % or greater).

[723]A cosmetic product containing the moisturizer described in [721] or [722].

[724] The cosmetic product according to [723], which has a content of the 1,3-butylene glycol product described in any one of [701] to [720] from 0.01 to 40 wt. % (or from 0.1 to 30 wt. %, from 0.2 to 20 wt. %, from 0.5 to 15 wt. %, or from 1 to 10 wt. %).

[725] The cosmetic product according to [723] or [724], which is a skin cosmetic product, a hair cosmetic, a sunscreen cosmetic product or a make-up cosmetic product.

[726]A method for producing 1,3-butylene glycol to yield purified 1,3-butylene glycol from a reaction crude liquid containing 1,3-butylene glycol,
the method including: performing dehydration including removing water by distillation, removing a high boiling point component including removing a high boiling point component by distillation, and performing product distillation to yield purified 1,3-butylene glycol,
wherein, in a product column for use in the performing product distillation, a 1,3-butylene glycol charged liquid is subjected to distillation, the 1,3-butylene glycol charged liquid having a content of acetaldehyde of 500 ppm or less (or 205 ppm or less, 200 ppm or less, 150 ppm or less, 120 ppm or less, 100 ppm or less, 90 ppm or less, 80 ppm or less, 70 ppm or less, 60 ppm or less, 50 ppm or less, 40 ppm or less, 30 ppm or less, 20 ppm or less, 10 ppm or less, 5 ppm or less, or less than 2 ppm), a content of crotonaldehyde of 200 ppm or less (or 150 ppm or less, 130 ppm or less, 110 ppm or less, 100 ppm or less, 80 ppm or less, 70 ppm or less, 60 ppm or less, 50 ppm or less, 40 ppm or less, 30 ppm or less, 20 ppm or less, 10 ppm or less, 5 ppm or less, 3 ppm or less, 2 ppm or less, or less than 1 ppm), a content of water of 0.7 wt. % or less (or 0.6 wt. % or less, 0.5 wt. % or less, 0.4 wt. % or less, 0.3 wt. % or less, 0.2 wt. % or less, or 0.1 wt. % or less), and a concentration of 1,3-butylene glycol of 97.6 area % or greater (or 97.8 area % or greater, 98 area % or greater, 98.2 area % or greater, 98.4 area % or greater, 98.6 area % or greater, 98.8 area % or greater, 99 area % or greater, 99.1 area % or greater, 99.2 area % or greater, 99.3 area % or greater, 99.4 area % or greater, 99.5 area % or greater, 99.6 area % or greater, 99.7 area % or greater, 99.8 area % or greater, or 99.9 area % or greater) according to a gas chromatographic analysis performed under conditions set forth below; and the distillation is performed under a condition that a reflux ratio is 0.3 or greater (or 0.4 or greater, 0.5 or greater, 1 or greater, 2 or greater, 3 or greater, 4 or greater, 5 or greater, 6 or greater, 7 or greater, 8 or greater, 9 or greater, 10 or greater, 20 or greater, 50 or greater, 400 or greater, or 500 or greater).

The conditions for the gas chromatographic analysis are as follows:
Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

[727] The method for producing 1,3-butylene glycol according to [726], wherein the distillation rate in the product column is less than 30 wt. % (or 29 wt. % or less, 28 wt. % or less, 27 wt. % or less, 26 wt. % or less, 25 wt. % or less, 24 wt. % or less, 23 wt. % or less, 22 wt. % or less, 21 wt. % or less, 20 wt. % or less, 19 wt. % or less, 18 wt. % or less, 17 wt. % or less, 16 wt. % or less, 15 wt. % or less, 12 wt. % or less, 10 wt. % or less, 8 wt. % or less, 5 wt. % or less, 3 wt. % or less, 2 wt. % or less, 1 wt. % or less, 0.8 wt. % or less, 0.6 wt. % or less, or 0.4 wt. % or less).

[728] The method for producing 1,3-butylene glycol according to [726] or [727], wherein at least a portion of a distillate of the product column is recycled to a step before the performing product distillation (for example, dehydration, dealcoholization, low boiling point component removal, or a step before these steps).

[729] The method for producing 1,3-butylene glycol according to [728], wherein an amount of the distillate of the product column, which is recycled to the step prior to the product distillation, is 0.01 wt. % or greater (or 0.05 wt. % or greater, 0.1 wt. % or greater, 0.5 wt. % or greater, 1 wt. % or greater, 1.5 wt. % or greater, 2 wt. % or greater, 3 wt. % or greater, 4 wt. % or greater, 5 wt. % or greater, 7 wt. % or greater, 10 wt. % or greater, or 20 wt. % or greater) and less than 30 wt. % with respect to a charged amount into the product column.

[730] The method for producing 1,3-butylene glycol according to any one of [726] to [729], wherein a recovery ratio of 1,3-butylene glycol in the product column is greater than 80% (or 85% or greater, 90% or greater, 95% or greater, or 99% or greater).

[731] The method for producing 1,3-butylene glycol according to any one of [726] to [730], wherein, in a high boiling point component removal column for use in the removing a high boiling point component, a charged liquid containing 1,3-butylene glycol is subjected to distillation, the charged liquid having a content of acetaldehyde of 500 ppm or less (or 205 ppm or less, 200 ppm or less, 100 ppm or less, 90 ppm or less, 80 ppm or less, 70 ppm or less, 60 ppm or less, 50 ppm or less, 40 ppm or less, 30 ppm or less, 20 ppm or less, 10 ppm or less, 5 ppm or less, less than 2 ppm, or less than 1 ppm), a content of crotonaldehyde of 200 ppm or less (or 110 ppm or less, 100 ppm or less, 80 ppm or less, 70 ppm or less, 60 ppm or less, 50 ppm or less, 40 ppm or less, 30 ppm or less, 20 ppm or less, 10 ppm or less, 5 ppm or less, 3 ppm or less, 2 ppm or less, or less than 1 ppm), a content of water of 3 wt. % or less (or 2 wt. % or less, 1.2 wt. % or less, 1.1 wt. % or less, 1.0 wt. % or less, 0.95 wt. % or less, 0.9 wt. % or less, 0.8 wt. % or less, 0 7.7 wt. % or less, 0.6 wt. % or less, 0.5 wt. % or less, 0.4 wt. % or less, 0.3 wt. % or less, 0.2 wt. % or less, or 0.1 wt. % or less), and a concentration of 1,3-butylene glycol of 95 area % or greater (or 96 area % or greater, 96.7 area % or greater, 97 area % or greater, 98 area % or greater, or 99 area % or greater) according to the gas chromatographic analysis performed under the conditions set forth above, and the distillation is performed under a condition that a reflux ratio is 0.03 or greater (or 0.05 or greater, 0.1 or greater, 0.2 or greater, 0.3 or greater, 0.4 or greater, 0.5 or greater, 0.6 or greater, 0.7 or greater, 0.8 or greater, 0.9 or greater, 1 or greater, 1.2 or greater, 1.5 or greater, 2 or greater, 3 or greater, 4 or greater, 5 or greater, 10 or greater, or 20 or greater).

[732] The method for producing 1,3-butylene glycol according to any one of [726] to [731], wherein a bottom ratio in the high boiling point component removal column for use in the removing a high boiling point component is less than 30 wt. % (or 25 wt. % or less, 20 wt. % or less, 15 wt. % or less, 10 wt. % or less, 7 wt. % or less, 5 wt. % or less, 4 wt. % or less, 3 wt. % or less, 2 wt. % or less, or 1 wt. % or less).

[733] The method for producing 1,3-butylene glycol according to any one of [726] to [732], wherein the bottom ratio in the high boiling point component removal column for use in the removing a high boiling point component is 0.01 wt. % or greater (or 0.1 wt. % or greater, 0.5 wt. % or greater, 1 wt. % or greater, 2 wt. % or greater, 3 wt. % or greater, 4 wt. % or greater, 5 wt. % or greater, 6 wt. % or greater, 7 wt. % or greater, 8 wt. % or greater, 9 wt. % or greater, 10 wt. % or greater, 15 wt. % or greater, or 20 wt. % or greater).

[734] The method for producing 1,3-butylene glycol according to any one of [726] to [733], wherein a recovery ratio of 1,3-butylene glycol in the high boiling point component removal column for use in the removing a high boiling point component is greater than 80% (or 85% or greater, 90% or greater, 95% or greater, or 99% or greater).

[735] The method for producing 1,3-butylene glycol according to any one of [726] to [734], wherein at least a portion of a bottom of the high boiling point component removal column for use in the removing a high boiling point component is recycled to a step before the removing a high boiling point component.

[736] The method for producing 1,3-butylene glycol according to [735], wherein an amount of the bottom of the high boiling point component removal column, which is recycled to the step prior to the removing a high boiling point component, is less than 30 wt. % (or 25 wt. %, 20 wt. % or less, 15 wt. % or less, 10 wt. % or less, 7 wt. % or less, 5 wt. % or less, 4 wt. % or less, 3 wt. % or less, 2 wt. % or less, or 1 wt. % or less) with respect to a charged amount into the high boiling point component removal column.

[737] The method for producing 1,3-butylene glycol according to [735] or [736], wherein the amount of the bottom of the high boiling point component removal column, which is recycled to the step prior to the removing a high boiling point component, is 0.01 wt. % or greater (or 0.1 wt. % or greater, 2 wt. % or greater, 3 wt. % or greater, 4 wt. % or greater, 5 wt. % or greater, 7 wt. % or greater, 10 wt. % or greater, or 20 wt. % or greater) with respect to the charged amount into the high boiling point component removal column.

[738]A method for producing 1,3-butylene glycol to yield purified 1,3-butylene glycol from a reaction crude liquid containing 1,3-butylene glycol,
the method including:
performing dehydration including removing water by distillation and removing a high boiling point component including removing a high boiling point component by distillation,
wherein, in a high boiling point component removal column for use in the removing a high boiling point component, a charged liquid containing 1,3-butylene glycol is subjected to distillation, the charged liquid having a content of acetaldehyde of 500 ppm or less (or 205 ppm or less, 200 ppm or less, 100 ppm or less, 90 ppm or less, 80 ppm or less, 70 ppm or less, 60 ppm or less, 50 ppm or less, 40 ppm or less, 30 ppm or less, 20 ppm or less, 10 ppm or less, 5 ppm or less, less than 2 ppm, or less than 1 ppm), a content of crotonaldehyde of 200 ppm or less (or 110 ppm or less, 100 ppm or less, 80 ppm or less, 70 ppm or less, 60 ppm or less, 50 ppm or less, 40 ppm or less, 30 ppm or less, 20 ppm or less, 10 ppm or less, 5 ppm or less, 3 ppm or less, 2 ppm or less, or less than 1 ppm), a content of water of 3 wt. % or less (or 2 wt. % or less, 1.2 wt. % or less, 0.4 wt. % or less, 0.3 wt. % or less, 0.2 wt. % or less, 0.1 wt. % or less, 0.05 wt. % or less, or 0.03 wt. % or less), and a concentration of 1,3-butylene glycol of 96.7 area % or greater (or 97% or greater, 98% or greater, or 99% or greater) according to a gas chromatographic analysis performed under conditions set forth below, and the distillation is performed under a condition that a reflux ratio is 0.03 or greater (or 0.1 or greater, 0.2 or greater, 0.3 or greater, 0.4 or greater, 0.5 or greater, 0.6 or greater, 0.7 or greater, 0.8 or greater, 0.9 or greater, 1 or greater, 1.2 or greater, 1.5 or greater, 2 or greater, 3 or greater, 4 or greater, 5 or greater, 10 or greater, or 20 or greater).

### The Conditions for the Gas Chromatographic Analysis are as Follows:

Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

[739] The method for producing 1,3-butylene glycol according to any one of [726] to [738], wherein, in a dehydration column for use in the performing dehydration, a charged liquid containing 1,3-butylene glycol is subjected to distillation, the charged liquid having a content of acetaldehyde of 1000 ppm or less (or 900 ppm or less, 800 ppm or less, 700 ppm or less, 600 ppm or less, 500 ppm or less, 400 ppm or less, 300 ppm or less, 200 ppm or less, 155 ppm or less, 140 ppm or less, 100 ppm or less, 90 ppm or less, 80 ppm or less, 70 ppm or less, 60 ppm or less, 50 ppm or less, 40 ppm or less, 30 ppm or less, 20 ppm or less, 10 ppm or less, 5 ppm or less, 3 ppm or less, 2 ppm or less, or 1 ppm or less), a content of crotonaldehyde of 400 ppm or less (or 300 ppm or less, 200 ppm or less, 100 ppm or less, 150 ppm or less, 130 ppm or less, 117 ppm or less, 100 ppm or less, 90 ppm or less, 80 ppm or less, 70 ppm or less, 60 ppm or less, 50 ppm or less, 40 ppm or less, 30 ppm or less, 20 ppm or less, 10 ppm or less, 5 ppm or less, 3 ppm or less, 2 ppm or less, or 1 ppm or less), a content of water of 90 wt. % or less (or 85 wt. % or less, 80 wt. % or less, 70 wt. % or less, 60 wt. % or less, 50 wt. % or less, 40 wt. % or less, 35 wt. % or less, 30 wt. % or less, 25 wt. % or less, 15 wt. % or less, or 10 wt. % or less), and a concentration of 1,3-butylene glycol of 95 area % or greater (or 96 area % or greater, 96.7 area % or greater, 97 area % or greater, 98 area % or greater, or 99 area % or greater) according to the gas chromatographic analysis performed under the conditions set forth above, and the distillation is performed under a condition that a reflux ratio is greater than 0.3 (or 0.4 or greater, 0.5 or greater, 0.6 or greater, 0.7 or greater, 0.8 or greater, 0.9 or greater, 1 or greater, 1.1 or greater, 1.2 or greater, 1.3 or greater, 1.4 or greater, 1.5 or greater, 1.6 or greater, 1.7 or greater, 1.8 or greater, 1.9 or greater, 2 or greater, 3 or greater, 4 or greater, 5 or greater, 6 or greater, 7 or greater, 8 or greater, 9 or greater, 10 or greater, 15 or greater, 20 or greater, 25 or greater, 30 or greater, or 40 or greater).

[740] The method for producing 1,3-butylene glycol according to any one of [726] to [739], wherein a distillation ratio in the dehydration column for use in the performing dehydration is 95 wt. % or less (or 90 wt. % or less, 85 wt. % or less, 80 wt. % or less, 75 wt. % or less, 70 wt. % or less, 65 wt. % or less, 60 wt. % or less, 55 wt. % or less, 50 wt. % or less, 45 wt. % or less, 40 wt. % or less, 35 wt. % or less, 30 wt. % or less, 25 wt. % or less, 20 wt. % or less, 15 wt. % or less, 10 wt. % or less, or 5 wt. % or less).

[741] The method for producing 1,3-butylene glycol according to any one of [726] to [740], wherein a recovery ratio of 1,3-butylene glycol in the dehydration column for use in the dehydration is 99.3% or greater.

[742] The method for producing 1,3-butylene glycol according to any one of [726] to [741], wherein the reaction crude liquid containing 1,3-butylene glycol is a reaction crude liquid produced by hydrogen reduction of an acetaldol.

[743] The method for producing 1,3-butylene glycol according to any one of [726] to [742], further including at least one step selected from alkali treatment including treating a process stream containing 1,3-butylene glycol with a base, desalting including removing a salt in a process stream containing 1,3-butylene glycol, and dealcoholization including removing a low boiling point substance containing an alcohol in a process stream containing 1,3-butylene glycol.

[801]A 1,3-butylene glycol product containing 1,3-butylene glycol, in which, after the 1,3-butylene glycol product has been kept at 180 °C for 3 hours in air atmosphere, at least one of contents of compounds represented by the following Formula (A) or (B) is less than 8 ppm (or 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm):

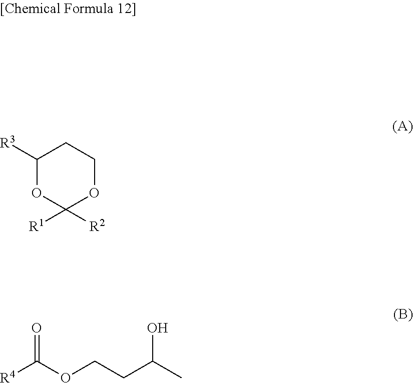

where R¹ to R⁴ are the same as or different from each other, and each of R¹ to R⁴ is a hydrogen atom, an alkyl group with from 1 to 4 carbon atoms which may be substituted with a hydroxy group, or an alkenyl group with 2 to 4 carbon atoms which may be substituted with a hydroxy group.

[802] The 1,3-butylene glycol product according to [801], in which, after the 1,3-butylene glycol product has been kept at 180 °C for 3 hours in air atmosphere, each of contents of two compounds (or three compounds, four compounds, five compounds, six compounds, seven compounds, eight compounds, nine compounds, or ten compounds), of the contents of the compounds represented by Formula (A) or (B), is less than 8 ppm (or 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

[803] The 1,3-butylene glycol product according to [801], in which, after the 1,3-butylene glycol product has been kept at 180 °C for 3 hours in air atmosphere, contents of at least four (four, five, six, seven, eight, nine, or ten) compounds, of the contents of the compounds represented by Formula (A) or (B), are less than 8 ppm (or 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

[804] The 1,3-butylene glycol product according to any one of [801] to [803], in which, after the 1,3-butylene glycol product has been kept at 180 °C for 3 hours in air atmosphere, a sum of the contents of the compounds represented by Formula (A) [or a sum of contents of compounds represented by the following Formulas (1) to (7)] is less than 66 ppm (or 60 ppm or less, 50 ppm or less, 40 ppm or less, 30 ppm or less, ppm or less, 18 ppm or less, 16 ppm or less, 14 ppm or less, 12 ppm or less, 10 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

[805] The 1,3-butylene glycol product according to any one of [801] to [804], in which, after the 1,3-butylene glycol product has been kept at 180 °C for 3 hours in air atmosphere, a sum of the contents of the compounds represented by Formula (B) [or a sum of contents of compounds represented by the following Formulas (8) to (10)] is less than 31 ppm (or 25 ppm or less, 20 ppm or less, 18 ppm or less, 16 ppm or less, 14 ppm or less, 13 ppm or less, 12 ppm or less, 11 ppm or less, 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

[806] The 1,3-butylene glycol product according to any one of [801] to [805], in which the compound represented by Formula (A) is any of the compounds represented by the following Formulas (1) to (7), and the compound represented by Formula (B) is any of the compounds represented by the following Formulas (8) to (10).

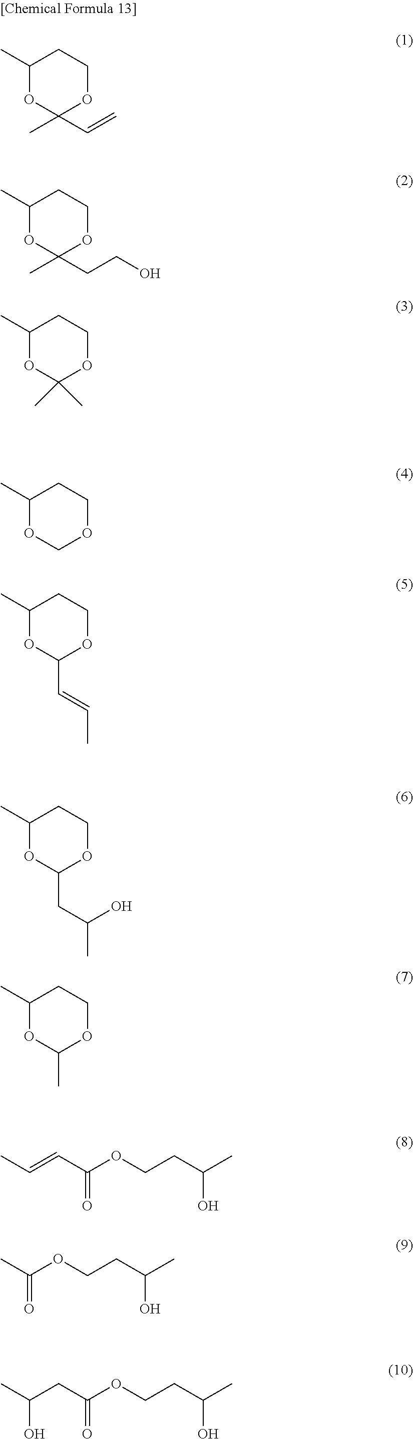

[807] The 1,3-butylene glycol product according to [806], in which, after the 1,3-butylene glycol product has been kept at 180 °C for 3 hours in air atmosphere, a total content of the compounds represented by Formulas (1) to (4) is less than 34 ppm (or, 30 ppm or less, 25 ppm or less, 20 ppm or less, 18 ppm or less, 16 ppm or less, 15 ppm or less, 14 ppm or less, 13 ppm or less, 12 ppm or less, 11 μm or less, 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

[808] The 1,3-butylene glycol product according to [806] or [807], in which, after the 1,3-butylene glycol product has been kept at 180 °C for 3 hours in air atmosphere, a total content of the compounds represented by Formulas (5) to (10) is less than 63 ppm (or, 60 ppm or less, 50 ppm or less, 40 ppm or less, 30 ppm or less, 20 ppm or less, 18 ppm or less, 16 ppm or less, 14 ppm or less, 12 ppm or less, 10 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

[809] The 1,3-butylene glycol product according to any one of [806] to [808], in which, after the 1,3-butylene glycol product has been kept at 180 °C for 3 hours in air atmosphere, a total content of the compounds represented by Formulas (1) to (10) is less than 97 ppm (or 80 ppm or less, 70 ppm or less, 60 ppm or less, 50 ppm or less, 40 ppm or less, 30 ppm or less, 20 ppm or less, 18 ppm or less, 16 ppm or less, 14 ppm or less, 12 ppm or less, 10 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

[810] The 1,3-butylene glycol product according to any one of [801] to [809], in which, after the 1,3-butylene glycol product has been kept at 180 °C for 3 hours in air atmosphere, a content of methyl vinyl ketone is less than 8 ppm (or 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm) and/or a content of 1-hydroxy-3-butanone is less than 1 ppm (0.5 ppm or less), and/or a content of acetone is less than 6 ppm (or 5 ppm or less, 4 ppm or less, 3 ppm or less, 2 ppm or less, 1 ppm or less, 0.5 ppm or less, 0.4 ppm or less, 0.3 ppm or less, or less than 0.2 ppm), and/or a content of formaldehyde is less than 2 ppm (or 1 ppm or less, 0.5 ppm or less, 0.4 ppm or less, 0.3 ppm or less, or less than 0.2 ppm), and/or a content of crotonaldehyde is less than 1 ppm (or 0.5 ppm or less), and/or a content of acetaldol is less than 1 ppm (or 0.5 ppm or less, 0.4 ppm or less, 0.3 ppm or less, or less than 0.2 ppm), and/or a content of acetaldehyde is less than 5 ppm (or 4 ppm or less, 3 ppm or less, 2 ppm or less, 1 ppm or less, 0.5 ppm or less, 0.4 ppm or less, 0.3 ppm or less, or less than 0.2 ppm), and/or a content of butylaldehyde is less than 4 ppm (or 3 ppm or less, 2 ppm or less, 1 ppm or less, 0.5 ppm or less, 0.4 ppm or less, 0.3 ppm or less, or less than 0.2 ppm).

[811] The 1,3-butylene glycol product according to any one of [801] to [810], in which, after the 1,3-butylene glycol product has been kept at 180 °C for 3 hours in air atmosphere, a total content of methyl vinyl ketone, 1-hydroxy-3-butanone, acetone, formaldehyde, crotonaldehyde, acetaldol and acetaldehyde is less than 24 ppm (or 20 ppm or less, 18 ppm or less, 16 ppm or less, 14 ppm or less, 12 ppm or less, 11 ppm or less, 10 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

[812] The 1,3-butylene glycol product according to any one of [801] to [811], in which, after the 1,3-butylene glycol product has been kept at 180 °C for 3 hours in air atmosphere, a total content of methyl vinyl ketone, acetone, and butylaldehyde is less than 18 ppm (or 16 ppm or less, 15 ppm or less, 14 ppm or less, 13 ppm or less, 12 ppm or less, 11 ppm or less, 10 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

[813] The 1,3-butylene glycol product according to any one of [801] to [812], in which, after the 1,3-butylene glycol product has been kept at 180 °C for 3 hours in air atmosphere, an APHA is less than 79 (or 65 or less, 60 or less, 55 or less, 50 or less, 45 or less, 40 or less, 35 or less, 30 or less, 25 or less, 20 or less, 18 or less, 15 or less, 14 or less, 13 or less, 12 or less, 11 or less, 10 or less, 9 or less, 8 or less, or 7 or less).

[814] The 1,3-butylene glycol product according to any one of [801] to [813], in which, after the 1,3-butylene glycol product has been kept at 180 °C for 3 hours in air atmosphere, an initial boiling point is higher than 203 °C (or 204 °C or higher, 205 °C or higher, 206 °C or higher, 207 °C or higher, or 208 °C or higher), and/or a dry point is 209 °C or lower.

[815] The 1,3-butylene glycol product according to any one of [801] to [814], in which, after the 1,3-butylene glycol product has been kept at 180 °C for 3 hours in air atmosphere, a potassium permanganate test value is 30 minutes or longer (or longer than 30 minutes, 32 minutes or longer, 35 minutes or longer, 40 minutes or longer, 50 minutes or longer, or 60 minutes or longer).

[816] The 1,3-butylene glycol product according to any one of [801] to [815], in which, after the 1,3-butylene glycol product has been kept at 180 °C for 3 hours in air atmosphere, an acid concentration (in terms of acetic acid) is less than 16 ppm (or 14 ppm or less, 12 ppm or less, 11 ppm or less, 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

[817] The 1,3-butylene glycol product according to any one of [801] to [816], in which, in a state where the 1,3-butylene glycol product has not undergone a test in which 1,3-butylene glycol product is kept at 180 °C for 3 hours in air atmosphere, a total content of 1-hydroxy-3-butanone and acetaldol is less than 19 ppm (or 18 ppm or less, 16 ppm or less, 14 ppm or less, 12 ppm or less, 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

[818] The 1,3-butylene glycol product according to [801] to [817], in which, in a state where the 1,3-butylene glycol product has not undergone a test in which 1,3-butylene glycol product is kept at 180 °C for 3 hours in air atmosphere, a content of methyl vinyl ketone is less than 10 ppm (or 8 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm), and/or a content of 1-hydroxy-3-butanone is less than 9 ppm (or 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm), and/or a content of acetone is less than 9 ppm (or 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm), and/or a content of formaldehyde is less than 3 ppm (or 2 ppm or less, 1 ppm or less, 0.5 ppm or less, 0.4 ppm or less, 0.3 ppm or less, or less than 0.2 ppm), and/or a content of crotonaldehyde is less than 2 ppm (or 1.5 ppm or less, 1 ppm or less, 0.5 ppm or less, 0.4 ppm or less, 0.3 ppm or less, or less than 0.2 ppm), and/or a content of acetaldol is less than 10 ppm (or 8 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm), and/or a content of acetaldehyde is or less 4 ppm (or 3 ppm or less, 2 ppm or less, 1 ppm or less, 0.5 ppm or less, 0.4 ppm or less, 0.3 ppm or less, or less than 0.2 ppm) and/or a content of butylaldehyde is less than 8 ppm (or 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

[819] The 1,3-butylene glycol product according to any one of [801] to [818], in which, in a state where the 1,3-butylene glycol product has not undergone a test in which 1,3-butylene glycol product is kept at 180 °C for 3 hours in air atmosphere, a total content of methyl vinyl ketone, 1-hydroxy-3-butanone, acetone, formaldehyde, crotonaldehyde, acetaldol and acetaldehyde is less than 47 ppm (or 40 ppm or less, ppm or less, 25 ppm or less, 20 ppm or less, 18 ppm or less, 16 ppm or less, 14 ppm or less, 13 ppm or less, 12 ppm or less, 11 μm or less, 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

[820] The 1,3-butylene glycol product according to any one of [801] to [819], in which, in a state where the 1,3-butylene glycol product has not undergone a test in which 1,3-butylene glycol product is kept at 180 °C for 3 hours in air atmosphere, an acid concentration (in terms of acetic acid) is less than 11 ppm (or ppm or less, 9 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

[821] The 1,3-butylene glycol product according to any one of [801] to [820], in which, in a state where the 1,3-butylene glycol product has not undergone a test in which 1,3-butylene glycol product is kept at 180 °C for 3 hours in air atmosphere, an acid concentration (in terms of acetic acid) after a 90 wt. % aqueous solution of the 1,3-butylene glycol product is kept at 100 °C for 1 week in air atmosphere is less than 23 ppm (20 ppm or less, 19 ppm or less, 18 ppm or less, 17 ppm or less, 16 ppm or less, 15 ppm or less, 14 ppm or less, 13 ppm or less, 12 ppm or less, 11 ppm or less, 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, 0.4 ppm or less, 0.3 ppm or less, or less than 0.2 ppm).

[822] The 1,3-butylene glycol product according to any one of [801] to [821], in which, in a state where the 1,3-butylene glycol product has not undergone a test in which 1,3-butylene glycol product is kept at 180 °C for 3 hours in air atmosphere, for the acid concentration (in terms of acetic acid) of the 90 wt. % aqueous solution of the 1,3-butylene glycol product, a ratio of the acid concentration after keeping at 100 °C for 1 week to the acid concentration before keeping [(acid concentration after keeping at 100 °C for 1 week)/(acid concentration before keeping)×100(%)] is 200% or less (or 150% or less or 120% or less).

[823] The 1,3-butylene glycol product according to any one of [801] to [822], in which, in a state where the 1,3-butylene glycol product has not undergone a test in which 1,3-butylene glycol product is kept at 180 °C for 3 hours in air atmosphere, an APHA is 6 or less (or 5 or less, 4 or less, 3 or less, or 2 or less).

[824] The 1,3-butylene glycol product according to any one of [801] to [823], in which, the 1,3-butylene glycol product has been kept at 100 °C for 75 days in air atmosphere, an APHA is 42 or less (35 or less, 30 or less, 25 or less, 20 or less, 18 or less, 16 or less, 15 or less, 14 or less, 13 or less, 12 or less, 11 or less, or less, 9 or less, 8 or less, 7 or less, 6 or less, 5 or less, 4 or less, 3 or less, or 2 or less).

[825] The 1,3-butylene glycol product according to any one of [801] to [824], in which, for the 1,3-butylene glycol product, a ratio of the APHA after keeping at 100 °C for 75 days to the APHA before keeping [(APHA after keeping at 100 °C for 75 days)/(APHA before keeping)] is 10 or less (or 8 or less, 7 or less, 6 or less, 5 or less, 4 or less, or 3 or less).

[826] The 1,3-butylene glycol product according to any one of [801] to [825], in which, in a state where the 1,3-butylene glycol product has not undergone a test in which 1,3-butylene glycol product is kept at 180 °C for 3 hours in air atmosphere, an initial boiling point is higher than 203 °C (or 204 °C or higher, 205 °C or higher, 206 °C or higher, 207 °C or higher, or 208 °C or higher), and/or a dry point of 209 °C or lower.

[827] The 1,3-butylene glycol product according to any one of [801] to [826], in which, in a state where the 1,3-butylene glycol product has not undergone a test in which 1,3-butylene glycol product is kept at 180 °C for 3 hours in air atmosphere, a potassium permanganate test value is 30 minutes or longer (or longer than 30 minutes, 32 minutes or longer, 35 minutes or longer, 40 minutes or longer, 50 minutes or longer, or 60 minutes or longer).

[828] The 1,3-butylene glycol product according to any one of [801] to [827], in which a content of 1,3-butylene glycol (GC area ratio under the following GC analysis conditions) is 98.6% or greater (or 98.7% or greater, 98.8% or greater, 98.9% or greater, 99.0% or greater, 99.1% or greater, 99.2% or greater, 99.3% or greater, 99.4% or greater, 99.5% or greater, 99.6% or greater, 99.7% or greater, or 99.8% or greater), in which the conditions for the gas chromatographic analysis (GC) are as follows:
Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample introduction Temperature: 250 °C
Carrier gas: Helium
Column Gas Flow Rate: 1 mL/min
Detector and Detection Temperature: a flame ionization detector (FID), 280 °C.

[829] The 1,3-butylene glycol product according to any one of [801] to [828], in which a total area ratio of peaks shorter in retention time than the peak of 1,3-butylene glycol is 0.28% or less (or 0.25% or less, 0.23% or less, 0.2% or less, 0.17% or less, 0.15% or less, 0.12% or less, 0.1% or less, 0.07% or less, 0.04% or less, 0.03% or less, 0.02% or less, 0.01% or less, 0.007% or less, 0.005% or less, or 0.002% or less).

[830] The 1,3-butylene glycol product according to any one of [801] to [829], in which, in the GC analysis, a total area ratio of peaks longer in retention time than the peak of 1,3-butylene glycol is 1% or less (or 0.9% or less, 0.8% or less, 0.7% or less, 0.6% or less, 0.5% or less, 0.4% or less, 0.3% or less, 0.2% or less, or 0.10% or less).

[831] The 1,3-butylene glycol product according to any one of [801] to [830], in which a content of water is less than 0.4 wt. % (or 0.3 wt. % or less, 0.2 wt. % or less, 0.1 wt. % or less, 0.07 wt. % or less, 0.05 wt. % or less, 0.03 wt. % or less, 0.02 wt. % or less, 0.01 wt. % or less, or 0.005 wt. % or less).

[832]A moisturizer containing the 1,3-butylene glycol product described in any one of [801] to [831].

[833] The moisturizer according to [832], in which a content of the 1,3-butylene glycol product described in any one of [801] to [831] is 10 wt. % or greater (or 30 wt. % or greater, 50 wt. % or greater, 80 wt. % or greater, or 90 wt. % or greater).

[834]A cosmetic product containing the moisturizer described in [832] or [833].

[835] The cosmetic product according to [834], in which a content of the 1,3-butylene glycol product described in any one of [801] to [831] is from 0.01 to 40 wt. % (or from 0.1 to 30 wt. %, from 0.2 to 20 wt. %, from 0.5 to 15 wt. %, or from 1 to 10 wt. %).

[836] The cosmetic product according to [834] or [835], which is a skin cosmetic product, a hair cosmetic, a sunscreen cosmetic product or a make-up cosmetic product.

[837]A method for producing 1,3-butylene glycol to yield a 1,3-butylene glycol product described in any one of [801] to [831] from a reaction crude liquid containing 1,3-butylene glycol,
the method including: performing dehydration including removing water by distillation, removing a high boiling point component including removing a high boiling point component by distillation, and performing product distillation to yield purified 1,3-butylene glycol (1,3-butylene glycol product),
wherein, in a product column for use in the performing product distillation, a 1,3-butylene glycol charged liquid is subjected to distillation to yield the 1,3-butylene glycol product, the 1,3-butylene glycol charged liquid having a content of acetaldehyde of 500 ppm or less (or 205 ppm or less, 200 ppm or less, 150 ppm or less, 120 ppm or less, 100 ppm or less, 90 ppm or less, 80 ppm or less, 70 ppm or less, 60 ppm or less, 50 ppm or less, 40 ppm or less, 30 ppm or less, 20 ppm or less, 10 ppm or less, 5 ppm or less, or less than 2 ppm), a content of crotonaldehyde of 200 ppm or less (or 150 ppm or less, 130 ppm or less, 110 ppm or less, 100 ppm or less, 80 ppm or less, 70 ppm or less, 60 ppm or less, 50 ppm or less, 40 ppm or less, 30 ppm or less, 20 ppm or less, 10 ppm or less, 5 ppm or less, 3 ppm or less, 2 ppm or less, or less than 1 ppm), a content of water of 0.7 wt % or less (or 0.6 wt. % or less, 0.5 wt. % or less, 0.4 wt. % or less, 0.3 wt. % or less, 0.2 wt. % or less, or 0.1 wt. % or less), and a concentration of 1,3-butylene glycol determined by gas chromatographic analysis under the following conditions of 97.6 area % or greater (or 97.8 area % or greater, 98 area % or greater, 98.2 area % or greater, 98.4 area % or greater, 98.6 area % or greater, 98.8 area % or greater, 99 area % or greater, 99.1 area % or greater, 99.2 area % or greater, 99.3 area % or greater, 99.4 area % or greater, 99.5 area % or greater, 99.6 area % or greater, 99.7 area % or greater, 99.8 area % or greater, or 99.9 area % or greater), and the distillation is performed under a condition that a reflux ratio is 0.3 or greater (or 0.4 or greater, 0.5 or greater, 1 or greater, 2 or greater, 3 or greater, 4 or greater, 5 or greater, 6 or greater, 7 or greater, 8 or greater, 9 or greater, 10 or greater, 20 or greater, 50 or greater, 400 or greater, or 500 or greater).

### The Conditions for the Gas Chromatographic Analysis are as Follows:

Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample introduction Temperature: 250 °C
Carrier gas: Helium
Column Gas Flow Rate: 1 mL/min
Detector and Detection Temperature: a flame ionization detector (FID), 280 °C.

[838]A method for producing 1,3-butylene glycol to yield a 1,3-butylene glycol product described in any one of [801] to [831] from a reaction crude liquid containing 1,3-butylene glycol,
the method including:
performing dehydration including removing water by distillation and removing a high boiling point component including removing a high boiling point component by distillation,
wherein, in a high boiling point component removal column for use in the removing a high boiling point component, a charged liquid is subjected to distillation to yield the 1,3-butylene glycol product containing 1,3-butylene glycol, the charged liquid having a content of acetaldehyde of 500 ppm or less (or 205 ppm or less, 200 ppm or less, 100 ppm or less, 90 ppm or less, 80 ppm or less, 70 ppm or less, 60 ppm or less, 50 ppm or less, 40 ppm or less, 30 ppm or less, 20 ppm or less, 10 ppm or less, 5 ppm or less, less than 2 ppm, or less than 1 ppm), a content of crotonaldehyde of 200 ppm or less (or 110 ppm or less, 100 ppm or less, 80 ppm or less, 70 ppm or less, 60 ppm or less, 50 ppm or less, 40 ppm or less, 30 ppm or less, 20 ppm or less, 10 ppm or less, 5 ppm or less, 3 ppm or less, 2 ppm or less, or less than 1 ppm), a content of water of 3 wt. % or less (or 2 wt % or less, 1.2 wt % or less, 0.4 wt % or less, 0.3 wt % or less, 0.2 wt % or less, 0.1 wt % or less, 0.05 wt % or less, or 0.03 wt % or less), and a concentration of 1,3-butylene glycol determined by gas chromatographic analysis under the following conditions of 96.7 area % or greater (or 97% or greater, 98% or greater, or 99% or greater), and the distillation is performed under a condition that a reflux ratio is 0.03 or greater (or 0.1 or greater, 0.2 or greater, 0.3 or greater, 0.4 or greater, 0.5 or greater, 0.6 or greater, 0.7 or greater, 0.8 or greater, 0.9 or greater, 1 or greater, 1.2 or greater, 1.5 or greater, 2 or greater, 3 or greater, 4 or greater, 5 or greater, 10 or greater, or 20 or greater).

### The Conditions for the Gas Chromatographic Analysis are as Follows:

Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

[901]A 1,3-butylene glycol product containing 1,3-butylene glycol, wherein a sum of contents of acetaldehyde, crotonaldehyde, methyl vinyl ketone, acetone, formaldehyde, butylaldehyde, acetaldol, 1-hydroxy-3-butanone, 2-butanol, a compound represented by Formula (1) below, a compound represented by Formula (2) below, a compound represented by Formula (3) below, a compound represented by Formula (4) below, a compound represented by Formula (5) below, a compound represented by Formula (6) below, a compound represented by Formula (7) below, a compound represented by Formula (8) below, a compound represented by Formula (9) below, and a compound represented by Formula (10) below is less than 65 ppm (or 50 ppm or less, 45 ppm or less, 40 ppm or less, 35 ppm or less, 30 ppm or less, 25 ppm or less, 20 ppm or less, 15 ppm or less, 13 ppm or less, 10 ppm or less, 8 ppm or less, 5 ppm or less, 4 ppm or less, 3 ppm or less, or 2 ppm or less).

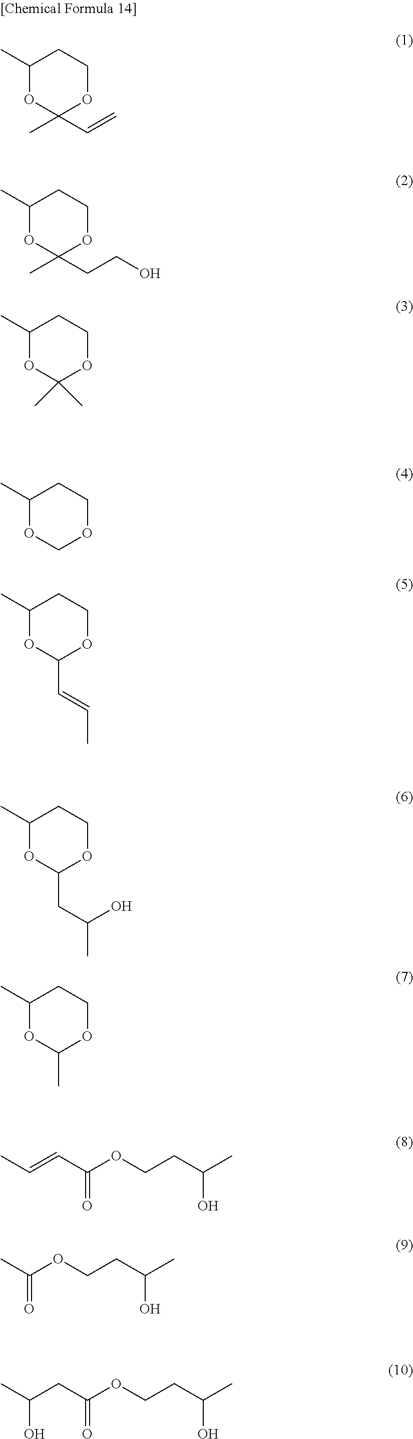

[902] The 1,3-butylene glycol product according to [901], wherein a sum of the contents of the compounds represented by Formulas (1) to (7) above is less than 28 ppm (or 25 ppm or less, 20 ppm or less, 15 ppm or less, 12 ppm or less, 10 ppm or less, 8 ppm or less, 6 ppm or less, 4 ppm or less, 2 ppm or less, or 1.4 ppm or less).

[903] The 1,3-butylene glycol product according to [901] or [902], wherein a sum of the contents of the compounds represented by Formulas (8) to (10) above is less than 6 ppm (or 5 ppm or less, 4 ppm or less, 3 ppm or less, 2 ppm or less, 1 ppm or less, 0.8 ppm or less, or 0.6 ppm or less).

[904] The 1,3-butylene glycol product according to any one of [901] to [903], wherein a sum of the contents of the compounds represented by Formulas (1) to (10) above is less than 34 ppm (or 30 ppm or less, 25 ppm or less, 20 ppm or less, 18 ppm or less, 16 ppm or less, 14 ppm or less, 12 ppm or less, 10 ppm or less, 8 ppm or less, 7 ppm or less, 6 ppm or less, 5 ppm or less, 4 ppm or less, 3 ppm or less, or 2 ppm or less).

[905] The 1,3-butylene glycol product according to any one of [901] to [904], wherein
the content of acetaldehyde is less than 1.6 ppm (or 1.5 ppm or less, 1.4 ppm or less, 1.2 ppm or less, 1.0 ppm or less, 0.8 ppm or less, 0.6 ppm or less, 0.4 ppm or less, or 0.2 ppm or less), and/or
the content of crotonaldehyde is less than 1 ppm (or 0.8 ppm or less, 0.6 ppm or less, 0.4 ppm or less, or 0.2 ppm or less), and/or
the content of methyl vinyl ketone is less than 6 ppm (or 5 ppm or less, 4 ppm or less, 3 ppm or less, 2 ppm or less, 1 ppm or less, 0.8 ppm or less, 0.6 ppm or less, 0.4 ppm or less, or 0.2 ppm or less), and/or
the content of acetone is less than 5 ppm (or 4 ppm or less, 3 ppm or less, 2 ppm or less, 1 ppm or less, 0.8 ppm or less, 0.6 ppm or less, 0.4 ppm or less, or 0.2 ppm or less), and/or
the content of formaldehyde is less than 1 ppm (or 0.8 ppm or less, 0.6 ppm or less, 0.4 ppm or less, 0.3 ppm or less, or 0.2 ppm or less), and/or
the content of butylaldehyde is less than 5 ppm (or 4 ppm or less, 3 ppm or less, 2 ppm or less, 1 ppm or less, 0.8 ppm or less, 0.6 ppm or less, 0.4 ppm or less, or 0.2 ppm or less), and/or
the content of acetaldol is less than 6 ppm (or 5 ppm or less, 4 ppm or less, 3 ppm or less, 2 ppm or less, 1 ppm or less, 0.8 ppm or less, 0.4 ppm or less, or 0.2 ppm or less), and/or
the content of 1-hydroxy-3-butanone is less than 6 ppm (or 5 ppm or less, 4 ppm or less, 3 ppm or less, 2 ppm or less, 1 ppm or less, 0.8 ppm or less, 0.6 ppm or less, 0.4 ppm or less, or 0.2 ppm or less), and/or
the content of 2-butanol is 0.3 ppm or less (or less than 0.2 ppm), and/or
the content of the compound represented by Formula (1) is less than 2 ppm (or 1.8 ppm or less, 1.6 ppm or less, 1.4 ppm or less, 1.2 ppm or less, 0.8 ppm or less, 0.6 ppm or less, 0.4 ppm or less, or 0.2 ppm or less), and/or
the content of the compound represented by Formula (2) is less than 1 ppm (or 0.8 ppm or less, 0.6 ppm or less, 0.4 ppm or less, 0.3 ppm or less, or 0.2 ppm or less), and/or
the content of the compound represented by Formula (3) is less than 4 ppm (or 3 ppm or less, 2 ppm or less, 1 ppm or less, 0.8 ppm or less, 0.6 ppm or less, 0.4 ppm or less, or 0.2 ppm or less), and/or
the content of the compound represented by Formula (4) is less than 3 ppm (or 2 ppm or less, 1 ppm or less, 0.8 ppm or less, 0.6 ppm or less, 0.4 ppm or less, or 0.2 ppm or less), and/or
the content of the compound represented by Formula (5) is less than 6 ppm (or 5 ppm or less, 4 ppm or less, 3 ppm or less, 2 ppm or less, 1 ppm or less, 0.8 ppm or less, 0.6 ppm or less, 0.4 ppm or less, or 0.2 ppm or less), and/or
the content of the compound represented by Formula (6) is less than 5 ppm (or 4 ppm or less, 3 ppm or less, 2 ppm or less, 1 ppm or less, 0.8 ppm or less, 0.6 ppm or less, 0.4 ppm or less, or 0.2 ppm or less), and/or
the content of the compound represented by Formula (7) is less than 7 ppm (or 6 ppm or less, 5 ppm or less, 4 ppm or less, 3 ppm or less, 2 ppm or less, 1 ppm or less, 0.8 ppm or less, 0.6 ppm or less, 0.4 ppm or less, or 0.2 ppm or less), and/or
the content of the compound represented by Formula (8) is less than 1 ppm (or 0.8 ppm or less, 0.6 ppm or less, 0.4 ppm or less, 0.3 ppm or less, or 0.2 ppm or less), and/or
the content of the compound represented by Formula (9) is less than 4 ppm (or 3 ppm or less, 2 ppm or less, 1 ppm or less, 0.8 ppm or less, 0.6 ppm or less, 0.4 ppm or less, or 0.2 ppm or less), and/or
the content of the compound represented by Formula (10) is less than 1 ppm (or 0.8 ppm or less, 0.6 ppm or less, 0.4 ppm or less, 0.3 ppm or less, or 0.2 ppm or less).

[906] The 1,3-butylene glycol product according to any one of [901] to [905], wherein a total content of acetaldehyde, crotonaldehyde, methyl vinyl ketone, acetone, formaldehyde, butylaldehyde, acetaldol, and 1-hydroxy-3-butanone is 31 ppm or less (or 30 ppm or less, 25 ppm or less, 20 ppm or less, 18 ppm or less, 16 ppm or less, 14 ppm or less, 13 ppm or less, 12 ppm or less, 11 μm or less, 10 ppm or less, 9 ppm or less, 8 ppm or less, 7 ppm or less, 6 ppm or less, 5 ppm or less, 4 ppm or less, 3 ppm or less, 2 ppm or less, or 1.5 ppm or less).

[907] The 1,3-butylene glycol product according to any one of [901] to [906], wherein a content of acetaldol and 1-hydroxy-3-butanone is less than 12 ppm (or 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, 0.5 ppm or less, or 0.4 ppm or less).

[908] The 1,3-butylene glycol product according to any one of [901] to [907], wherein an acid concentration (in terms of acetic acid) is less than 6 ppm (or 5 ppm or less, 4 ppm or less, 3 ppm or less, 2 ppm or less, or 1 ppm or less).

[909] The 1,3-butylene glycol product according to any one of [901] to [908], wherein an acid concentration (in terms of acetic acid) is less than 9 ppm (or 8 ppm or less, 7 ppm or less, 6 ppm or less, 5 ppm or less, 4 ppm or less, 3 ppm or less, 2 ppm or less, or 1 ppm or less), after a 90 wt. % aqueous solution of the 1,3-butylene glycol product has been kept at 100 °C for 1 week.

[910] The 1,3-butylene glycol product according to any one of [901] to [909], wherein, for the acid concentration (in terms of acetic acid) of the 90 wt. % aqueous solution of the 1,3-butylene glycol product, a ratio of the acid concentration after the product has been kept at 100 °C for 1 week to the acid concentration before the product has been kept [(acid concentration after the product has been kept at 100 °C for 1 week)/(acid concentration before the product has been kept)×100(%)] is 150% or less (or 120% or less, or 110% or less).

[911] The 1,3-butylene glycol product according to any one of [901] to [910], wherein an APHA (Hazen color number) is 3 or less (or 2 or less, or 1 or less).

[912] The 1,3-butylene glycol product according to any one of [901] to [911], wherein an APHA is 11 or less (or 10 or less, 8 or less, 7 or less, 6 or less, 5 or less, 4 or less, 3 or less, or 2 or less), after the 1,3-butylene glycol product has been kept at 100 °C for 75 days in air atmosphere.

[913] The 1,3-butylene glycol product according to any one of [901] to [912], wherein, for the APHA of the 1,3-butylene glycol product, a ratio of the APHA after the product has been kept at 100 °C for 75 days to the APHA before the product has been kept [(APHA after the product has been kept at 100 °C for 75 days)/(APHA before the product has been kept)] is less than 3 (or 2.5 or less, 2 or less, 1.5 or less, or 1.2 or less).

[914] The 1,3-butylene glycol product according to any one of [901] to [913], wherein an initial boiling point is 204 °C or greater (or 204.5 °C or higher, 205 °C or higher, 206 °C or higher, 207 °C or higher, or 208 °C or higher).

[915] The 1,3-butylene glycol product according to any one of [901] to [914], which has a dry point of lower than 209 °C (or 208.8 °C or lower).

[916] The 1,3-butylene glycol product according to any one of [901] to [915], wherein a potassium permanganate test value (PMT) is 36 minutes or longer (or 38 minutes or longer, 40 minutes or longer, 50 minutes or longer, or 60 minutes or longer).

[917] The 1,3-butylene glycol product according to any one of [901] to [916], wherein a content of 1,3-butylene glycol (GC area ratio under the following GC analysis conditions) is 99.3% or greater (or 99.4% or greater, 99.5% or greater, 99.6% or greater, 99.7% or greater, or 99.8% or greater),
in which the conditions for the gas chromatographic analysis are as follows:
Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

[918] The 1,3-butylene glycol product according to any one of [901] to [917], wherein, in the GC analysis, a total area ratio of peaks shorter in retention time than a peak of 1,3-butylene glycol is 0.09% or less (or 0.08% or less, 0.07% or less, 0.04% or less, 0.03% or less, 0.02% or less, 0.01% or less, 0.007% or less, 0.005% or less, or 0.003% or less).

[919] The 1,3-butylene glycol product according to any one of [901] to [918], wherein, in the GC analysis, a total area ratio of peaks longer in retention time than the peak of 1,3-butylene glycol is 0.7% or less (or 0.6% or less, 0.5% or less, 0.4% or less, 0.3% or less, 0.2% or less, or 0.1% or less).

[920] The 1,3-butylene glycol product according to any one of [901] to [919], wherein a content of water is 0.2 wt. % or less (or 0.15 wt. % or less, 0.1 wt. % or less, 0.07 wt. % or less, 0.05 wt. % or less, 0.03 wt. % or less, 0.02 wt. % or less, 0.01 wt. % or less, or 0.005 wt. % or less).

[921] The 1,3-butylene glycol product according to any one of [901] to [920], wherein a sum of the contents of acetaldehyde, crotonaldehyde, methyl vinyl ketone, acetone, formaldehyde, butylaldehyde, acetaldol, 1-hydroxy-3-butanone, 2-butanol and the compounds represented by Formulas (1) to (10) is less than 70 ppm (or 65 ppm or less, 60 ppm or less, 55 ppm or less, 50 ppm or less, 45 ppm or less, 40 ppm or less, 35 ppm or less, 30 ppm or less, 25 ppm or less, 20 ppm or less, 18 ppm or less, 15 ppm or less, 13 ppm or less, 10 ppm or less, 8 ppm or less, 5 ppm or less, 4 ppm or less, or 3.5 ppm or less), after the 1,3-butylene glycol product has been kept at 180 °C for 3 hours in air atmosphere.

[922] The 1,3-butylene glycol product according to any one of [901] to [921], wherein a sum of the contents of the compounds represented by Formulas (1) to (7) above is less than 40 ppm (or 35 ppm or less, 30 ppm or less, 25 ppm or less, 20 ppm or less, 15 ppm or less, 12 ppm or less, 10 ppm or less, 8 ppm or less, 6 ppm or less, 5 ppm or less, 4 ppm or less, 3 ppm or less, 2 ppm or less, or 1.4 ppm or less), after the 1,3-butylene glycol product has been kept at 180 °C for 3 hours in air atmosphere.

[923] The 1,3-butylene glycol product according to any one of [901] to [922], wherein a sum of the contents of the compounds represented by Formulas (8) to (10) above is less than 18 ppm (or 16 ppm or less, 14 ppm or less, 13 ppm or less, 12 ppm or less, 11 μm or less, 10 ppm or less, 9 ppm or less, 8 ppm or less, 7 ppm or less, 6 ppm or less, 5 ppm or less, 4 ppm or less, 3 ppm or less, 2 ppm or less, 1 ppm or less, or 0.6 ppm or less), after the 1,3-butylene glycol product has been kept at 180 °C for 3 hours in air atmosphere.

[924] The 1,3-butylene glycol product according to any one of [901] to [923], wherein a sum of the contents of the compounds represented by Formulas (1) to (10) above is less than 59 ppm (or 55 ppm or less, 50 ppm or less, 40 ppm or less, 30 ppm or less, 20 ppm or less, 18 ppm or less, 16 ppm or less, 14 ppm or less, 12 ppm or less, 10 ppm or less, 8 ppm or less, 7 ppm or less, 6 ppm or less, 5 ppm or less, 4 ppm or less, 3 ppm or less, or 2 ppm or less), after the 1,3-butylene glycol product has been kept at 180 °C for 3 hours in air atmosphere.

[925] The 1,3-butylene glycol product according to any one of [901] to [924], wherein a content of acetaldehyde is less than 1 ppm (or 0.9 ppm or less, 0.8 ppm or less, 0.7 ppm or less, 0.6 ppm or less, 0.5 ppm or less, 0.4 ppm or less, 0.3 ppm or less, or 0.2 ppm or less), and/or
a content of crotonaldehyde is less than 0.5 ppm (or 0.4 ppm or less, 0.3 ppm or less, 0.2 ppm or less, or 0.1 ppm or less), and/or
a content of methyl vinyl ketone is less than 4 ppm (or 3 ppm or less, 2 ppm or less, 1 ppm or less, 0.8 ppm or less, 0.6 ppm or less, 0.4 ppm or less, 0.3 ppm or less, or 0.2 ppm or less), and/or
a content of acetone is less than 2 ppm (or 1 ppm or less, 0.8 ppm or less, 0.6 ppm or less, 0.4 ppm or less, 0.3 ppm or less, or 0.2 ppm or less), and/or
a content of formaldehyde is less than 0.5 ppm (or 0.4 ppm or less, 0.3 ppm or less, or 0.2 ppm or less), and/or
a content of butylaldehyde is less than 3 ppm (or 2 ppm or less, 1 ppm or less, 0.8 ppm or less, 0.6 ppm or less, 0.4 ppm, 0.3 ppm or less, or 0.2 ppm or less), and/or
a content of acetaldol is less than 0.5 ppm (or 0.4 ppm or less, 0.3 ppm or less, or 0.2 ppm or less), and/or
a content of 1-hydroxy-3-butanone is less than 0.5 ppm (or 0.4 ppm or less, 0.3 ppm or less, or 0.2 ppm or less), and/or
a content of 2-butanol is less than 0.2 ppm, and/or
a content of the compound represented by Formula (1) is less than 6 ppm (or 5 ppm or less, 4 ppm or less, 3 ppm or less, 2 ppm or less, 1 ppm or less, 0.8 ppm or less, 0.6 ppm or less, 0.4 ppm or less, 0.3 ppm or less, or 0.2 ppm or less), and/or
a content of the compound represented by Formula (2) is less than 5 ppm (or 4 ppm or less, 3 ppm or less, 2 ppm or less, 1 ppm or less, 0.8 ppm or less, 0.6 ppm or less, 0.4 ppm or less, 0.3 ppm or less, or 0.2 ppm or less), and/or
a content of the compound represented by Formula (3) is less than 4 ppm (or 3 ppm or less, 2 ppm or less, 1 ppm or less, 0.8 ppm or less, 0.6 ppm or less, 0.4 ppm or less, 0.3 ppm or less, or 0.2 ppm or less), and/or
a content of the compound represented by Formula (4) is less than 5 ppm (or 4 ppm or less, 3 ppm or less, 2 ppm or less, 1 ppm or less, 0.8 ppm or less, 0.6 ppm or less, 0.4 ppm or less, 0.3 ppm or less, or 0.2 ppm or less), and/or
a content of the compound represented by Formula (5) is less than 7 ppm (or 6 ppm or less, 5 ppm or less, 4 ppm or less, 3 ppm or less, 2 ppm or less, 1 ppm or less, 0.8 ppm or less, 0.6 ppm or less, 0.4 ppm or less, 0.3 ppm or less, or 0.2 ppm or less), and/or
a content of the compound represented by Formula (6) is less than 6 ppm (or 5 ppm or less, 4 ppm or less, 3 ppm or less, 2 ppm or less, 1 ppm or less, 0.8 ppm or less, 0.6 ppm or less, 0.4 ppm or less, 0.3 ppm or less, or 0.2 ppm or less), and/or
a content of the compound represented by Formula (7) is less than 8 ppm (or 7 ppm or less, 6 ppm or less, ppm or less, 4 ppm or less, 3 ppm or less, 2 ppm or less, 1 ppm or less, 0.8 ppm or less, 0.6 ppm or less, 0.4 ppm or less, 0.3 ppm or less, or 0.2 ppm or less), and/or
a content of the compound represented by Formula (8) is less than 6 ppm (or 5 ppm or less, 4 ppm or less, 3 ppm or less, 2 ppm or less, 1 ppm or less, 0.8 ppm or less, 0.6 ppm or less, 0.4 ppm or less, 0.3 ppm or less, or 0.2 ppm or less), and/or
a content of the compound represented by Formula (9) is less than 5 ppm (or 4 ppm or less, 3 ppm or less, 2 ppm or less, 1 ppm or less, 0.8 ppm or less, 0.6 ppm or less, 0.4 ppm or less, 0.3 ppm or less, or 0.2 ppm or less), and/or
a content of the compound represented by Formula (10) is less than 7 ppm (or 6 ppm or less, 5 ppm or less, 4 ppm or less, 3 ppm or less, 2 ppm or less, 1 ppm or less, 0.8 ppm or less, 0.6 ppm or less, 0.4 ppm or less, 0.3 ppm or less, or 0.2 ppm or less), after the 1,3-butylene glycol product has been kept at 180 °C for 3 hours in air atmosphere.

[926] The 1,3-butylene glycol product according to any one of [901] to [925], wherein a sum of the contents of acetaldehyde, crotonaldehyde, methyl vinyl ketone, acetone, formaldehyde, butylaldehyde, acetaldol, and 1-hydroxy-3-butanone is less than 12 ppm (or 10 ppm or less, 8 ppm or less, 7 ppm or less, 6 ppm or less, 5 ppm or less, 4 ppm or less, 3 ppm or less, 2 ppm or less, or 1.6 ppm or less), after the 1,3-butylene glycol product has been kept at 180 °C for 3 hours in air atmosphere.

[927] The 1,3-butylene glycol product according to any one of [901] to [926], wherein a sum of the contents of acetaldol and 1-hydroxy-3-butanone is less than 1 ppm (or 0.9 ppm or less, 0.8 ppm or less, 0.7 ppm or less, 0.6 ppm or less, 0.5 ppm or less, or 0.4 ppm or less), after the 1,3-butylene glycol product has been kept at 180 °C for 3 hours in air atmosphere.

[928] The 1,3-butylene glycol product according to any one of [901] to [927], wherein an APHA is less than 25 (or 20 or less, 18 or less, 15 or less, 14 or less, 13 or less, 12 or less, 11 or less, 10 or less, 9 or less, 8 or less, or 7 or less), after the 1,3-butylene glycol product has been kept at 180 °C for 3 hours in air atmosphere.

[929] The 1,3-butylene glycol product according to any one of [901] to [928], wherein an initial boiling point is 204 °C or higher (or 205 °C or higher, 206 °C or higher, or 207 °C or higher), after the 1,3-butylene glycol product has been kept at 180 °C for 3 hours in air atmosphere.

[930] The 1,3-butylene glycol product according to any one of [901] to [929], wherein a dry point is lower than 209 °C, after the 1,3-butylene glycol product has been kept at 180 °C for 3 hours in air atmosphere.

[931] The 1,3-butylene glycol product according to any one of [901] to [930], wherein a potassium permanganate test value (PMT) is longer than 30 minutes (or 32 minutes or longer, 35 minutes or longer, 40 minutes or longer, 50 minutes or longer, or 60 minutes or longer), after the 1,3-butylene glycol product has been kept at 180 °C for 3 hours in air atmosphere.

[932] The 1,3-butylene glycol product according to any one of [901] to [931], wherein an acid concentration (in terms of acetic acid) is less than 8 ppm (or 7 ppm or less, 6 ppm or less, 5 ppm or less, 4 ppm or less, 3 ppm or less, 2 ppm or less, or 1 ppm or less), after the 1,3-butylene glycol product has been kept at 180 °C for 3 hours in air atmosphere.

[933]A moisturizer containing the 1,3-butylene glycol product described in any one of [901] to [932].

[934] The moisturizer according to [933], wherein a content of the 1,3-butylene glycol product described in any one of [901] to [932] is 10 wt. % or greater (or 30 wt. % or greater, 50 wt. % or greater, 80 wt. % or greater, or 90 wt. % or greater).

[935]A cosmetic product containing the moisturizer described in [933] or [934].

[936] The cosmetic product according to [935], wherein a content of the 1,3-butylene glycol product described in any one of [901] to [932] is from 0.01 to 40 wt. % (or from 0.1 to 30 wt. %, from 0.2 to 20 wt. %, from 0.5 to 15 wt. %, or from 1 to 10 wt. %).

[937] The cosmetic product according to [935] or [936], wherein the cosmetic product is a skin cosmetic product, a hair cosmetic, a sunscreen cosmetic product or a make-up cosmetic product.

[938]A method for producing 1,3-butylene glycol to yield a 1,3-butylene glycol product described in any one of [901] to [932] from a reaction crude liquid containing 1,3-butylene glycol,
the method including: performing dehydration including removing water by distillation, removing a high boiling point component including removing a high boiling point component by distillation, and performing product distillation to yield purified 1,3-butylene glycol (1,3-butylene glycol product),
wherein, in a product column for use in the performing product distillation, a 1,3-butylene glycol charged liquid is subjected to distillation at a reflux ratio to yield the 1,3-butylene glycol product, the 1,3-butylene glycol charged liquid having a content of acetaldehyde of 500 ppm or less (or 205 ppm or less, 200 ppm or less, 150 ppm or less, 120 ppm or less, 100 ppm or less, 90 ppm or less, 80 ppm or less, 70 ppm or less, 60 ppm or less, 50 ppm or less, 40 ppm or less, 30 ppm or less, 20 ppm or less, 10 ppm or less, 5 ppm or less, or less than 2 ppm), a content of crotonaldehyde of 200 ppm or less (or 150 ppm or less, 130 ppm or less, 110 ppm or less, 100 ppm or less, 80 ppm or less, 70 ppm or less, 60 ppm or less, 50 ppm or less, 40 ppm or less, 30 ppm or less, 20 ppm or less, 10 ppm or less, 5 ppm or less, 3 ppm or less, 2 ppm or less, or less than 1 ppm), a content of water of 0.7 wt. % or less (or 0.6 wt. % or less, 0.5 wt. % or less, 0.4 wt. % or less, 0.3 wt. % or less, 0.2 wt. % or less, or 0.1 wt. % or less), and a concentration of 1,3-butylene glycol of 97.6 area % or greater (or 97.8 area % or greater, 98 area % or greater, 98.2 area % or greater, 98.4 area % or greater, 98.6 area % or greater, 98.8 area % or greater, 99 area % or greater, 99.1 area % or greater, 99.2 area % or greater, 99.3 area % or greater, 99.4 area % or greater, 99.5 area % or greater, 99.6 area % or greater, 99.7 area % or greater, 99.8 area % or greater, or 99.9 area % or greater) by gas chromatographic analysis under the following conditions, and the reflux ratio being 0.3 or greater (or 0.4 or greater, 0.5 or greater, 1 or greater, 2 or greater, 3 or greater, 4 or greater, 5 or greater, 6 or greater, 7 or greater, 8 or greater, 9 or greater, 10 or greater, 20 or greater, 50 or greater, 400 or greater, or 500 or greater).

### The Conditions for the Gas Chromatographic Analysis are as Follows:

Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

[939]A method for producing 1,3-butylene glycol to obtain a 1,3-butylene glycol product described in any one of [901] to [932] from a reaction crude liquid containing 1,3-butylene glycol,
the method including:
performing dehydration including removing water by distillation and removing a high boiling point component including removing a high boiling point component by distillation,
wherein, in a high boiling point component removal column for use in the removing a high boiling point component, a charged liquid containing 1,3-butylene glycol is subjected to distillation at a reflux ratio to yield the 1,3-butylene glycol product, the charged liquid having a content of acetaldehyde of 500 ppm or less (or 205 ppm or less, 200 ppm or less, 100 ppm or less, 90 ppm or less, 80 ppm or less, 70 ppm or less, 60 ppm or less, 50 ppm or less, 40 ppm or less, 30 ppm or less, 20 ppm or less, 10 ppm or less, 5 ppm or less, less than 2 ppm, or less than 1 ppm), a content of crotonaldehyde of 200 ppm or less (or 110 ppm or less, 100 ppm or less, 80 ppm or less, 70 ppm or less, 60 ppm or less, 50 ppm or less, 40 ppm or less, 30 ppm or less, 20 ppm or less, 10 ppm or less, 5 ppm or less, 3 ppm or less, 2 ppm or less, or less than 1 ppm), a content of water of 3 wt. % or less (or 2 wt. % or less, 1.2 wt. % or less, 0.4 wt. % or less, 0.3 wt. % or less, 0.2 wt. % or less, 0.1 wt. % or less, 0.05 wt. % or less, or 0.03 wt. % or less), and a concentration of 1,3-butylene glycol of 96.7 area % or greater (or 97% or greater, 98% or greater, or 99% or greater) by gas chromatographic analysis under the following conditions, the reflux ratio being 0.03 or greater (or 0.1 or greater, 0.2 or greater, 0.3 or greater, 0.4 or greater, 0.5 or greater, 0.6 or greater, 0.7 or greater, 0.8 or greater, 0.9 or greater, 1 or greater, 1.2 or greater, 1.5 or greater, 2 or greater, 3 or greater, 4 or greater, 5 or greater, 10 or greater, or 20 or greater),

### The Conditions for the Gas Chromatographic Analysis are as Follows:

Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

### INDUSTRIAL APPLICABILITY

With the 1,3-butylene glycol product according to the first aspect of the present disclosure, high-purity 1,3-butylene glycol having a very low content of a high boiling point component and a low dry point can be produced with a high recovery ratio. This 1,3-butylene glycol product has excellent moisturizing performance and can be used as a raw material for a moisturizer and a cosmetic product that can retain high quality for a long period of time.

With the 1,3-butylene glycol product according to the second aspect of the present disclosure, high-purity 1,3-butylene glycol having a very low content of the low boiling point component and a high initial boiling point, with a high recovery ratio can be produced. This 1,3-butylene glycol product has excellent moisturizing performance and can be used as a raw material for a moisturizer and a cosmetic product that can retain high quality for a long period of time.

The 1,3-butylene glycol product according to the third aspect of the present disclosure has a high potassium permanganate test value, and is suitably used as a moisturizer and in an application as a raw material for cosmetics. The 1,3-butylene glycol product can be used as a raw material for a moisturizer and a cosmetic product that have excellent moisturizing performance and can maintain high quality for a long period of time.

According to the manufacturing method of the fourth aspect of the present disclosure, it is possible to industrially efficiently manufacture high-purity 1,3-butylene glycol having a high potassium permanganate test value, a very low content of low boiling point components, and a high initial boiling point. Also, the 1,3-butylene glycol product of the present disclosure has a high potassium permanganate test value, a very low content of the low boiling point component, a high initial boiling point, and high purity, and thus is suitably used as a moisturizer and in an application as a raw material for cosmetics. Further, the moisturizer and cosmetic product of the present disclosure are excellent in moisturizing performance and have an extremely low content of reducing materials and low boiling point components, and thus can maintain high quality for a long period of time.

According to the manufacturing method of the fifth aspect of the present disclosure, high-purity 1,3-butylene glycol having a very low content of low boiling point components and high boiling point components, a high initial boiling point and a low dry point can be industrially efficiently manufactured. Furthermore, it is possible to industrially efficiently manufacture high-purity 1,3-butylene glycol having a high potassium permanganate test value, a very low content of low boiling point components and high boiling point components, a high initial boiling point, and a low dry point. Further, the 1,3-butylene glycol product of the present disclosure has a very low content of low boiling point components and high boiling point components, a high initial boiling point, a low dry point, and high purity. Further, the 1,3-butylene glycol product of the present disclosure has a high potassium permanganate test value. Therefore, it is suitably used as a moisturizer, and in an application as a raw material for cosmetics. Further, the moisturizers and cosmetic product of the present disclosure are excellent in moisturizing performance, have an extremely low content of low boiling point components and high boiling point components, and preferably have extremely low content of reducing substances, and thus can maintain high quality for a long period of time.

The manufacturing method of the sixth aspect of the present disclosure can be used to industrially efficiently manufacture high-purity 1,3-butylene glycol having a very low content of low boiling point components and high boiling point components, a high initial boiling point, and a low dry point, high-purity 1,3-butylene glycol having a very low content of low boiling point components, a high initial boiling point, a high potassium permanganate test value, and further high-purity 1,3-butylene glycol having a high potassium permanganate test value, a very low content of low boiling point components and high boiling point components, a high initial boiling point, and a low dry point.

The 1,3-butylene glycol product according to the present disclosure has high purity and is colorless and odorless (or almost colorless and odorless), unlikely to cause coloration and odor over time, and/or unlikely to cause an acid concentration increase over time also in a state containing water. This 1,3-butylene glycol product can be used as a raw material for a moisturizer and a cosmetic product that have excellent moisturizing performance and can retain high quality for a long period of time.

The 1,3-butylene glycol product according to the present disclosure has high purity and is colorless and odorless (or almost colorless and odorless), unlikely to cause or increase coloration and odor over time, and/or unlikely to cause an acid concentration increase over time even in a state containing water. This 1,3-butylene glycol product can be used as a raw material for a moisturizer and a cosmetic product that have excellent moisturizing performance and can retain high quality for a long period of time.

The 1,3-butylene glycol product according to the present disclosure has high purity and is colorless and odorless (or almost colorless and odorless), unlikely to cause or increase coloration and odor over time, and/or unlikely to cause an acid concentration increase over time even in a state containing water. This 1,3-butylene glycol product can be used as a raw material for a moisturizer and a cosmetic product that have excellent moisturizing performance and can retain high quality for a long period of time.

### REFERENCE SIGNS LIST

A: Dehydration column
B: Desalting column
C: Distillation column for removing high boiling point substance (high boiling point component removal column)
D: Alkaline reactor
E: Dealkalization column
F: Product distillation column (product column)
A-1, B-1, C-1, E-1, F-1: Condenser
A-2, C-2, F-2: Reboiler
X-1: Crude 1,3-butylene glycol
X-2: Water (discharged water)
X-3: A salt, a high boiling point substance, and a portion of 1,3-butylene glycol
X-4: A high boiling point substance and a portion of 1,3-butylene glycol
X-5: Sodium hydroxide, a high boiling point substance, and a portion of 1,3-butylene glycol
X-6: A low boiling point substance and a portion of 1,3-butylene glycol
Y: 1,3-butylene glycol product

1. A 1,3-butylene glycol product in which at least one of eight contents: a content of methyl vinyl ketone, a content of acetone, a content of butylaldehyde, a content of acetaldol, a content of a compound represented by Formula (1) below, a content of a compound represented by Formula (2) below, a content of a compound represented by Formula (3) below, and a total content of a compound represented by Formula (4) below and a compound represented by Formula (5) below, is less than 8 ppm

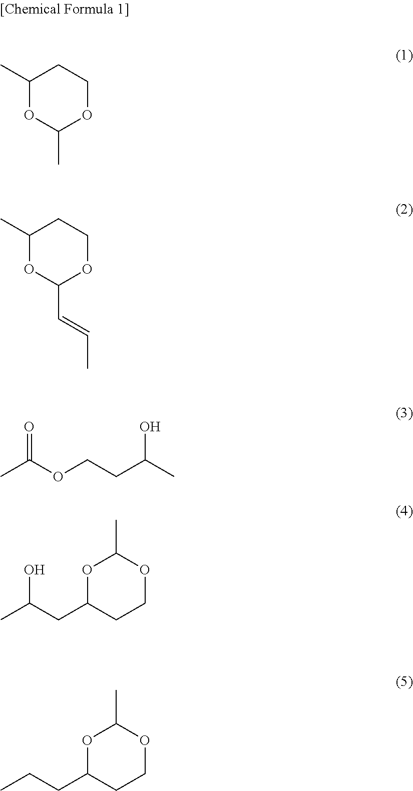

2. The 1,3-butylene glycol product according to claim 1, wherein a sum of the content of methyl vinyl ketone, the content of acetone, the content of butylaldehyde, the content of acetaldol, the content of the compound represented by Formula (1), the content of the compound represented by Formula (2), the content of the compound represented by Formula (3), the content of the compound represented by Formula (4), and the compound represented by Formula (5), is less than 71 ppm.

3. The 1,3-butylene glycol product according to claim 1, wherein at least the content of acetaldol is less than 8 ppm.

4. The 1,3-butylene glycol product according to claim 1, wherein at least the content of the compound represented by Formula (3) is less than 8 ppm.

5. The 1,3-butylene glycol product according to any one of claims 1 to 4, wherein a total content of methyl vinyl ketone, acetone, and butylaldehyde is 24 ppm or less.

6. The 1,3-butylene glycol product according to claim 1, wherein a total content of the compound represented by Formula (1), the compound represented by Formula (2), the compound represented by Formula (4), and the compound represented by Formula (5) is 24 ppm or less.

7. The 1,3-butylene glycol product according to claim 1, which has a content of acetaldehyde of less than 4 ppm and a content of crotonaldehyde of less than 2 ppm.

8. The 1,3-butylene glycol product according to claim 1, wherein an acid concentration is less than 11 ppm in terms of acetic acid, and, after a 90 wt. % aqueous solution of the 1,3-butylene glycol product has been kept at 100 °C for 1 week, the aqueous solution has an acid concentration of less than 23 ppm in terms of acetic acid.

9. The 1,3-butylene glycol product according to claim 1, wherein an APHA is 6 or less, and, after the 1,3-butylene glycol product has been kept at 180 °C for 3 hours in air atmosphere, an APHA of 78 or less.

10. The 1,3-butylene glycol product according to claim 1, wherein an initial boiling point is higher than 203 °C and/or a dry point is 209 °C or lower.

11. The 1,3-butylene glycol product according to claim 1, wherein a potassium permanganate test value is 30 minutes or longer.

12. A moisturizer comprising the 1,3-butylene glycol product described in claim 1.

13. A cosmetic product comprising the moisturizer described in claim 12.

14. A method for producing 1,3-butylene glycol to yield purified 1,3-butylene glycol from a reaction crude liquid including 1,3-butylene glycol,
the method comprising: performing dehydration including removing water by distillation, removing a high boiling point component including removing a high boiling point component by distillation, and performing product distillation to yield purified 1,3-butylene glycol,
wherein, in a product column for use in the performing product distillation, a 1,3-butylene glycol charged liquid is subjected to distillation under a condition that a reflux ratio is 0.3 or greater, the 1,3-butylene glycol charged liquid having a content of acetaldehyde of 500 ppm or less, a content of crotonaldehyde of 200 ppm or less, a content of water of 0.7 wt. % or less, and a concentration of 1,3-butylene glycol of 97.6 area % or greater, according to a gas chromatographic analysis performed under conditions, and the conditions for the gas chromatographic analysis are as follows:
Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

15. The method for producing 1,3-butylene glycol according to claim 14, wherein at least a portion of a distillate of the product column is recycled to a step before the performing the product distillation, the step including dehydration, dealcoholization, low boiling point component removal, or a step before the dehydration, the dealcoholization, the low boiling point component removal.

16. A method for producing 1,3-butylene glycol to yield purified 1,3-butylene glycol from a reaction crude liquid including 1,3-butylene glycol,
the method comprising: performing dehydration including removing water by distillation and removing a high boiling point component including removing a high boiling point component by distillation,
wherein, in a high boiling point component removal column for use in the removing a high boiling point component, a 1,3-butylene glycol-containing charged liquid is subjected to distillation under a condition that a reflux ratio is 0.03 or greater, the 1,3-butylene glycol-containing charged liquid having a content of acetaldehyde of 500 ppm or less, a content of crotonaldehyde of 200 ppm or less, a content of water of 3 wt. % or less, and a concentration of 1,3-butylene glycol of 96.7 area % or greater, according to a gas chromatographic analysis performed under conditions, and the conditions for the gas chromatographic analysis are as follows:
Analytical Column: a column with dimethylpolysiloxane as a stationary phase, having a length of 30 m, an inner diameter of 0.25 mm, and a film thickness of 1.0 μm
Heating conditions: heating from 80 °C to 120 °C at 5 °C/min, then heating again to 160 °C at 2 °C/min and maintaining for 2 minutes, and further heating to 230 °C at 10 °C/min and maintaining at 230 °C for 18 minutes
Sample Introduction Temperature: 250 °C
Carrier Gas: helium
Column Gas Flow Rate: 1 mL/min
Detector and Detection Temperature: a flame ionization detector (FID), 280 °C.

17. The method for producing 1,3-butylene glycol according to claim 14, wherein the reaction crude liquid including 1,3-butylene glycol is a reaction crude liquid produced by hydrogen reduction of an acetaldol.

18. The method for producing 1,3-butylene glycol according to claim 14, further comprising at least one step selected from alkali treatment including treating a process stream including 1,3-butylene glycol with a base, desalting including removing a salt in a process stream including 1,3-butylene glycol, and dealcoholization including removing a low boiling point substance in a process stream including 1,3-butylene glycol, the low boiling point substance including an alcohol.
